(19) United States
(12) Patent Application Publication
(10) Pub. No.: US 2026/0325540 A1
(43) Pub. Date: Oct. 22, 2026

(54) 3CLPRO INHIBITORS AND METHODS THEREOF

(71) Applicant: Agency for Science, Technology and Research, Singapore (SG)

(72) Inventors: Yi Yang SEE, Singapore (SG); Choon Heng LOW, Singapore (SG); Li Hong TAN, Singapore (SG); Subramanyam VANKADARA, Singapore (SG); Qian Wen TAN, Singapore (SG); Klement Jihao FOO, Singapore (SG); Cheng San Brian CHIA, Singapore (SG)

(21) Appl. No.: 19/482,679

(22) PCT Filed: May 8, 2024
(86) PCT No.: PCT/SG2024/050298

§ 371 Date: Nov. 7, 2025

(30) Foreign Application Priority Data
May 8, 2023 (SG) 10202301271W

(51) Int. Cl. A61K 31/55; A61K 31/407; A61K 31/422; A61K 31/427; A61K 31/437; A61K 31/438; A61K 31/439; A61K 31/506; A61P 31/14; C07D 471/04; C07D 471/14; C07D 471/20; C07D 471/22; C07D 487/04; C07D 487/14; C07D 487/20

(52) U.S. Cl. CPC A61K 31/55; A61K 31/407; A61K 31/422; A61K 31/427; A61K 31/437; A61K 31/438; A61K 31/439; A61K 31/506; A61P 31/14; C07D 471/04; C07D 471/14; C07D 471/20; C07D 471/22; C07D 487/04; C07D 487/14; C07D 487/20

(57) ABSTRACT

The present disclosure concerns a compound of Formula (I) or a salt, solvate, stereoisomer or prodrug thereof as a 3CLpro inhibitors. The compounds may be used for treating a coronavirus related disease or condition. (I)

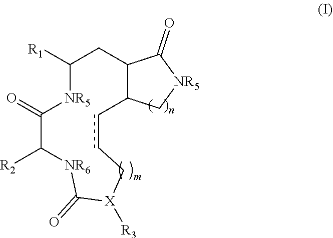

### TECHNICAL FIELD

The present disclosure relates, in general terms, to 3CLpro inhibitors and their methods of manufacture thereof.

### BACKGROUND

The severe acute respiratory syndrome coronavirus 2 (SARS-CoV-2) which emerged in late 2019 has caused a pandemic with great morbidity and mortality worldwide due to its high transmissibility and pathogenicity. Even with the advent of SARS-CoV-2 vaccines, the COVID-19 pandemic raged across the world in 2022 due to emergence of new SARS-CoV-2 variants that are more likely to evade immune protection generated from existing vaccines. To date, there is still limited antiviral agents that are clinically approved to combat SARS-CoV-2 and other coronavirus infections. The history of SARS-CoV-1, MERS and SARS-CoV-2 outbreaks in the past two decades has hinted that future pandemics are inevitable and the need for new antiviral drugs is urgent. Furthermore, there are four existing human coronaviruses (OC43, NL63, HKU1, 229E) that cause common cold respiratory diseases in humans. It is of strategic priority to develop new compounds against coronavirus, targeting viral proteins that are highly conserved and crucial for virus replication.

The SARS-CoV-2 genome encodes for more than 20 proteins, with two proteases, the papain-like protease (PLpro) and 3-chymotrypsin-like protease (3CLpro), that function to cleave viral translated polyproteins into individual proteins vital for virus replication. The 3CLpro is the main protease and considered a major druggable target. Viral replication can be blocked by the inhibition of viral polyproteins' proteolytic processing. Functionally, the 3CLpro proteolytically cleaves coronavirus polyproteins at more than 10 junctions to produce non-structural proteins critical for virus replication. As the 3CLpro is highly conserved across known coronavirus strains, the design of inhibitors against 3CLpro will improve our chances of having a drug ready for the next coronavirus outbreak. 3CLpro is also a virally encoded protein that has no close human analogs, reducing the likelihood of off-target activity.

It would be desirable to overcome or ameliorate at least one of the above-described problems.

### SUMMARY

The present disclosure concerns compounds that may inhibit the activity of coronavirus 3CL proteases, as demonstrated herein with activity against SARS-CoV-2, MERS, HCoV-229E and HCoV-OC43.

The present disclosure provides a compound of Formula (I) or a salt, solvate, stereoisomer or prodrug thereof:

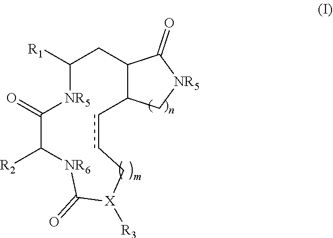

wherein R₁ is selected from optionally substituted acyl and cyano;
R₂ is selected from optionally substituted alkyl, and optionally substituted alkenyl;
X is selected from N or C—R₄;
R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino;
R₄ is selected from H, and optionally substituted alkyl;
R₅ is independently selected from H, and optionally substituted alkyl;
R₆ is selected from H and optionally substituted alkyl;
or R₂ and R₆ are linked to form an optionally substituted heterocyclyl;
m is an integer selected from 1 to 4; and
n is an integer selected from 1 to 4.

In some embodiments, R₁ is selected from HC(O)— and cyano.

In some embodiments, R₂ is C₁-C₅ alkyl.

In some embodiments, R₂ is selected from butyl, iso-butyl, pentyl, iso-pentyl, neo-pentyl, halopentyl, haloiso-pentyl, haloneo-pentyl, cyclopentylmethyl, and cyclopentyl.

In some embodiments, R₃ is selected from H, optionally substituted phenylmethyl, optionally substituted halophenylmethyl, optionally substituted trihalomethylphenylmethyl, optionally substituted cyclopentyl, optionally substituted cyclopentylmethyl, optionally substituted tert-butyloxy, optionally substituted cyclohexyloxy, optionally substituted tetrahydropyranylacylamino, optionally substituted tert-butylacylamino, optionally substituted trihalotert-butylacylamino, optionally substituted trihalomethylacylamino, optionally substituted neo-pentylacylamino, optionally substituted dihalocyclobutylmethylacylamino, optionally substituted cyclopentylmethylacylamino, optionally substituted pyrimidinylacylamino, optionally substituted phenylacylamino, optionally substituted halophenylacylamino, optionally substituted dihalophenylacylamino, optionally substituted isoxazolylacylamino, optionally substituted methylisoxazolylacylamino, optionally substituted trihalomethylisoxazolylacylamino, optionally substituted thiazolylacylamino, optionally substituted methylthiazolylacylamino, optionally substituted oxazolylacylamino, optionally substituted methyloxazolylacylamino, optionally substituted phenylacylamino, optionally substituted halophenylacylamino, optionally substituted dihalophenylacylamino, optionally substituted trihalomethylphenylacylamino, optionally substituted halotrihalomethylphenylacylamino, optionally substituted halophenylpropylacylamino, optionally substituted cyclopropylacylamino, optionally substituted trihalomethylcyclopropylacylamino, optionally substituted cyclobutylacylamino, optionally substituted methylcyclobutylacylamino, optionally substituted dihalocyclobutylacylamino, optionally substituted bicyclo[1.1.1]pentylacylamino, optionally substituted trihalomethylbicyclo[1.1.1]pentylacylamino, optionally substituted oxetanylacylamino, optionally substituted methyloxetanylacylamino, optionally substituted bicycl[2.1.1]hexylacylamino, optionally substituted cyclohexylacylamino, optionally substituted dihalocyclohexylacylamino, optionally substituted cyclohexylacylamino, optionally substituted spiro[3.3]heptylacylamino, optionally substituted tert-butyloxyacylamino (or Boc-NH or Boc-N(methyl)), optionally substituted cyclopentyloxyacyamino, optionally substituted neo-pentyloxyacylamino, optionally substituted cyclobutyloxyacylamino, optionally substituted trihalomethylcyclobutyloxyacylamino, optionally substituted phenylsulfonylamino, optionally substituted dihalophenylsulfonylamino, optionally substituted cyclopropylsulfonylamino, optionally substituted methylsulfonylamino, optionally substituted trihalomethylsulfonylamino, optionally substituted cyclobutylsulfonylamino, optionally substituted amino, optionally substituted pyrimidinylamino, optionally substituted trihalomethylpyrimidinylamino, and optionally substituted tert-butylaminoacyl.

In some embodiments, R₄ is H or methyl.

In some embodiments, R₅ is H or methyl.

In some embodiments, the compound is a compound of Formula (Ia):

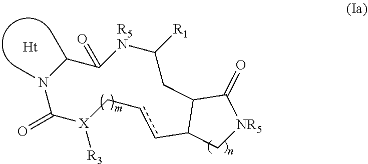

wherein R₁ is selected from optionally substituted acyl and cyano;
Ht is an optionally substituted heterocyclyl;
X is selected from N or C—R₄;
R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino;
R₄ is selected from H, and optionally substituted alkyl;
R₅ is independently selected from H, and optionally substituted alkyl;
m is an integer selected from 1 to 4; and
n is an integer selected from 1 to 4.

In some embodiments, Ht is an optionally substituted 5-7 membered heterocyclyl.

In some embodiments, the optional substituent on the heterocyclyl is selected from halo, methyl, methoxy, ethoxy, cyclopropyl, fused dimethylcyclopropyl, fused cyclopentyl, bridged methylene, and bridged ethylene.

In some embodiments, m is an integer selected from 1 to 3.

In some embodiments, n is an integer selected from 1 to 2.

The present disclosure also provides a pharmaceutical composition comprising an effective amount of a compound of Formula (I) or a pharmaceutically acceptable salt, solvate, stereoisomer or prodrug thereof, and optionally in combination with a pharmaceutically acceptable carrier, excipient or diluent.

The present disclosure provides a method of treating a coronavirus related disease or condition in a patient in need thereof, the method comprising administering to the patient a therapeutically effective amount of a compound of formula (I) or a pharmaceutically acceptable salt, solvate, stereoisomer or prodrug thereof.

The present disclosure provides a use of a compound of formula (I) or a pharmaceutically acceptable salt, solvate, stereoisomer or prodrug thereof in the manufacture of a medicament for treating a coronavirus related disease or condition.

The present disclosure provides a compound of formula (I) or a pharmaceutically acceptable salt, solvate, stereoisomer or prodrug thereof for use in treating a coronavirus related disease or condition.

In some embodiments, the compound of Formula (I) is a 3CLpro inhibitor.

In some embodiments, the compound of Formula (I) is characterised by a IC₅₀ value of less than 20 μM.

In some embodiments, the coronavirus related disease or condition is caused by a virus selected from SARS-CoV-1, SARS-CoV-2, MERS-CoV, HCoV-229E, and HCoV-OC43.

In some embodiments, the compound of Formula (I) is characterised by a protease inhibition assay IC₅₀ value of less than 20 μM.

In some embodiments, the compound of Formula (I) is characterised by a viral cytopathic effect assay EC₅₀ value of less than 20 μM.

The present disclosure provides a method of fabricating a compound of Formula (I), comprising:
i) reacting a compound of Formula (II) with a compound of Formula (III) under dehydrating conditions in order to form an amide bond:

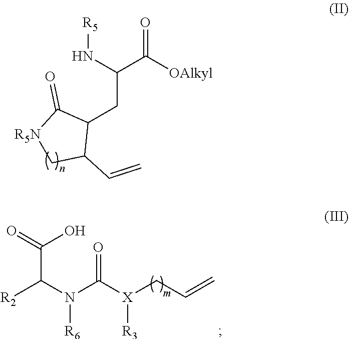

ii) performing a ring closing olefin metathesis reaction in order to form a compound of Formula (IV):

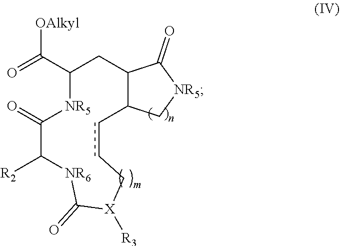

iii) converting the alkyloxyacyl moiety in Formula (IV) into a R₁ moiety in order to form a compound of Formula (I).

In some embodiments, step ii) further comprises performing a hydrogenation reaction after the olefin metathesis reaction.

### DETAILED DESCRIPTION

The presently disclosed compounds may be used as 3CLpro inhibitors, acting as competitive inhibitors to the protease's active site and demonstrate pan-coronavirus activity. 3CLpro almost exclusively cleaves substrates after a P1-Gln, and the P2 substrate residue is typically a Leu, with exceptions such as Met, Val and Phe. There is a consensus cleavage site for 3CLpro across known coronaviruses P3-XLQ-(S/G/A//N)-P1′. With high sequence conservation and specificity in cleavage site recognition amongst 3CLpro of known human coronaviruses, it is believed that the compounds may be used as a Pan-coronavirus 3CLpro inhibitor.

“Alkyl” refers to monovalent alkyl groups which may be straight chained or branched and preferably have from 1 to 10 carbon atoms or more preferably 1 to 6 carbon atoms.

Examples of such alkyl groups include methyl, ethyl, n-propyl, iso-propyl, n-butyl, iso-butyl, n-hexyl, and the like.

“Alkylene” refers to divalent alkyl groups preferably having from 1 to 10 carbon atoms and more preferably 1 to 6 carbon atoms. Examples of such alkylene groups include methylene (—CH₂—), ethylene (—CH₂CH₂—), and the propylene isomers (e.g., —CH₂CH₂CH₂- and —CH(CH₃)CH₂—), and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Cycloalkoxy” refers to the group cycloalkyl-O— where the cycloalkyl group is as described herein.

“Halo” or “halogen” refers to fluoro, chloro, bromo and iodo.

“Oxo/hydroxy” refers to groups ═O, HO—.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like.

“Heteroaryl” refers to a monovalent aromatic heterocyclic group which fulfils the Hückel criteria for aromaticity (ie. contains 4n+2 n electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (eg. pyridyl, pyrrolyl or N-oxides thereof or furyl) or multiple condensed rings (eg. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

“Aryloxy” refers to the group aryl-O— wherein the aryl group is as described above.

“Arylalkyl” refers to -alkylene-aryl groups preferably having from 1 to 10 carbon atoms in the alkylene moiety and from 6 to 10 carbon atoms in the aryl moiety. Such arylalkyl groups are exemplified by benzyl, phenethyl and the like.

“Acyl” refers to groups H—C(O)—, alkyl-C(O)—, cycloalkyl-C(O)—, aryl-C(O)—, heteroaryl-C(O)—and heterocyclyl-C(O)—, where alkyl, cycloalkyl, aryl, heteroaryl and heterocyclyl are as described herein.

“Acylalkyl” refers to the group acyl-alkyl- wherein the acyl group and alkyl group are as described above.

“Oxyacyl” refers to groups HOC(O)—, alkyl-OC(O)—, cycloalkyl-OC(O)—, aryl-OC(O)—, heteroaryl-OC(O)—, and heterocyclyl-OC(O)—, where alkyl, cycloalkyl, aryl, heteroaryl and heterocyclyl are as described herein.

“Amino” refers to the group —NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminoacyl” refers to the group —C(O)NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Acylamino” refers to the group —NR″C(O)R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Acyloxy” refers to the groups —OC(O)-alkyl, —OC(O)-aryl, —C(O)O-heteroaryl, and —C(O)O-heterocyclyl where alkyl, aryl, heteroaryl and heterocyclyl are as described herein.

“Aminoacyloxy” refers to the groups —OC(O)NR″-alkyl, —OC(O)NR″-aryl, —OC(O)NR″-heteroaryl, and —OC(O)NR″-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxyacylamino” refers to the groups —NR″C(O)O-alkyl, —NR″C(O)O-aryl, —NR″C(O)O— heteroaryl, and NR″C(O)O-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxyacyloxy” refers to the groups —OC(O)O-alkyl, —O—C(O)O-aryl, —OC(O)O— heteroaryl, and —OC(O)O-heterocyclyl where alkyl, cycloalkyl, aryl, heteroaryl, and heterocyclyl are as described herein.

“Cycloalkyl” refers to cyclic alkyl groups having a single cyclic ring or multiple condensed rings, preferably incorporating 3 to 11 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, indanyl, 1,2,3,4-tetrahydronapthalenyl and the like.

“Cycloalkenyl” refers to cyclic alkenyl groups having a single cyclic ring or multiple condensed rings, and at least one point of internal unsaturation, preferably incorporating 4 to 11 carbon atoms. Examples of suitable cycloalkenyl groups include, for instance, cyclobut-2-enyl, cyclopent-3-enyl, cyclohex-4-enyl, cyclooct-3-enyl, indenyl and the like.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Sulfinyl” refers to groups H—S(O)—, alkyl-S(O)—, cycloalkyl-S(O)—, aryl-S(O)—, heteroaryl-S(O)—, and heterocyclyl-S(O)—, where alkyl, cycloalkyl, aryl, heteroaryl and heterocyclyl are as described herein.

“Sulfonyl” refers to groups H—S(O)₂—, alkyl-S(O)₂—, cycloalkyl-S(O)₂—, aryl-S(O)₂—, heteroaryl-S(O)₂—, and heterocyclyl-S(O)₂—, where alkyl, cycloalkyl, aryl, heteroaryl and heterocyclyl are as described herein.

“Sulfinylamino” refers to groups H—S(O)—NR″—, alkyl-S(O)—NR″—, cycloalkyl-S(O)—NR″—, aryl-S(O)—NR″—, heteroaryl-S(O)—NR″—, and heterocyclyl-S(O)—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Sulfonylamino” refers to groups H—S(O)₂—NR″—, alkyl-S(O)₂—NR″—, cycloalkyl-S(O)₂—NR″—, aryl-S(O)₂—NR″—, heteroaryl-S(O)₂—NR″—, and heterocyclyl-S(O)₂—NR″—, where

R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminosulfonyl” refers to groups R″R″N—S(O)₂—, where each R″ is independently hydrogen, alkyl, cycloalkyl, aryl, heteroaryl, and heterocyclic and where each of alkyl, cycloalkyl, aryl, heteroaryl and heterocyclyl is as described herein.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroarylalkyl, heteroaryloxy, heterocyclyl, heterocyclylalkyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

“Isomer” includes especially optical isomers (for example essentially pure enantiomers, essentially pure diastereomers, and mixtures thereof) as well as conformation isomers (i.e. isomers that differ only in their angles of at least one chemical bond), position isomers (particularly tautomers), and geometric isomers (e.g. cis-trans isomers).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. The invention additionally encompasses compounds described herein as individual isomers substantially free of other isomers, and alternatively, as mixtures of various isomers. “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972).

The present disclosure provides a compound of Formula (I) or a salt, solvate, stereoisomer or prodrug thereof:

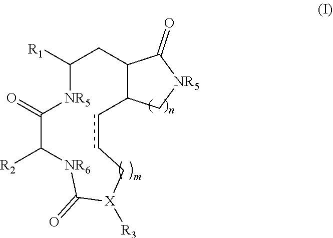

wherein R₁ is selected from optionally substituted acyl and cyano;
R₂ is selected from optionally substituted alkyl, and optionally substituted alkenyl;
X is selected from N or C—R₄;
R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino;
R₄ is selected from H, optionally substituted alkyl;
R₅ is independently selected from H, and optionally substituted alkyl;
R₆ is selected from H and optionally substituted alkyl;
or R₂ and R₆ are linked to form an optionally substituted heterocyclyl; m is an integer selected from 1 to 4; and
n is an integer selected from 1 to 4.

In some embodiments, R₁ is selected from acyl and cyano. In some embodiments, R₁ is selected from oxymethyl and cyano. In some embodiments, R₁ is selected from carbonyl and cyano. In some embodiments, R₁ is HC(O)—. In some embodiments, R₁ is cyano.

In some embodiments, R₂ is selected from optionally substituted alkyl. In some embodiments, R₂ is selected from alkyl. In some embodiments, R₂ is C₁-C₅ alkyl. The optional substituent may be halo. In some embodiments, R₂ is selected from butyl, iso-butyl, pentyl, iso-pentyl, neo-pentyl, halopentyl, haloiso-pentyl, haloneo-pentyl, cyclopentylmethyl, and cyclopentyl.

In some embodiments, X is N. Accordingly, the compound may be presented by Formula (I′):

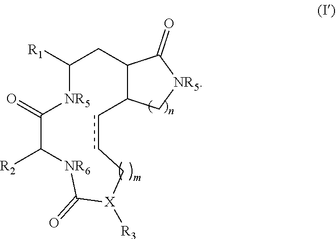

In some embodiments, X is C—R₄. Accordingly, the compound may be presented by Formula (I″):

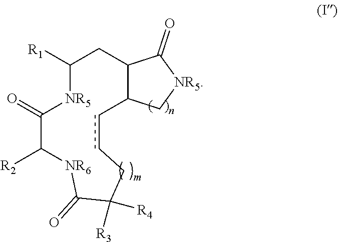

In some embodiments, R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino. In some embodiments, R₃ is selected from H, optionally substituted alkyl, optionally substituted cycloalkyl, optionally substituted amino, optionally substituted aryl, optionally substituted oxyacylamino, optionally substituted aminoacyloxy, optionally substituted acylamino, optionally substituted aminoacyl, and optionally substituted sulfonylamino. In some embodiments, R₃ is selected from H, optionally substituted alkyl, optionally substituted cycloalkyl, optionally substituted amino, optionally substituted alkyoxy, optionally substituted cycloalkyoxy, optionally substituted oxyacylamino, optionally substituted acylamino, optionally substituted aminoacyl, and optionally substituted sulfonylamino.

In some embodiments, the optional substituent on R₃ is selected from haloalkyl, alkyl, cycloalkyl, alkylcycloalkyl, halocycloalkyl, haloalkylcycloalkyl, cycloalkylalkyl, heterocyclyl, haloheterocyclyl, alkylheterocyclyl, haloalkylheterocyclyl, heterocyclylalkyl, haloheterocyclylalkyl, heteroaryl, haloheteroaryl, alkylheteroaryl, haloalkylheteroaryl, heteroarylalkyl, haloheteroarylalkyl, aryl, haloaryl, alkylaryl, haloalkylaryl, arylalkyl, haloarylalkyl, bridged cycloalkyl optionally substituted with halo and/or alkyl, spiro cycloalkyl optionally substituted with halo and/or alkyl, and protecting group. In some embodiments, the optional substituent on R₃ is selected from trihalomethyl, butyl, iso-butyl, trihalomethylpropyl, cyclopropyl, cyclobutyl, cyclopentyl, cyclohexyl, methylcyclopropyl, trihalomethylcyclopropyl, methylcyclobutyl, dihalocyclobutyl, trihalomethylcyclobutyl, dihalocyclobutylmethyl, cyclopentyl, dihalocyclohexyl, tetrahydrofuranyl, pyrimidinyl, trihalomethylpyrimidinyl, phenyl, halophenylpropyl, halophenyl, dihalophenyl, trihalomethylhalophenyl, trihalomethylisoxazolyl, methylisoxazolyl, bicycle[2.1.1]hexyl, trihalomethylbicyclo[1.1.1]pentyl, methyloxetanyl, spiro[3.3]septyl, and tert-butyloxyacylamino.

In some embodiments, R₃ is selected from H, optionally substituted phenylmethyl, optionally substituted halophenylmethyl, optionally substituted trihalomethylphenylmethyl, optionally substituted cyclopentyl, optionally substituted cyclopentylmethyl, optionally substituted tert-butyloxy, optionally substituted cyclohexyloxy, optionally substituted tetrahydropyranylacylamino, optionally substituted tert-butylacylamino, optionally substituted trihalotert-butylacylamino, optionally substituted trihalomethylacylamino, optionally substituted neo-pentylacylamino, optionally substituted dihalocyclobutylmethylacylamino, optionally substituted cyclopentylmethylacylamino, optionally substituted pyrimidinylacylamino, optionally substituted phenylacylamino, optionally substituted halophenylacylamino, optionally substituted dihalophenylacylamino, optionally substituted isoxazolylacylamino, optionally substituted methylisoxazolylacylamino, optionally substituted trihalomethylisoxazolylacylamino, optionally substituted thiazolylacylamino, optionally substituted methylthiazolylacylamino, optionally substituted oxazolylacylamino, optionally substituted methyloxazolylacylamino, optionally substituted phenylacylamino, optionally substituted halophenylacylamino, optionally substituted dihalophenylacylamino, optionally substituted trihalomethylphenylacylamino, optionally substituted halotrihalomethylphenylacylamino, optionally substituted halophenylpropylacylamino, optionally substituted cyclopropylacylamino, optionally substituted trihalomethylcyclopropylacylamino, optionally substituted cyclobutylacylamino, optionally substituted methylcyclobutylacylamino, optionally substituted dihalocyclobutylacylamino, optionally substituted bicyclo[1.1.1]pentylacylamino, optionally substituted trihalomethylbicyclo[1.1.1]pentylacylamino, optionally substituted oxetanylacylamino, optionally substituted methyloxetanylacylamino, optionally substituted bicycl[2.1.1]hexylacylamino, optionally substituted cyclohexylacylamino, optionally substituted dihalocyclohexylacylamino, optionally substituted cyclohexylacylamino, optionally substituted spiro[3.3]heptylacylamino, optionally substituted tert-butyloxyacylamino (or Boc-NH or Boc-N(methyl)), optionally substituted cyclopentyloxyacyamino, optionally substituted neo-pentyloxyacylamino, optionally substituted cyclobutyloxyacylamino, optionally substituted trihalomethylcyclobutyloxyacylamino, optionally substituted phenylsulfonylamino, optionally substituted dihalophenylsulfonylamino, optionally substituted cyclopropylsulfonylamino, optionally substituted methylsulfonylamino, optionally substituted trihalomethylsulfonylamino, optionally substituted cyclobutylsulfonylamino, optionally substituted amino, optionally substituted pyrimidinylamino, optionally substituted trihalomethylpyrimidinylamino, and optionally substituted tert-butylaminoacyl.

In some embodiments, when X is N, R₃ is optionally substituted oxyacylamino.

In some embodiments, when X is CR₄, R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino.

In some embodiments, R₄ is selected from H, and optionally substituted alkyl. In some embodiments, R₄ is selected from H, and optionally substituted C₁-C₅alkyl. In some embodiments, R₄ is selected from H, and C₁-C₅alkyl. In some embodiments, R₄ is H.

In some embodiments, R₅ is independently selected from H, and optionally substituted C₁-C₅ alkyl. In some embodiments, R₅ is independently selected from H, and C₁-C₅ alkyl.

In some embodiments, R₅ is independently H or methyl. In some embodiments, R₅ is independently H.

In some embodiments, R₆ is selected from H, and optionally substituted C₁-C₅ alkyl. In some embodiments, R₆ is selected from H, and C₁-C₅ alkyl. In some embodiments, R₆ is H.

R₂ and R₆ may be linked to form an optionally substituted heterocyclyl. In this regard, R₂ may form an optionally substituted heterocyclyl with the N at a p position relative to R₂. Accordingly, in some embodiments, the compound is a compound of Formula (Ia):

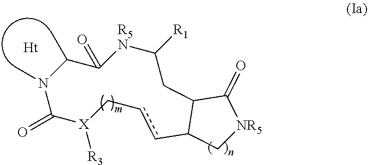

wherein R₁ is selected from optionally substituted acyl and cyano;
Ht is an optionally substituted heterocyclyl;
X is selected from N or C—R₄;
R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino;
R₄ is selected from H, and optionally substituted alkyl;
R₅ is independently selected from H, and optionally substituted alkyl;
m is an integer selected from 1 to 4; and
n is an integer selected from 1 to 4.

In some embodiments, the optionally substituted heterocyclyl (Ht) is an optionally substituted 5-7 membered heterocyclyl. In some embodiments, the heterocyclyl (Ht) is a 5-6 membered heterocyclyl. In some embodiments, the heterocyclyl (Ht) is a 5 membered heterocyclyl. In some embodiments, the heterocyclyl (Ht) is a 6 membered heterocyclyl.

In some embodiments, the optional substituent on the heterocyclyl is selected from halo, alkyl, alkoxy, cycloalkyl, alkylcycloalkyl, and bridged alkylene. In some embodiments, the optional substituent on the heterocyclyl is selected from halo, methyl, methoxy, ethoxy, cyclopropyl, fused dimethylcyclopropyl, fused cyclopentyl, bridged methylene, and bridged ethylene.

In some embodiments, m is an integer selected from 1 to 3.

In some embodiments, n is an integer selected from 1 to 2.

In some embodiments, the compound is a compound of Formula (I′″):

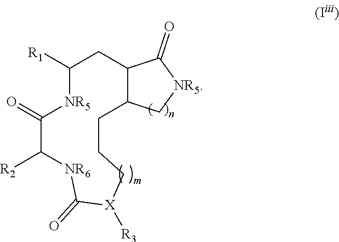

In some embodiments, the compound is a compound of Formula (Iiv):

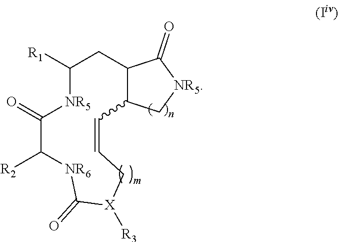

In some embodiments, the compound is a compound of Formula (I^{iv}a) or (I^{iv}b):

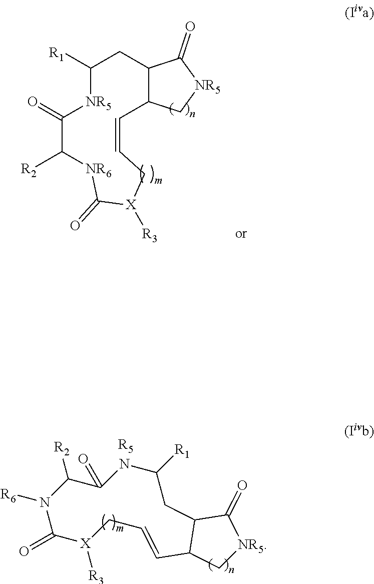

Formula (I^{iv}a) is a cis isomer of Formula (I) and Formula (I^{iv}b) is a trans isomer of Formula (I). Both isomers are found to be potent, however, it is believed that the trans isomer is more potent than the cis isomer.

Based on the structural activity relationship, it is believed that at least one of the chiral centers at the the lactam ring may drive the potency of these compounds. This motif is also different from previously reported structures. In some embodiments, the compound of Formula (I) or a salt, solvate, stereoisomer or prodrug thereof is represented by Formula (I^{v}a) or (I^{v}b):

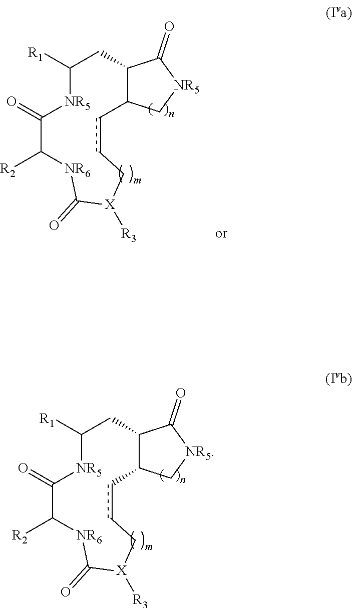

In some embodiments, the compound of Formula (I) or a salt, solvate, stereoisomer or prodrug thereof is represented by Formula (I^{vi}a) or (I^{vi}b):

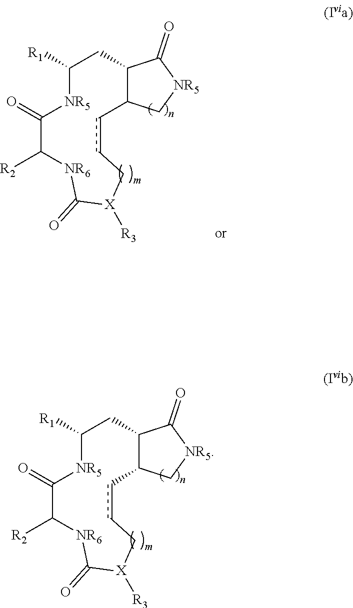

In some embodiments, the compound of Formula (I) or a salt, solvate, stereoisomer or prodrug thereof is represented by Formula (I^{vii}a) or (I^{vii}b):

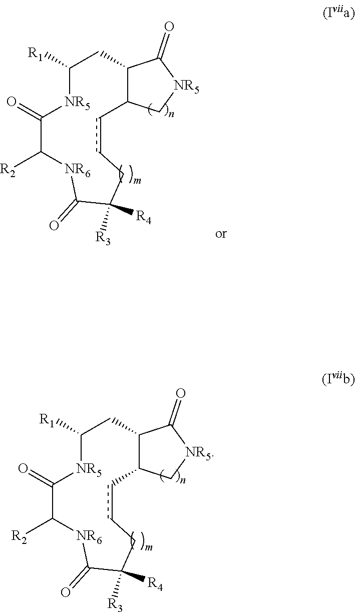

In some embodiments, the compound of Formula (I) or a salt, solvate, stereoisomer or prodrug thereof is represented by Formula (I^{iix}a) or (I^{iix}b):

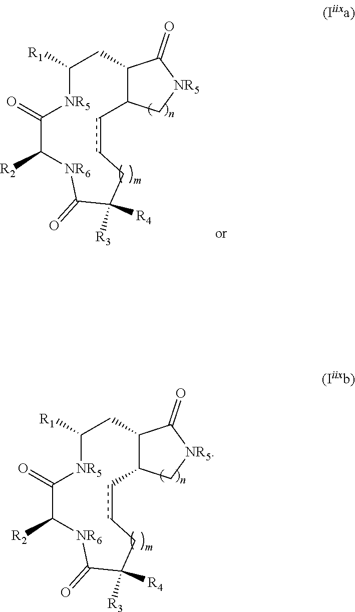

In some embodiments, the compound of Formula (Ia) or a salt, solvate, stereoisomer or prodrug thereof is represented by Formula (Iaⁱ):

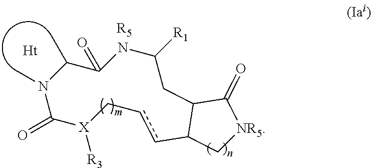

In some embodiments, the compound of Formula (Ia) or a salt, solvate, stereoisomer or prodrug thereof is represented by Formula (Iaⁱⁱⁱ):

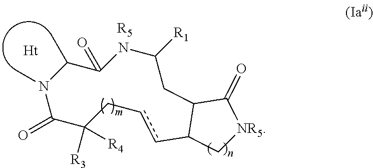

In some embodiments, the compound of Formula (Ia) or a salt, solvate, stereoisomer or prodrug thereof is represented by Formula (Iaⁱⁱⁱ):

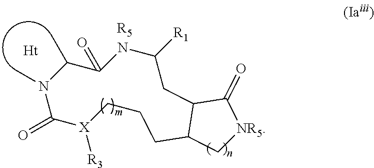

In some embodiments, the compound of Formula (Ia) or a salt, solvate, stereoisomer or prodrug thereof is represented by Formula (Ia^{iv}):

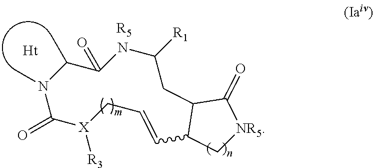

In some embodiments, the compound of Formula (Ia) or a salt, solvate, stereoisomer or prodrug thereof is represented by Formula (Ia^{v}a) or (Ia^{v}b):

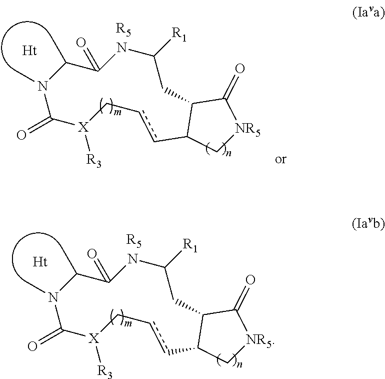

In some embodiments, the compound of Formula (Ia) or a salt, solvate, stereoisomer or prodrug thereof is represented by Formula (Ia^{vi}a) or (Ia^{vi}b):

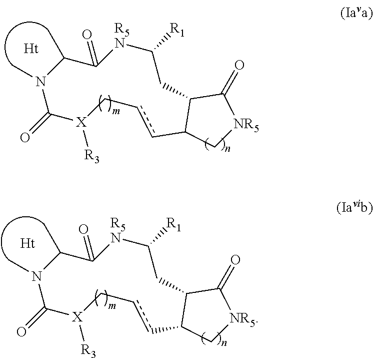

In some embodiments, the compound of Formula (Ia) or a salt, solvate, stereoisomer or prodrug thereof is represented by Formula (Ia^{vii}a) or (Ia^{vii}b):

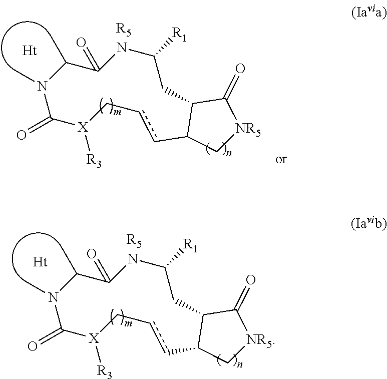

In some embodiments, the compound of Formula (Ia) or a salt, solvate, stereoisomer or prodrug thereof is represented by Formula (Ia^{iix}a) or (Ia^{iix}b):

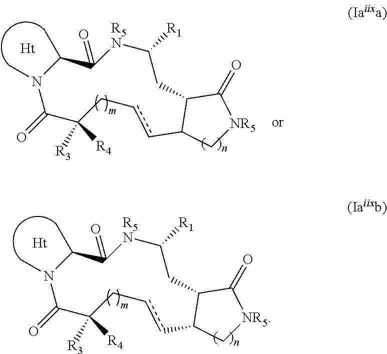

In some embodiments, the compound of Formula (I) is selected from:

| Cpd No. | Structure |
|---|---|
| 1 | 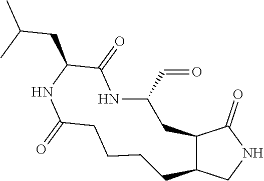 [SMILES: CC(C)C[C@@H]1NC(=O)CCCC[C@@H]2CNC(=O)[C@@H]2C[C@@H](C=O)NC1=O] |
| 2 | 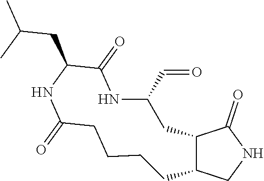 [SMILES: CC(C)C[C@@H]1NC(=O)CCCC[C@H]2CNC(=O)[C@H]2C[C@@H](C=O)NC1=O] |
| 3 | 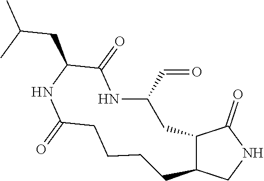 [SMILES: CC(C)C[C@@H]1NC(=O)CCCC[C@@H]2CNC(=O)[C@H]2C[C@@H](C=O)NC1=O] |
| 4 | 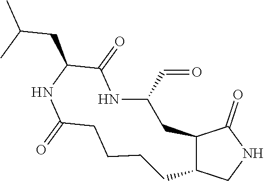 [SMILES: CC(C)C[C@@H]1NC(=O)CCCC[C@H]2CNC(=O)[C@@H]2C[C@@H](C=O)NC1=O] |
| 5 | 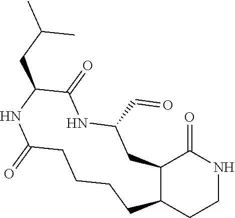 [SMILES: CC(C)C[C@@H]1NC(=O)CCCC[C@@H]2CCNC(=O)[C@@H]2C[C@@H](C=O)NC1=O] |
| 6 | 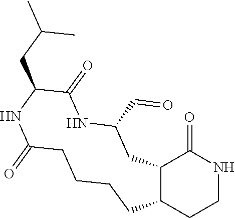 [SMILES: CC(C)C[C@@H]1NC(=O)CCCC[C@H]2CCNC(=O)[C@H]2C[C@@H](C=O)NC1=O] |
| 7 | 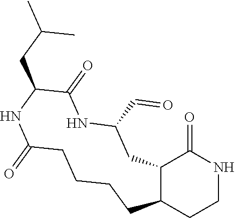 [SMILES: CC(C)C[C@@H]1NC(=O)CCCC[C@@H]2CCNC(=O)[C@H]2C[C@@H](C=O)NC1=O] |
| 8 | 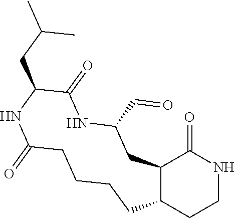 [SMILES: CC(C)C[C@@H]1NC(=O)CCCC[C@H]2CCNC(=O)[C@@H]2C[C@@H](C=O)NC1=O] |
| 9 | 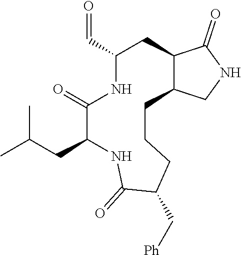 [SMILES: CC(C)C[C@@H]1NC(=O)[C@@H](Cc2ccccc2)CCC[C@@H]2CNC(=O)[C@@H]2C[C@@H](C=O)NC1=O] |
| 10 | 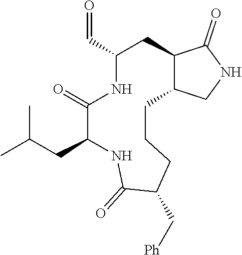 [SMILES: CC(C)C[C@@H]1NC(=O)[C@@H](Cc2ccccc2)CCC[C@H]2CNC(=O)[C@@H]2C[C@@H](C=O)NC1=O] |
| 11 | 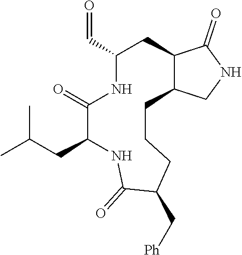 [SMILES: CC(C)C[C@@H]1NC(=O)[C@H](Cc2ccccc2)CCC[C@@H]2CNC(=O)[C@@H]2C[C@@H](C=O)NC1=O] |
| 12 | 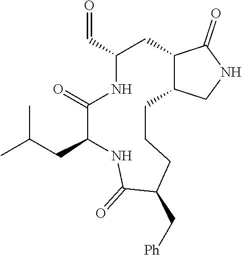 [SMILES: CC(C)C[C@@H]1NC(=O)[C@H](Cc2ccccc2)CCC[C@H]2CNC(=O)[C@H]2C[C@@H](C=O)NC1=O] |
| 13 | 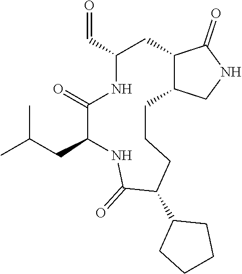 [SMILES: CC(C)C[C@@H]1NC(=O)[C@@H](C2CCCC2)CCC[C@H]2CNC(=O)[C@H]2C[C@@H](C=O)NC1=O] |
| 14 | 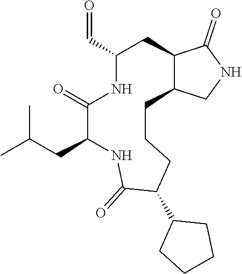 [SMILES: CC(C)C[C@@H]1NC(=O)[C@@H](C2CCCC2)CCC[C@@H]2CNC(=O)[C@@H]2C[C@@H](C=O)NC1=O] |
| 15 | 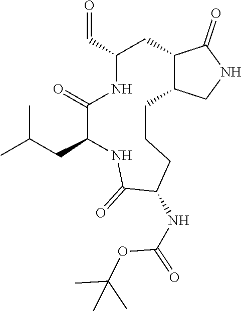 [SMILES: CC(C)C[C@@H]1NC(=O)[C@@H](NC(=O)OC(C)(C)C)CCC[C@H]2CNC(=O)[C@H]2C[C@@H](C=O)NC1=O] |
| 16 | 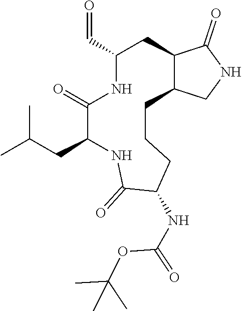 [SMILES: CC(C)C[C@@H]1NC(=O)[C@@H](NC(=O)OC(C)(C)C)CCC[C@@H]2CNC(=O)[C@@H]2C[C@@H](C=O)NC1=O] |
| 17 | 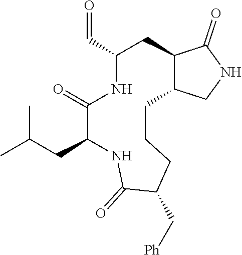 [SMILES: CC(C)C[C@@H]1NC(=O)[C@@H](Cc2ccccc2)CCC[C@H]2CNC(=O)[C@@H]2C[C@@H](C=O)NC1=O] |
| 18 | 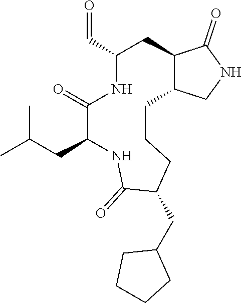 [SMILES: CC(C)C[C@@H]1NC(=O)[C@@H](CC2CCCC2)CCC[C@H]2CNC(=O)[C@@H]2C[C@@H](C=O)NC1=O] |
| 19 | 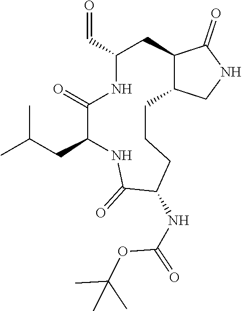 [SMILES: CC(C)C[C@@H]1NC(=O)[C@@H](NC(=O)OC(C)(C)C)CCC[C@H]2CNC(=O)[C@@H]2C[C@@H](C=O)NC1=O] |
| 20 | 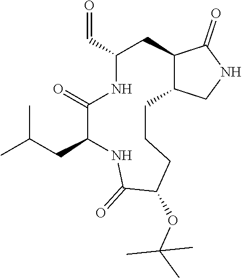 [SMILES: CC(C)C[C@@H]1NC(=O)[C@@H](OC(C)(C)C)CCC[C@H]2CNC(=O)[C@@H]2C[C@@H](C=O)NC1=O] |
| 21 | 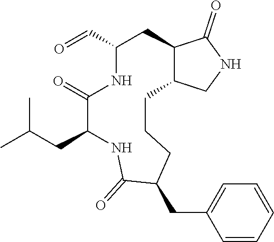 [SMILES: CC(C)C[C@@H]1NC(=O)[C@H](Cc2ccccc2)CCC[C@H]2CNC(=O)[C@@H]2C[C@@H](C=O)NC1=O] |
| 22 | 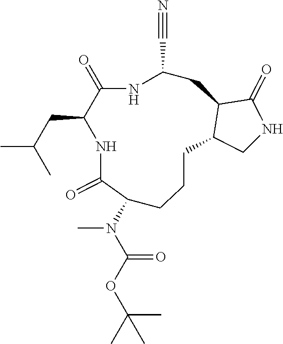 [SMILES: CC(C)C[C@@H]1NC(=O)[C@@H](N(C)C(=O)OC(C)(C)C)CCC[C@H]2CNC(=O)[C@@H]2C[C@@H](C#N)NC1=O] |
| 23 | 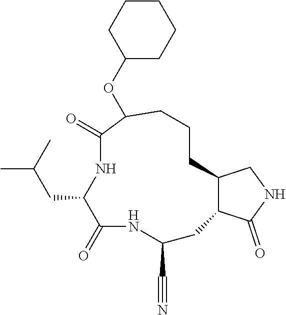 [SMILES: CC(C)C[C@@H]1NC(=O)C(OC2CCCCC2)CCC[C@H]2CNC(=O)[C@@H]2C[C@@H](C#N)NC1=O] |
| 24 | 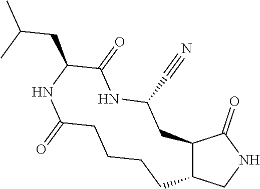 [SMILES: CC(C)C[C@@H]1NC(=O)CCCC[C@H]2CNC(=O)[C@@H]2C[C@@H](C#N)NC1=O] |
| 25 | 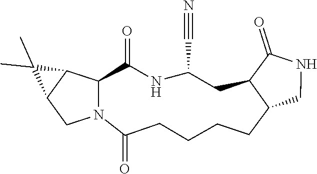 [SMILES: CC1(C)[C@@H]2[C@H]3C(=O)N[C@H](C#N)C[C@H]4C(=O)NC[C@@H]4CCCCC(=O)N3C[C@@H]21] |
| 26 | 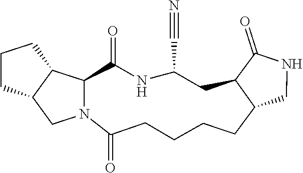 [SMILES: N#C[C@@H]1C[C@H]2C(=O)NC[C@@H]2CCCCC(=O)N2C[C@@H]3CCC[C@@H]3[C@H]2C(=O)N1] |
| 27 | 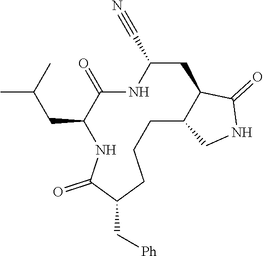 [SMILES: CC(C)C[C@@H]1NC(=O)[C@@H](Cc2ccccc2)CCC[C@H]2CNC(=O)[C@@H]2C[C@@H](C#N)NC1=O] |
| 28 | 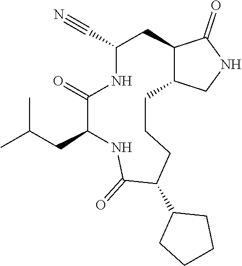 [SMILES: CC(C)C[C@@H]1NC(=O)[C@@H](C2CCCC2)CCC[C@H]2CNC(=O)[C@@H]2C[C@@H](C#N)NC1=O] |
| 29 | 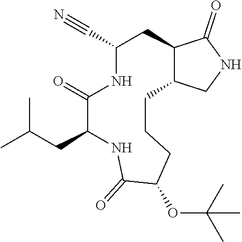 [SMILES: CC(C)C[C@@H]1NC(=O)[C@@H](OC(C)(C)C)CCC[C@H]2CNC(=O)[C@@H]2C[C@@H](C#N)NC1=O] |
| 30 | 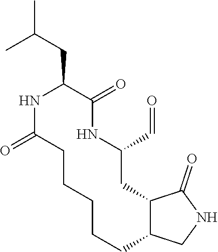 [SMILES: CC(C)C[C@@H]1NC(=O)CCCCC[C@H]2CNC(=O)[C@H]2C[C@@H](C=O)NC1=O] |
| 31 | 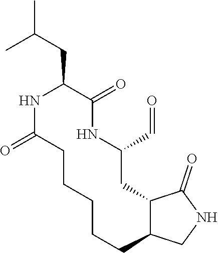 [SMILES: CC(C)C[C@@H]1NC(=O)CCCCC[C@@H]2CNC(=O)[C@H]2C[C@@H](C=O)NC1=O] |
| 32 | 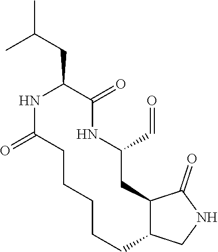 [SMILES: CC(C)C[C@@H]1NC(=O)CCCCC[C@H]2CNC(=O)[C@@H]2C[C@@H](C=O)NC1=O] |
| 33 | 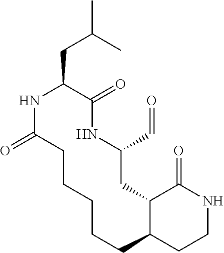 [SMILES: CC(C)C[C@@H]1NC(=O)CCCCC[C@@H]2CCNC(=O)[C@H]2C[C@@H](C=O)NC1=O] |
| 34 | 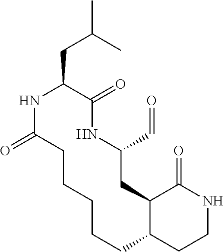 [SMILES: CC(C)C[C@@H]1NC(=O)CCCCC[C@H]2CCNC(=O)[C@@H]2C[C@@H](C=O)NC1=O] |
| 35 | 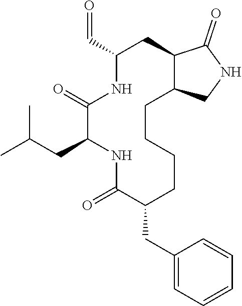 [SMILES: CC(C)C[C@@H]1NC(=O)[C@@H](Cc2ccccc2)CCCC[C@@H]2CNC(=O)[C@@H]2C[C@@H](C=O)NC1=O] |
| 36 | 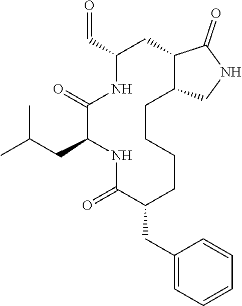 [SMILES: CC(C)C[C@@H]1NC(=O)[C@@H](Cc2ccccc2)CCCC[C@H]2CNC(=O)[C@H]2C[C@@H](C=O)NC1=O] |
| 37 | 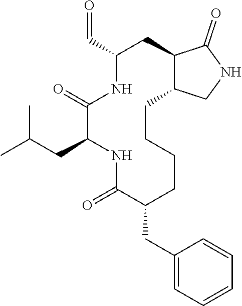 [SMILES: CC(C)C[C@@H]1NC(=O)[C@@H](Cc2ccccc2)CCCC[C@H]2CNC(=O)[C@@H]2C[C@@H](C=O)NC1=O] |
| 38 | 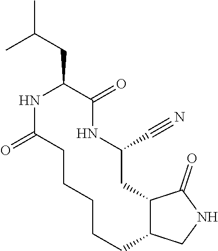 [SMILES: CC(C)C[C@@H]1NC(=O)CCCCC[C@H]2CNC(=O)[C@H]2C[C@@H](C#N)NC1=O] |
| 39 | 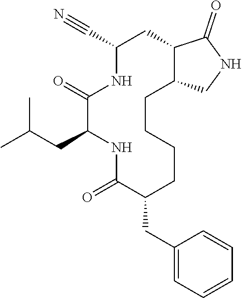 [SMILES: CC(C)C[C@@H]1NC(=O)[C@@H](Cc2ccccc2)CCCC[C@H]2CNC(=O)[C@H]2C[C@@H](C#N)NC1=O] |
| 40 | 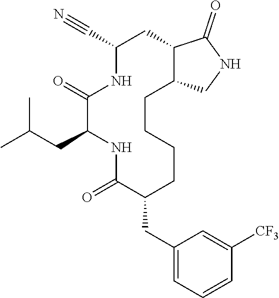 [SMILES: CC(C)C[C@@H]1NC(=O)[C@@H](Cc2cccc(C(F)(F)F)c2)CCCC[C@H]2CNC(=O)[C@H]2C[C@@H](C#N)NC1=O] |
| 41 | 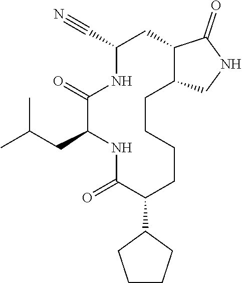 [SMILES: CC(C)C[C@@H]1NC(=O)[C@@H](C2CCCC2)CCCC[C@H]2CNC(=O)[C@H]2C[C@@H](C#N)NC1=O] |
| 42 | 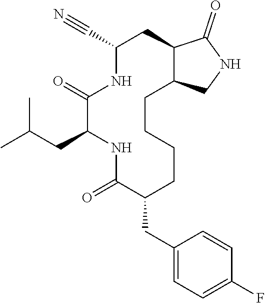 [SMILES: CC(C)C[C@@H]1NC(=O)[C@@H](Cc2ccc(F)cc2)CCCC[C@@H]2CNC(=O)[C@@H]2C[C@@H](C#N)NC1=O] |
| 43 | 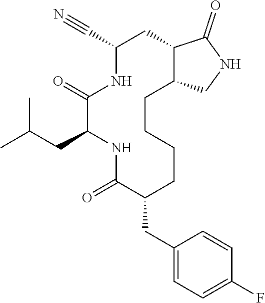 [SMILES: CC(C)C[C@@H]1NC(=O)[C@@H](Cc2ccc(F)cc2)CCCC[C@H]2CNC(=O)[C@H]2C[C@@H](C#N)NC1=O] |
| 44 | 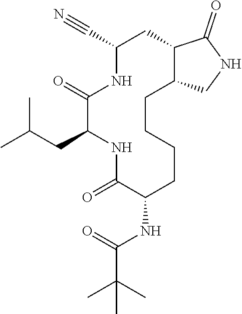 [SMILES: CC(C)C[C@@H]1NC(=O)[C@@H](NC(=O)C(C)(C)C)CCCC[C@H]2CNC(=O)[C@H]2C[C@@H](C#N)NC1=O] |
| 45 | 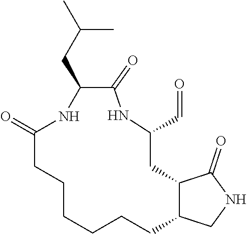 [SMILES: CC(C)C[C@@H]1NC(=O)CCCCCC[C@H]2CNC(=O)[C@H]2C[C@@H](C=O)NC1=O] |
| 46 | 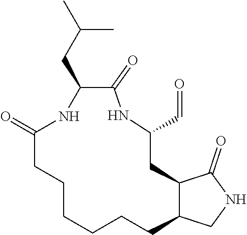 [SMILES: CC(C)C[C@@H]1NC(=O)CCCCCC[C@@H]2CNC(=O)[C@@H]2C[C@@H](C=O)NC1=O] |
| 47 | 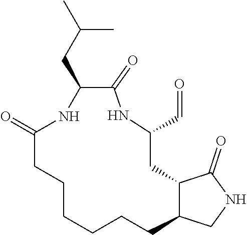 [SMILES: CC(C)C[C@@H]1NC(=O)CCCCCC[C@@H]2CNC(=O)[C@H]2C[C@@H](C=O)NC1=O] |
| 48 | 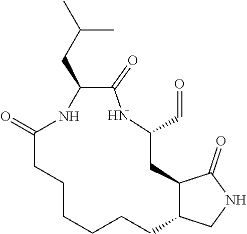 [SMILES: CC(C)C[C@@H]1NC(=O)CCCCCC[C@H]2CNC(=O)[C@@H]2C[C@@H](C=O)NC1=O] |
| 49 | 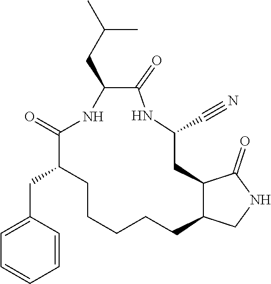 [SMILES: CC(C)C[C@@H]1NC(=O)[C@@H](Cc2ccccc2)CCCCC[C@@H]2CNC(=O)[C@@H]2C[C@@H](C#N)NC1=O] |
| 50 | 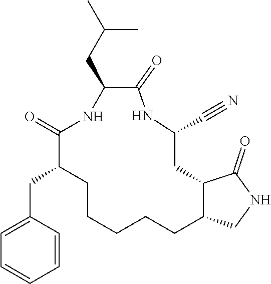 [SMILES: CC(C)C[C@@H]1NC(=O)[C@@H](Cc2ccccc2)CCCCC[C@H]2CNC(=O)[C@H]2C[C@@H](C#N)NC1=O] |
| 51 | 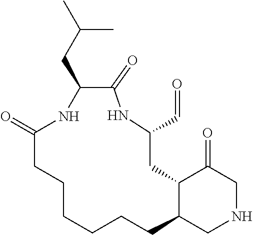 [SMILES: CC(C)C[C@@H]1NC(=O)CCCCCC[C@@H]2CNCC(=O)[C@H]2C[C@@H](C=O)NC1=O] |
| 52 | 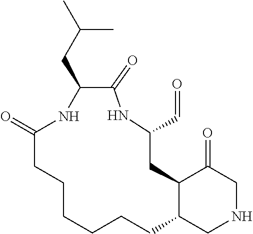 [SMILES: CC(C)C[C@@H]1NC(=O)CCCCCC[C@H]2CNCC(=O)[C@@H]2C[C@@H](C=O)NC1=O] |
| 53 | 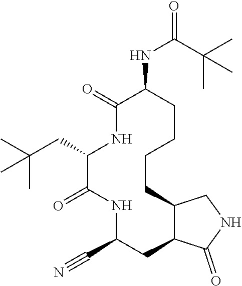 [SMILES: CC(C)(C)C[C@@H]1NC(=O)[C@@H](NC(=O)C(C)(C)C)CCCC[C@H]2CNC(=O)[C@H]2C[C@@H](C#N)NC1=O] |
| 54 | 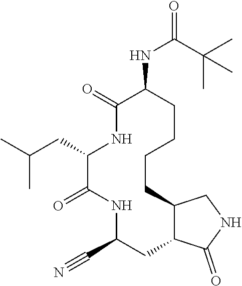 [SMILES: CC(C)C[C@@H]1NC(=O)[C@@H](NC(=O)C(C)(C)C)CCCC[C@H]2CNC(=O)[C@@H]2C[C@@H](C#N)NC1=O] |
| 55 | 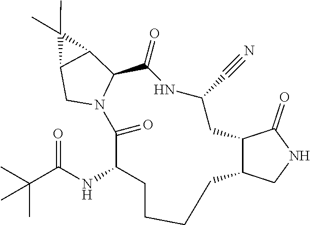 [SMILES: CC(C)(C)C(=O)N[C@H]1CCCC[C@H]2CNC(=O)[C@H]2C[C@@H](C#N)NC(=O)[C@@H]2[C@@H]3[C@H](CN2C1=O)C3(C)C] |
| 56 | 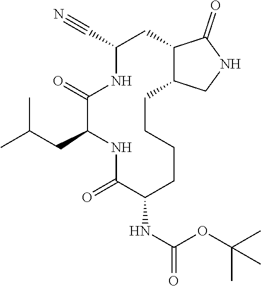 [SMILES: CC(C)C[C@@H]1NC(=O)[C@@H](NC(=O)OC(C)(C)C)CCCC[C@H]2CNC(=O)[C@H]2C[C@@H](C#N)NC1=O] |
| 57 | 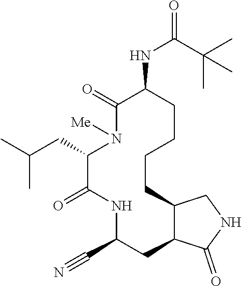 [SMILES: CC(C)C[C@H]1C(=O)N[C@H](C#N)C[C@@H]2C(=O)NC[C@@H]2CCCC[C@H](NC(=O)C(C)(C)C)C(=O)N1C] |
| 58 | 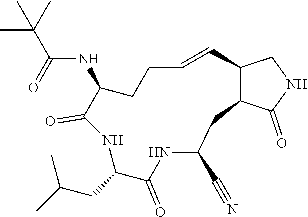 [SMILES: CC(C)C[C@@H]1NC(=O)[C@@H](NC(=O)C(C)(C)C)CC/C=C/[C@H]2CNC(=O)[C@H]2C[C@@H](C#N)NC1=O] |
| 59 | 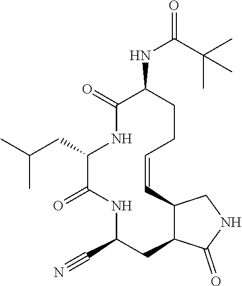 [SMILES: CC(C)C[C@@H]1NC(=O)[C@@H](NC(=O)C(C)(C)C)CC/C=C\[C@H]2CNC(=O)[C@H]2C[C@@H](C#N)NC1=O] |
| 60 | 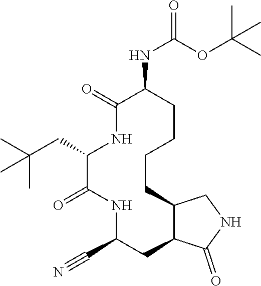 [SMILES: CC(C)(C)C[C@@H]1NC(=O)[C@@H](NC(=O)OC(C)(C)C)CCCC[C@H]2CNC(=O)[C@H]2C[C@@H](C#N)NC1=O] |
| 61 | 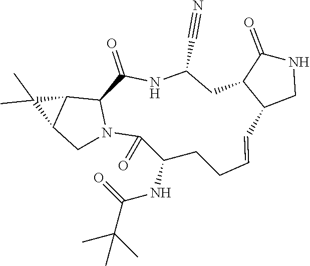 [SMILES: CC(C)(C)C(=O)N[C@H]1CC/C=C/[C@H]2CNC(=O)[C@H]2C[C@@H](C#N)NC(=O)[C@@H]2[C@@H]3[C@H](CN2C1=O)C3(C)C] |
| 62 | 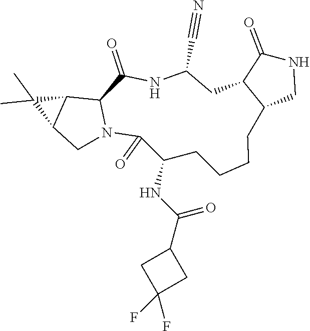 [SMILES: CC1(C)[C@@H]2[C@H]3C(=O)N[C@H](C#N)C[C@@H]4C(=O)NC[C@@H]4CCCC[C@H](NC(=O)C4CC(F)(F)C4)C(=O)N3C[C@@H]21] |
| 63 | 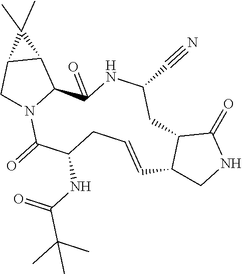 [SMILES: CC(C)(C)C(=O)N[C@H]1C/C=C/[C@H]2CNC(=O)[C@H]2C[C@@H](C#N)NC(=O)[C@@H]2[C@@H]3[C@H](CN2C1=O)C3(C)C] |
| 64 | 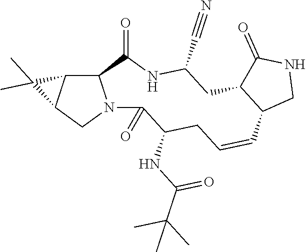 [SMILES: CC(C)(C)C(=O)N[C@H]1C/C=C\[C@H]2CNC(=O)[C@H]2C[C@@H](C#N)NC(=O)[C@@H]2[C@@H]3[C@H](CN2C1=O)C3(C)C] |
| 65 | 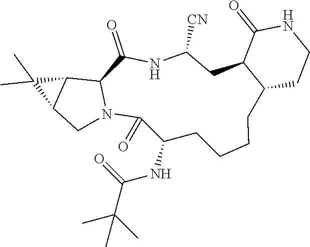 [SMILES: CC(C)(C)C(=O)N[C@H]1CCCC[C@H]2CCNC(=O)[C@@H]2C[C@@H](C#N)NC(=O)[C@@H]2[C@@H]3[C@H](CN2C1=O)C3(C)C] |
| 66 | 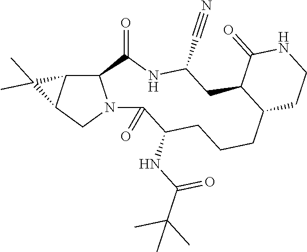 [SMILES: CC(C)(C)C(=O)N[C@H]1CCC[C@H]2CCNC(=O)[C@@H]2C[C@@H](C#N)NC(=O)[C@@H]2[C@@H]3[C@H](CN2C1=O)C3(C)C] |
| 67 | 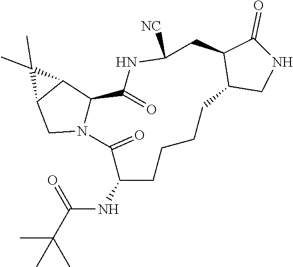 [SMILES: CC(C)(C)C(=O)N[C@H]1CCCC[C@H]2CNC(=O)[C@@H]2C[C@@H](C#N)NC(=O)[C@@H]2[C@@H]3[C@H](CN2C1=O)C3(C)C] |
| 68 | 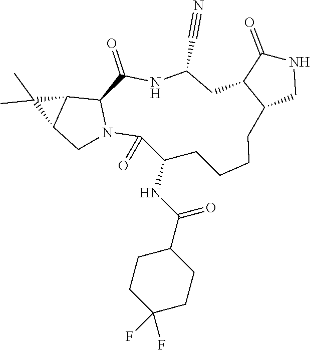 [SMILES: CC1(C)[C@@H]2[C@H]3C(=O)N[C@H](C#N)C[C@@H]4C(=O)NC[C@@H]4CCCC[C@H](NC(=O)C4CCC(F)(F)CC4)C(=O)N3C[C@@H]21] |
| 69 | 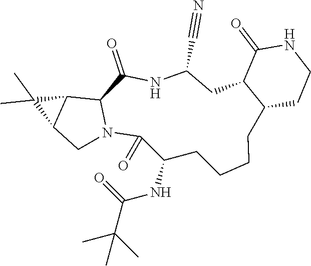 [SMILES: CC(C)(C)C(=O)N[C@H]1CCCC[C@H]2CCNC(=O)[C@H]2C[C@@H](C#N)NC(=O)[C@@H]2[C@@H]3[C@H](CN2C1=O)C3(C)C] |
| 70 | 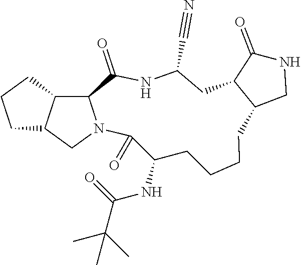 |
| 71 | 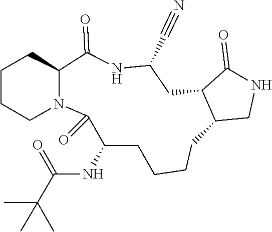 [SMILES: CC(C)(C)C(=O)N[C@H]1CCCC[C@H]2CNC(=O)[C@H]2C[C@@H](C#N)NC(=O)[C@@H]2CCCCN2C1=O] |
| 72 | 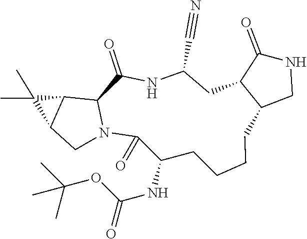 [SMILES: CC(C)(C)OC(=O)N[C@H]1CCCC[C@H]2CNC(=O)[C@H]2C[C@@H](C#N)NC(=O)[C@@H]2[C@@H]3[C@H](CN2C1=O)C3(C)C] |
| 73 | 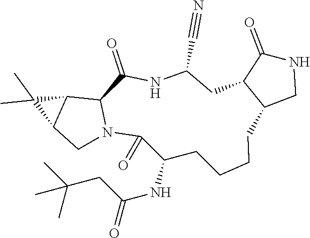 [SMILES: CC(C)(C)CC(=O)N[C@H]1CCCC[C@H]2CNC(=O)[C@H]2C[C@@H](C#N)NC(=O)[C@@H]2[C@@H]3[C@H](CN2C1=O)C3(C)C] |
| 74 | 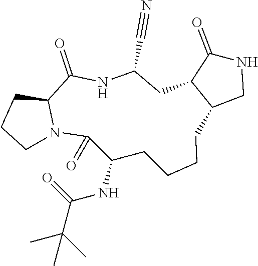 [SMILES: CC(C)(C)C(=O)N[C@H]1CCCC[C@H]2CNC(=O)[C@H]2C[C@@H](C#N)NC(=O)[C@@H]2CCCN2C1=O] |
| 75 | 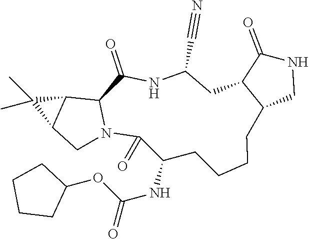 [SMILES: CC1(C)[C@@H]2[C@H]3C(=O)N[C@H](C#N)C[C@@H]4C(=O)NC[C@@H]4CCCC[C@H](NC(=O)OC4CCCC4)C(=O)N3C[C@@H]21] |
| 76 | 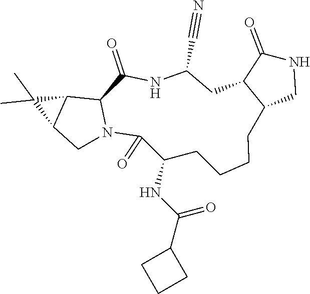 [SMILES: CC1(C)[C@@H]2[C@H]3C(=O)N[C@H](C#N)C[C@@H]4C(=O)NC[C@@H]4CCCC[C@H](NC(=O)C4CCC4)C(=O)N3C[C@@H]21] |
| 77 | 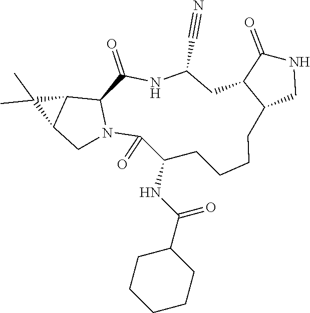 [SMILES: CC1(C)[C@@H]2[C@H]3C(=O)N[C@H](C#N)C[C@@H]4C(=O)NC[C@@H]4CCCC[C@H](NC(=O)C4CCCCC4)C(=O)N3C[C@@H]21] |
| 78 | 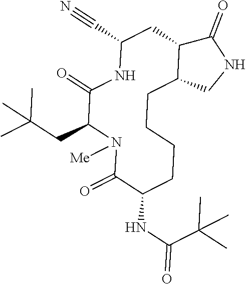 [SMILES: CN1C(=O)[C@@H](NC(=O)C(C)(C)C)CCCC[C@H]2CNC(=O)[C@H]2C[C@@H](C#N)NC(=O)[C@@H]1CC(C)(C)C] |
| 79 | 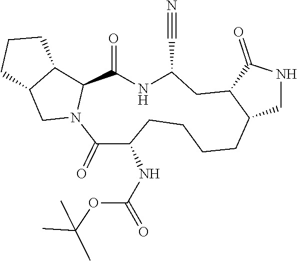 [SMILES: CC(C)(C)OC(=O)N[C@H]1CCCC[C@H]2CNC(=O)[C@H]2C[C@@H](C#N)NC(=O)[C@@H]2[C@H]3CCC[C@H]3CN2C1=O] |
| 80 | 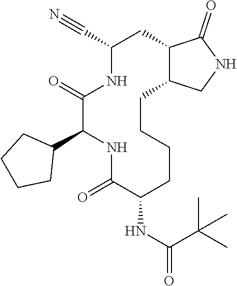 [SMILES: CC(C)(C)C(=O)N[C@H]1CCCC[C@H]2CNC(=O)[C@H]2C[C@@H](C#N)NC(=O)[C@H](C2CCCC2)NC1=O] |
| 81 | 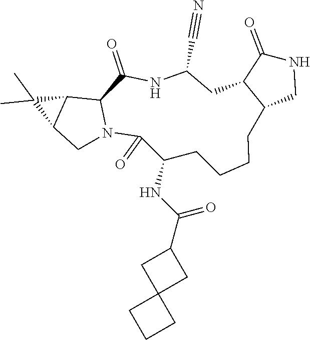 [SMILES: CC1(C)[C@@H]2[C@H]3C(=O)N[C@H](C#N)C[C@@H]4C(=O)NC[C@@H]4CCCC[C@H](NC(=O)C4CC5(CCC5)C4)C(=O)N3C[C@@H]21] |
| 82 | 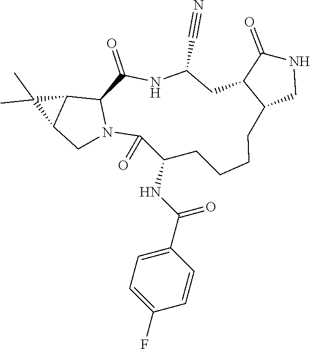 [SMILES: CC1(C)[C@@H]2[C@H]3C(=O)N[C@H](C#N)C[C@@H]4C(=O)NC[C@@H]4CCCC[C@H](NC(=O)c4ccc(F)cc4)C(=O)N3C[C@@H]21] |
| 83 | 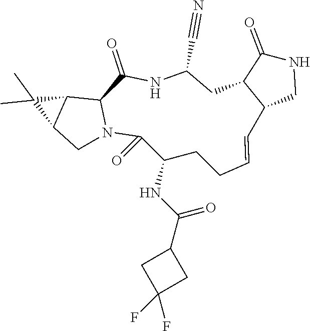 [SMILES: CC1(C)[C@@H]2[C@H]3C(=O)N[C@H](C#N)C[C@@H]4C(=O)NC[C@@H]4/C=C/CC[C@H](NC(=O)C4CC(F)(F)C4)C(=O)N3C[C@@H]21] |
| 84 | 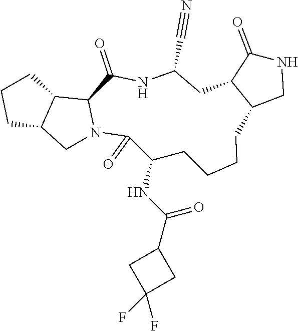 [SMILES: N#C[C@@H]1C[C@@H]2C(=O)NC[C@@H]2CCCC[C@H](NC(=O)C2CC(F)(F)C2)C(=O)N2C[C@@H]3CCC[C@@H]3[C@H]2C(=O)N1] |
| 85 | 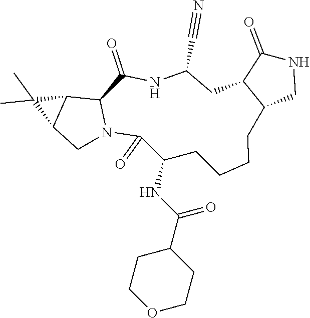 [SMILES: CC1(C)[C@@H]2[C@H]3C(=O)N[C@H](C#N)C[C@@H]4C(=O)NC[C@@H]4CCCC[C@H](NC(=O)C4CCOCC4)C(=O)N3C[C@@H]21] |
| 86 | 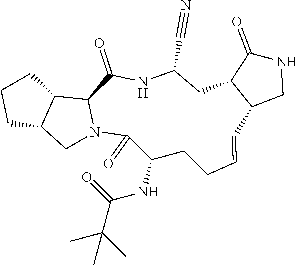 [SMILES: CC(C)(C)C(=O)N[C@H]1CC/C=C/[C@H]2CNC(=O)[C@H]2C[C@@H](C#N)NC(=O)[C@@H]2[C@H]3CCC[C@H]3CN2C1=O] |
| 87 | 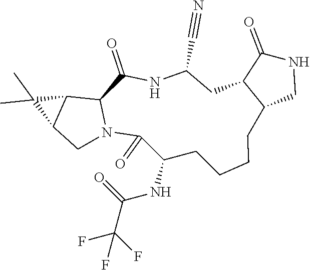 [SMILES: CC1(C)[C@@H]2[C@H]3C(=O)N[C@H](C#N)C[C@@H]4C(=O)NC[C@@H]4CCCC[C@H](NC(=O)C(F)(F)F)C(=O)N3C[C@@H]21] |
| 88 | 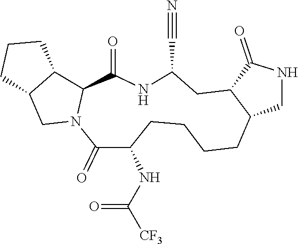 [SMILES: N#C[C@@H]1C[C@@H]2C(=O)NC[C@@H]2CCCC[C@H](NC(=O)C(F)(F)F)C(=O)N2C[C@@H]3CCC[C@@H]3[C@H]2C(=O)N1] |
| 89 | 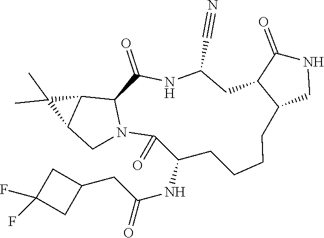 [SMILES: CC1(C)[C@@H]2[C@H]3C(=O)N[C@H](C#N)C[C@@H]4C(=O)NC[C@@H]4CCCC[C@H](NC(=O)CC4CC(F)(F)C4)C(=O)N3C[C@@H]21] |
| 90 | 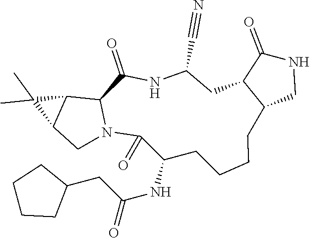 [SMILES: CC1(C)[C@@H]2[C@H]3C(=O)N[C@H](C#N)C[C@@H]4C(=O)NC[C@@H]4CCCC[C@H](NC(=O)CC4CCCC4)C(=O)N3C[C@@H]21] |
| 91 | 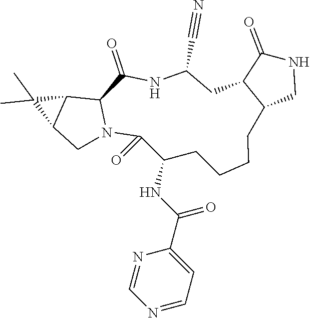 [SMILES: CC1(C)[C@@H]2[C@H]3C(=O)N[C@H](C#N)C[C@@H]4C(=O)NC[C@@H]4CCCC[C@H](NC(=O)c4ccncn4)C(=O)N3C[C@@H]21] |
| 92 | 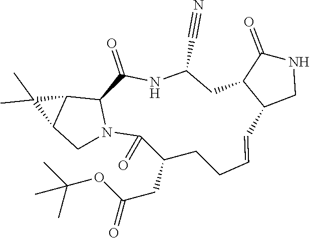 [SMILES: CC(C)(C)OC(=O)C[C@H]1CC/C=C/[C@H]2CNC(=O)[C@H]2C[C@@H](C#N)NC(=O)[C@@H]2[C@@H]3[C@H](CN2C1=O)C3(C)C] |
| 93 | 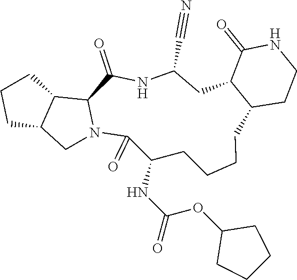 [SMILES: N#C[C@@H]1C[C@@H]2C(=O)NCC[C@@H]2CCCC[C@H](NC(=O)OC2CCCC2)C(=O)N2C[C@@H]3CCC[C@@H]3[C@H]2C(=O)N1] |
| 94 | 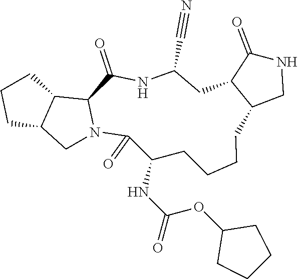 [SMILES: N#C[C@@H]1C[C@@H]2C(=O)NC[C@@H]2CCCC[C@H](NC(=O)OC2CCCC2)C(=O)N2C[C@@H]3CCC[C@@H]3[C@H]2C(=O)N1] |
| 95 | 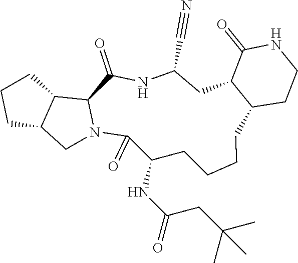 [SMILES: CC(C)(C)CC(=O)N[C@H]1CCCC[C@H]2CCNC(=O)[C@H]2C[C@@H](C#N)NC(=O)[C@@H]2[C@H]3CCC[C@H]3CN2C1=O] |
| 96 | 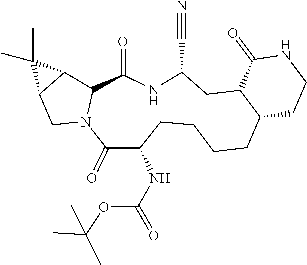 |
| 97 | 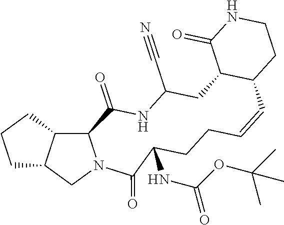 [SMILES: CC(C)(C)OC(=O)N[C@H]1CC/C=C\[C@H]2CCNC(=O)[C@H]2CC(C#N)NC(=O)[C@@H]2[C@H]3CCC[C@H]3CN2C1=O] |
| 98 | 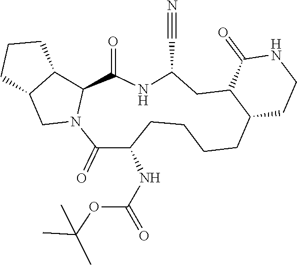 |
| 99 | 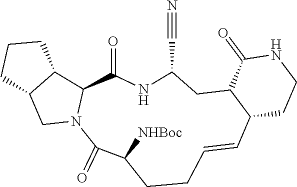 |
| 100 | 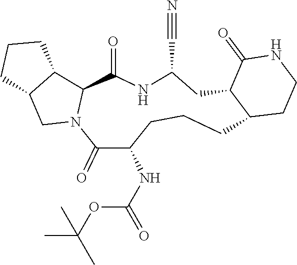 [SMILES: CC(C)(C)OC(=O)N[C@H]1CCC[C@H]2CCNC(=O)[C@H]2C[C@@H](C#N)NC(=O)[C@@H]2[C@H]3CCC[C@H]3CN2C1=O] |
| 101 | 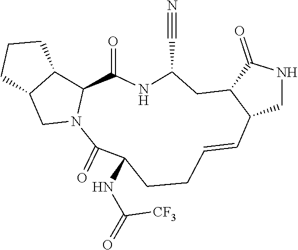 [SMILES: N#C[C@@H]1C[C@@H]2C(=O)NC[C@@H]2/C=C/CC[C@H](NC(=O)C(F)(F)F)C(=O)N2C[C@@H]3CCC[C@@H]3[C@H]2C(=O)N1] |
| 102 | 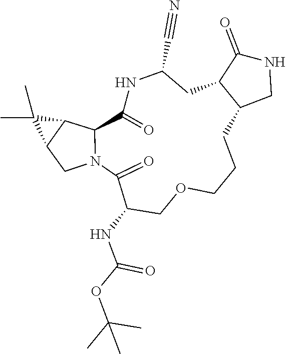 [SMILES: CC(C)(C)OC(=O)N[C@H]1COCCC[C@H]2CNC(=O)[C@H]2C[C@@H](C#N)NC(=O)[C@@H]2[C@@H]3[C@H](CN2C1=O)C3(C)C] |
| 103 | 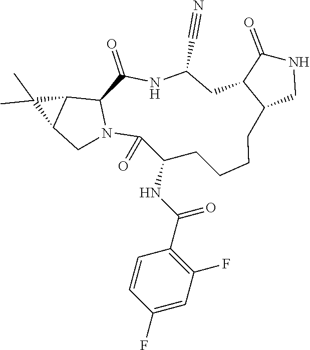 [SMILES: CC1(C)[C@@H]2[C@H]3C(=O)N[C@H](C#N)C[C@@H]4C(=O)NC[C@@H]4CCCC[C@H](NC(=O)c4ccc(F)cc4F)C(=O)N3C[C@@H]21] |
| 104 | 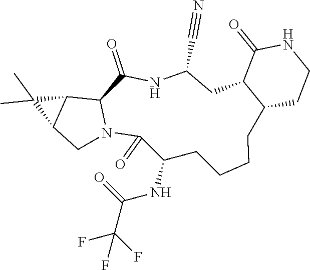 [SMILES: CC1(C)[C@@H]2[C@H]3C(=O)N[C@H](C#N)C[C@@H]4C(=O)NCC[C@@H]4CCCC[C@H](NC(=O)C(F)(F)F)C(=O)N3C[C@@H]21] |
| 105 | 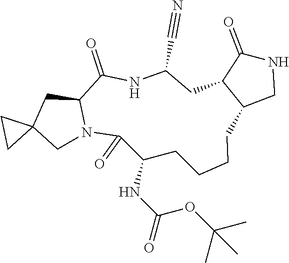 [SMILES: CC(C)(C)OC(=O)N[C@H]1CCCC[C@H]2CNC(=O)[C@H]2C[C@@H](C#N)NC(=O)[C@@H]2CC3(CC3)CN2C1=O] |
| 106 | 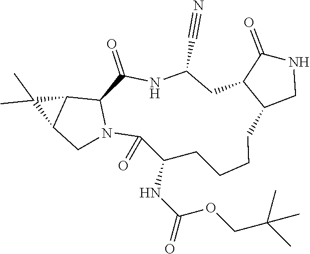 [SMILES: CC(C)(C)COC(=O)N[C@H]1CCCC[C@H]2CNC(=O)[C@H]2C[C@@H](C#N)NC(=O)[C@@H]2[C@@H]3[C@H](CN2C1=O)C3(C)C] |
| 107 | 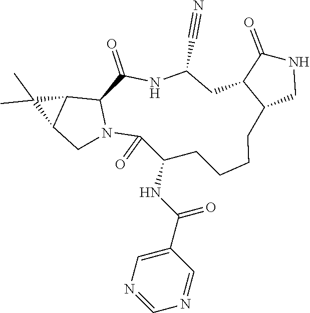 [SMILES: CC1(C)[C@@H]2[C@H]3C(=O)N[C@H](C#N)C[C@@H]4C(=O)NC[C@@H]4CCCC[C@H](NC(=O)c4cncnc4)C(=O)N3C[C@@H]21] |
| 108 | 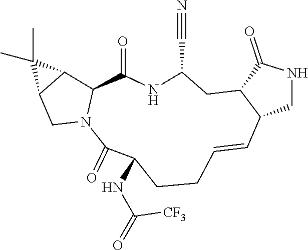 [SMILES: CC1(C)[C@@H]2[C@H]3C(=O)N[C@H](C#N)C[C@@H]4C(=O)NC[C@@H]4/C=C/CC[C@H](NC(=O)C(F)(F)F)C(=O)N3C[C@@H]21] |
| 109 | 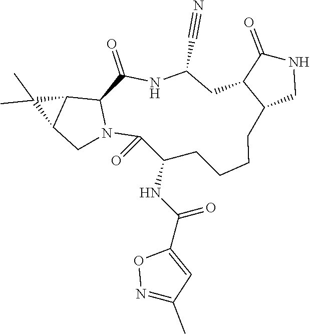 [SMILES: Cc1cc(C(=O)N[C@H]2CCCC[C@H]3CNC(=O)[C@H]3C[C@@H](C#N)NC(=O)[C@@H]3[C@@H]4[C@H](CN3C2=O)C4(C)C)on1] |
| 110 | 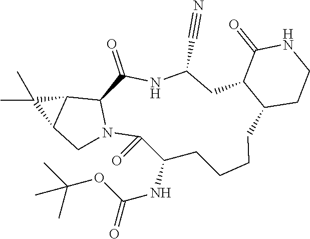 [SMILES: CC(C)(C)OC(=O)N[C@H]1CCCC[C@H]2CCNC(=O)[C@H]2C[C@@H](C#N)NC(=O)[C@@H]2[C@@H]3[C@H](CN2C1=O)C3(C)C] |
| 111 | 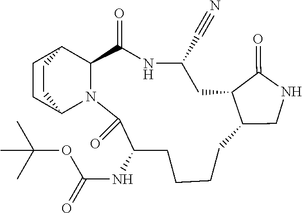 [SMILES: CC(C)(C)OC(=O)N[C@H]1CCCC[C@H]2CNC(=O)[C@H]2C[C@@H](C#N)NC(=O)[C@@H]2[C@H]3CC[C@H](CC3)N2C1=O] |
| 112 | 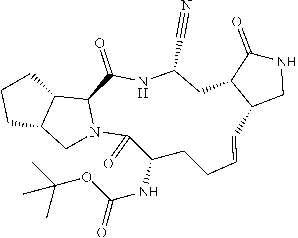 [SMILES: CC(C)(C)OC(=O)N[C@H]1CC/C=C/[C@H]2CNC(=O)[C@H]2C[C@@H](C#N)NC(=O)[C@@H]2[C@H]3CCC[C@H]3CN2C1=O] |
| 113 | 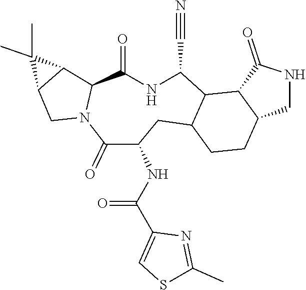 [SMILES: Cc1nc(C(=O)N[C@H]2CC3CC[C@H]4CNC(=O)[C@H]4C3[C@@H](C#N)NC(=O)[C@@H]3[C@@H]4[C@H](CN3C2=O)C4(C)C)cs1] |
| 114 | 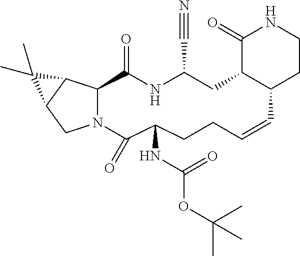 [SMILES: CC(C)(C)OC(=O)N[C@H]1CC/C=C\[C@H]2CCNC(=O)[C@H]2C[C@@H](C#N)NC(=O)[C@@H]2[C@@H]3[C@H](CN2C1=O)C3(C)C] |
| 115 | 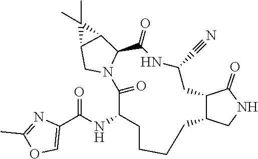 [SMILES: Cc1nc(C(=O)N[C@H]2CCCC[C@H]3CNC(=O)[C@H]3C[C@@H](C#N)NC(=O)[C@@H]3[C@@H]4[C@H](CN3C2=O)C4(C)C)co1] |
| 116 | 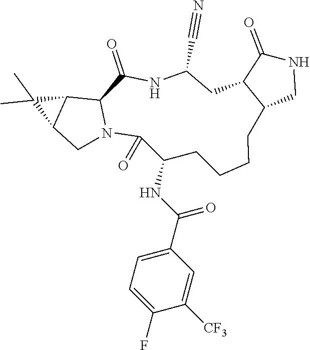 [SMILES: CC1(C)[C@@H]2[C@H]3C(=O)N[C@H](C#N)C[C@@H]4C(=O)NC[C@@H]4CCCC[C@H](NC(=O)c4ccc(F)c(C(F)(F)F)c4)C(=O)N3C[C@@H]21] |
| 117 | 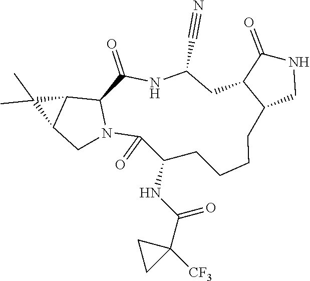 [SMILES: CC1(C)[C@@H]2[C@H]3C(=O)N[C@H](C#N)C[C@@H]4C(=O)NC[C@@H]4CCCC[C@H](NC(=O)C4(C(F)(F)F)CC4)C(=O)N3C[C@@H]21] |
| 118 | 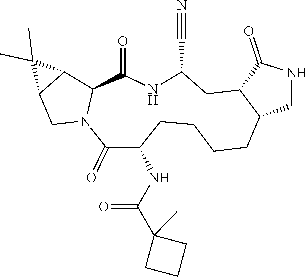 [SMILES: CC1(C(=O)N[C@H]2CCCC[C@H]3CNC(=O)[C@H]3C[C@@H](C#N)NC(=O)[C@@H]3[C@@H]4[C@H](CN3C2=O)C4(C)C)CCC1] |
| 119 | 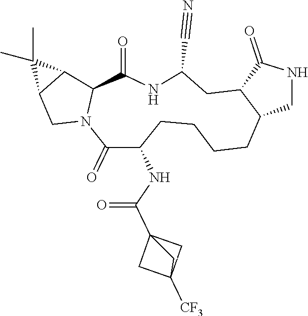 [SMILES: CC1(C)[C@@H]2[C@H]3C(=O)N[C@H](C#N)C[C@@H]4C(=O)NC[C@@H]4CCCC[C@H](NC(=O)C45CC(C(F)(F)F)(C4)C5)C(=O)N3C[C@@H]21] |
| 120 | 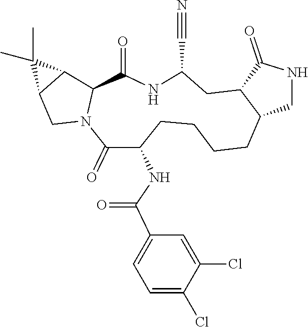 [SMILES: CC1(C)[C@@H]2[C@H]3C(=O)N[C@H](C#N)C[C@@H]4C(=O)NC[C@@H]4CCCC[C@H](NC(=O)c4ccc(Cl)c(Cl)c4)C(=O)N3C[C@@H]21] |
| 121 | 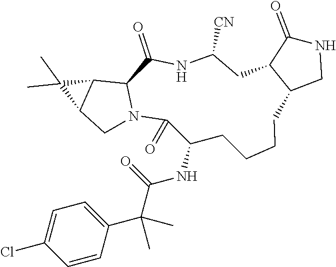 [SMILES: CC(C)(C(=O)N[C@H]1CCCC[C@H]2CNC(=O)[C@H]2C[C@@H](C#N)NC(=O)[C@@H]2[C@@H]3[C@H](CN2C1=O)C3(C)C)c1ccc(Cl)cc1] |
| 122 | 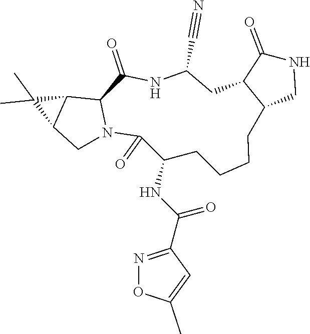 [SMILES: Cc1cc(C(=O)N[C@H]2CCCC[C@H]3CNC(=O)[C@H]3C[C@@H](C#N)NC(=O)[C@@H]3[C@@H]4[C@H](CN3C2=O)C4(C)C)no1] |
| 123 | 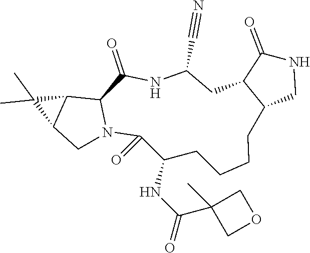 [SMILES: CC1(C(=O)N[C@H]2CCCC[C@H]3CNC(=O)[C@H]3C[C@@H](C#N)NC(=O)[C@@H]3[C@@H]4[C@H](CN3C2=O)C4(C)C)COC1] |
| 124 | 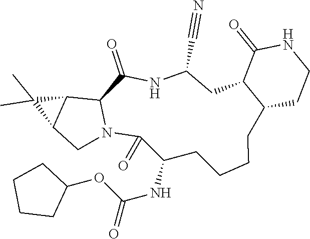 [SMILES: CC1(C)[C@@H]2[C@H]3C(=O)N[C@H](C#N)C[C@@H]4C(=O)NCC[C@@H]4CCCC[C@H](NC(=O)OC4CCCC4)C(=O)N3C[C@@H]21] |
| 125 | 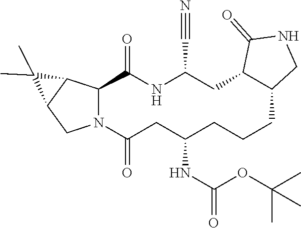 [SMILES: CC(C)(C)OC(=O)N[C@H]1CCC[C@H]2CNC(=O)[C@H]2C[C@@H](C#N)NC(=O)[C@@H]2[C@@H]3[C@H](CN2C(=O)C1)C3(C)C] |
| 126 | 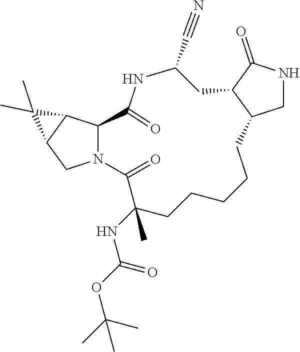 [SMILES: CC(C)(C)OC(=O)N[C@@]1(C)CCCCC[C@H]2CNC(=O)[C@H]2C[C@@H](C#N)NC(=O)[C@@H]2[C@@H]3[C@H](CN2C1=O)C3(C)C] |
| 127 | 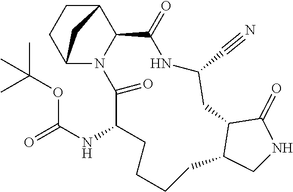 [SMILES: CC(C)(C)OC(=O)N[C@H]1CCCC[C@H]2CNC(=O)[C@H]2C[C@@H](C#N)NC(=O)[C@@H]2[C@H]3CC[C@H](C3)N2C1=O] |
| 128 | 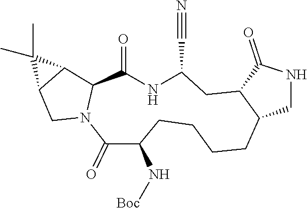 [SMILES: CC(C)(C)OC(=O)N[C@@H]1CCCC[C@H]2CNC(=O)[C@H]2C[C@@H](C#N)NC(=O)[C@@H]2[C@@H]3[C@H](CN2C1=O)C3(C)C] |
| 129 | 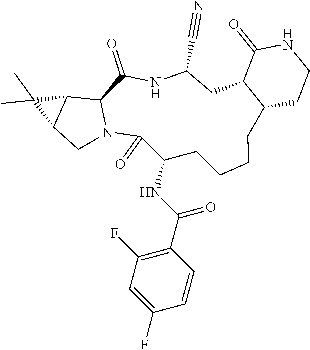 [SMILES: CC1(C)[C@@H]2[C@H]3C(=O)N[C@H](C#N)C[C@@H]4C(=O)NCC[C@@H]4CCCC[C@H](NC(=O)c4ccc(F)cc4F)C(=O)N3C[C@@H]21] |
| 130 | 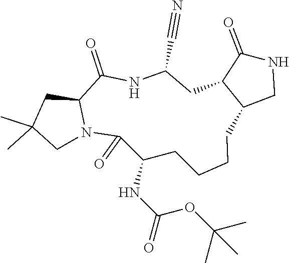 [SMILES: CC1(C)C[C@H]2C(=O)N[C@H](C#N)C[C@@H]3C(=O)NC[C@@H]3CCCC[C@H](NC(=O)OC(C)(C)C)C(=O)N2C1] |
| 131 | 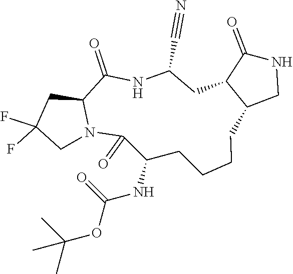 [SMILES: CC(C)(C)OC(=O)N[C@H]1CCCC[C@H]2CNC(=O)[C@H]2C[C@@H](C#N)NC(=O)[C@@H]2CC(F)(F)CN2C1=O] |
| 132 | 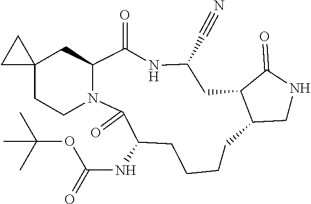 [SMILES: CC(C)(C)OC(=O)N[C@H]1CCCC[C@H]2CNC(=O)[C@H]2C[C@@H](C#N)NC(=O)[C@@H]2CC3(CCN2C1=O)CC3] |
| 133 | 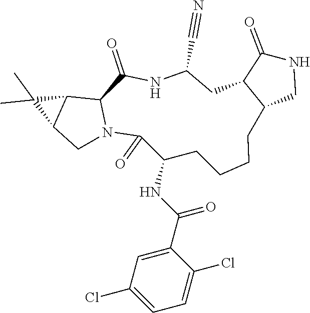 [SMILES: CC1(C)[C@@H]2[C@H]3C(=O)N[C@H](C#N)C[C@@H]4C(=O)NC[C@@H]4CCCC[C@H](NC(=O)c4cc(Cl)ccc4Cl)C(=O)N3C[C@@H]21] |
| 134 | 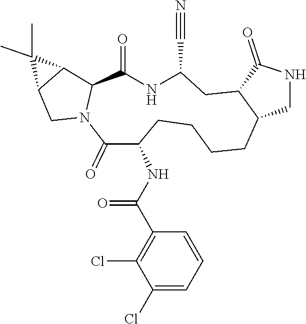 [SMILES: CC1(C)[C@@H]2[C@H]3C(=O)N[C@H](C#N)C[C@@H]4C(=O)NC[C@@H]4CCCC[C@H](NC(=O)c4cccc(Cl)c4Cl)C(=O)N3C[C@@H]21] |
| 135 | 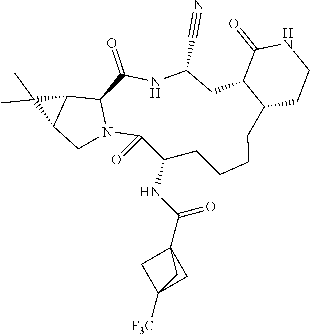 [SMILES: CC1(C)[C@@H]2[C@H]3C(=O)N[C@H](C#N)C[C@@H]4C(=O)NCC[C@@H]4CCCC[C@H](NC(=O)C45CC(C(F)(F)F)(C4)C5)C(=O)N3C[C@@H]21] |
| 136 | 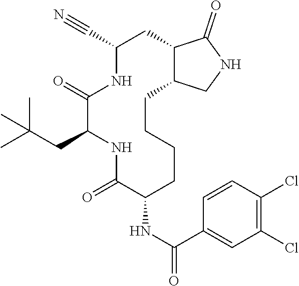 [SMILES: CC(C)(C)C[C@@H]1NC(=O)[C@@H](NC(=O)c2ccc(Cl)c(Cl)c2)CCCC[C@H]2CNC(=O)[C@H]2C[C@@H](C#N)NC1=O] |
| 137 | 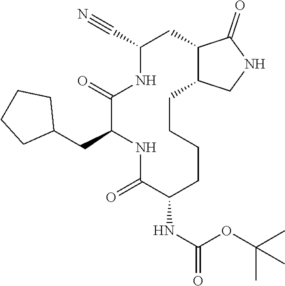 [SMILES: CC(C)(C)OC(=O)N[C@H]1CCCC[C@H]2CNC(=O)[C@H]2C[C@@H](C#N)NC(=O)[C@H](CC2CCCC2)NC1=O] |
| 138 | 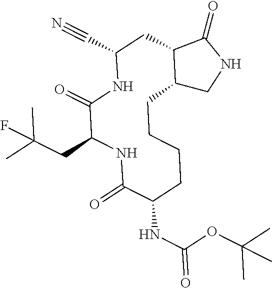 [SMILES: CC(C)(F)C[C@@H]1NC(=O)[C@@H](NC(=O)OC(C)(C)C)CCCC[C@H]2CNC(=O)[C@H]2C[C@@H](C#N)NC1=O] |
| 139 | 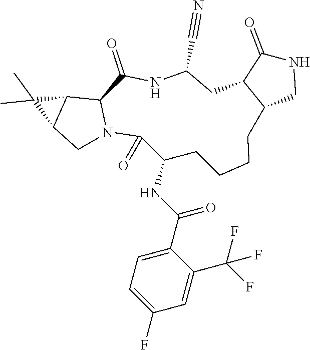 [SMILES: CC1(C)[C@@H]2[C@H]3C(=O)N[C@H](C#N)C[C@@H]4C(=O)NC[C@@H]4CCCC[C@H](NC(=O)c4ccc(F)cc4C(F)(F)F)C(=O)N3C[C@@H]21] |
| 140 | 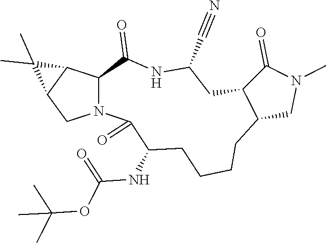 |
| 141 | 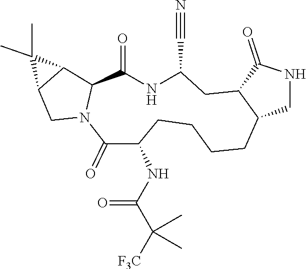 [SMILES: CC1(C)[C@@H]2[C@H]3C(=O)N[C@H](C#N)C[C@@H]4C(=O)NC[C@@H]4CCCC[C@H](NC(=O)C(C)(C)C(F)(F)F)C(=O)N3C[C@@H]21] |
| 142 | 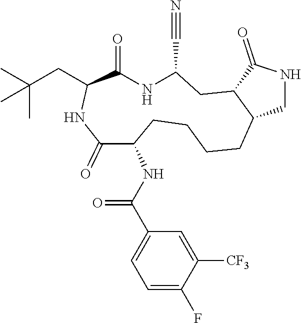 [SMILES: CC(C)(C)C[C@@H]1NC(=O)[C@@H](NC(=O)c2ccc(F)c(C(F)(F)F)c2)CCCC[C@H]2CNC(=O)[C@H]2C[C@@H](C#N)NC1=O] |
| 143 | 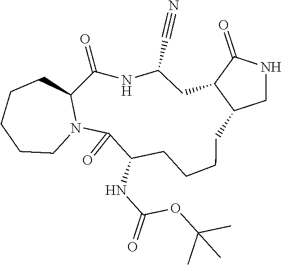 [SMILES: CC(C)(C)OC(=O)N[C@H]1CCCC[C@H]2CNC(=O)[C@H]2C[C@@H](C#N)NC(=O)[C@@H]2CCCCCN2C1=O] |
| 144 | 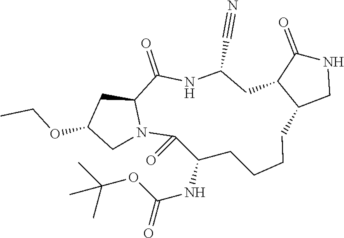 [SMILES: CCO[C@@H]1C[C@H]2C(=O)N[C@H](C#N)C[C@@H]3C(=O)NC[C@@H]3CCCC[C@H](NC(=O)OC(C)(C)C)C(=O)N2C1] |
| 145 | 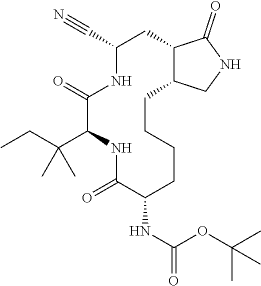 [SMILES: CCC(C)(C)[C@@H]1NC(=O)[C@@H](NC(=O)OC(C)(C)C)CCCC[C@H]2CNC(=O)[C@H]2C[C@@H](C#N)NC1=O] |
| 146 | 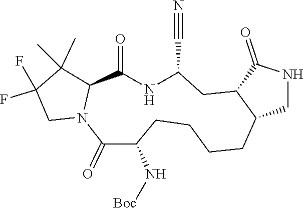 [SMILES: CC(C)(C)OC(=O)N[C@H]1CCCC[C@H]2CNC(=O)[C@H]2C[C@@H](C#N)NC(=O)[C@H]2N(CC(F)(F)C2(C)C)C1=O] |
| 147 | 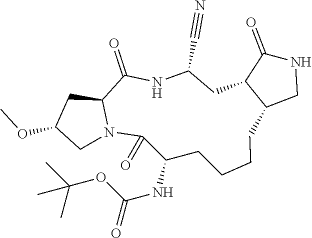 [SMILES: CO[C@@H]1C[C@H]2C(=O)N[C@H](C#N)C[C@@H]3C(=O)NC[C@@H]3CCCC[C@H](NC(=O)OC(C)(C)C)C(=O)N2C1] |
| 148 | 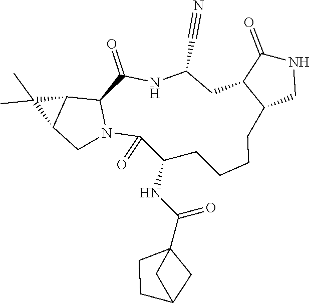 [SMILES: CC1(C)[C@@H]2[C@H]3C(=O)N[C@H](C#N)C[C@@H]4C(=O)NC[C@@H]4CCCC[C@H](NC(=O)C45CCC(C4)C5)C(=O)N3C[C@@H]21] |
| 149 | 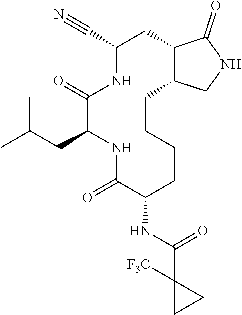 [SMILES: CC(C)C[C@@H]1NC(=O)[C@@H](NC(=O)C2(C(F)(F)F)CC2)CCCC[C@H]2CNC(=O)[C@H]2C[C@@H](C#N)NC1=O] |
| 150 | 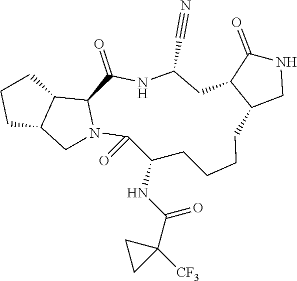 [SMILES: N#C[C@@H]1C[C@@H]2C(=O)NC[C@@H]2CCCC[C@H](NC(=O)C2(C(F)(F)F)CC2)C(=O)N2C[C@@H]3CCC[C@@H]3[C@H]2C(=O)N1] |
| 151 | 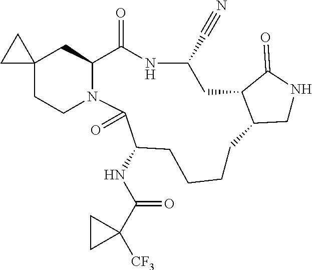 [SMILES: N#C[C@@H]1C[C@@H]2C(=O)NC[C@@H]2CCCC[C@H](NC(=O)C2(C(F)(F)F)CC2)C(=O)N2CCC3(CC3)C[C@H]2C(=O)N1] |
| 152 | 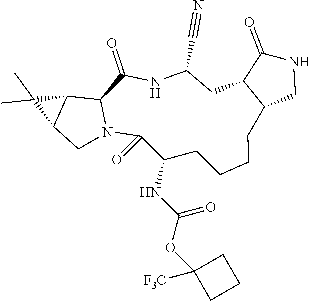 [SMILES: CC1(C)[C@@H]2[C@H]3C(=O)N[C@H](C#N)C[C@@H]4C(=O)NC[C@@H]4CCCC[C@H](NC(=O)OC4(C(F)(F)F)CCC4)C(=O)N3C[C@@H]21] |
| 153 | 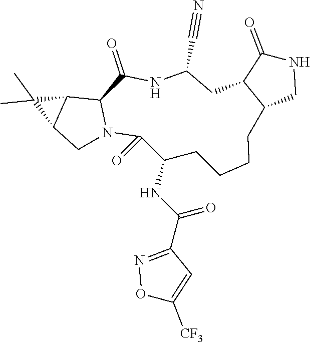 [SMILES: CC1(C)[C@@H]2[C@H]3C(=O)N[C@H](C#N)C[C@@H]4C(=O)NC[C@@H]4CCCC[C@H](NC(=O)c4cc(C(F)(F)F)on4)C(=O)N3C[C@@H]21] |
| 154 | 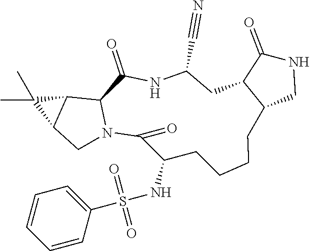 [SMILES: CC1(C)[C@@H]2[C@H]3C(=O)N[C@H](C#N)C[C@@H]4C(=O)NC[C@@H]4CCCC[C@H](NS(=O)(=O)c4ccccc4)C(=O)N3C[C@@H]21] |
| 155 | 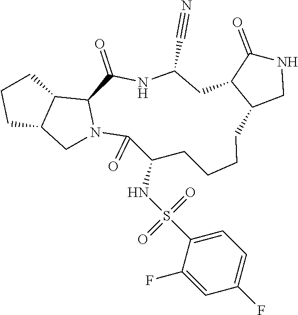 [SMILES: N#C[C@@H]1C[C@@H]2C(=O)NC[C@@H]2CCCC[C@H](NS(=O)(=O)c2ccc(F)cc2F)C(=O)N2C[C@@H]3CCC[C@@H]3[C@H]2C(=O)N1] |
| 156 | 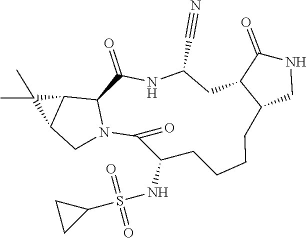 [SMILES: CC1(C)[C@@H]2[C@H]3C(=O)N[C@H](C#N)C[C@@H]4C(=O)NC[C@@H]4CCCC[C@H](NS(=O)(=O)C4CC4)C(=O)N3C[C@@H]21] |
| 157 | 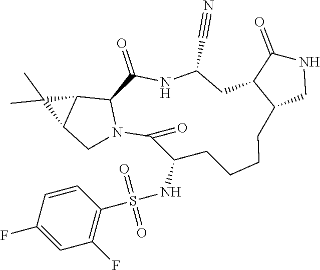 [SMILES: CC1(C)[C@@H]2[C@H]3C(=O)N[C@H](C#N)C[C@@H]4C(=O)NC[C@@H]4CCCC[C@H](NS(=O)(=O)c4ccc(F)cc4F)C(=O)N3C[C@@H]21] |
| 158 | 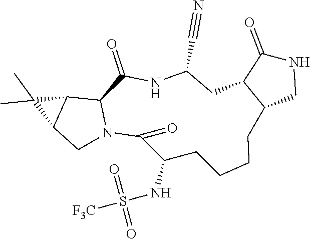 [SMILES: CC1(C)[C@@H]2[C@H]3C(=O)N[C@H](C#N)C[C@@H]4C(=O)NC[C@@H]4CCCC[C@H](NS(=O)(=O)C(F)(F)F)C(=O)N3C[C@@H]21] |
| 159 | 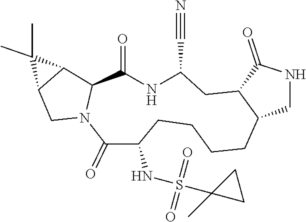 [SMILES: CC1(C)[C@@H]2[C@H]3C(=O)N[C@H](C#N)C[C@@H]4C(=O)NC[C@@H]4CCCC[C@H](NS(=O)(=O)C4(C)CC4)C(=O)N3C[C@@H]21] |
| 160 | 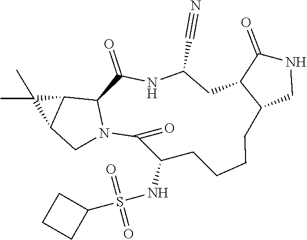 [SMILES: CC1(C)[C@@H]2[C@H]3C(=O)N[C@H](C#N)C[C@@H]4C(=O)NC[C@@H]4CCCC[C@H](NS(=O)(=O)C4CCC4)C(=O)N3C[C@@H]21] |
| 161 | 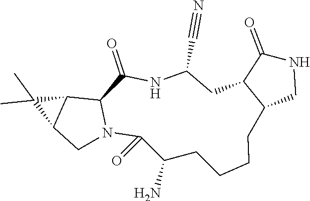 [SMILES: CC1(C)[C@@H]2[C@H]3C(=O)N[C@H](C#N)C[C@@H]4C(=O)NC[C@@H]4CCCC[C@H](N)C(=O)N3C[C@@H]21] |
| 162 | 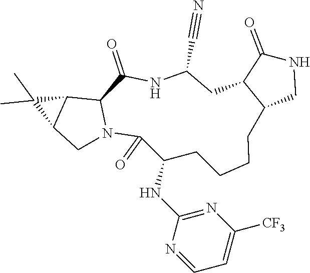 [SMILES: CC1(C)[C@@H]2[C@H]3C(=O)N[C@H](C#N)C[C@@H]4C(=O)NC[C@@H]4CCCC[C@H](Nc4nccc(C(F)(F)F)n4)C(=O)N3C[C@@H]21] |
| 163 | 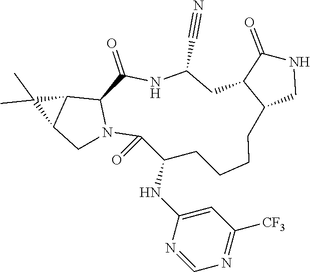 [SMILES: CC1(C)[C@@H]2[C@H]3C(=O)N[C@H](C#N)C[C@@H]4C(=O)NC[C@@H]4CCCC[C@H](Nc4cc(C(F)(F)F)ncn4)C(=O)N3C[C@@H]21] |
| 164 | 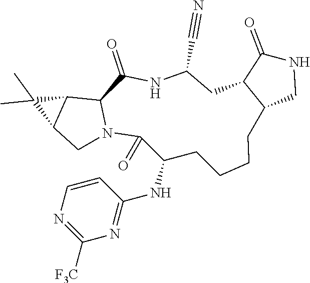 [SMILES: CC1(C)[C@@H]2[C@H]3C(=O)N[C@H](C#N)C[C@@H]4C(=O)NC[C@@H]4CCCC[C@H](Nc4ccnc(C(F)(F)F)n4)C(=O)N3C[C@@H]21] |
| 165 | 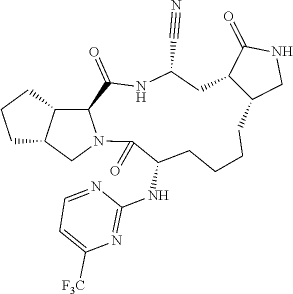 [SMILES: N#C[C@@H]1C[C@@H]2C(=O)NC[C@@H]2CCCC[C@H](Nc2nccc(C(F)(F)F)n2)C(=O)N2C[C@@H]3CCC[C@@H]3[C@H]2C(=O)N1] |
| 166 | 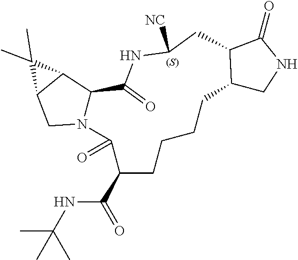 |
| 167 | 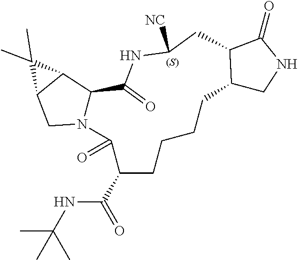 |
| 168 | 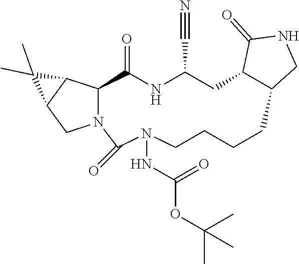 [SMILES: CC(C)(C)OC(=O)NN1CCCC[C@H]2CNC(=O)[C@H]2C[C@@H](C#N)NC(=O)[C@@H]2[C@@H]3[C@H](CN2C1=O)C3(C)C] |

In some embodiments, the compound of Formula (I) or a salt, solvate, stereoisomer or prodrug thereof is selected from:

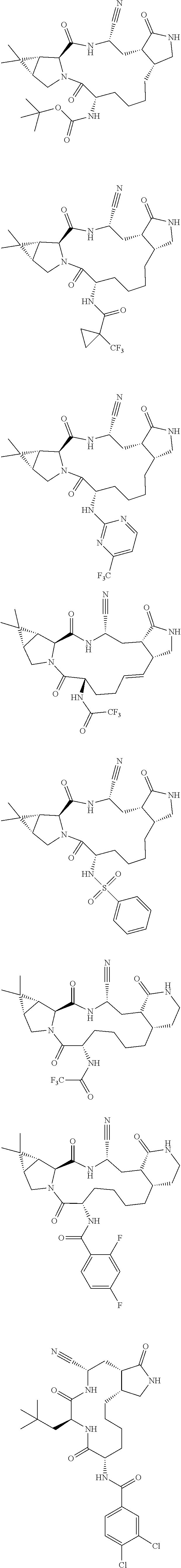

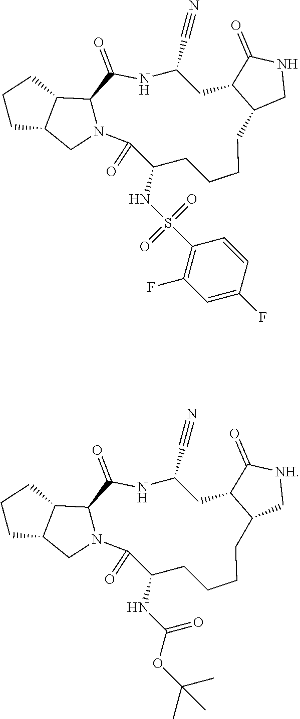 [SMILES: N#C[C@@H]1C[C@@H]2C(=O)NC[C@@H]2CCCC[C@H](NS(=O)(=O)c2ccc(F)cc2F)C(=O)N2C[C@@H]3CCC[C@@H]3[C@H]2C(=O)N1 ; CC(C)(C)OC(=O)N[C@H]1CCCC[C@H]2CNC(=O)[C@H]2C[C@@H](C#N)NC(=O)[C@@H]2[C@H]3CCC[C@H]3CN2C1=O]

The present disclosure also provides a pharmaceutical composition comprising an effective amount of a compound of Formula (I) or a pharmaceutically acceptable salt, solvate, stereoisomer or prodrug thereof, and optionally in combination with a pharmaceutically acceptable carrier, excipient or diluent.

The compound of the invention can be administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will be appreciated that any compound that is a prodrug of the compound of formula (I) is also within the scope and spirit of the invention. Thus the compound of the invention can be administered to a subject in the form of a pharmaceutically acceptable pro-drug. The term “pro-drug” is used in its broadest sense and encompasses those derivatives that are converted in vivo to the compound of the invention. Such derivatives would readily occur to those skilled in the art. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers). For example, amino moieties may be protected. Oxy groups may also be esterified.

The compound of the invention may be in crystalline form either as the free compound or as a solvate (e.g. hydrate) and it is intended that both forms are within the scope of the present invention. Methods of solvation are generally known within the art.

The compound of the invention, or a pharmaceutically acceptable salt, solvate or prodrug thereof is administered to the patient in a therapeutically effective amount. As used herein, a therapeutically effective amount is intended to include at least partially attaining the desired effect, or delaying the onset of, or inhibiting the progression of, or halting or reversing altogether the onset or progression of macular degeneration.

As used herein, the term “effective amount” relates to an amount of compound which, when administered according to a desired dosing regimen, provides the desired therapeutic activity. Dosing may occur at intervals of minutes, hours, days, weeks, months or years or continuously over any one of these periods. Suitable dosages may lie within the range of about 0.1 ng per kg of body weight to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

Suitable dosage amounts and dosing regimens can be determined by the attending physician and may depend on the severity of the condition as well as the general age, health and weight of the patient to be treated.

The compound of the invention may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Modes of administration including topical or intravenous administration may be possible. For example, solutions or suspensions of the compound, composition or combinations of the invention may be formulated. Topical application typically involves administering the compound of the invention in an amount between 0.1 ng and 10 mg.

The compound or composition of the invention may also be suitable for intravenous administration. For example, a compound of formula (I) or a pharmaceutically acceptable salt, solvate or prodrug thereof may be administered intravenously at a dose of up to 16 mg/m².

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

The compound or composition of the invention may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste. In another embodiment, the compound of formula (I) or a pharmaceutically acceptable salt, solvate or prodrug is orally administerable.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The compound or composition of the invention may be suitable for topical administration in the mouth including lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The compound or composition of the invention may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The compound or composition of the invention may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage composition or combinations are those containing a daily dose or unit, daily sub-dose, as herein above described, or an appropriate fraction thereof, of the active ingredient.

It should be understood that in addition to the active ingredients particularly mentioned above, the composition of this invention may include other agents conventional in the art having regard to the type of composition or combination in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

A person skilled in the art will appreciate that other means for injecting and/or administering the compound, composition or combinations can also be used. These other means can include, for example, medical delivery devices. These devices and methods can include, for example, biodegradable polymer delivery members that are inserted for long term delivery of medicaments.

The present disclosure provides a method of fabricating a compound of Formula (I), comprising:
i) reacting a compound of Formula (II) with a compound of Formula (III) under dehydrating conditions in order to form an amide bond:

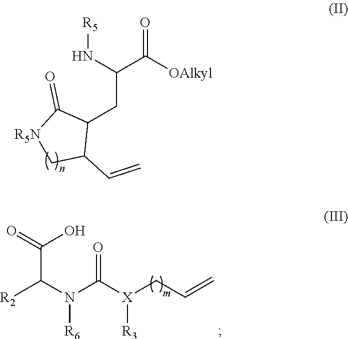

ii) performing a ring closing olefin metathesis reaction in order to form a compound of Formula (IV):

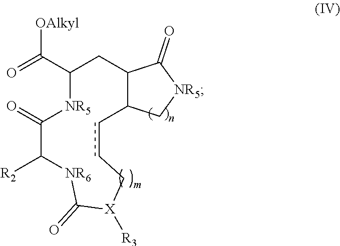

and
iii) converting the alkyloxyacyl moiety in Formula (IV) into a R₁ moiety in order to form a compound of Formula (I).

In some embodiments, the amide bond is formed via an activated ester intermediate. In some embodiments, the amide bond is formed in the presence of HATU and a base. The base may be N,N-diisopropylethylamine.

In some embodiments, the olefin metathesis reaction is performed in the presence of Grubbs (II) catalyst.

In some embodiments, step ii) further comprises performing a hydrogenation reaction after the olefin metathesis reaction. The hydrogenation reaction may be performed using palladium on carbon in the presence of hydrogen.

In some embodiments, the alkyloxyacyl moiety is converted to a primary amide and reacting the primary amide with Burgess reagent.

In some embodiments, the alkyloxyacyl moiety is converted to an acylalkyl moiety in the presence of a reducing agent and Dess-Martin periodinane.

In some embodiments, the method further comprises a step of synthesising the compound of Formula (II), comprising:
a) reacting a compound of Formula (V) with a base in order to form a compound of Formula (VI)

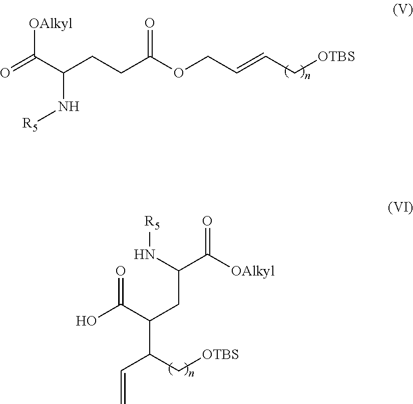

c) reacting the compound of Formula (VI) with phthalimide in order to form a compound of Formula (VII)

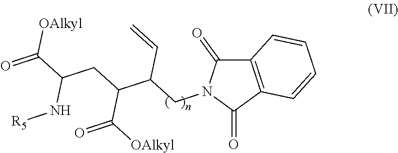

d) reacting the compound of Formula (VI) with methylamine hydrochloride in order to form a compound of Formula (II).

The present disclosure provides a method of treating a coronavirus related disease or condition in a patient in need thereof, the method comprising administering to the patient a therapeutically effective amount of a compound of formula (I) or a pharmaceutically acceptable salt, solvate, stereoisomer or prodrug thereof.

The present disclosure provides a use of a compound of formula (I) or a pharmaceutically acceptable salt, solvate, stereoisomer or prodrug thereof in the manufacture of a medicament for treating a coronavirus related disease or condition in a patient in need thereof.

The present disclosure provides a compound of formula (I) or a pharmaceutically acceptable salt, solvate, stereoisomer or prodrug thereof for use in treating a coronavirus related disease or condition in a patient in need thereof.

In some embodiments, the compound of Formula (I) is a 3CLpro inhibitor. In contrast, PLpro recognizes Gly-Gly at P2-P1.

In some embodiments, the coronavirus related disease or condition is caused by a virus selected from SARS-CoV-1, SARS-CoV-2, MERS-CoV, CoV-229E, and HCoV-OC43. Coronavirus may include the four coronavirus genera: alpha, beta, gamma or delta coronavirus. It may be one which affects human, such as but is not limited to, coronavirus 229E, coronavirus NL63, coronavirus OC43, or coronavirus HKU1. SARS-CoV-2 encompasses the initially discovered virus strain in China as well as variants which emerge later, such as but not limited to, B.1.1.7 (Alpha), B.1.351 (Beta), P.1 (Gamma), B.1.617.2 (Delta), B.1.427/B.1.429 (Epsilon), P.2 (Zeta), B.1.525 (Eta), P.3 (Theta), B.1.526 (Iota), B.1.617.1 (Kappa), C.37 (Lambda), B.1.621 (Mu), B.1.1.529 (Omicron) and Omicron variants: BA.1, BA.2, BA.2 subvariants such as XBB including XBB.1.16 (Arcturus) and XE, BA.3, BA.4, BA.5 and BA.5 subvariants such as BQ.1 and BQ.1.1.

In some embodiments, the compound of Formula (I) is characterised by a protease inhibition assay IC₅₀ value of less than 20 μM. In some embodiments, the compound of Formula (I) is characterised by an SARS CoV-2 IC₅₀ value of less than 20 μM. In some embodiments, the compound of Formula (I) is characterised by a 229E IC₅₀ value of less than 20 μM. In some embodiments, the compound of Formula (I) is characterised by a MERS IC₅₀ value of less than 20 μM.

In some embodiments, the compound of Formula (I) is characterised by a viral cytopathic effect assay EC₅₀ value of less than 20 μM. In some embodiments, the compound of Formula (I) is characterised by a 229E EC₅₀ value of less than 20 μM. In some embodiments, the compound of Formula (I) is characterised by a OC43 EC₅₀ value of less than 20 μM. In some embodiments, the compound of Formula (I) is characterised by an SARS CoV-2EC₅₀ value of less than 20 μM. In some embodiments, the compound of Formula (I) is characterised by a MERS EC₅₀ value of less than 20 μM.

### EXAMPLES

### Experimental Procedures

All reactions were carried out under continuous stirring under an atmosphere of nitrogen gas unless otherwise stated. When appropriate, reaction apparatus were dried in an oven at 100° C. for 16 hours prior to use, and anhydrous solvents (Sure-Seal™ products from Aldrich Chemical Company, Milwaukee, Wisconsin) were employed. Otherwise states, solvents and reagents were purchased from commercial sources and used without further purification. Acetonitrile was purchased from VWR (Germany). Deuterated solvents were purchased from Cambridge Isotope Laboratories (USA).

Reaction progress was monitored using thin-layer chromatography (TLC), and/or liquid chromatography-mass spectrometry (LCMS) analysis. TLC was performed on precoated silica gel plates with a fluorescence indicator (254 nm excitation wavelength) and visualized under UV light and/or with KMnO₄, phosphomolybdic acid, and/or cerium ammonium molybdate stains. LCMS data were recorded on an Agilent 1290 Infinity series, single quad spectrometer (ESI ion source) using acetonitrile/water or methanol/water gradients and either trifluoroacetic acid or formic acid modifiers. Flash chromatography was performed on an automated system (Teledyne Isco Combiflash RF200) using a silica column (RediSep RF, Silica 230-400 Mesh, 60 Å average pore size. Catalog #69-2203-312). Crude target inhibitors were purified using a reverse-phase C18 column (Phenomenex Luna 5 μm C18(2), 100×4.6 mm, 100 Å) on a high performance liquid chromatography (HPLC) system with an ultraviolet detector. The mobile phase consisted of water and acetonitrile with 0.1% formic acid unless otherwise stated.

NMR spectra were recorded on a 400 MHz spectrometer (Bruker Ascend 400, Germany). Chemical shifts were expressed as parts per million (ppm) relative to the solvent peak. The peak shapes are described as follows: s, singlet; d, doublet; t, triplet; q, quartet; quin, quintet; m, multiplet; br s, broad singlet; 30 app, apparent. The compounds' purities were 295% as determined by a HPLC instrument (Shimadzu) using acetonitrile/water (with 0.1% formic acid) as eluent.

### General Route 1

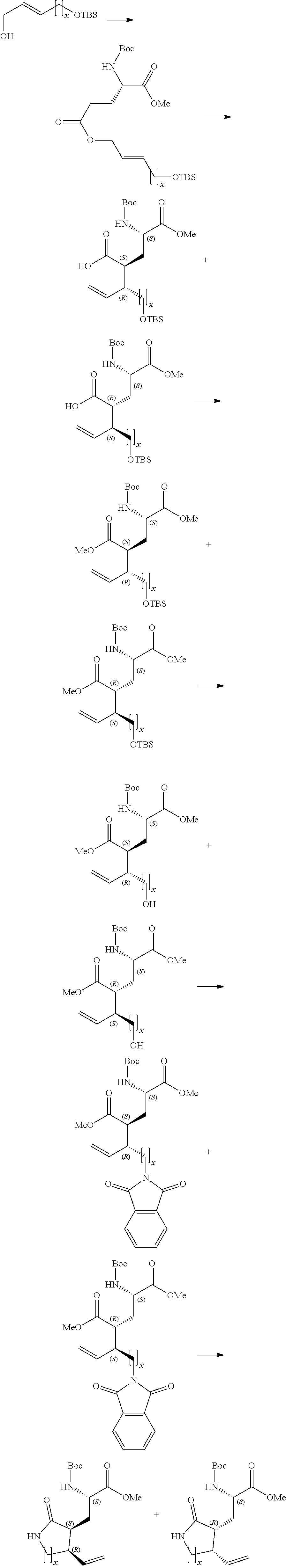

### Example 1

### Exemplification of General Route 1

### methyl (S)-2-((tert-butoxycarbonyl)amino)-3-((3S,4R)-2-oxo-4-vinylpyrrolidin-3-yl)propanoate

### and

### methyl (S)-2-((tert-butoxycarbonyl)amino)-3-((3R,4S)-2-oxo-4-vinylpyrrolidin-3-yl)propanoate

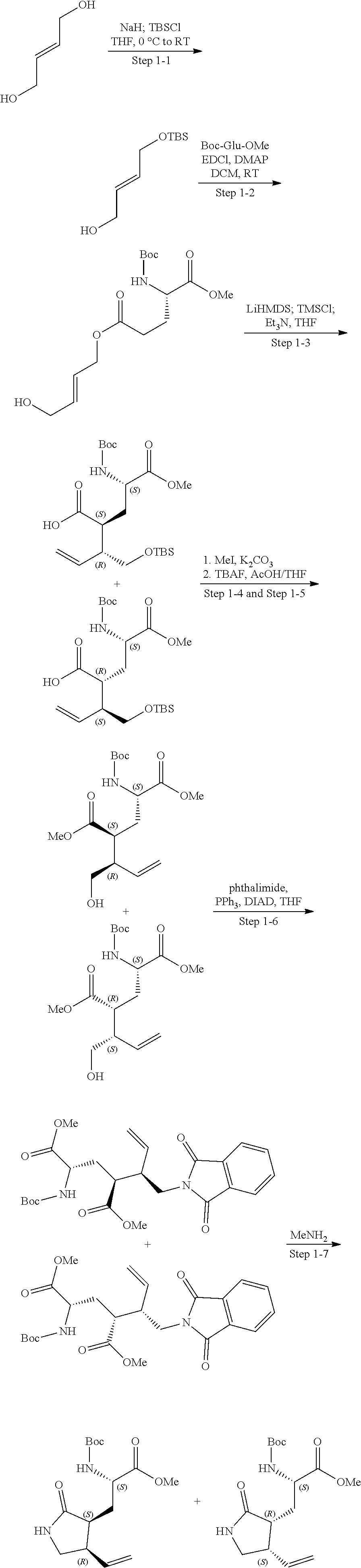

### Step 1-1. (E)-4-((tert-butyldimethylsilyl)oxy)but-2-en-1-ol

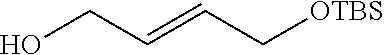 [SMILES: CC(C)(C)[Si](C)(C)OC/C=C/CO]

A solution of 2-butene-1,4-diol (trans) (100 g, 11.34 mol, 1 equiv) In THF (2200 ml) was added NaH (54 g, 60 wt %) in portions at 0° C. under nitrogen atmosphere and stirred for 4 h at room temperature. Then TBDMSCl (171 g, 11.34 mol, 1.00 equiv) was added dropwise at 0° C. The resulting mixture was stirred for 12 h at room temperature.

After complete reaction as assessed by TLC (20% EtOAc/Hexanes, stained with KMnO₄), the reaction was quenched by the slow addition of saturated aqueous sodium bicarbonate (2000 mL). The resulting mixture was extracted with EtOAc (3×2000 mL), dried over anhydrous sodium sulfate. After filtration, the filtrate was concentrated under reduced pressure. The residue was purified by silica gel column chromatography, eluted with ethyl acetate:hexanes (1:10) to afford (2E)-4-[(tert-butyldimethylsilyl)oxy]but-2-en-1-ol (155 g, 64%) as a colorless oil.

### Step 1-2. (E)-5-(4-((tert-butyldimethylsilyl)oxy)but-2-en-1-yl) 1-methyl (tert-butoxycarbonyl)-L-glutamate

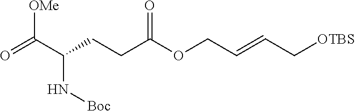 [SMILES: COC(=O)[C@H](CCC(=O)OC/C=C/CO[Si](C)(C)C(C)(C)C)NC(=O)OC(C)(C)C]

A solution of (2E)-4-[(tert-butyldimethylsilyl)oxy]but-2-en-1-ol (155 g, 7.65 mol, 1 equiv) in CH₂Cl₂ (3000 ml) was added Boc-L-glutamic acid α-methyl ester (199.91 g, 7.65 mol, 1 equiv), N-Ethyl-N′-(3-dimethylaminopropyl)carbodiimide hydrochloride (220.24 g, 11.48 mol, 1.5 equiv) and DMAP (46.79 g, 3.82 mmol, 0.5 equiv) at 0° C. under nitrogen atmosphere and stirred for 12 h at room temperature. Starting material was completely consumed as checked by LC/MS. The reaction was quenched by the addition of sat. ammonium chloride (2000 ml). The resulting mixture was extracted with CH₂Cl₂ (3×3000 mL), dried over anhydrous sodium sulfate. After filtration, the filtrate was concentrated under reduced pressure. The residue was purified by silica gel column chromatography, eluted with ethyl acetate:hexanes (1:5) to afford 5-(2E)-4-[(tert-butyldimethylsilyl)oxy]but-2-en-1-yl 1-methyl (2S)-2-[(tert-butoxycarbonyl)amino]pentanedioate (310 g, 82%) as a colorless oil. MS (ESI) m/z 446 [C₁₂H₃₉NO₇Si+H]⁺.

### Step 1-3. Synthesis of (2S,3R)-2-((S)-2-((tert-butoxycarbonyl)amino)-3-methoxy-3-oxopropyl)-3-(((tert-butyldimethylsilyl)oxy)methyl)pent-4-enoic acid and (2R,3S)-2-((S)-2-((tert-butoxycarbonyl)amino)-3-methoxy-3-oxopropyl)-3-(((tert-butyldimethylsilyl)oxy)methyl)pent-4-enoic acid

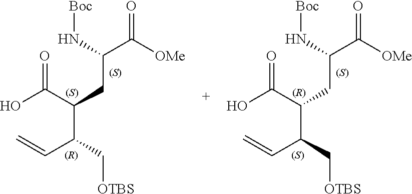

A solution of 5-(2E)-4-[(tert-butyldimethylsilyl)oxy]but-2-en-1-yl 1-methyl (2S)-2-[(tert-butoxycarbonyl)amino]pentanedioate (310 g, 6.97 mol, 1 equiv) in anhydrous THF (3800 ml) was treated with LiHMDS (2089 ml, 1 M in THF, 2.1 mol) for 60 min at −78° C. under nitrogen atmosphere. Freshly distilled TMSCl (264.5 g, 24.5 mol, 3.5 equiv) was then added dropwise at −78° C. The resulting mixture was stirred for 1 h at −78° C. Then, anhydrous triethlyamine (246.4 g, 24.3 mol, 3.5 equiv) was added dropwise at −78° C. The resulting mixture was stirred for 12 h at room temperature. The reaction was quenched by the addition of saturated ammonium chloride (2000 ml). The mixture was adjusted to pH=3 with 2 M HCl. The resulting mixture was extracted with EtOAc (3×3000 mL), dried over anhydrous sodium sulfate. After filtration, the filtrate was concentrated under reduced pressure. The residue was purified by silica gel column chromatography, eluted with ethyl acetate:hexanes (1:1) to afford 2-[(2S)-2-[(tert-butoxycarbonyl)amino]-3-methoxy-3-oxopropyl]-3-{[(tert-butyldimethylsilyl)oxy]methyl}pent-4-enoic acid (290 g, 93%) as a light yellow oil. MS (ESI) m/z 446 [C₁₂H₃₉NO₇Si+H]⁺.

### Step 1-4. Synthesis of dimethyl (2S,4S)-2-((tert-butoxycarbonyl)amino)-4-((R)-1-((tert-butyldimethylsilyl)oxy)but-3-en-2-yl)pentanedioate and dimethyl (2S,4R)-2-((tert-butoxycarbonyl)amino)-4-((S)-1-((tert-butyldimethylsilyl)oxy)but-3-en-2-yl)pentanedioate

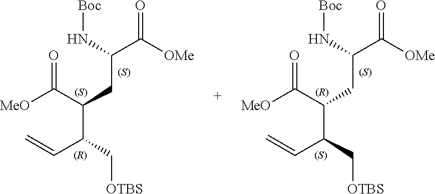

A solution of 2-[(2S)-2-[(tert-butoxycarbonyl)amino]-3-methoxy-3-oxopropyl]-3-{[(tert-butyldimethylsilyl)oxy]methyl}pent-4-enoic acid (290 g, 6.5 mol, 1 equiv) in anhydrous DMF (1500 ml) was treated with potassium carbonate (179.9 g, 13.01 mol, 2 equiv) for 30 min at 0° C. under nitrogen atmosphere followed by addition of iodomethane (184.7 g, 13.01 mol, 2 equiv) dropwise 0° C. The resulting mixture was stirred for 3 h at room temperature under nitrogen atmosphere. The reaction was quenched by the addition of water (1500 ml). The resulting mixture was extracted with EtOAc (3×2000 mL), dried over anhydrous sodium sulfate. After filtration, the filtrate was concentrated under reduced pressure. The residue was purified by silica gel column chromatography, eluted with ethyl acetate:hexanes (1:10) to afford 1,5-dimethyl (2S)-2-[(tert-butoxycarbonyl)amino]-4-{1-[(tert-butyldimethylsilyl)oxy]but-3-en-2-yl}pentanedioate (265 g, 88%) as a colorless oil. MS (ESI) m/z 460 [C₂₂H₄₁NO₇Si M+H]⁺.

### Step 1-5. Synthesis of dimethyl (2S,4S)-2-((tert-butoxycarbonyl)amino)-4-((R)-1-hydroxybut-3-en-2-yl)pentanedioate and dimethyl (2S,4R)-2-((tert-butoxycarbonyl)amino)-4-((S)-1-hydroxybut-3-en-2-yl)pentanedioate

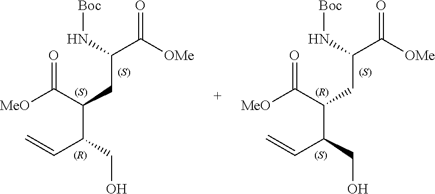

A solution of anhydrous TBAF (3460 mL, 1 M in THF) was mixed with glacial acetic acid (436 mL) at 0° C. under nitrogen atmosphere. To the above mixture was added 1,5-dimethyl (2S)-2-[(tert-butoxycarbonyl)amino]-4-{1-[(tert-butyldimethylsilyl)oxy]but-3-en-2-yl}pentanedioate (265 g, 5.76 mol, 1 equiv) dropwise for 60 min at −5° C. The resulting mixture was stirred for additional 12 h at 0° C. The mixture basified to pH=7 with saturated sodium bicarbonate solution. The resulting mixture was extracted with EtOAc (3×3000 mL), dried over anhydrous sodium sulfate. After filtration, the filtrate was concentrated under reduced pressure. The residue was purified by silica gel column chromatography, eluted with ethyl acetate:hexanes (2:1) to afford 1,5-dimethyl (2S)-2-[(tert-butoxycarbonyl)amino]-4-(1-hydroxybut-3-en-2-yl)pentanedioate (168 g, 84%) as a colorless oil. MS (ESI) m/z 346 [C₁₆H₂₇NO₇+H]⁺.

### Step 1-6. Synthesis of dimethyl (2S,4S)-2-((tert-butoxycarbonyl)amino)-4-((R)-1-(1,3-dioxoisoindolin-2-yl)but-3-en-2-yl)pentanedioate and dimethyl (2S,4R)-2-((tert-butoxycarbonyl)amino)-4-((S)-1-(1,3-dioxoisoindolin-2-yl)but-3-en-2-yl)pentanedioate

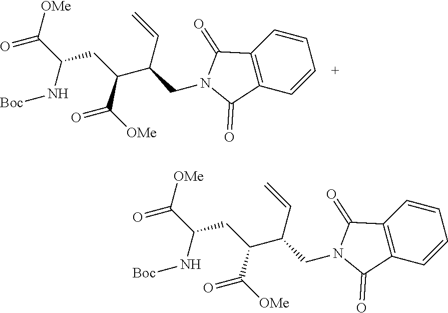 [SMILES: C=C[C@@H](CN1C(=O)c2ccccc2C1=O)[C@H](C[C@H](NC(=O)OC(C)(C)C)C(=O)OC)C(=O)OC ; C=C[C@H](CN1C(=O)c2ccccc2C1=O)[C@@H](C[C@H](NC(=O)OC(C)(C)C)C(=O)OC)C(=O)OC]

A solution of 1,5-dimethyl (2S)-2-[(tert-butoxycarbonyl)amino]-4-(1-hydroxybut-3-en-2-yl)pentanedioate (265 g, 7.67 mol, 1 equiv) in THE (2600 ml) was treated with 2,3-dihydro-1H-isoindole-1,3-dione (226 g, 1.53 mmol, 2 equiv) and triphenylphosphine (302 g, 1.15 mmol, 1.5 equiv) for 30 min at 0° C. under nitrogen atmosphere. DIAD (240 g, 11.9 mol, 1.55 equiv) was then dropwise at 0° C. The resulting mixture was stirred for 3 h at room temperature under nitrogen atmosphere. The reaction was quenched by the addition of saturated ammonium chloride (3000 mL) solution at room temperature. The resulting mixture was extracted with EtOAc (3×3000 mL) and dried over anhydrous sodium sulfate. After filtration, the filtrate was concentrated under reduced pressure. The residue was purified by silica gel column chromatography, eluted with ethyl acetate:hexanes (1:1) to afford 1,5-dimethyl (2S)-2-[(tert-butoxycarbonyl)amino]-4-[1-(1,3-dioxoisoindol-2-yl)but-3-en-2-yl]pentanedioate (405 g, crude) as a yellow solid. MS (ESI) m/z 475 [C₂₄H₃₀N₂O₈+H]⁺.

### Step 1-7. Synthesis of methyl (S)-2-((tert-butoxycarbonyl)amino)-3-((3R,4S)-2-oxo-4-vinylpyrrolidin-3-yl)propanoate and methyl (S)-2-((tert-butoxycarbonyl)amino)-3-((3S,4R)-2-oxo-4-vinylpyrrolidin-3-yl)propanoate

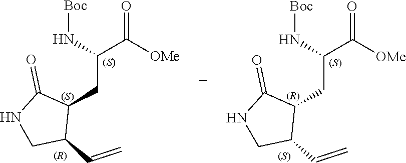

A solution of 1,5-dimethyl (2S)-2-[(tert-butoxycarbonyl)amino]-4-[1-(1,3-dioxoisoindol-2-yl)but-3-en-2-yl]pentanedioate (405 g, crude) in MeOH (4000 ml) was treated with methylamine hydrochloride (170 g, 25.2 mol) and N,N-diisopropylethylamine (320 g, 24.8 mol). The resulting mixture was stirred for 3 h at 80° C. under nitrogen atmosphere. The mixture was allowed to cool down to room temperature. The resulting mixture was concentrated under reduced pressure. The reaction was quenched with sat. ammonium chloride (3000 ml) solution. The resulting mixture was extracted with EtOAc (3×3000 mL), dried over anhydrous sodium sulfate. After filtration, the filtrate was concentrated under reduced pressure. The residue was purified by silica gel column chromatography, eluted with ethyl acetate/hexanes (2:1) to afford methyl (S)-2-((tert-butoxycarbonyl)amino)-3-((3S,4R)-2-oxo-4-vinylpyrrolidin-3-yl)propanoate (115 g) as a yellow oil. LCMS (ESI) m/z 313 [C₁₅H₂₄N₂O₅+H]⁺.

The material was treated with MTBE (3 times) to remove triphenylphosphine oxide (about 35 g) and then silica gel column two times, eluting with dichloromethane/ethyl acetate (1:1) to afford 65 g of crude product.

The crude product (300 mg batches) was purified by Prep-Chiral-SFC with the following conditions,

Column: CHIRALPAK IH, 3×25 cm, 5 um; Mobile Phase A: CO2, Mobile Phase B: MEOH(0.1% 2M NH3-MEOH); Flow rate: 100 mL/min; Gradient: isocratic 15% B; Column Temperature(° C.): 35; Back Pressure (bar): 100; Wave Length: 210 nm; RT1: 3.8 min; RT: 8.652 min; Sample Solvent: MeOH—Preparative; Injection Volume: 1 mL.

Isomer 1 was isolated as an off white solid (73 mg), ¹H NMR (CDCl₃, 400 MHz) δ (ppm): 6.12 (s, 1H), 5.87-5.77 (m, 2H), 5.23-5.19 (m, 2H), 4.46 (s, 1H), 3.76-3.74 (d, J=7.2 Hz, 3H), 3.59 (s, 1H), 3.18-3.10 (m, 2H), 2.62 (s, 1H), 2.12-2.08 (m, 1H), 1.84-1.80 (m, 1H), 1.46 (s, 9H). LCMS (ES, m/z): 313 [M+H]⁺.

Isomer 2 was isolated as an off white solid (47.5 mg), ¹H NMR (CDCl₃, 400 MHz) δ (ppm): 6.32 (s, 1H), 5.85-5.76 (m, 1H), 5.69-5.67 (m, 1H), 5.30-5.20 (m, 2H), 4.32-4.31 (m, 1H), 3.75-3.73 (d, J=6.8 Hz, 3H), 3.59-3.56 (m, 1H), 3.24-3.14 (m, 2H), 2.63-2.58 (m, 1H), 1.94-1.90 (m, 2H), 1.45 (s, 9H). LCMS (ES, m/z): 313 [M+H]⁺.

### General Route 2

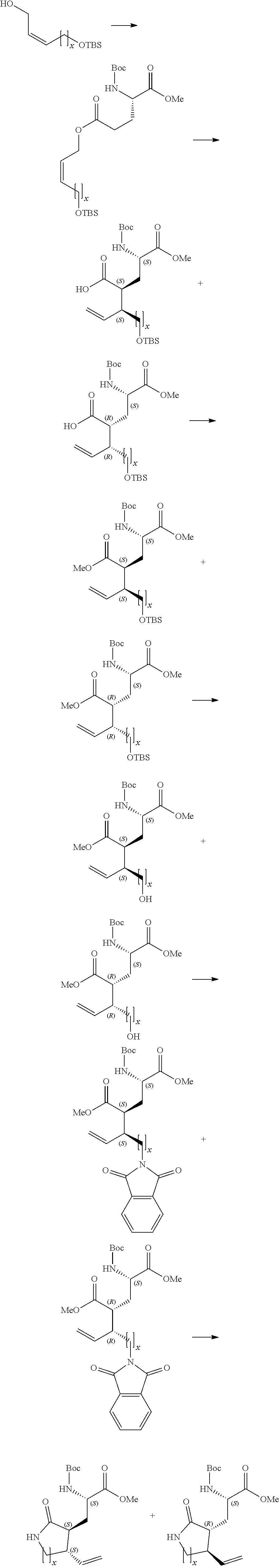

### Example 2

### Specific Exemplification of General Route 2

### Methyl (S)-2-((tert-butoxycarbonyl)amino)-3-((3S,4S)-2-oxo-4-vinylpyrrolidin-3-yl)propanoate

### and

### Methyl (S)-2-((tert-butoxycarbonyl)amino)-3-((3R,4R)-2-oxo-4-vinylpyrrolidin-3-yl)propanoate

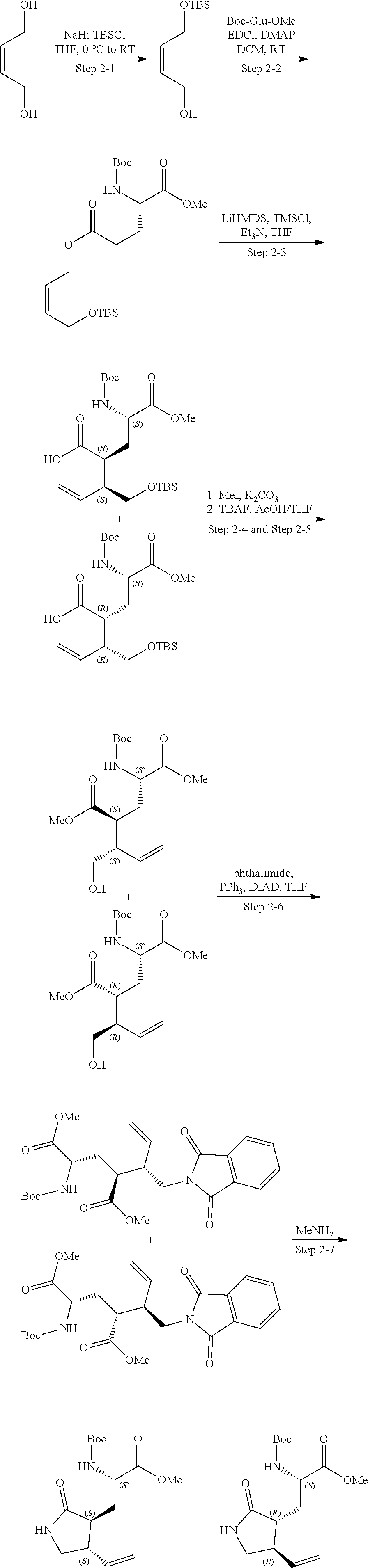

### Step 2-1. Synthesis of (Z)-4-((tert-butyldimethylsilyl)oxy)but-2-en-1-ol

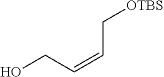 [SMILES: CC(C)(C)[Si](C)(C)OC/C=C\CO]

NaH (60 wt %, 3.0 g, 75 mmol) was suspended in anhydrous THF (125 mL) under N₂ and cooled to 0° C. Cis-but-2-ene-1,4-diol (6.67 g, 75 mmol, 1.0 equiv) was added in one portion, maintaining the temperature <5° C. The stirring was continued for 15 min at 0° C. and the reaction was warmed to room temperature and stirred 4 h forming a thick white slurry. The reaction was recooled to 0° C., after which TBSCl (11.41 g, 75 mmol, 1.0 equiv) was added in one portion and stirred 15 min at this temperature before warming to room temperature over 30 min and stirred an additional 4 h. After complete reaction as assessed by TLC (20% EtOAc/Hexanes, stained with KMnO₄), the reaction was quenched by the slow addition of saturated aqueous sodium bicarbonate (150 mL).

The crude reaction was diluted with Et₂O (100 mL). The organic phase was removed, and the resulting aqueous phase extracted with Et₂O (3×50 mL). The combined organics were washed with brine, dried with anhydrous sodium sulfate, and concentrated in vacuo to give a thick pale yellow oil. Purification was done using flash chromatography over silica, with product eluting at ethyl acetate: hexanes (4:1) and concentrated to yield desired product as a clear colorless oil (11.54 g, 81.8%).

### Step 2-2. Synthesis of (Z)-5-(4-((tert-butyldimethylsilyl)oxy)but-2-en-1-yl) 1-methyl (tert-butoxycarbonyl)-L-glutamate

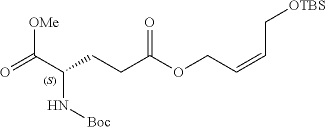

A mixture of (S)-4-((tert-Butoxycarbonyl)amino)-5-methoxy-5-oxopentanoic acid (13.22 g, 50.6 mmol, 1.0 equiv), cis-2-butene-1,4-diol mono TBS ester (10.24 g, 50.6 mmol, 1.0 equiv), and N-ethyl-N′-(3-dimethylaminopropyl)carbodiimide hydrochloride (19.4 g, 101.2 mmol, 1.5 equiv) was dissolved in anhydrous DCM (300 mL). 4-Dimethylaminopyridine (3 g, 25.3 mmol, 0.5 equiv) was added in 1 portion and stirred at room temperature for 12 h. Starting material was completely consumed as checked by LC/MS. The reaction was diluted with saturated aqueous ammonium chloride (200 mL) and extracted with CH₂Cl₂ (5×60 ml). The organic extracts were then dried over anhydrous sodium sulfate, filtered, and the volatiles were removed in vacuo to yield the crude product as a colorless oil. Purification was carried out by column chromatography over silica with product eluting at ethyl acetate:hexanes (3:7), and concentrated to yield the desired product as a colorless oil (20.3 g, 90%). MS (ESI) m/z 346.2 [M-Boc+H]⁺.

### Step 2-3. Synthesis of (2S,3S)-2-((S)-2-((tert-butoxycarbonyl)amino)-3-methoxy-3-oxopropyl)-3-(((tert-butyldimethylsilyl)oxy)methyl)pent-4-enoic acid and (2R,3R)-2-((S)-2-((tert-butoxycarbonyl)amino)-3-methoxy-3-oxopropyl)-3-(((tert-butyldimethylsilyl)oxy)methyl)pent-4-enoic acid

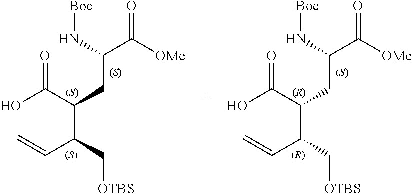

5-(2Z)-4-[(tert-butyldimethylsilyl)oxy]but-2-en-1-yl 1-methyl (2S)-2-[(tert-butoxycarbonyl)amino]pentanedioate (20.0 g, 44.9 mmol, 1.0 equiv.) was dissolved in anhydrous THF (250 mL) and cooled to −78° C. under N₂. LiHMDS (100 mL, 1 M solution in THF, 100 mmol, 2.1 equiv.) was added dropwise and the pale orange reaction mixture allowed to stir at −78° C. for 1 h. Anhydrous TMSCl (17.1 mL, 134.6 mmol, 3.0 equiv., freshly distilled from CaH₂) was then added, and the reaction stirred an additional 30 min at −78° C., followed by addition of anhydrous triethylamine (18.8 mL, 134.6 mmol, 3.0 equiv.). The reaction was warmed to room temperature and stirred overnight. After overnight stirring, the reaction was warmed to 50° C. for 4 h. The reaction was cooled to room temperature and quenched by addition of saturated aqueous ammonium chloride (200 mL), acidified to pH 3 with 2 M HCl and allowed to stir at room temperature for 30 min. The mixture was then extracted with EtOAc (3×100 mL). The combined organic extracts were dried over anhydrous sodium sulfate, filtered, and concentrated in vacuo. Purification was carried out by column chromatography over silica with starting material eluting at ethyl acetate:hexanes (3:7), and desired product eluting at ethyl acetate:hexanes (1:1), obtained as a viscous pale yellow oil (13.8 g, 69.0%). MS (ESI) m/z 346.2 [M-Boc+H]⁺.

### Step 2-4. Synthesis of dimethyl (2S,4S)-2-((tert-butoxycarbonyl)amino)-4-((S)-1-((tert-butyldimethylsilyl)oxy)but-3-en-2-yl)pentanedioate and dimethyl (2S,4R)-2-((tert-butoxycarbonyl)amino)-4-((R)-1-((tert-butyldimethylsilyl)oxy)but-3-en-2-yl)pentanedioate

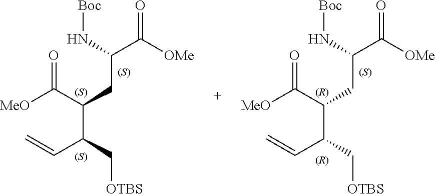

The starting material acid (13.8 g, 31.0 mmol, 1.0 equiv., mixture of (S),(S),(S) and (S),(R),(R) diastereomers) and potassium carbonate (8.62 g, 61.9 mmol, 2.0 equiv.) were suspended in anhydrous DMF (60 mL) and cooled to 0° C. Iodomethane (3.86 mL, 61.9 mmol, 2.0 equiv.) was then added in 1 portion and the mixture was allowed to stir, protected from light, at room temperature for 3 h. TLC showed complete consumption of the starting material and formation of desired product at Rr 0.7 (ethyl acetate:hexanes (1:1), KMnO₄ stain). The reaction was quenched by addition of water (100 mL) and extracted with Et₂O (3×100 mL). The combined organic extracts was dried over anhydrous sodium sulfate, filtered, and concentrated in vacuo to yield a desired product as yellow oil (17.8 g) which was assumed quantitative and used in the next step without further purification.

### Step 2-5. Synthesis of dimethyl (2S,4S)-2-((tert-butoxycarbonyl)amino)-4-((S)-1-hydroxybut-3-en-2-yl)pentanedioate and dimethyl (2S,4R)-2-((tert-butoxycarbonyl)amino)-4-((R)-1-hydroxybut-3-en-2-yl)pentanedioate

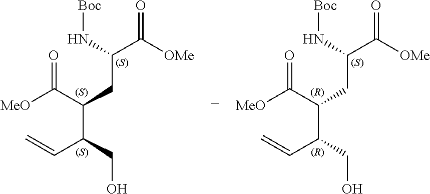

A solution of anhydrous TBAF (310 mL, 1 M in THF, 310 mmol, 10.0 equiv.) was cooled to 0° C. under N₂. Glacial AcOH (18.6 mL, 325 mmol, 10.5 equiv.) was added and stirred until internal temperature is <5° C. This solution was then poured directly into a flask containing TBS ether from Step 4 (17.8 g crude from previous step) in anhydrous THF (20 mL) under N₂. The reaction was allowed to stir at 0° C. for 3 to 6 h. Reaction was tracked by TLC (Rr 0.4 in ethyl acetate:hexanes (1:1); KMnO4 stain) and quenched immediately after near complete consumption of starting material was observed. The reaction was diluted by addition of water (200 mL) and then extracted with EtOAc (3×200 mL). The combined organic extracts was dried over anhydrous sodium sulfate, filtered, and concentrated in vacuo. Purification by flash column chromatography over silica, eluting at ethyl acetate:hexanes (7:3), provided desired product as a colorless oil (8.75 g, 81.7% over 2 steps).

### Step 2-6. Synthesis of dimethyl (2S,4S)-2-((tert-butoxycarbonyl)amino)-4-((S)-1-(1,3-dioxoisoindolin-2-yl)but-3-en-2-yl)pentanedioate and dimethyl (2S,4R)-2-((tert-butoxycarbonyl)amino)-4-((R)-1-(1,3-dioxoisoindolin-2-yl)but-3-en-2-yl)pentanedioate

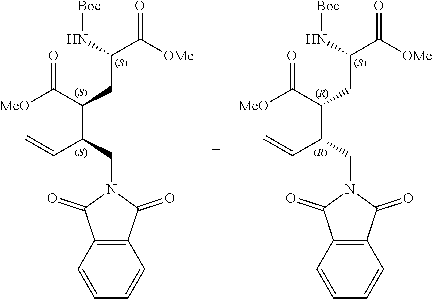

1,5-dimethyl (2S)-2-[(tert-butoxycarbonyl)amino]-4-(1-hydroxybut-3-en-2-yl)pentanedioate (6.90 g, 20.0 mmol, 1.0 equiv.), phthalimide (5.88 g, 40.0 mmol, 2.0 equiv.), and triphenylphosphine (7.86 g, 30.0 mmol, 1.5 equiv.) were dissolved in anhydrous THE (150 mL) and cooled to 0° C. DIAD (6.10 mL, 31.0 mmol, 1.55 equiv.) was added dropwise, resulting in the formation of a yellow solution. The reaction was stirred at 0° C. for an additional 15 min, before warming to room temperature and stirring for 2 h. Complete consumption of starting material observed by TLC (product RF 0.6 in ethyl acetate:hexanes (1:1), UV active and stains by KMnO₄). The reaction was diluted with saturated aqueous ammonium chloride (200 mL) and extracted with EtOAc (3×100 mL). The combined organic extracts was dried over anhydrous sodium sulfate, filtered, and concentrated in vacuo. Purification by flash column chromatography over silica, eluting at ethyl acetate:hexanes (1:1), provided the desired product as a sticky pale yellow solid (15 g). NMR showed presence of some phthalimide and DIAD by-products. Material was moved into the next step assuming quantitative yield.

### Step 2-7. Synthesis of methyl (S)-2-((tert-butoxycarbonyl)amino)-3-((3S,4S)-2-oxo-4-vinylpyrrolidin-3-yl)propanoate and methyl (S)-2-((tert-butoxycarbonyl)amino)-3-((3R,4R)-2-oxo-4-vinylpyrrolidin-3-yl)propanoate

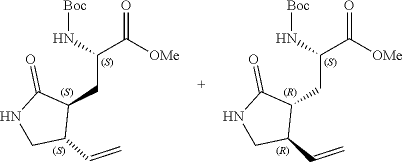

To a solution of the phthalimides from Step 1 (15 g from previous step) in MeOH (120 mL) in a 150 mL pressure vessel was added methylamine hydrochloride (6.74 g, 100 mmol, 5.0 equiv.) followed by N,N-diisopropylethylamine (17.4 mL, 100 mmol). The vessel was sealed and heated to 80° C. for 3 h. LC/MS analysis showed that reaction was complete. Reaction volume was reduced under vacuum to (approximately 40 mL).

Saturated aqueous ammonium chloride (100 mL) was added, and the mixture extracted with EtOAc (3×100 mL). The combined organic extracts was dried over anhydrous sodium sulfate, filtered, and concentrated in vacuo. Purification by flash column chromatography over silica, eluting at ethyl acetate:hexanes (3:1), provided desired products as two separate fractions. Methyl (S)-2-((tert-butoxycarbonyl)amino)-3-((3S,4S)-2-oxo-4-vinylpyrrolidin-3-yl)propanoate was isolated as the less polar fraction: 2.02 g, 32.4% clear sticky gum and methyl (S)-2-((tert-butoxycarbonyl)amino)-3-((3R,4R)-2-oxo-4-vinylpyrrolidin-3-yl)propanoate was isolated as the more polar fraction: 1.56 g, 25.0%, pale yellow solid, combined 57% over 2 steps).

Isomer 1: 1H NMR (400 MHz, CDCl₃) δ 6.68 (d, J=7.4 Hz, 1H), 5.81-5.53 (m, 2H), 5.24-5.08 (m, 2H), 4.49-4.40 (m, 1H), 3.72 (s, 3H), 3.44 (ddd, J=9.5, 8.0, 1.4 Hz, 1H), 3.15 (t, J=9.5 Hz, 1H), 2.77 (p, J=8.9 Hz, 1H), 2.30 (ddd, J=11.2, 7.4, 4.1 Hz, 1H), 2.12-1.97 (m, 2H), 1.44 (s, 9H). MS (APCI) m/z 213.1 [M-Boc+H]⁺.

Isomer 2: 1H NMR (400 MHz, CDCl₃) δ 6.18 (d, J=6.9 Hz, 1H), 5.78 (dddd, J=14.9, 13.0, 9.0, 4.1 Hz, 1H), 5.61 (s, 1H), 5.28-5.13 (m, 2H), 4.27 (dt, J=10.8, 5.9 Hz, 1H), 3.81-3.67 (m, 3H), 3.62-3.40 (m, 1H), 3.15 (t, J=9.3 Hz, 1H), 2.78 (p, J=8.6 Hz, 1H), 2.30 (dt, J=11.1, 5.9 Hz, 1H), 2.13-1.89 (m, 2H), 1.44 (d, J=3.7 Hz, 9H). MS (APCI) m/z 213.1 [M-Boc+H]⁺.

### General Route 3

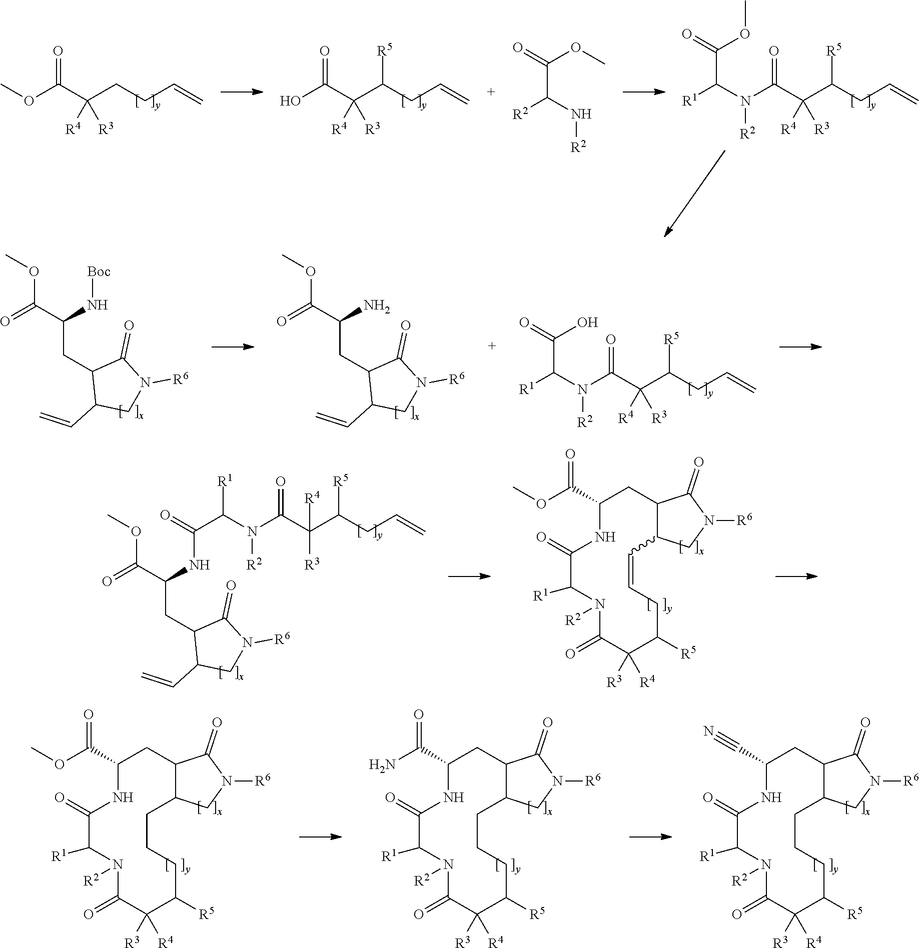

### Example 3

### Specific Exemplification of General Route 3

### tert-butyl ((3aR,8S,11aS,12aR,12bS,15S,16aS)-15-cyano-12,12-dimethyl-1,9,13-trioxoicosahydrocyclopropa[3,4]pyrrolo[1,2-a]pyrrolo[3,4-g][1,4]diazacyclotetradecin-8-yl)carbamate

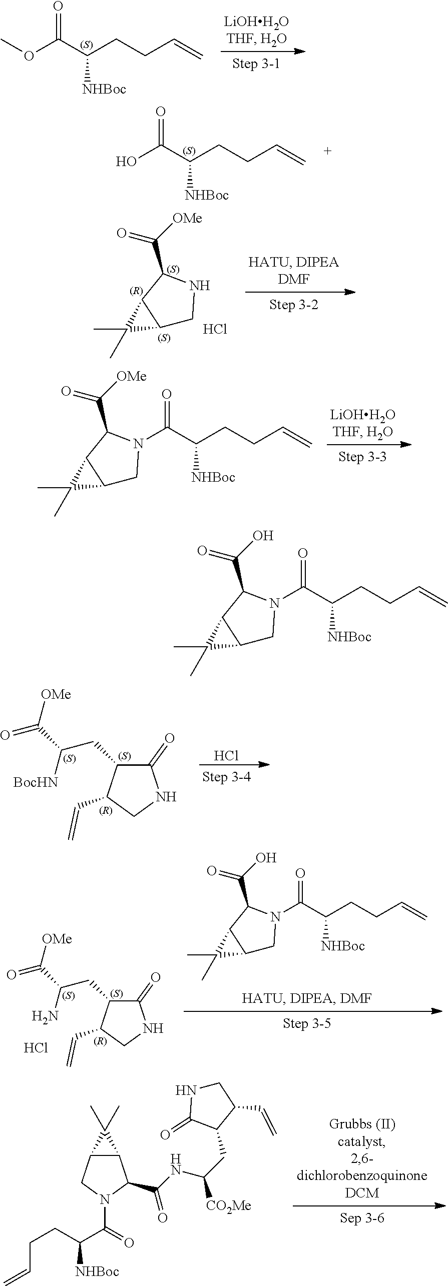

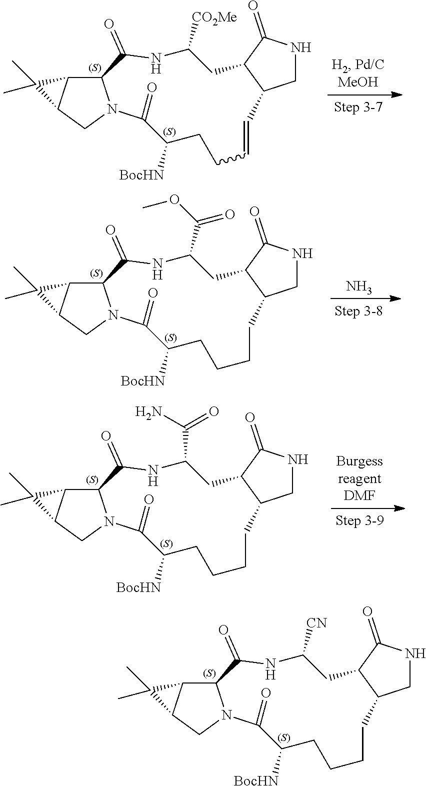

### Step 3-1. Synthesis of (S)-2-((tert-butoxycarbonyl)amino)hex-5-enoic acid

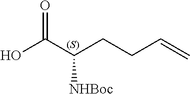

Methyl (S)-2-((tert-butoxycarbonyl)amino)hex-5-enoate (1 g, 4.11 mmol, 1.0 equiv.) was dissolved in a mixture of tetrahydrofuran:water (4:1, 20 mL) and cooled to 0° C. To the solution was added lithium hydroxide monohydrate (519 mg, 12.33 mmol, 3 equiv.) in water (3 mL). The reaction was stirred at 0° C. for 4 hours before allowing it to warm to room temperature. The completed reaction was neutralized with 0.2 M HCl to pH 2 and the resulting suspension was extracted with ethyl acetate (3×100 mL).

The organic layers were combined, dried over sodium sulfate and concentrated in vacuo to obtain the crude titled compound as a thick yellow oil; ¹H NMR (400 MHz, CDCl₃) δ 5.80 (ddt, J=16.8, 10.2, 6.6 Hz, 1H), 5.07 (dq, J=17.1, 1.6 Hz, 1H), 5.02 (dq, J=10.2, 1.4 Hz, 1H), 4.38-4.29 (m, 1H), 2.17 (td, J=7.8, 4.0 Hz, 2H), 2.03-1.91 (m, 1H), 1.84-1.71 (m, 1H), 1.45 (s, 9H); MS (ESI) m/z 130.1 [C₆H₁₁NO₂+H]⁺.

### Step 3-2. Synthesis of methyl (1R,2S,5S)-3-((S)-2-((tert-butoxycarbonyl)amino)hex-5-enoyl)-6,6-dimethyl-3-azabicyclo[3.1.0]hexa ne-2-carboxylate

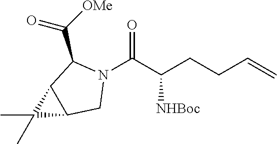 [SMILES: C=CCC[C@H](NC(=O)OC(C)(C)C)C(=O)N1C[C@H]2[C@@H]([C@H]1C(=O)OC)C2(C)C]

(S)-2-((tert-butoxycarbonyl)amino)hex-5-enoic acid (crude from step 1; 1 g, 4.36 mmol, 1 equiv.) was dissolved in anhydrous N,N-dimethylformamide (10 mL). Methyl (1R,2S,5S)-6,6-dimethyl-3-azabicyclo[3.1.0]hexane-2-carboxylate hydrochloride (986 mg, 4.8 mmol, 1.1 equiv.) and HATU (1825 mg, 1.1 mmol, 1.1 equiv.) was added and the reaction was cooled to 0° C. N,N-diisopropylethylamine (3.80 mL, 5.0 mmol, 5 equiv.) was added and the resulting yellow solution was stirred at 0° C. for 30 minutes and then room temperature for an additional 15 minutes. The completed reaction was quenched with saturated ammonium chloride solution (50 mL) and the resulting mixture was extracted with ethyl acetate (4×10 mL). The organic layers were combined, washed with saturated sodium bicarbonate, water and then brine sequentially before drying over anhydrous magnesium sulfate. Purification by flash column chromatography over silica, eluting at ethyl acetate:hexanes (3:2), afforded the titled compound as a clear gum (1.35 g, 3.55 mmol, 78%); 1H NMR (400 MHz, CDCl₃) δ 5.82 (ddt, J=16.9, 10.1, 6.6 Hz, 1H), 5.16-4.96 (m, 3H), 4.45 (s, 1H), 4.39 (dt, J=9.0, 6.7 Hz, 1H), 3.92-3.72 (m, 5H), 2.22-2.13 (m, 1H), 1.86 (ddt, J=15.0, 8.5, 6.6 Hz, 1H), 1.72-1.60 (m, 1H), 1.52-1.40 (m, 11H), 1.05 (s, 3H), 0.94 (s, 3H); MS (ESI) m/z 381.1 [C₂₀H₃₂N₂O₅+H]⁺.

### Step 3-3. Synthesis of (1R,2S,5S)-3-((S)-2-((tert-butoxycarbonyl)amino)hex-5-enoyl)-6,6-dimethyl-3-azabicyclo[3.1.0]hexane-2-carboxylic acid

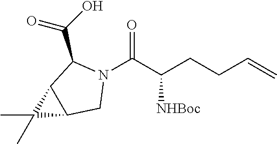 [SMILES: C=CCC[C@H](NC(=O)OC(C)(C)C)C(=O)N1C[C@H]2[C@@H]([C@H]1C(=O)O)C2(C)C]

Methyl (1R,2S,5S)-3-((S)-2-((tert-butoxycarbonyl)amino)hex-5-enoyl)-6,6-dimethyl-3-azabicyclo[3.1.0]hexane-2-carboxylate (250 mg, 0.657 mmol, 1 equiv.) dissolved in tetrahydrofuran (3.3 mL) and cooled to 0° C. To the solution was added lithium hydroxide monohydrate (82.7 mg, 1.97 mmol, 3 equiv.) in water (0.8 mL). The reaction was allowed to warm to room temperature and stirred for 20.5 hours. The completed reaction was cooled back to 0° C. and quenched with saturated ammonium chloride. The resulting solution was acidified with 2M HCl to pH 2 and extracted with ethyl acetate (4×25 mL). The organic layers were combined and dried over anhydrous sodium sulfate.

The solution was concentrated in vacuo and purification by flash column chromatography over silica, eluting at ethyl acetate:hexanes (9:1) afforded the titled compound as a clear oil (294.5 mg, 0.803 mmol, quantitative with trace solvent); 1H NMR (400 MHz, MeOD) δ 5.85 (ddt, J=16.9, 10.2, 6.7 Hz, 1H), 5.10 (dq, J=17.2, 1.6 Hz, 1H), 5.02 (ddt, J=10.2, 2.2, 1.2 Hz, 1H), 4.34 (s, 1H), 4.32-4.26 (m, 1H), 3.98 (d, J=10.4 Hz, 1H), 3.86 (dd, J=10.3, 5.2 Hz, 1H), 2.22-2.13 (m, 2H), 1.82 (dtd, J=13.7, 7.6, 5.9 Hz, 1H), 1.70-1.61 (m, 1H), 1.58 (dd, J=7.4, 5.0 Hz, 1H), 1.51 (d, J=7.5 Hz, 1H), 1.43 (s, 9H), 1.10 (s, 3H), 1.01 (s, 3H); MS (ESI) m/z 365.0 [C₁₉H₃₀N₂O₅—H]⁻.

### Step 3-4. Synthesis of methyl (S)-2-amino-3-((3S,4R)-2-oxo-4-vinylpyrrolidin-3-yl)propanoate hydrochloride

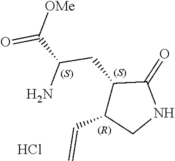

Methyl (S)-2-((tert-butoxycarbonyl)amino)-3-((3S,4R)-2-oxo-4-vinylpyrrolidin-3-yl)propanoate (150 mg, 0.480 mmol, 1 equiv.) was dissolved in 4M HCl in dioxane (2.4 mL, 9.60 mmol, 20 equiv.) and was stirred at room temperature for 30 minutes. The reaction mixture was concentrated in vacuo to afford the crude titled compound. MS (ESI) m/z 213.0 [C₁₀H₁₆N₂O₃+H]⁺.

Step 3-5. Synthesis of methyl (S)-2-((1R,2S,5S)-3-((S)-2-((tert-butoxycarbonyl)amino)hex-5-enoyl)-6,6-dimethyl-3-azabicyclo[3.1.0]hexane-2-carboxamido)-3-((3S,4R)-2-oxo-4-vinylpyrrolidin-3-yl)propanoate.

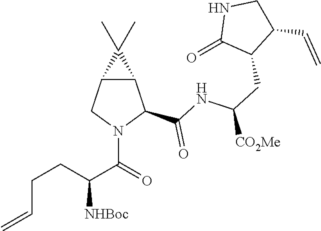 [SMILES: C=CCC[C@H](NC(=O)OC(C)(C)C)C(=O)N1C[C@H]2[C@@H]([C@H]1C(=O)N[C@@H](C[C@@H]1C(=O)NC[C@@H]1C=C)C(=O)OC)C2(C)C]

Methyl (S)-2-amino-3-((3S,4R)-2-oxo-4-vinylpyrrolidin-3-yl)propanoate hydrochloride (crude from step 3-4, 0.480 mmol, 1 equiv.) and (1R,2S,5S)-3-((S)-2-((tert-butoxycarbonyl)amino)hex-5-enoyl)-6,6-dimethyl-3-azabicyclo[3.1.0]hexane-2-carboxylic acid (211.1 mg, 0.576 mmol, 1.2 equiv.) were dissolved in N,N-dimethylformamide (4.80 mL) and cooled to 0° C. N,N-diisopropylethylamine (0.418 mL, 2.40 mmol, 5 equiv.) and HATU (200.8 mg, 0.528 mmol, 1.1 equiv) was added and the resulting yellow solution was stirred for 30 minutes. The completed reaction was diluted with ethyl acetate and washed with saturated ammonium chloride and brine sequentially. The aqueous layers were combined and back extracted with ethyl acetate (2×25 mL). The organic fractions were combined and dried with anhydrous sodium sulfate. The solution was concentrated in vacuo and purified via semi-preparative reverse phase HPLC (30-60% MeCN/H₂O, 0.1% formic acid) to afford the titled compound as a clear oil (199.4 mg, 0.356 mmol, 74%); 1H NMR (400 MHz, MeOD) δ 8.87 (d, J=7.4 Hz, 1H), 5.92-5.78 (m, 2H), 5.23 (dd, J=17.1, 1.7 Hz, 1H), 5.18 (dd, J=10.2, 1.8 Hz, 1H), 5.09 (dq, J=17.1, 1.7 Hz, 1H), 5.01 (dd, J=10.2, 2.0 Hz, 1H), 4.69 (ddd, J=9.6, 6.6, 4.5 Hz, 1H), 4.35 (s, 1H), 4.29 (dt, J=8.7, 5.4 Hz, 1H), 3.94 (d, J=3.4 Hz, 2H), 3.74 (s, 3H), 3.57 (dd, J=10.0, 6.0 Hz, 1H), 3.21-3.09 (m, 2H), 2.65 (td, J=8.0, 5.6 Hz, 1H), 2.20-2.10 (m, 2H), 2.06 (ddd, J=14.0, 8.1, 5.7 Hz, 1H), 1.88-1.75 (m, 2H), 1.70-1.61 (m, 1H), 1.58 (ddd, J=6.2, 4.3, 2.3 Hz, 1H), 1.50 (d, J=7.7 Hz, 1H), 1.43 (s, 9H), 1.10 (s, 3H), 1.01 (s, 3H); MS (ESI) m/z 561.2 [C₂₉H₄₄N₄O₇+H]⁺.

### Step 3-6. Synthesis of methyl (3aR,8S,11 aS,12aR,12bS,15S,16 aS)-8-((tert-butoxycarbonyl)amino)-12,12-dimethyl-1,9,13-trioxo-1,2,3,3a,6,7,8,9,11,11a,12,12a,12b,13,14,15,16,16a-octadecahydrocyclopropa[3,4]pyrrolo[1,2-a]pyrrolo[3,4-g][1,4]diazacyclotetradecine-15-carboxylate

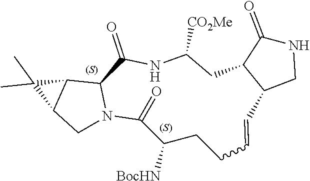

Methyl (S)-2-((1R,2S,5S)-3-((S)-2-((tert-butoxycarbonyl)amino)hex-5-enoyl)-6,6-dimethyl-3-azabicyclo[3.1.0]hexa ne-2-carboxamido)-3-((3S,4R)-2-oxo-4-vinylpyrrolidin-3-yl)propanoate (199.4 mg, 0.355 mmol, 1 equiv.) was dissolved in anhydrous dichloromethane (71.1 mL) under nitrogen. Grubbs catalyst, 2^{nd} Generation (30.2 mg, 0.0355 mmol, 10 mol %) and 2,6-dichlorobenzoquinone (6.3 mg, 0.0355 mmol, 10 mol %) was added and the reaction was stirred at 45° C. for 16.5 hours. Si-TMT (1.5 g) was added to the reaction after cooling to room temperature and stirred for an hour. The resulting suspension was filtered over a bed of celite and rinsed with methanol. The filtrate was concentrated in vacuo to afford the crude titled compound as a brown solid. MS (APCI) m/z 533.3 [C₂₇H₄₀N₄O₇+H]⁺.

### Step 3-7. Synthesis of methyl (3aR,8S,11 aS,12aR,12bS,15S,16aS)-8-((tert-butoxycarbonyl)amino)-12,12-dimethyl-1,9,13-trioxoicosahydrocyclopropa[3,4]pyrrolo[1,2-a]pyrrolo[3,4-g][1,4]diazacyclotetradecine-15-carboxylate

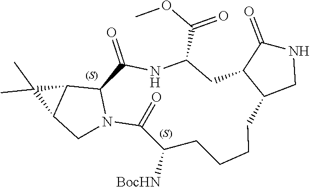

Methyl (3aR,8S,11aS,12aR,12bS,15S,16aS)-8-((tert-butoxycarbonyl)amino)-12,12-dimethyl-1,9,13-trioxo-1,2,3,3a,6,7,8,9,11,11a,12,12a,12b,13,14,15,16,16a-octadecahydrocyclopropa[3,4]pyrrolo[1,2-a]pyrrolo[3,4-g][1,4]diazacyclotetradecine-15-carboxylate (crude from step 3-6, 0.355 mmol, 1 equiv.) was dissolved in methanol (20 mL) under nitrogen. Palladium on carbon (75.6 mg, 0.0711 mmol, 10 w/w %, 0.2 equiv.) and hydrogen gas (via a balloon) was added and the reaction was stirred at room temperature for 16.5 hours. The completed reaction was filtered over a bed of celite and rinsed with methanol. The filtrate was concentrated in vacuo and purified via semi-preparative reverse phase HPLC (30-60% MeCN/H₂O, 0.1% formic acid) to afford the titled compound as a white solid (65.4 mg, 0.122 mmol, 35%); ¹H NMR (400 MHz, MeOD) δ 8.33 (d, 3=8.9 Hz, 1H), 4.74 (td, J=8.5, 3.4 Hz, 1H), 4.58 (s, 1H), 4.28 (dd, J=9.2, 3.5 Hz, 1H), 4.12 (d, J=10.7 Hz, 1H), 3.84 (dd, J=10.6, 5.3 Hz, 1H), 3.77 (s, 3H), 3.39-3.29 (m, 1H), 3.01 (dd, J=9.9, 5.8 Hz, 1H), 2.55 (q, J=7.2 Hz, 1H), 2.41 (h, J=6.8 Hz, 1H), 2.21 (ddd, J=14.7, 7.3, 3.5 Hz, 1H), 1.87 (ddd, J=14.6, 11.1, 5.8 Hz, 1H), 1.79-1.56 (m, 4H), 1.47 (d, J=6.8 Hz, 1H), 1.43 (s, 9H), 1.40-1.18 (m, 5H), 1.10 (s, 3H), 0.96 (s, 3H); MS (APCI) m/z 535.3 [C₂₇H₄₂N₄O₇+H]⁺.

### Step 3-8. Synthesis of tert-butyl ((3aR,8S,11aS,12aR,12bS,15S,16aS)-15-carbamoyl-12,12-dimethyl-1,9,13-trioxoicosahydrocyclopropa [3,4]pyrrolo[1,2-a]pyrrolo[3,4-g][1,4]diazacyclotetradecin-8-yl)carbamate

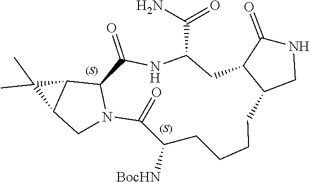

Methyl (3aR,8S,11aS,12aR,12bS,15S,16aS)-8-((tert-butoxycarbonyl)amino)-12,12-dimethyl-1,9,13-trioxoicosahydrocyclopropa [3,4]pyrrolo[1,2-a]pyrrolo[3,4-g][1,4]diazacyclotetradecine-15-carboxylate (32.7 mg, 0.0611 mmol, 1 equiv.) was dissolved in 7N ammonia in methanol (3 mL) and the reaction was stirred at 60° C. More 7N ammonia in methanol (2×2 mL) was added to the reaction periodically (4.5 h and 7.5 h) and the reaction was completed after a total of 23.5 hours. The completed reaction was concentrated in vacuo and purified via semi-preparative reverse phase HPLC (30-60% MeCN/H₂O, 0.1% formic acid) to afford the titled compound as a white solid (20.8 mg, 0.040 mmol, 65%); 1H NMR (400 MHz, MeOD) δ 8.23 (d, J=8.7 Hz, 1H), 4.70-4.62 (m, 1H), 4.59 (s, 1H), 4.30 (dd, J=8.4, 3.2 Hz, 1H), 4.05 (d, J=10.7 Hz, 1H), 3.83 (dd, J=10.6, 5.3 Hz, 1H), 3.38-3.29 (m, 1H), 3.00 (dd, J=9.9, 6.0 Hz, 1H), 2.68 (q, J=7.1 Hz, 1H), 2.41 (h, J=6.9 Hz, 1H), 2.09 (ddd, J=14.7, 7.4, 3.6 Hz, 1H), 1.99-1.88 (m, 1H), 1.78-1.57 (m, 4H), 1.52-1.47 (m, 1H), 1.43 (s, 9H), 1.37-1.19 (m, 5H), 1.10 (s, 3H), 0.97 (s, 3H); MS (APCI) m/z 520.3 [C₂₆H₄₁N₅O₆+H]⁺.

### Step 3-9. Synthesis of tert-butyl ((3aR,8S,11aS,12aR,12bS,15S,16aS)-15-cyano-12,12-dimethyl-1,9,13-trioxoicosahydrocyclopropa [3,4]pyrrolo[1,2-a]pyrrolo[3,4-g][1,4]diazacyclotetradecin-8-yl)carbamate

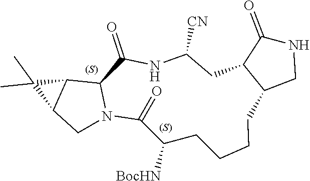

Tert-butyl ((3aR,8S,11aS,12aR,12bS,15S,16aS)-15-carbamoyl-12,12-dimethyl-1,9,13-trioxoicosahydrocyclopropa [3,4]pyrrolo[1,2-a]pyrrolo[3,4-g][1,4]diazacyclotetradecin-8-yl)carbamate (20.8 mg, 0.040 mmol, 1 equiv.) was dissolved in anhydrous N,N-dimethylformamide (0.8 mL). Burgess reagent (28.6 mg, 0.120 mmol, 3 equiv.) was added and the reaction was stirred at room temperature for 2 hours. The completed reaction was quenched with water (0.1 mL) and purified via semi-preparative reverse phase HPLC (25-55% MeCN/H₂O, 0.1% formic acid) to afford the titled compound as a white solid upon lyophilization (10.6 mg, 0.021 mmol, 53%); ¹H NMR (400 MHz, MeOD) δ 5.14 (dd, J=7.5, 4.2 Hz, 1H), 4.54 (s, 1H), 4.28 (dd, J=8.9, 3.6 Hz, 1H), 4.08 (d, J=10.7 Hz, 1H), 3.84 (dd, J=10.7, 5.1 Hz, 1H), 3.38-3.34 (m, 1H), 3.00 (dd, J=9.9, 6.1 Hz, 1H), 2.52-2.36 (m, 2H), 2.17 (ddd, J=14.9, 7.4, 4.3 Hz, 1H), 2.10-1.98 (m, 1H), 1.95-1.81 (m, 1H), 1.72-1.57 (m, 3H), 1.43 (s, 9H), 1.52-1.39 (m, 1H), 1.39-1.14 (m, 5H), 1.11 (s, 3H), 0.96 (s, 3H); MS (ESI) m/z 502.2 [C₂₆H₃₉N₅O₅+H]⁺.

### Example 4

### Specific Exemplification of General Route 3

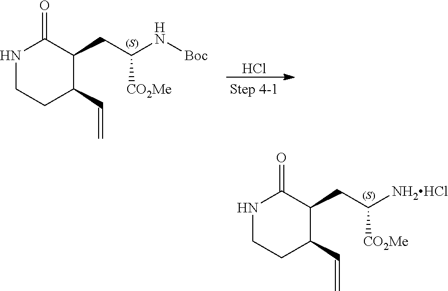

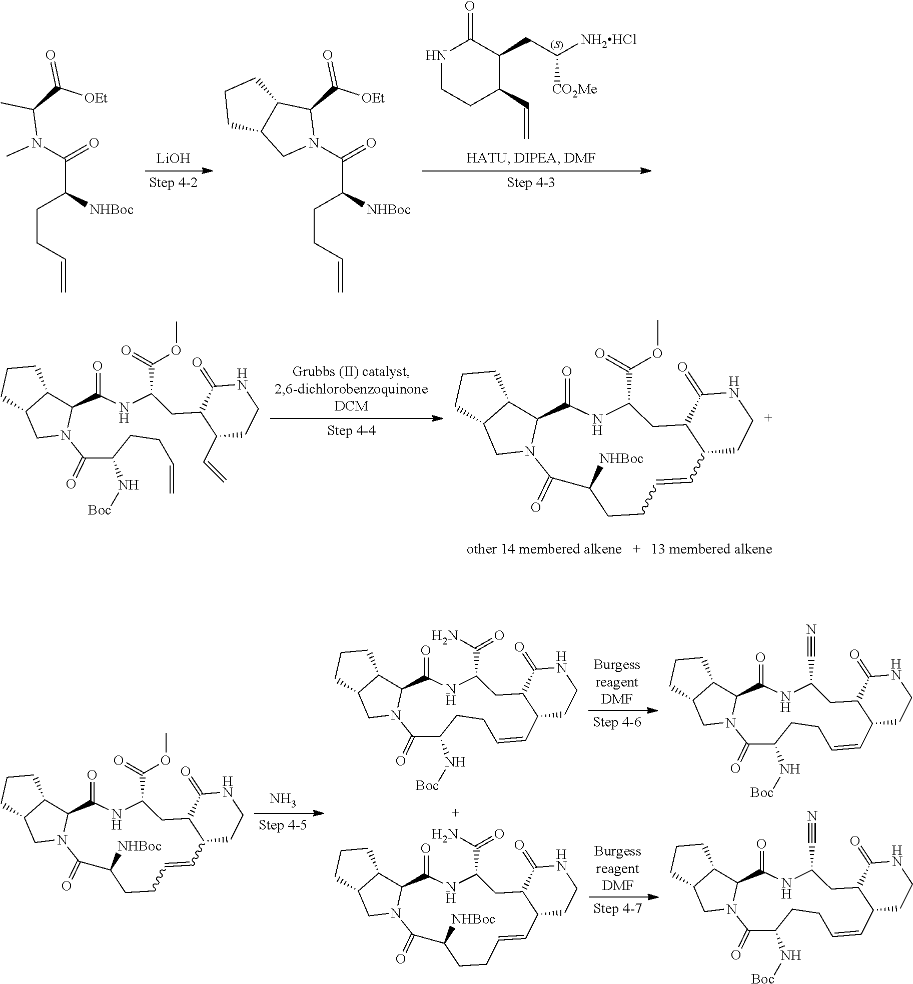

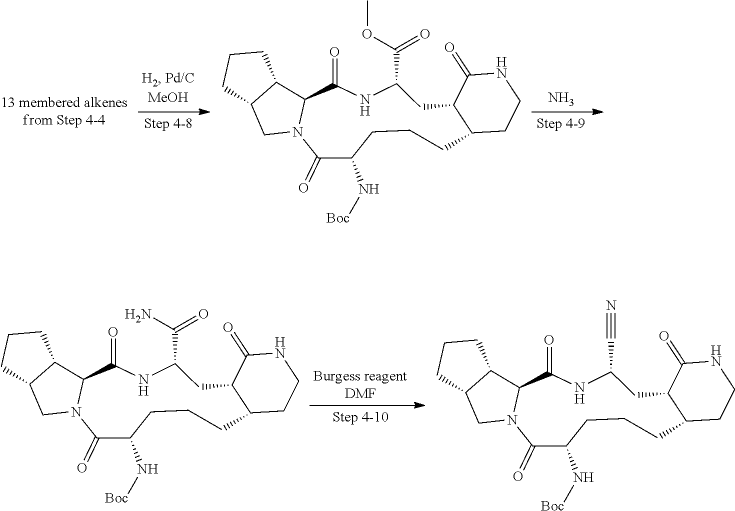

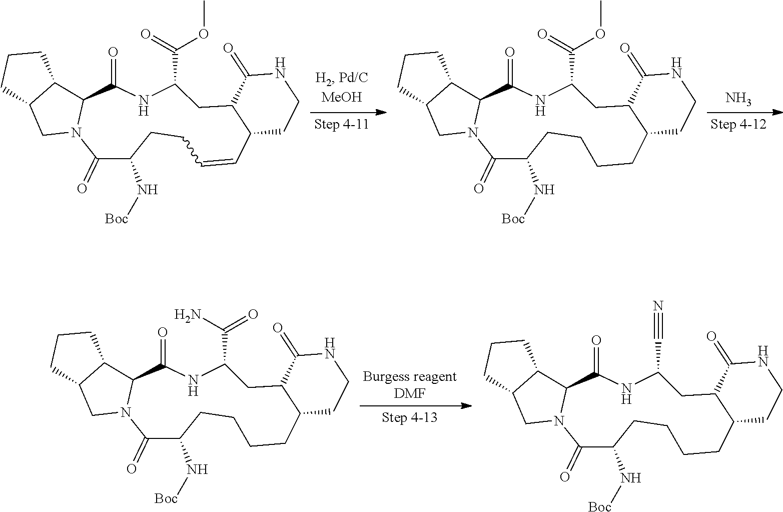

### Step 4-1. Synthesis of methyl (S)-2-amino-3-((3S,4R)-2-oxo-4-vinylpiperidin-3-yl)propanoate hydrochloride

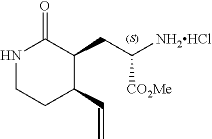

To a solution of methyl (S)-2-((tert-butoxycarbonyl)amino)-3-((3S,4R)-2-oxo-4-vinylpiperidin-3-yl)propanoate (200 mg, 0.61 mmol) in 1,4-dioxane (0.1 mL) was added 4.0 M HCl in 1,4-dioxane (2.0 mL) at 0° C. The reaction was stirred at room temperature for 1 h. The reaction mixture was then concentrated under vacuo and used without further purification; MS (ESI) m/z 227.1 [C₁₁H₁₈N₂O₃+H]⁺.

### Step 4-2. Synthesis of (1S,3aR,6aS)-2-((S)-2-((tert-butoxycarbonyl)amino)hex-5-enoyl)octahydrocyclopenta[c]pyrrole-1-carboxylic acid

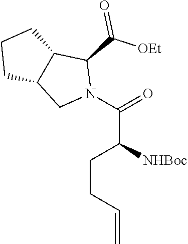 [SMILES: C=CCC[C@H](NC(=O)OC(C)(C)C)C(=O)N1C[C@@H]2CCC[C@@H]2[C@H]1C(=O)OCC]

To a solution of ethyl (1S,3aR,6aS)-2-((S)-2-((tert-butoxycarbonyl)amino)hex-5-enoyl)octahydrocyclopenta[c]pyrrole-1-carboxylate (400 mg, 1.01 mmol) in THE (4 mL) was added a solution of LiOH (73 mg, 3.04 mmol) in water (1 mL) dropwise at 0° C. The reaction was stirred at 0° C. and slowly warmed to room temperature over 5 h. The reaction mixture was re-cooled to 0° C. and quenched by the addition of saturated ammonium chloride (15 mL). The crude mixture was acidified to pH 3 by the addition of 0.5 M HCl solution and extracted by ethyl acetate (3×50 mL). The combined organic layers were washed with brine, dried over anhydrous sodium sulfate, filtered, and concentrated under vacuo to give a light yellow paste. Purification by flash column chromatography over silica, eluting at ethyl acetate:hexanes (1:1), afforded the titled compound as a colorless gum (355 mg, 0.97 mmol); 1H NMR (400 MHz, MeOD) δ 5.85 (ddt, J=16.9, 10.1, 6.7 Hz, 1H), 5.10 (dq, J=17.1, 1.7 Hz, 1H), 5.02 (dq, J=10.3, 1.2 Hz, 1H), 4.43-4.28 (m, 2H), 3.80 (qd, J=10.6, 5.8 Hz, 2H), 2.89-2.68 (m, 2H), 2.18 (q, J=7.5 Hz, 2H), 2.08-1.98 (m, 1H), 1.97-1.89 (m, 1H), 1.88-1.75 (m, 2H), 1.73-1.54 (m, 4H), 1.46 (s, 9H); MS (ESI) m/z 365.2 [C₁₉H₃₀N₂O₅+H]⁺.

### Step 4-3. Synthesis of methyl (S)-2-((1S,3aR,6aS)-2-((S)-2-((tert-butoxycarbonyl)amino)hex-5-enoyl)octahydrocyclopenta[c]pyrrole-1-carboxamido)-3-((3S,4R)-2-oxo-4-vinylpiperid in-3-yl)propanoate

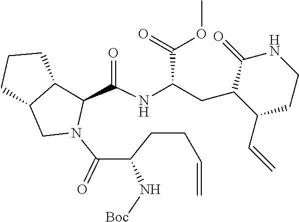

To a mixture of tert-butyl (2S)-2-aminopentanoate hydrochloride (161 mg, 0.61 mmol), (2R)-2-{[(9H-fluoren-9-ylmethoxy)carbonyl]amino}-3-phenylpropanoic acid (227 mg, 0.62), N,N-diisopropylethylamine (0.61 mL, 3.05 mmol) in anhydrous DMF (2 mL) was added HATU (236 mg, 0.62 mmol) at 0° C. The reaction was stirred at 0° C. for 0.5 h. Water (5 mL) was added to the reaction mixture and the aqueous layer was extracted twice with ethyl acetate. The combined organic layers were washed with brine, dried over anhydrous sodium sulfate, filtered, and concentrated under vacuo. The crude product was purified by semi-preparative reverse phase HPLC (25-55% MeCN/H₂O, 0.1% formic acid) to give the titled compound as a white solid (155 mg, 0.27 mmol); MS (ESI) m/z 575.3 [C₃₀H₄₆N₄O₇+H]⁺.

### Step 4-4. Synthesis of methyl (9S,12aR,15aS,15bS,18S,19aS)-9-((tert-butoxycarbonyl)amino)-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecine-18-carboxylate

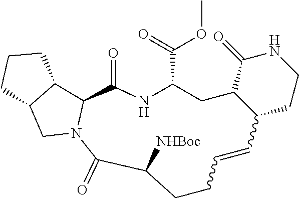

A degassed mixture of methyl (S)-2-((1S,3aR,6aS)-2-((S)-2-((tert-butoxycarbonyl)amino)hex-5-enoyl)octahydrocyclopenta[c]pyrrole-1-carboxamido)-3-((3S,4R)-2-oxo-4-vinylpiperidin-3-yl)propanoate (155 mg, 0.27 mmol), Grubbs Catalyst 2nd generation (23 mg, 0.027 mmol) and, 2,6-dichlorocyclohexa-2,5-diene-1,4-dione (5 mg, 0.027 mmol) was dissolved in anhydrous dichloromethane (100 mL). The reaction media was degassed with nitrogen, then heated to 45° C. for 18 h. The reaction was then cooled to room temperature. SI-TMT (571 mg) was added and the resulting suspension was stirred at room temperature for 2 h before it was filtered over celite. The filter plug was washed with MeOH (50 mL) and the combined filtrate was concentrated in vacuo. The residue was purified by semi-preparative reverse phase HPLC (25-65% MeCN/H₂O, 0.1% formic acid) to afford the titled compound as a white solid (23 mg, 0.042 mmol); MS (ESI) m/z 547.2 [C₂₈H₄₂N₄O₇+H]⁺. A mixture of other 14-membered alkenes (12 mg, 0.021 mmol), MS (ESI) m/z 547.2 [C₂₈H₄₂N₄O₇+H]⁺, and a mixture of 13-membered alkenes (37 mg, 0.069 mmol), MS (ESI) m/z 533.2 [C₂₇H₄₀N₄O₇+H]⁺ were also collected.

Step 4-5. Synthesis of tert-butyl ((4aR,9S,12aR,15aS,15bS,18S,19aS,Z)-18-carbamoyl-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecin-9-yl)carbamate and tert-butyl ((4aR,9S,12 aR,15aS,15bS,18S,19 aS,E)-18-carbamoyl-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecin-9-yl)carbamate.

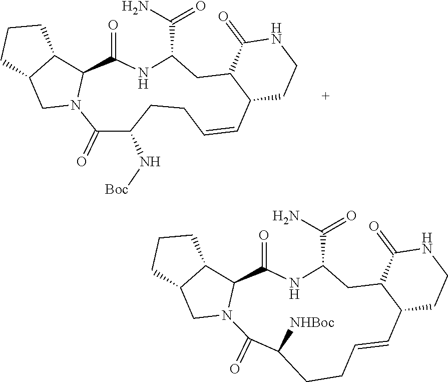

Methyl (9S,12aR,15aS,15bS,18S,19aS)-9-((tert-butoxycarbonyl)amino)-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecine-18-carboxylate (23 mg, 0.042 mmol) was dissolved in 7N ammonia in methanol (3 mL). The reaction media was stirred at 55° C. for 16 hr. The reaction was concentrated and purified by semi-preparative reverse phase HPLC (25-65% MeCN/H₂O, 0.1% formic acid) to give tert-butyl ((4aR,9S,12aR,15aS,15bS,18S,19aS,Z)-18-carbamoyl-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecin-9-yl)carbamate (15 mg, 0.028 mmol) as a white solid; ¹H NMR (400 MHz, MeOD) δ 5.50-5.35 (m, 2H), 4.53 (d, J=2.5 Hz, 1H), 4.44-4.34 (m, 2H), 3.90 (dd, J=10.9, 4.3 Hz, 1H), 3.77 (dd, J=10.8, 8.1 Hz, 1H), 3.78-3.23 (m, 2H), 3.09-2.99 (m, 1H), 2.97-2.85 (m, 1H), 2.85-2.76 (m, 1H), 2.66-2.52 (m, 2H), 2.44-2.32 (m, 1H), 2.26-2.04 (m, 2H), 2.03-1.87 (m, 3H), 1.87-1.71 (m, 3H), 1.70-1.39 (m, 13H); MS (ESI) m/z 532.3 [C₂₇H₄₁N₅O₆+H]⁺.

tert-butyl ((4aR,9S,12aR,15aS,15bS,18S,19aS,E)-18-carbamoyl-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecin-9-yl)carbamate was also obtained as a white solid (5 mg, 0.0094 mmol); ¹H NMR (400 MHz, MeOD) b 5.46 (dd, J=15.2, 8.2 Hz, 1H), 5.29 (ddd, J=14.6, 8.3, 4.9 Hz, 1H), 4.72 (dp, J=11.9, 4.0 Hz, 1H), 4.61 (s, 1H), 4.48 (d, J=2.5 Hz, 1H), 4.37 (d, J=6.9 Hz, 1H), 3.95 (dd, J=10.5, 8.1 Hz, 1H), 3.70 (dd, J=10.6, 3.5 Hz, 1H), 3.22 (ddd, J=13.5, 6.2, 3.0 Hz, 2H), 2.98 (tq, J=8.0, 4.2 Hz, 1H), 2.77 (tdd, J=8.2, 5.2, 2.4 Hz, 1H), 2.59-2.52 (m, 2H), 2.29 (dd, J=11.6, 8.2 Hz, 1H), 2.21-1.68 (m, 9H), 1.67-1.39 (m, 12H); MS (ESI) m/z 532.3 [C27H₄₁N₅O₆+H]⁺.

### Step 4-6. Synthesis of tert-butyl ((4aR,9S,12aR,15aS,15bS,18S,19aS,Z)-18-cyano-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecin-9-yl)carbamate

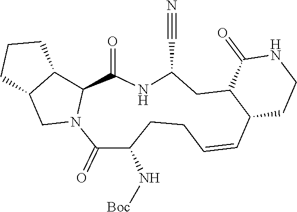

To a solution of tert-butyl ((4aR,9S,12aR,15aS,15bS,18S,19aS,Z)-18-carbamoyl-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecin-9-yl)carbamate (15 mg, 0.028 mmol) in anhydrous DMF (3 mL) was added Burgess reagent (20 mg, 0.084 mmol). The reaction media was stirred at rt for 1 h, quenched by water (0.05 mL) and purified by semi-preparative reverse phase HPLC (25-65% MeCN/H₂O, 0.1% formic acid) to afford titled compound as a white solid upon lyophilization (10 mg, 0.019 mmol); 1H NMR (400 MHz, MeOD) δ 5.45-5.28 (m, 2H), 5.01 (dd, J=7.2, 4.2 Hz, 1H), 4.39 (dd, J=8.2, 2.4 Hz, 1H), 4.32 (d, J=2.3 Hz, 1H), 3.82 (d, J=4.4 Hz, 2H), 3.39-3.34 (m, 1H), 3.28 (ddt, J=12.6, 6.7, 3.0 Hz, 1H), 3.05-2.69 (m, 4H), 2.47 (dt, J=10.7, 4.0 Hz, 1H), 2.36 (dt, J=15.8, 8.1 Hz, 1H), 2.27-1.51 (m, 12H), 1.45 (s, 9H); MS (ESI): m/z=514.3 [M+H⁺; C₂₇H₃₉N₅O₅+H⁺]

### Step 4-7. Synthesis of tert-butyl ((4aR,9S,12 aR,15aS,15bS,18S,19 aS,E)-18-cyano-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecin-9-yl)carbamate

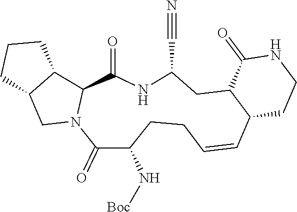

Procedure is same as Step 4-6. Titled compound (3 mg) was obtained from the corresponding starting material (5 mg).

¹H NMR (400 MHz, MeOD) δ 5.43 (dd, J=15.2, 8.5 Hz, 1H), 5.27 (ddd, J=17.6, 10.4, 4.9 Hz, 2H), 4.61 (s, 1H), 4.43-4.35 (m, 1H), 4.33 (d, J=3.1 Hz, 1H), 3.97 (dd, J=10.5, 7.8 Hz, 1H), 3.69 (dd, J=10.5, 3.3 Hz, 1H), 3.32-3.26 (m, 1H), 3.20 (ddd, J=12.6, 5.9, 2.3 Hz, 1H), 2.97 (tq, J=11.3, 4.3 Hz, 1H), 2.73 (tdd, J=8.1, 5.0, 3.0 Hz, 1H), 2.51 (ddt, J=22.9, 8.3, 3.9 Hz, 2H), 2.35-1.86 (m, 8H), 1.85-1.58 (m, 4H), 1.46 (s, 10H). MS (ESI): m/z=514.3 [M+H⁺; C₂₇H₃₉N₅O₅+H⁺].

### Step 4-8. Synthesis of methyl (4aS,8S,11aR,14aS,14bS,17S,18aS)-8-((tert-butoxycarbonyl)amino)-1,9,15-trioxodocosahydrocyclopenta[3,4]pyrrolo[1,2-a]pyrido[3,4-g][1,4]diazacyclotridecine-17-carboxylate

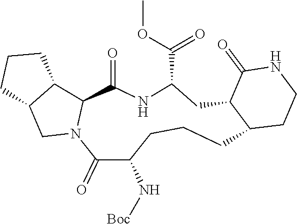 [SMILES: COC(=O)[C@@H]1C[C@@H]2C(=O)NCC[C@@H]2CCC[C@H](NC(=O)OC(C)(C)C)C(=O)N2C[C@@H]3CCC[C@@H]3[C@H]2C(=O)N1]

To a solution of 13-membered alkene mixture from Step 4-4 (37 mg, 0.069 mmol) in methanol (4 mL) was added Pd/C (10 wt %, 7 mg, 0.0069 mmol) under nitrogen. The reaction was purged with hydrogen (15 min), and stirred under hydrogen at room temperature for 5 h. The reaction mixture was then filtered over Celite, and the filter plug washed with methanol (5 mL). The combined filtrate was concentrated in vacuo and purified by semi-preparative reverse phase HPLC (25-65% MeCN/H₂O, 0.1% formic acid) to afford the titled compound as a white solid (29 mg, 0.054 mmol); ¹H NMR (400 MHz, MeOD) δ 5.25-5.15 (m, 1H), 4.85-4.82 (m, 1H), 4.35 (dd, J=11.7, 4.3 Hz, 1H), 4.01 (dd, J=11.0, 3.9 Hz, 1H), 3.77-3.58 (m, 4H), 3.30-3.23 (m, 2H), 3.19 (tdd, J=8.2, 6.4, 1.5 Hz, 1H), 2.89 (tt, J=8.3, 4.2 Hz, 1H), 2.32-2.23 (m, 1H), 2.08-1.37 (m, 23H), 1.36-1.16 (m, 2H), 1.15-1.01 (m, 1H); MS (ESI): m/z=535.3 [M+H⁺; C₂₇H₄₂N₄O₇+H⁺]

### Step 4-9. Synthesis of tert-butyl ((4aS,9S,12aR,15aS,15bS,18S,19aS)-18-carbamoyl-1,10,16-trioxodocosahydro-1H-cyclopenta[3,4]pyrrolo[1,2-a]pyrido[3,4-g][1,4]diazacyclotetradecin-9-yl)carbamate

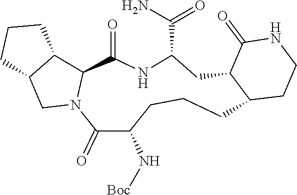 [SMILES: CC(C)(C)OC(=O)N[C@H]1CCC[C@H]2CCNC(=O)[C@H]2C[C@@H](C(N)=O)NC(=O)[C@@H]2[C@H]3CCC[C@H]3CN2C1=O]

Procedure is same as Step 4-5.

¹H NMR (400 MHz, MeOD) δ 7.33 (d, J=9.8 Hz, 1H), 5.11-5.01 (m, 1H), 4.35 (dd, J=11.6, 4.3 Hz, 1H), 4.00 (dd, J=11.0, 3.8 Hz, 1H), 3.63 (dd, J=10.9, 8.5 Hz, 1H), 3.30-3.23 (m, 2H), 3.19 (tdd, J=8.2, 6.5, 1.6 Hz, 1H), 2.90 (tq, J=8.3, 4.1 Hz, 1H), 2.34-2.26 (m, 1H), 2.00-1.37 (m, 24H), 1.27 (tdd, J=12.9, 10.7, 4.7 Hz, 2H), 1.09 (q, J=11.1 Hz, 1H); MS (ESI) m/z 520.2 [C₂₆H₄₁N₅O₆+H]⁺.

### Step 4-10. Synthesis of tert-butyl ((4aS,8S,11aR,14aS,14bS,17S,18aS)-17-cyano-1,9,15-trioxodocosahydrocyclopenta[3,4]pyrrolo[1,2-a]pyrido[3,4-g][1,4]diazacyclotridecin-8-yl)carbamate

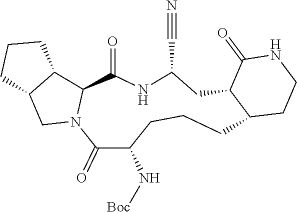 [SMILES: CC(C)(C)OC(=O)N[C@H]1CCC[C@H]2CCNC(=O)[C@H]2C[C@@H](C#N)NC(=O)[C@@H]2[C@H]3CCC[C@H]3CN2C1=O]

Procedure is same as Step 4-6.

¹H NMR (400 MHz, MeOD) δ 5.62 (dd, J=9.3, 2.6 Hz, 1H), 4.85-4.81 (m, 1H), 4.33 (dd, J=11.5, 4.2 Hz, 1H), 3.99 (dd, J=10.9, 3.8 Hz, 1H), 3.61 (dd, J=11.0, 8.5 Hz, 1H), 3.37 (m, 4H), 3.30-3.23 (m, 2H), 3.22-3.14 (m, 1H), 2.90 (dq, J=8.3, 4.2 Hz, 1H), 2.17 (dt, J=8.5, 3.9 Hz, 1H), 2.12-1.35 (m, 19H), 1.35-1.17 (m, 2H), 1.01 (q, J=11.3 Hz, 1H); MS (ESI): m/z=502.3 [M+H⁺; C₂₆H₃₉N₅O₅+H⁺]

### Step 4-11. Synthesis of methyl (4as,9S,12aR,15aS,15bS,18S,19aS)-9-((tert-butoxycarbonyl)amino)-1,10,16-trioxodocosahydro-1H-cyclopenta[3,4]pyrrolo[1,2-a]pyrido[3,4-g][1,4]diazacyclotetradecine-18-carboxylate

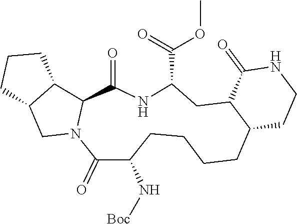

Procedure is similar to Step 4-8.

¹H NMR (400 MHz, MeOD) δ 6.83 (d, J=7.4 Hz, 1H), 5.00 (dd, J=9.0, 2.9 Hz, 1H), 4.62 (s, 1H), 4.47-4.38 (m, 1H), 3.74 (m, 5H), 3.31-3.24 (m, 2H), 3.10 (tdd, J=8.5, 6.4, 2.2 Hz, 1H), 2.90 (ddt, J=12.0, 8.2, 4.3 Hz, 1H), 2.46 (dt, J=8.7, 4.1 Hz, 1H), 2.18 (ddd, J=14.7, 9.0, 2.8 Hz, 1H), 2.06-1.84 (m, 4H), 1.82-1.54 (m, 7H), 1.54-1.25 (m, 14H), 1.19 (p, J=7.6 Hz, 2H); MS (ESI): m/z=549.3 [M+H⁺; C₂₈H₄₄N₄O₇+H⁺]

### Step 4-12. Synthesis of tert-butyl ((4aS,9S,12aR,15aS,15bS,18S,19aS)-18-carbamoyl-1,10,16-trioxodocosahydro-1H-cyclopenta[3,4]pyrrolo[1,2-a]pyrido[3,4-g][1,4]diazacyclotetradecin-9-yl)carbamate

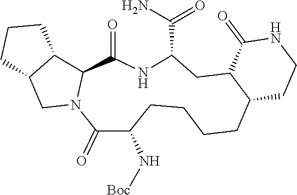

Methyl (9S,12aR,15aS,15bS,18S,19aS)-9-((tert-butoxycarbonyl)amino)-1,10,16-trioxo-2,3,4,4a,7,8,9,10,12,12a,13,14,15,15a,15b,16,17,18,19,19a-icosahydro-1H-cyclopenta[3,4]pyrrolo[1,2-a]pyrido[3,4-g][1,4]diazacyclotetradecine-18-carboxylate (6 mg, 0.011 mmol) was dissolved in 7N ammonia in methanol (1 mL). The reaction media was stirred at 55° C. for 16 hr. The reaction was concentrated and used without further purification; ¹H NMR (400 MHz, MeOD) δ 4.72 (d, J=3.9 Hz, 1H), 4.52 (d, J=2.1 Hz, 1H), 4.30 (dd, J=7.6, 3.3 Hz, 1H), 3.71-3.53 (m, 2H), 3.17 (td, J=6.5, 2.6 Hz, 2H), 3.01-2.89 (m, 1H), 2.79 (tt, J=8.2, 4.0 Hz, 1H), 2.43 (dt, J=8.9, 4.2 Hz, 1H), 2.01-1.71 (m, 5H), 1.69-1.14 (m, 21H), 1.12-0.99 (m, 2H); MS (ESI) m/z 534.2 [C₂₇H₄₃N₅O₆+H]⁺.

### Step 4-12. Synthesis of tert-butyl ((4aS,9S,12 aR,15aS,15bS,18S,19aS)-18-cyano-1,10,16-trioxodocosahydro-1H-cyclopenta[3,4]pyrrolo[1,2-a]pyrido[3,4-g][1,4]diazacyclotetradecin-9-yl)carbamate

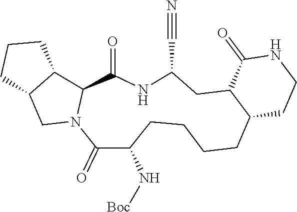

Procedure is similar to Step 4-6.

¹H NMR (400 MHz, MeOD) δ 6.86 (d, J=7.4 Hz, 1H), 5.47 (dd, J=8.1, 3.2 Hz, 1H), 4.56 (d, J=2.2 Hz, 1H), 4.41 (tt, J=7.4, 3.2 Hz, 1H), 3.74 (qd, J=10.9, 5.9 Hz, 2H), 3.31-3.22 (m, 2H), 3.07 (tdd, J=8.4, 6.2, 2.2 Hz, 1H), 2.92 (tq, J=8.2, 4.3 Hz, 1H), 2.39 (dt, J=8.8, 4.0 Hz, 1H), 2.14 (ddd, J=14.8, 9.6, 3.3 Hz, 1H), 2.08-1.84 (m, 5H), 1.82-1.24 (m, 19H), 1.13 (p, J=7.8 Hz, 2H); MS (ESI): m/z=516.3 [M+H⁺; C₂₇H₄₁N₅O₅+ H⁺]; MS (ESI): m/z=502.3 [M+H⁺; C₂₆H₃₉N₅O₅++H⁺]

### General Route 4

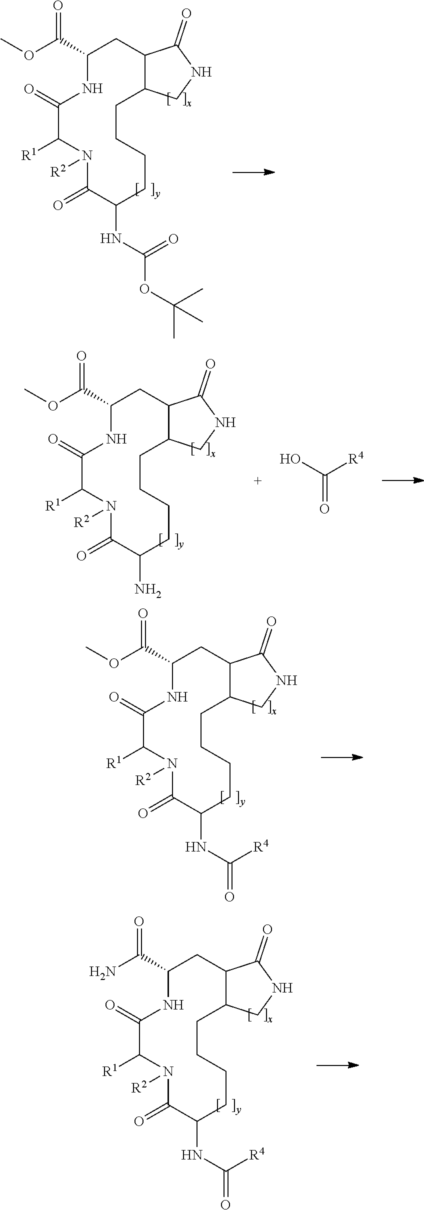

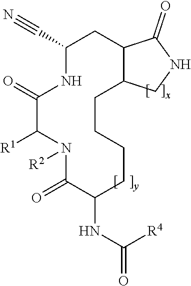

### Example 5

### Specific Exemplification of General Route 4

### N-((3aR,8S,11aS,12aR,12bS,15S,16aS)-15-cyano-12,12-dimethyl-1,9,13-trioxoicosahydrocyclopropa[3,4]pyrrolo[1,2-a]pyrrolo[3,4-g][1,4]diazacyclotetradecin-8-yl)-3,3-dimethylbutanamide

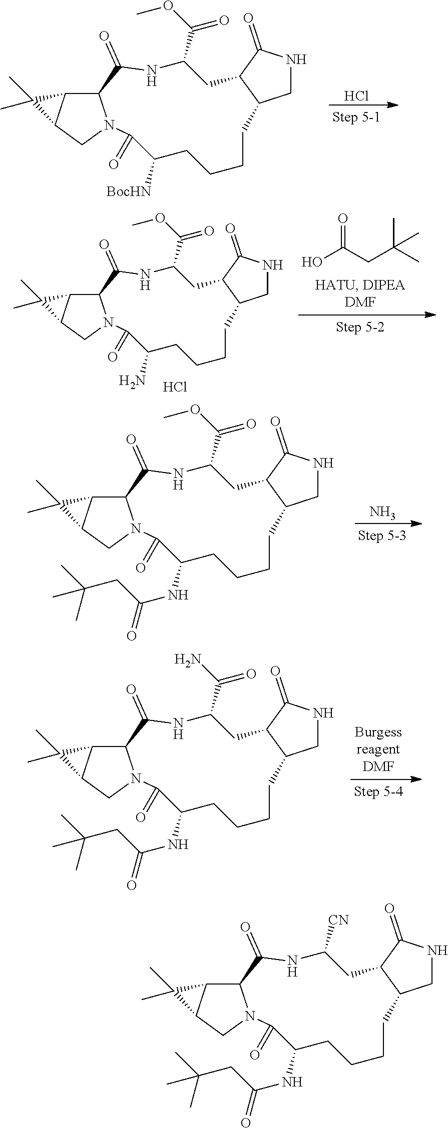

### Step 5-1. Synthesis of methyl (3aR,8S,11 aS,12aR,12bS,15S,16 aS)-8-amino-12,12-dimethyl-1,9,13-trioxoicosahydrocyclopropa [3,4]pyrrolo[1,2-a]pyrrolo[3,4-g][1,4]diazacyclotetradecine-15-carboxylate hydrochloride

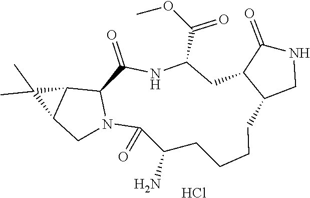 [SMILES: COC(=O)[C@@H]1C[C@@H]2C(=O)NC[C@@H]2CCCC[C@H](N)C(=O)N2C[C@H]3[C@@H]([C@H]2C(=O)N1)C3(C)C ; Cl]

Methyl (3aR,8S,11aS,12aR,12bS,15S,16aS)-8-((tert-butoxycarbonyl)amino)-12,12-dimethyl-1,9,13-trioxoicosahydrocyclopropa [3,4]pyrrolo[1,2-a]pyrrolo[3,4-g][1,4]diazacyclotetradecine-15-carboxylate (30 mg, 0.0561 mmol, 1 equiv.) was dissolved in 4M HCl in dioxane (0.280 mL, 1.12 mmol, 20 equiv.) and stirred at room temperature for 30 minutes. The reaction mixture was concentrated in vacuo to afford the crude titled compound. MS (ESI) m/z 435.2 [C₂₂H₃₄N₄O₅+ H]⁺.

### Step 5-2. Synthesis of methyl (3aR,8S,11aS,12aR,12bS,15S,16aS)-8-(3,3-dimethylbutanamido)-12,12-dimethyl-1,9,13-trioxoicosahydrocyclopropa[3,4]pyrrolo[1,2-a]pyrrolo[3,4-g][1,4]diazacyclotetradecine-15-carboxylate

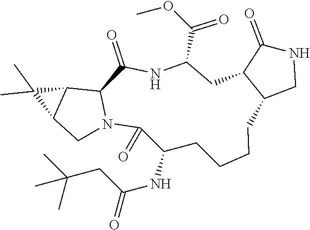 [SMILES: COC(=O)[C@@H]1C[C@@H]2C(=O)NC[C@@H]2CCCC[C@H](NC(=O)CC(C)(C)C)C(=O)N2C[C@H]3[C@@H]([C@H]2C(=O)N1)C3(C)C]

Methyl (3aR,8S,11 aS,12aR,12bS,15S,16 aS)-8-amino-12,12-dimethyl-1,9,13-trioxoicosahydrocyclopropa[3,4]pyrrolo[1,2-a]pyrrolo[3,4-g][1,4]diazacyclotetradecine-15-carboxylate hydrochloride (crude from step 5-1, 0.0561 mmol, 1 equiv.) and 3,3-dimethylbutanoic acid (8.6 μL, 0.0673 mmol, 1.2 equiv.) were dissolved in anhydrous DMF (0.561 mL) and cooled to 0° C. N,N-diisopropylethylamine (48.8 μL, 0.280 mmol, 5 equiv.) and HATU (23.4 mg, 0.0617 mmol, 1.1 equiv) was added and the resulting yellow solution was stirred for 30 minutes. The completed reaction was diluted with ethyl acetate and washed with saturated ammonium chloride and brine sequentially. The organic fraction was dried with anhydrous sodium sulfate, concentrated in vacuo and purified via semi-preparative reverse phase HPLC (25-55% MeCN/H₂O, 0.1% formic acid) to afford the titled compound as a white solid (20.8 mg, 0.039 mmol, 70%); 1H NMR (400 MHz, MeOD) δ 8.37 (d, J=8.9 Hz, 1H), 8.11 (d, J=7.3 Hz, 1H), 4.77-4.67 (m, 1H), 4.57 (s, 1H), 4.53 (dd, J=9.2, 3.6 Hz, 1H), 4.13 (d, J=10.7 Hz, 1H), 3.87 (dd, J=10.6, 5.2 Hz, 1H), 3.77 (s, 3H), 3.37-3.34 (m, 1H), 3.02 (dd, J=10.0, 5.6 Hz, 1H), 2.58 (q, J=7.2 Hz, 1H), 2.42 (h, J=6.7 Hz, 1H), 2.23 (ddd, J=14.7, 7.2, 3.7 Hz, 1H), 2.14-2.04 (m, 2H), 1.99-1.85 (m, 1H), 1.80-1.59 (m, 4H), 1.57-1.45 (m, 1H), 1.42-1.19 (m, 5H), 1.10 (s, 3H), 1.01 (s, 9H), 0.96 (s, 3H); MS (ESI) m/z 533.2 [C₂₈H₄₄N₄O₆+H]⁺.

### Step 5-3. Synthesis of (3aR,8S,11aS,12aR,12bS,15S,16aS)-8-(3,3-dimethylbutanamido)-12,12-dimethyl-1,9,13-trioxoicosahydrocyclopropa[3,4]pyrrolo[1,2-a]pyrrolo[3,4-g][1,4]diazacyclotetradecine-15-carboxamide

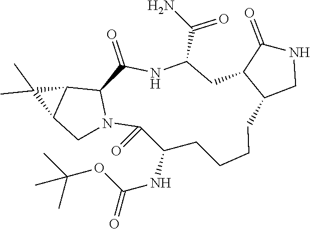 [SMILES: CC(C)(C)OC(=O)N[C@H]1CCCC[C@H]2CNC(=O)[C@H]2C[C@@H](C(N)=O)NC(=O)[C@@H]2[C@@H]3[C@H](CN2C1=O)C3(C)C]

The titled compound was prepared analogous to Step 3-8. The compound was purified via semi-preparative reverse phase HPLC (25-55% MeCN/H₂O, 0.1% formic acid) to afford the titled compound as a white solid (16.6 mg, 0.032 mmol, 82%); ¹H NMR (400 MHz, MeOD) δ 8.28 (d, J=8.6 Hz, 1H), 8.12 (d, J=7.2 Hz, 1H), 4.70-4.60 (m, 1H), 4.58 (s, 1H), 4.57-4.52 (m, 1H), 4.07 (d, J=10.7 Hz, 1H), 3.85 (dd, J=10.6, 5.3 Hz, 1H), 3.35 (d, J=6.7 Hz, 1H), 3.01 (dd, J=10.0, 5.7 Hz, 1H), 2.71 (q, J=7.1 Hz, 1H), 2.42 (h, J=6.7 Hz, 1H), 2.16-2.05 (m, 3H), 1.97 (ddd, J=13.8, 8.2, 6.2 Hz, 1H), 1.79-1.64 (m, 3H), 1.61 (dd, J=7.7, 5.1 Hz, 1H), 1.59-1.48 (m, 1H), 1.41-1.20 (m, 5H), 1.10 (s, 3H), 1.01 (s, 9H), 0.96 (s, 3H); MS (APCI) m/z 518.4 [C₂₇H₄₃N₅O₅+H]⁺.

Step 5-4. Synthesis of N-((3aR,8S,11 aS,12aR,12bS,15S,16 aS)-15-cyano-12,12-dimethyl-1,9,13-trioxoicosahydrocyclopropa [3,4]pyrrolo[1,2-a]pyrrolo[3,4-g][1,4]diazacyclotetradecin-8-yl)-3,3-dimethylbutanamide1

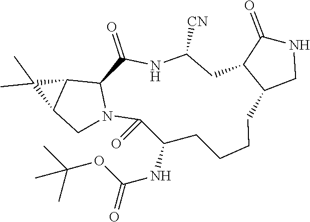 [SMILES: CC(C)(C)OC(=O)N[C@H]1CCCC[C@H]2CNC(=O)[C@H]2C[C@@H](C#N)NC(=O)[C@@H]2[C@@H]3[C@H](CN2C1=O)C3(C)C]

The titled compound was prepared analogous to Step 3-9. The compound was purified via semi-preparative reverse phase HPLC (20-70% MeCN/H₂O, 0.1% formic acid) to afford the titled compound as a white solid (12.4 mg, 0.025 mmol, 46%); ¹H NMR (400 MHz, MeOD) δ (ppm) 8.70 (d, J=8.6 Hz, 0.3H), 8.09 (d, J=7.2 Hz, 1H), 5.18-5.08 (m, 1H), 4.59-4.47 (m, 2H), 4.09 (d, J=10.7 Hz, 1H), 3.86 (dd, J=10.6, 5.0 Hz, 1H), 3.42-3.35 (m, 1H), 3.01 (dd, J=10.0, 6.0 Hz, 1H), 2.47 (dq, J=23.4, 7.1 Hz, 2H), 2.19 (ddd, J=14.8, 7.4, 4.4 Hz, 1H), 2.12-1.99 (m, 3H), 1.98-1.86 (m, 1H), 1.72-1.59 (m, 3H), 1.56-1.40 (m, 1H), 1.40-1.18 (m, 5H), 1.10 (s, 3H), 1.01 (s, 9H), 0.97 (s, 3H); MS (ESI) m/z 500.2 [C₂₇H₄₁N₅O₄+ H]⁺.

### General Route 5

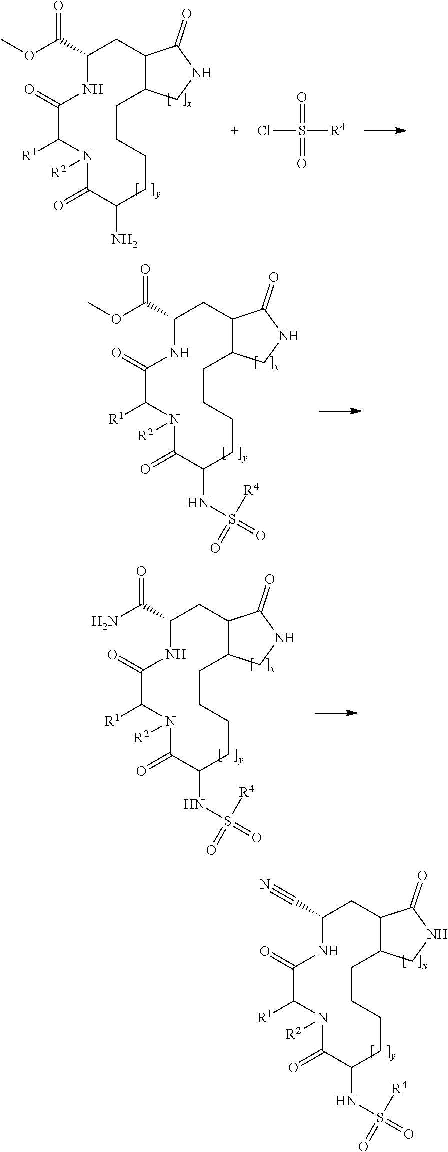

### Example 6

### Specific Exemplification of General Route 5

### N-((3aR,8S,11aS,12aR,12bS,15S,16aS)-15-cyano-12,12-dimethyl-1,9,13-trioxoicosahydrocyclopropa[3,4]pyrrolo[1,2-a]pyrrolo[3,4-g][1,4]diazacyclotetradecin-8-yl)benzenesulfonamide

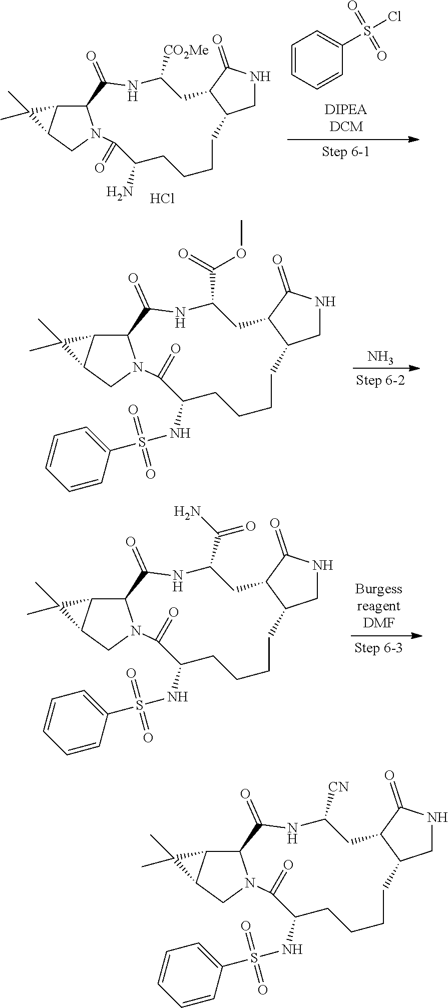

### Step 6-1. Synthesis of methyl (3aR,8S,11aS,12aR,12bS,15S,16aS)-12,12-dimethyl-1,9,13-trioxo-8-(phenylsulfonamido)icosahydrocyclopropa [3,4]pyrrolo[1,2-a]pyrrolo[3,4-g][1,4]diazacyclotetradecine-15-carboxylate

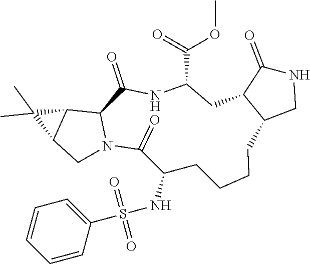 [SMILES: COC(=O)[C@@H]1C[C@@H]2C(=O)NC[C@@H]2CCCC[C@H](NS(=O)(=O)c2ccccc2)C(=O)N2C[C@H]3[C@@H]([C@H]2C(=O)N1)C3(C)C]

Methyl (3aR,8S,11aS,12aR,12bS,15S,16aS)-8-amino-12,12-dimethyl-1,9,13-trioxoicosahydrocyclopropa[3,4]pyrrolo[1,2-a]pyrrolo[3,4-g][1,4]diazacyclotetradecine-15-carboxylate hydrochloride (prepared similar to step 5-1, 0.0841 mmol, 1 equiv.) was dissolved in dichloromethane (0.841 mL) and cooled to 0° C. Benzenesulfonyl chloride (12.9 μL, 0.101 mmol, 1.2 equiv.) and N,N-diisopropylethylamine (44 μL, 0.252 mmol, 3 equiv.) was added and the reaction was allowed to stir for 3.5 hours while warming up to room temperature. The completed reaction was quenched with saturated ammonium chloride solution and extracted with dichloromethane (4×5 mL). The organic layers were combined and dried with anhydrous sodium sulfate, concentrated in vacuo and purified via semi-preparative reverse phase HPLC (25-55% MeCN/H₂O, 0.1% formic acid) to afford the titled compound as a white solid (38.7 mg, 0.067 mmol, 80%); 1H NMR (400 MHz, MeOD) δ 8.28 (d, J=8.9 Hz, 1H), 7.92-7.85 (m, 2H), 7.68-7.60 (m, 1H), 7.60-7.47 (m, 2H), 4.75 (tt, J=7.6, 4.6 Hz, 1H), 4.46 (s, 1H), 4.04 (dd, J=7.5, 3.3 Hz, 1H), 3.82-3.72 (m, 1H), 3.75 (s, 3H), 3.68 (d, J=10.8 Hz, 1H), 2.97 (dd, J=9.9, 5.9 Hz, 1H), 2.56 (q, J=7.1 Hz, 1H), 2.38 (h, J=6.7 Hz, 1H), 2.20 (ddd, J=14.6, 7.4, 4.5 Hz, 1H), 1.88 (dq, J=13.2, 6.3 Hz, 1H), 1.73-1.61 (m, 2H), 1.58-1.46 (m, 2H), 1.40-1.16 (m, 5H), 1.10 (d, J=8.1 Hz, 1H), 1.04 (s, 3H), 0.66 (s, 3H); MS (ESI) m/z 575.1 [C₂₈H₃₈N₄O₇S+H]⁺.

### Step 6-2. Synthesis of (3aR,8S,11aS,12aR,12bS,15S,16aS)-12,12-dimethyl-1,9,13-trioxo-8-(phenylsulfonamido)icosahydrocyclopropa [3,4]pyrrolo[1,2-a]pyrrolo[3,4-g][1,4]diazacyclotetradecine-15-carboxamide

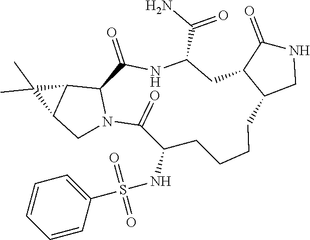 [SMILES: CC1(C)[C@@H]2[C@H]3C(=O)N[C@H](C(N)=O)C[C@@H]4C(=O)NC[C@@H]4CCCC[C@H](NS(=O)(=O)c4ccccc4)C(=O)N3C[C@@H]21]

The titled compound was prepared in the same way as step 5-3 of general route 5. The compound was purified via semi-preparative reverse phase HPLC (25-55% MeCN/H₂O, 0.1% formic acid) to afford the titled compound as a white solid (28.4 mg, 0.051 mmol, 75%); ¹H NMR (400 MHz, MeOD) δ 8.20 (d, J=8.6 Hz, 1H), 7.92-7.85 (m, 2H), 7.65-7.52 (m, 4H), 4.69-4.60 (m, 1H), 4.45 (s, 1H), 4.04 (dd, J=6.9, 3.1 Hz, 1H), 3.75 (dd, J=10.7, 5.1 Hz, 1H), 3.64 (d, J=10.7 Hz, 1H), 2.96 (dd, J=9.9, 6.7 Hz, 1H), 2.69 (td, J=7.8, 5.8 Hz, 1H), 2.39 (h, J=6.9 Hz, 1H), 2.09-1.96 (m, 1H), 1.91 (dt, J=13.0, 6.7 Hz, 1H), 1.69 (dt, J=14.7, 6.3 Hz, 1H), 1.63 (d, J=7.6 Hz, 1H), 1.58-1.49 (m, 2H), 1.45-1.33 (m, 1H), 1.31-1.20 (m, 4H), 1.18-1.08 (m, 1H), 1.04 (s, 3H), 0.64 (s, 3H); MS (APCI) m/z 560.3 [C₂₇H₃₇N₅O₆S+H]⁺.

### Step 6-3. Synthesis of N-((3aR,8S,11aS,12aR,12bS,15S,16aS)-15-cyano-12,12-dimethyl-1,9,13-trioxoicosahydrocyclopropa [3,4]pyrrolo[1,2-a]pyrrolo[3,4-g][1,4]diazacyclotetradecin-8-yl)benzenesulfonamide

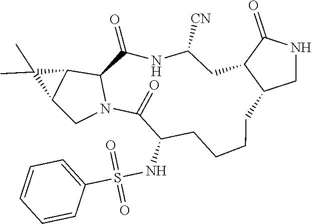 [SMILES: CC1(C)[C@@H]2[C@H]3C(=O)N[C@H](C#N)C[C@@H]4C(=O)NC[C@@H]4CCCC[C@H](NS(=O)(=O)c4ccccc4)C(=O)N3C[C@@H]21]

The titled compound was prepared in the same way as step 5-4 of general route 5. The compound was purified via semi-preparative reverse phase HPLC (25-55% MeCN/H₂O, 0.1% formic acid) to afford the titled compound as a white solid (23.1 mg, 0.043 mmol, 84%); 1H NMR (400 MHz, MeOD) δ (ppm) 7.87 (dd, J=7.2, 1.7 Hz, 2H), 7.67-7.59 (m, 1H), 7.59-7.50 (m, 2H), 5.12 (dd, J=6.5, 4.9 Hz, 1H), 4.39 (s, 1H), 4.04 (dd, J=7.5, 3.2 Hz, 1H), 3.76 (dd, J=10.6, 4.8 Hz, 1H), 3.68 (d, J=10.6 Hz, 1H), 3.37-3.34 (m, 1H), 2.97 (dd, J=9.9, 6.2 Hz, 1H), 2.41 (dp, J=27.2, 7.0 Hz, 2H), 2.26-2.11 (m, 1H), 1.96 (dt, J=14.9, 6.4 Hz, 1H), 1.85 (dq, J=13.4, 6.6 Hz, 1H), 1.66-1.54 (m, 2H), 1.54-1.39 (m, 1H), 1.39-1.14 (m, 5H), 1.12-0.96 (m, 4H), 0.70 (s, 3H); MS (ESI) m/z 542.1 [C₂₇H₃₅N₅O₅S+H]⁺.

### General Route 6

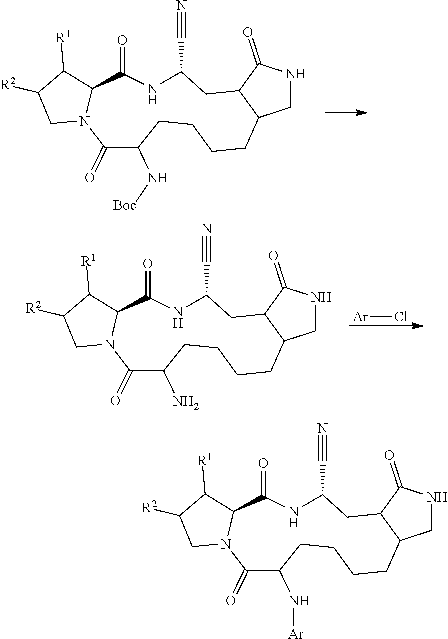

### Example 7

### Specific Exemplification of General Route 6

### (3aR,8S,11 aS,12aR,12bS,15S,16aS)-12,12-dimethyl-1,9,13-trioxo-8-((4-(trifluoromethyl)pyrimidin-2-yl)amino)icosahydrocyclopropa[3,4]pyrrolo[1,2-a]pyrrolo[3,4-g][1,4]diazacyclotetradecine-15-carbonitrile

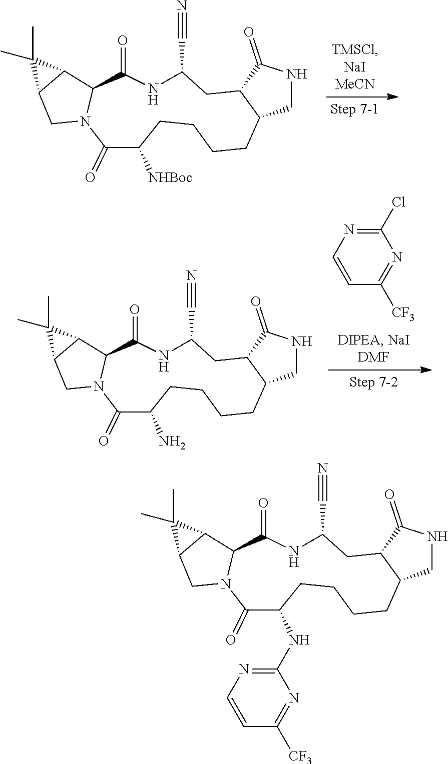 [SMILES: CC(C)(C)OC(=O)N[C@H]1CCCC[C@H]2CNC(=O)[C@H]2C[C@@H](C#N)NC(=O)[C@@H]2[C@@H]3[C@H](CN2C1=O)C3(C)C ; CC1(C)[C@@H]2[C@H]3C(=O)N[C@H](C#N)C[C@@H]4C(=O)NC[C@@H]4CCCC[C@H](N)C(=O)N3C[C@@H]21 ; FC(F)(F)c1ccnc(Cl)n1 ; CC1(C)[C@@H]2[C@H]3C(=O)N[C@H](C#N)C[C@@H]4C(=O)NC[C@@H]4CCCC[C@H](Nc4nccc(C(F)(F)F)n4)C(=O)N3C[C@@H]21]

### Step 7-1. Synthesis of (3aR,8S,11aS,12aR,12bS,15S,16aS)-8-amino-12,12-dimethyl-1,9,13-trioxoicosahydrocyclopropa[3,4]pyrrolo[1,2-a]pyrrolo[3,4-g][1,4]diazacyclotetradecine-15-carbonitrile

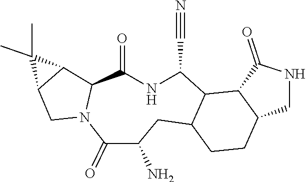 [SMILES: CC1(C)[C@@H]2[C@H]3C(=O)N[C@H](C#N)C4C(CC[C@H]5CNC(=O)[C@@H]45)C[C@H](N)C(=O)N3C[C@@H]21]

A solution of trimethylsilyl iodide was prepared by the addition of sodium iodide (85.2 mg, 0.568 mmol, 5.0 equiv.) to a solution of trimethylsilyl chloride (62 mg, 0.568 mmol, 5.0 equiv.) in anhydrous acetonitrile (0.5 mL) under nitrogen. The reaction was stirred at room temperature for 30 min, during which white solid precipitation was observed. To the above cloudy solution was added tert-butyl N-[(1S,4S,6S,10R,15S,19S,21R)-4-cyano-20,20-dimethyl-2,7,16-trioxo-3,8,17-triazatetracyclo[15.4.0.0\hat{ }{6,10}0.0\hat{ }{19,21}]henicosan-15-yl]carbamate (57 mg, 0.114 mmol, 1.0 equiv) dissolved in anhydrous acetonitrile (1.0 mL). The resulting suspension was stirred at room temperature for 15 min. The crude reaction mixture was then filtered and concentrated in vacuo for 3 hrs to afford the titled compound as a clear film. MS (ESI) m/z 402.3 [C₂₁H₃₁N₅O₃+ H]⁺.

The material could be purified further by semi-preparative reverse phase HPLC (5-35% MeCN/H₂O, 0.1% formic acid) to afford titled material as a white solid upon lyophilization; 1H NMR (400 MHz, MeOD) δ 5.21 (dd, J=6.5, 4.9 Hz, 1H), 4.57 (s, 1H), 3.96 (t, J=4.4 Hz, 1H), 3.83 (dd, J=10.7, 5.1 Hz, 1H), 3.75 (d, J=10.7 Hz, 1H), 3.42-3.35 (m, 1H), 2.99 (dd, J=9.9, 6.8 Hz, 1H), 2.57-2.33 (m, 2H), 2.25-2.11 (m, 1H), 2.00-1.91 (m, 1H), 1.91-1.76 (m, 2H), 1.72 (d, J=7.6 Hz, 1H), 1.65 (dd, J=7.7, 4.9 Hz, 1H), 1.41-1.28 (m, 5H), 1.19-1.13 (m, 1H), 1.12 (s, 3H), 0.98 (s, 3H).

### Step 7-2. Synthesis of (3aR,8S,11aS,12aR,12bS,15S,16aS)-12,12-dimethyl-1,9,13-trioxo-8-((4-(trifluoromethyl)pyrimidin-2-yl)amino)icosahydrocyclopropa[3,4]pyrrolo[1,2-a]pyrrolo[3,4-g][1,4]diazacyclotetradecine-15-carbonitrile

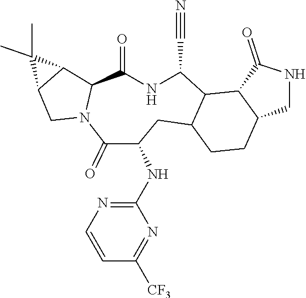 [SMILES: CC1(C)[C@@H]2[C@H]3C(=O)N[C@H](C#N)C4C(CC[C@H]5CNC(=O)[C@@H]45)C[C@H](Nc4nccc(C(F)(F)F)n4)C(=O)N3C[C@@H]21]

(1S,4S,6S,10R,15S,19S,21R)-15-amino-20,20-dimethyl-2,7,16-trioxo-3,8,17-triazatetracyclo[15.4.0.0\hat{ }{6,10}0.0\hat{ }{19,21}]henicosane-4-carbonitrile (crude from Step 1; 45 mg, 0.112 mmol, 1.0 equiv), 2-chloro-4-(trifluoromethyl)-pyrimidine (24.6 mg, 0.134 mmol, 1.2 equiv)) and N,N-diisopropylethylamine (99 μL, 0.56 mmol, 5.0 equiv.) was dissolved in anhydrous dimethylacetamide (1.1 mL). Sodium iodide (1 crystal, catalytic amount) was added and the reaction stirred at room temperature for 24 hr under nitrogen. Water (0.2 mL) was added and the crude material was purified by by semi-preparative reverse phase HPLC (20-70% MeCN/H₂O, 0.1% formic acid) to afford titled material as a white solid upon lyophilization (22 mg, 0.040 mmol, 36%) 1H NMR (400 MHz, MeOD) δ 8.67-8.31 (m, 1H), 6.96 (d, J=4.9 Hz, 1H), 5.27-5.05 (m, 1H), 4.83-4.59 (m, 1H), 4.52 (s, 1H), 4.15 (d, J=10.6 Hz, 1H), 3.92 (dt, J=10.6, 2.6 Hz, 1H), 3.42-3.35 (m, 1H), 3.01 (dd, J=9.9, 6.3 Hz, 1H), 2.63-2.40 (m, 2H), 2.31-2.12 (m, 1H), 2.12-1.94 (m, 2H), 1.87-1.72 (m, 1H), 1.67 (s, 2H), 1.60-1.45 (m, 1H), 1.45-1.19 (m, 6H), 1.15-1.01 (m, 4H), 0.97-0.75 (m, 2H) (Compound exists as a mixture of tautomeric form); MS (ESI) m/z 548.1 [C₂₁H₃₁N₅O₃+H]⁺.

### General Route 7

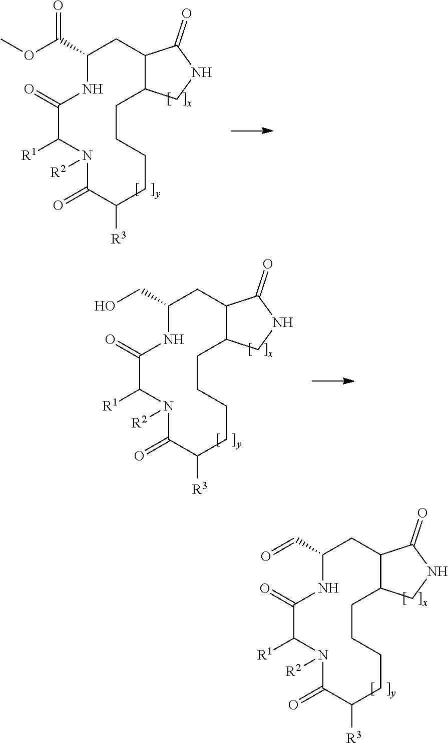

### Example 8

### Specific Exemplification of General Route 7 (3aR,5S,8S,11S,14aS)-11-benzyl-8-isobutyl-3,7,10-trioxohexadecahydropyrrolo[3,4-g][1,4]diazacyclotridecine-5-carbaldehyde

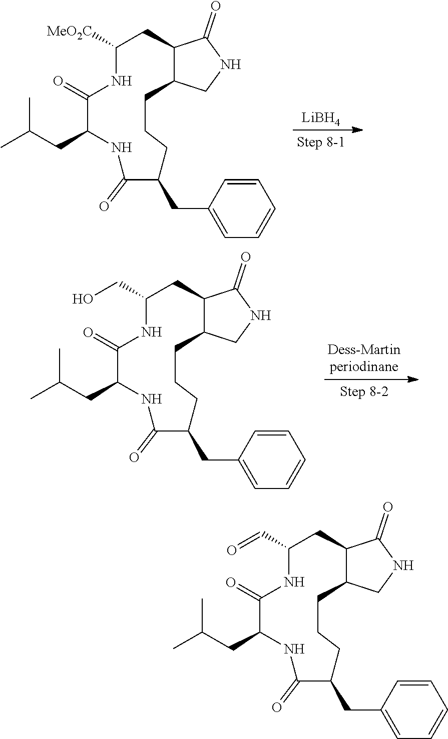

### Step 8-1. Synthesis of (3aR,5S,8S,11S,14aS)-11-benzyl-5-(hydroxymethyl)-8-isobutyldodecahydropyrrolo[3,4-g][1,4]diazacyclotridecine-3,7,10(2H)-trione

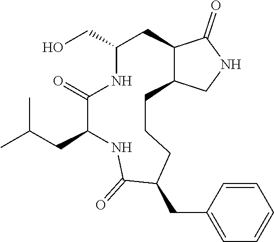 [SMILES: CC(C)C[C@@H]1NC(=O)[C@H](Cc2ccccc2)CCC[C@@H]2CNC(=O)[C@@H]2C[C@@H](CO)NC1=O]

Methyl (3aR,5S,8S,11S,14aS)-11-benzyl-8-isobutyl-3,7,10-trioxohexadecahydropyrrolo[3,4-g][1,4]diazacyclotridecine-5-carboxylate (13.9 mg, 0.0295 mmol, 1 equiv.) was dissolved in 2M LiBH₄ in THF (0.147 mL) and the reaction was stirred at room temperature for 30 minutes. The completed reaction was diluted with ethyl acetate and washed with saturated ammonium chloride solution four times.

The aqueous layers were combined and back extracted with a fresh solution of ethyl acetate. The organic layers were combined, dried with anhydrous sodium sulfate and concentrated in vacuo to afford the crude titled compound as a white solid (12.3 mg, 0.028 mmol, 94%). MS (APCI) m/z 444.3 [C₂₅H₃₇N₃O₄+ H]⁺.

### Step 8-2. Synthesis of (3aR,5S,8S,11S,14aS)-11-benzyl-8-isobutyl-3,7,10-trioxohexadecahydropyrrolo[3,4-g][1,4]diazacyclotridecine-5-carbaldehyde

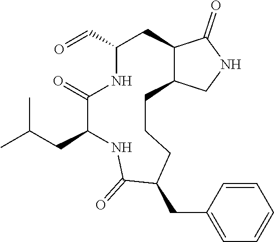 [SMILES: CC(C)C[C@@H]1NC(=O)[C@H](Cc2ccccc2)CCC[C@@H]2CNC(=O)[C@@H]2C[C@@H](C=O)NC1=O]

(3aR,5S,8S,11S,14aS)-11-benzyl-5-(hydroxymethyl)-8-isobutyldodecahydropyrrolo[3,4-g][1,4]diazacyclotridecine-3,7,10(2H)-trione (3.7 mg, 0.00834 mmol, 1 equiv.) was dissolved in DMF (0.2 mL) and cooled to 0° C. Dess-Martin periodinane (7.07 mg, 0.0166 mmol, 2 equiv.) was added and the reaction was stirred at room temperature for 2 hours. A second portion of Dess-Martin periodinane was also added after the first hour. The completed reaction was filtered through a syringe filter and rinsed with methanol. The filtrate was purified via semi-preparative reverse phase HPLC (20-70% MeCN/H₂O, 0.1% formic acid) to afford the titled compound as a white solid upon lyophilization (2.4 mg, 0.005 mmol, 65%); 1H NMR (400 MHz, DMSO-d₆): δ (ppm) 9.59-9.54 (m, 0.01H), 7.87 (d, J=7.7 Hz, 0.3H), 7.51 (d, J=7.1 Hz, 0.2H), 7.37 (t, 3=7.3 Hz, 0.2H), 7.31-7.12 (m, 5H), 7.04 (t, 3=7.6 Hz, 0.2H), 4.67-4.54 (m, 1H), 4.17-4.04 (m, 1H), 3.28-3.22 (m, 1H), 3.07-2.99 (m, 1H), 2.93-2.50 (m, 4H), 2.47-2.38 (m, 1H), 2.05-1.89 (m, 1H), 1.89-1.74 (m, 1H), 1.66-1.12 (m, 9H), 0.92-0.64 (m, 6H); MS (APCI) m/z 442.3 [C₂₅H₃₅N₃O₄+ H]⁺.

Some compounds herein may exist as mixtures of hydrates and parent compound.

| Cpd No. | General Route | Structure | NMR and m/z |
|---|---|---|---|
| 1 | 6 | 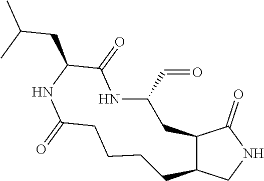 [SMILES: CC(C)C[C@@H]1NC(=O)CCCC[C@@H]2CNC(=O)[C@@H]2C[C@@H](C=O)NC1=O] | ¹H NMR (400 MHz, MeOD) δ 4.59 (m, 1H), 4.55 (m, 1H), 4.06 (dq, J = 7.7, 4.0 Hz, 1H), 3.32-3.25 (m, 1H), 3.08 (dd, J = 10.1, 5.1 Hz, 1H), 2.76 (ddt, J = 12.6, 8.7, 3.9 Hz, 1H), 2.66 (dt, J = 12.3, 6.2 Hz, 1H), 2.31 (ddd, J = 13.5, 6.7, 3.1 Hz, 1H), 2.24-2.12 (m, 1H), 2.02-1.91 (m, 1H), 1.85 (th, J = 11.4, 3.4 Hz, 2H), 1.71-1.53 (m, 5H), 1.34 (ddd, J = 18.6, 9.4, 5.6 Hz, 2H), 1.24 (m, 1H), 1.03-0.87 (m, 6H). MS (ESI) m/z 352.2 [C₁₈H₂₉N₃O₄ + H]⁺ |
| 2 | 6 | 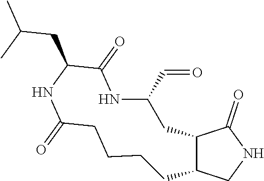 [SMILES: CC(C)C[C@@H]1NC(=O)CCCC[C@H]2CNC(=O)[C@H]2C[C@@H](C=O)NC1=O] | ¹H NMR (400 MHz, MeOD) δ 4.56- 4.53 (m, 2H), 4.12-3.91 (m, 1H), 3.33 (m, 1H), 3.12-3.01 (m, 1H), 2.48 (tt, J = 7.6, 3.9 Hz, 1H), 2.45- 2.33 (m, 1H), 2.33-2.19 (m, 2H), 2.11-1.99 (m, 1H), 1.87-1.75 (m, 1H), 1.66 (m, 5H), 1.56-1.15 (m, 4H), 1.08-0.86 (m, 6H). MS (ESI) m/z 352.2 [C₁₈H₂₉N₃O₄ + H]⁺ |
| 3 | 6 | 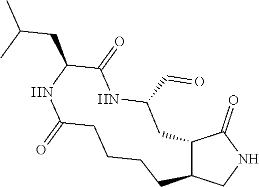 [SMILES: CC(C)C[C@@H]1NC(=O)CCCC[C@@H]2CNC(=O)[C@H]2C[C@@H](C=O)NC1=O] | ¹H NMR (400 MHz, MeOD) δ 4.71- 4.47 (m, 2H), 4.41-4.27 (m, 1H), 3.40-3.33 (m, 1H), 2.93-2.85 (m, 1H), 2.38-2.22 (m, 2H), 2.13-1.86 (m, 4H), 1.71-1.38 (m, 9H), 0.96 (dd, J = 6.2, 2.0 Hz, 3H), 0.91 (d, J = 6.1 Hz, 3H). MS (ESI) m/z 352.2 [M + H +; C₁₈H₃₀N₃O₄]⁺ |
| 4 | 6 | 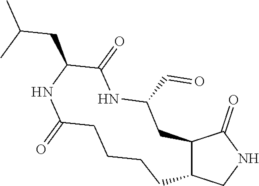 [SMILES: CC(C)C[C@@H]1NC(=O)CCCC[C@H]2CNC(=O)[C@@H]2C[C@@H](C=O)NC1=O] | ¹H NMR (400 MHz, MeOD) δ 4.71- 4.48 (m, 2H), 3.96-3.81 (m, 1H), 3.45-3.33 (m, 1H), 2.92 (ddd, J = 9.9, 6.6, 5.1 Hz, 1H), 2.33-2.10 (m, 3H), 2.09-1.80 (m, 3H), 1.76-1.28 (m, 8H), 1.23-1.06 (m, 1H), 0.95 (dd, J = 13.7, 5.9 Hz, 6H). MS (ESI) m/z 352.2 [M + H+; C₁₈H₃₀N₃O₄]⁺ |
| 5 | 6 | 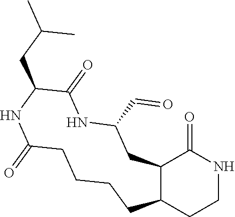 [SMILES: CC(C)C[C@@H]1NC(=O)CCCC[C@@H]2CCNC(=O)[C@@H]2C[C@@H](C=O)NC1=O] | ¹H NMR (400 MHz, MeOD) δ 4.76 (d, J = 4.9 Hz, 1H), 4.66-4.56 (m, 1H), 4.00 (dt, J = 7.4, 3.7 Hz, 1H), 3.30- 3.20 (m, 2H), 2.67 (p, J = 3.8 Hz, 1H), 2.35-2.17 (m, 2H), 1.96-1.78 (m, 4H), 1.75-1.49 (m, 6H), 1.41 (m, 2H), 1.22 (dh, J = 14.2, 7.1 Hz, 2H), 0.99 (d, J = 5.9 Hz, 3H), 0.94 (d, J = 5.6 Hz, 3H). MS (ESI) m/z 366.2 [C₁₉H₃₁N₃O₄ + H]⁺ |
| 6 | 6 | 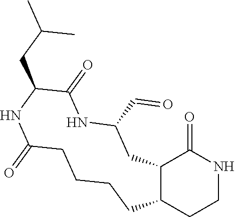 [SMILES: CC(C)C[C@@H]1NC(=O)CCCC[C@H]2CCNC(=O)[C@H]2C[C@@H](C=O)NC1=O] | ¹H NMR (400 MHz, MeOD) δ 4.60 (dt, J = 14.7, 6.2 Hz, 1H), 4.54-4.38 (m, 2H), 3.32-3.20 (m, 2H), 2.54-2.14 (m, 3H), 2.04-1.69 (m, 4H), 1.72- 1.54 (m, 5H), 1.53-1.09 (m, 5H), 1.11-0.78 (m, 6H). MS (ESI) m/z 366.2 [C₁₉H₃₁N₃O₄ + H]⁺ |
| 7 | 6 | 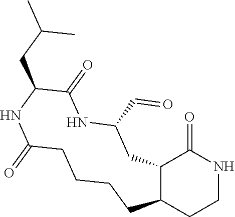 [SMILES: CC(C)C[C@@H]1NC(=O)CCCC[C@@H]2CCNC(=O)[C@H]2C[C@@H](C=O)NC1=O] | ¹H NMR (400 MHz, MeOD) δ 4.68- 4.54 (m, 1H), 4.51-4.27 (m, 1H), 3.32-3.17 (m, 1H), 2.73-1.89 (m, 6H), 1.87-1.12 (m, 12H), 1.04-0.89 (m, 6H). MS (ESI) m/z 366.2 [C₁₉H₃₁N₃O₄ + H]⁺ |
| 8 | 6 | 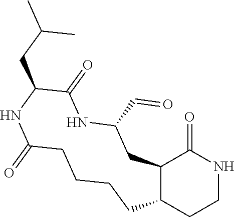 [SMILES: CC(C)C[C@@H]1NC(=O)CCCC[C@H]2CCNC(=O)[C@@H]2C[C@@H](C=O)NC1=O] | 1H NMR (400 MHz, MeOD) δ 4.63- 4.36 (m, 2H), 3.30-3.15 (m, 2H), 2.25 (s, 1H), 2.19-1.88 (m, 3H), 1.87-1.27 (m, 11H), 1.26-1.05 (m, 1H), 1.00-0.90 (m, 6H). MS (ESI) m/z 366.2 [C₁₉H₃₁N₃O₄ + H]⁺ |
| 9 | | 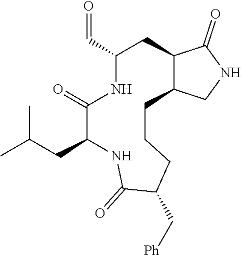 [SMILES: CC(C)C[C@@H]1NC(=O)[C@@H](Cc2ccccc2)CCC[C@@H]2CNC(=O)[C@@H]2C[C@@H](C=O)NC1=O] | ¹H NMR (400 MHz, MeOD) δ 8.37 (d, J = 7.8 Hz, 1H), 7.27-7.19 (m, 2H), 7.19-7.11 (m, 3H), 4.48 (dd, J = 4.0, 2.9 Hz, 1H), 4.40-4.25 (m, 1H), 4.03- 3.89 (m, 1H), 3.39 (d, J = 9.0 Hz, 1H), 3.22 (dd, J = 9.9, 6.2 Hz, 1H), 3.03 (ddt, J = 10.0, 3.7, 2.0 Hz, 1H), 2.83 (dd, J = 13.2, 10.9 Hz, 1H), 2.67 (dd, J = 13.3, 4.4 Hz, 1H), 2.53 (tt, J = 11.0, 3.7 Hz, 1H), 2.41 (tt, J = 8.1, 4.5 Hz, 1H), 2.26 (s, 1H), 2.09-1.92 (m, 1H), 1.84-1.63 (m, 2H), 1.62- 0.83 (m, 8H), 0.77 (d, J = 6.6 Hz, 3H), 0.68-0.56 (m, 3H). MS (ESI) m/z 442.3 [C₂₅H₃₅N₃O₄ + H]⁺ |
| 10 | 6 | 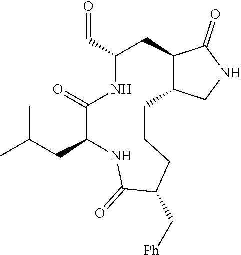 [SMILES: CC(C)C[C@@H]1NC(=O)[C@@H](Cc2ccccc2)CCC[C@H]2CNC(=O)[C@@H]2C[C@@H](C=O)NC1=O] | ¹H NMR (400 MHz, MeOD) δ 7.27- 7.20 (m, 2H), 7.16 (ddd, J = 8.4, 6.2, 1.7 Hz, 3H), 4.60 (d, J = 4.1 Hz, 1H), 4.33 (ddd, J = 17.3, 10.3, 5.0 Hz, 1H), 4.01 (p, J = 3.9 Hz, 1H), 3.26 (dt, J = 6.7, 3.5 Hz, 1H), 3.03 (ddd, J = 9.7, 5.9, 2.6 Hz, 1H), 2.96-2.56 (m, 4H), 2.52 (s, 1H), 2.03-1.66 (m, 3H), 1.57 (s, 2H), 1.51-1.18 (m, 5H), 1.00-0.85 (m, 1H), 0.83-0.72 (m, 3H), 0.73-0.58 (m, 3H). MS (ESI) m/z 442.2 [C₂₅H₃₅N₃O₄ + H]⁺ |
| 11 | 6 | 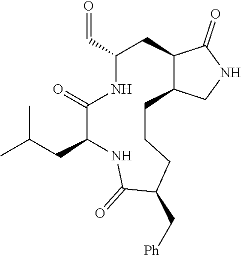 [SMILES: CC(C)C[C@@H]1NC(=O)[C@H](Cc2ccccc2)CCC[C@@H]2CNC(=O)[C@@H]2C[C@@H](C=O)NC1=O] | ¹H NMR (400 MHz, MeOD-d₄): δ (ppm) 9.59-9.54 (m, 0.01H), 7.87 (d, J = 7.7 Hz, 0.3H), 7.51 (d, J = 7.1 Hz, 0.2H), 7.37 (t, J = 7.3 Hz, 0.2H), 7.31-7.12 (m, 5H), 7.04 (t, J = 7.6 Hz, 0.2H), 4.67-4.54 (m, 1H), 4.17-4.04 (m, 1H), 3.28-3.22 (m, 1H), 3.07-2.99 (m, 1H), 2.93-2.50 (m, 4H), 2.47- 2.38 (m, 1H), 2.05-1.89 (m, 1H), 1.89-1.74 (m, 1H), 1.66-1.12 (m, 9H), 0.92-0.64 (m, 6H) MS (ESI) m/z = 442.3 [C₂₅H₃₅N₃O₄ + H]⁺ |
| 12 | 6 | 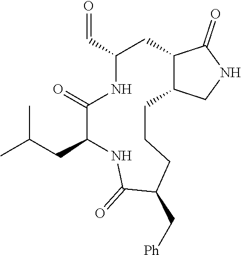 [SMILES: CC(C)C[C@@H]1NC(=O)[C@H](Cc2ccccc2)CCC[C@H]2CNC(=O)[C@H]2C[C@@H](C=O)NC1=O] | ¹H NMR (400 MHz, MeOD-d₄): δ (ppm) 9.51 (s, 0.03H), 8.28 (d, J = 8.2 Hz, 0.8H), 8.00 (dd, J = 7.9, 1.0 Hz, 0.2H), 7.76 (dd, J = 7.7, 1.6 Hz, 0.2H), 7.45 (td, J = 11.3, 1.0 Hz, 0.2H), 7.30-7.16 (m, 5H), 4.49 (d, J = 2.8 Hz, 1H), 4.27-4.10 (m, 1H), 3.31- 3.23 (m, 1H), 3.12-3.02 (m, 1H), 2.95-2.75 (m, 2H), 2.72-2.50 (m, 2H), 2.44-2.30 (m, 1H), 1.98-1.13 (m, 11H), 0.94-0.80 (m, 3H), 0.78- 0.69 (m, 3H). MS (ESI) m/z 442.3 [C₂₅H₃₅N₃O₄ + H]⁺ |
| 13 | 6 | 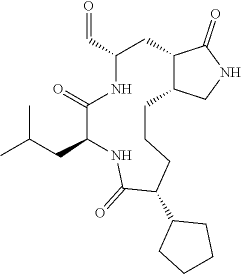 [SMILES: CC(C)C[C@@H]1NC(=O)[C@@H](C2CCCC2)CCC[C@H]2CNC(=O)[C@H]2C[C@@H](C=O)NC1=O] | ¹H NMR (400 MHz, MeOD-d₄): δ (ppm) 9.54-9.36 (m, 0.05H), 8.61-8.41 (m, 1H), 7.73-7.50 (m, 1H), 4.68-4.47 (m, 2H), 4.17-3.99 (m, 1H), 3.29- 3.21 (m, 1H), 3.08-2.96 (m, 1H), 2.75-2.58 (m, 1H), 2.03-1.80 (m, 5H), 1.80-1.46 (m, 11H), 1.46-1.32 (m, 1H), 1.32-1.20 (m, 2H), 1.20- 1.06 (m, 2H), 1.01-0.95 (m, 3H), 0.74-0.88 (m, 3H). MS (ESI) m/z 420.2 [C₂₃H₃₇N₃O₄ + H]⁺ |
| 14 | 6 | 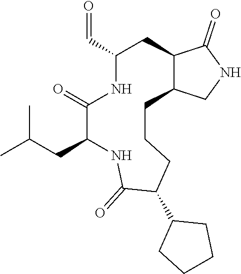 [SMILES: CC(C)C[C@@H]1NC(=O)[C@@H](C2CCCC2)CCC[C@@H]2CNC(=O)[C@@H]2C[C@@H](C=O)NC1=O] | ¹H NMR (400 MHz, MeOD-d₄): δ (ppm) 9.57-9.51 (m, 0.1H), 8.65-7.02 (m, 3H), 4.68-4.48 (m, 3H), 4.13-3.91 (m, 1H), 3.30-3.21 (m, 1H), 3.09- 2.98 (m, 1H), 2.74-2.23 (m, 2H), 2.07-1.82 (m, 4H), 1.81-1.09 (m, 15H), 1.06-0.88 (m, 6H). MS (ESI) m/z 420.2 [C₂₃H₃₇N₃O₄ + H]⁺ |
| 15 | 6 | 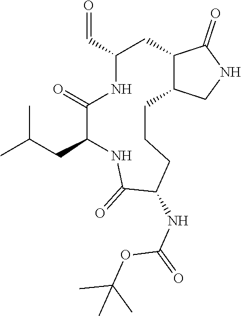 [SMILES: CC(C)C[C@@H]1NC(=O)[C@@H](NC(=O)OC(C)(C)C)CCC[C@H]2CNC(=O)[C@H]2C[C@@H](C=O)NC1=O] | ¹H NMR (400 MHz, MeOD) δ 4.61- 4.46 (m, 2H), 4.06-3.94 (m, 2H), 3.23 (ddd, J = 10.1, 6.1, 1.8 Hz, 1H), 3.04 (ddt, J = 10.1, 3.5, 1.7 Hz, 1H), 2.51-2.39 (m, 1H), 2.33 (s, 1H), 2.08-1.94 (m, 1H), 1.75-1.60 (m, 6H), 1.43 (d, J = 3.2 Hz, 9H), 1.41-1.33 (m, 1H), 1.28 (d, J = 9.6 Hz, 2H), 1.19-1.13 (m, 1H), 0.97 (dd, J = 6.4, 2.3 Hz, 3H), 0.89 (d, J = 6.4 Hz, 3H). MS (ESI) m/z = 467.3 [C₂₃H₃₈N₄O₆ + H]⁺ |
| 16 | 6 | 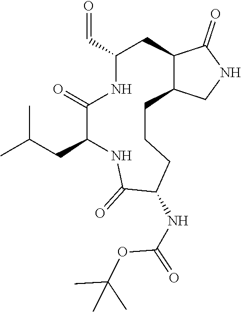 [SMILES: CC(C)C[C@@H]1NC(=O)[C@@H](NC(=O)OC(C)(C)C)CCC[C@@H]2CNC(=O)[C@@H]2C[C@@H](C=O)NC1=O] | ¹H NMR (400 MHz, MeOD) δ 4.65 (d, J = 4.3 Hz, 1H), 4.58 (d, J = 12.5 Hz, 2H), 4.00 (qd, J = 8.5, 3.9 Hz, 2H), 3.30-3.20 (m, 1H), 3.00 (dd, J = 9.9, 6.8 Hz, 1H), 2.58 (dt, J = 18.1, 6.8 Hz, 2H), 1.94-1.73 (m, 4H), 1.63 (d, J = 7.9 Hz, 2H), 1.43 (s, 9H), 1.30 (m, 4H), 1.02-0.88 (m, 6H). MS (ESI) m/z = 467.3 [C₂₃H₃₈N₄O₆ + H]⁺ |
| 17 | 6 | 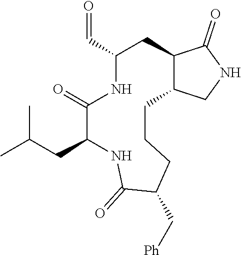 [SMILES: CC(C)C[C@@H]1NC(=O)[C@@H](Cc2ccccc2)CCC[C@H]2CNC(=O)[C@@H]2C[C@@H](C=O)NC1=O] | ¹H NMR (400 MHz, MeOD): δ (ppm) 9.53 (s, 0.1H), 8.45-7.94 (m, 2H), 7.29-7.14 (m, 5H), 4.60-4.47 (m, 1H), 3.94-3.78 (m, 1H), 3.48-3.36 (m, 1H), 2.96-2.78 (m, 2H), 2.71- 2.50 (m, 2H), 2.31-2.12 (m, 1H), 2.06-1.85 (m, 3H), 1.79-1.67 (m, 1H), 1.61-1.37 (m, 5H), 1.37-1.26 (m, 1H), 1.16-0.99 (m, 2H), 0.83- 0.74 (m, 6H). MS (ESI) m/z 442.2 [C₂₅H₃₅N₃O₄ + H]⁺ |
| 18 | 6 | 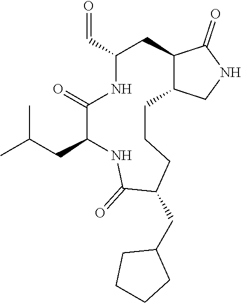 [SMILES: CC(C)C[C@@H]1NC(=O)[C@@H](CC2CCCC2)CCC[C@H]2CNC(=O)[C@@H]2C[C@@H](C=O)NC1=O] | ¹H NMR (400 MHz, MeOD) δ 4.71- 4.59 (m, 1H), 4.51 (dd, J = 3.9, 2.9 Hz, 1H), 3.95-3.80 (m, 1H), 3.48- 3.38 (m, 1H), 2.90 (dt, J = 10.1, 5.3 Hz, 1H), 2.37-2.07 (m, 2H), 2.07- 1.25 (m, 19H), 1.19 (tdd, J = 8.3, 4.4, 2.2 Hz, 1H), 1.14-1.01 (m, 3H), 0.96 (dd, J = 16.6, 6.0 Hz, 6H). MS (ESI) m/z 434.2 [C₂₄H₃₉N₃O₄ + H]⁺ |
| 19 | 6 | 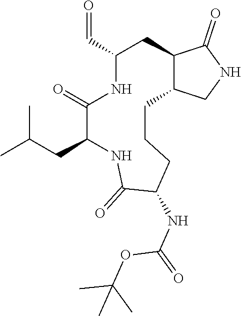 [SMILES: CC(C)C[C@@H]1NC(=O)[C@@H](NC(=O)OC(C)(C)C)CCC[C@H]2CNC(=O)[C@@H]2C[C@@H](C=O)NC1=O] | ¹H NMR (400 MHz, MeOD) δ 4.68- 4.56 (m, 1H), 4.49 (dd, J = 3.9, 0.9 Hz, 1H), 4.35-4.23 (m, 1H), 3.90 (tq, J = 12.8, 4.4 Hz, 1H), 3.42 (ddd, J = 10.0, 7.5, 4.6 Hz, 1H), 2.93 (ddd, J = 9.9, 5.4, 3.9 Hz, 1H), 2.26-2.09 (m, 1H), 2.07 (t, J = 7.6 Hz, 1H), 1.99-1.83 (m, 2H), 1.78 (td, J = 10.9, 5.7 Hz, 1H), 1.73-1.50 (m, 4H), 1.40 (q, J = 7.3 Hz, 3H), 1.18 (d, J = 0.8 Hz, 9H), 1.14-1.06 (m, 1H), 1.04-0.86 (m, 6H). MS (ESI) m/z 467.2 [C₂₃H₃₈N₄O₆ + H]⁺ |
| 20 | 3 | 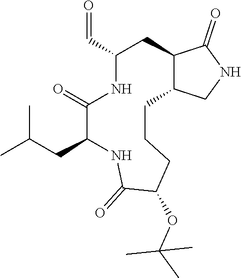 [SMILES: CC(C)C[C@@H]1NC(=O)[C@@H](OC(C)(C)C)CCC[C@H]2CNC(=O)[C@@H]2C[C@@H](C=O)NC1=O] | ¹H NMR (400 MHz, MeOD) δ 8.50- 8.12 (m, 1H), 4.69-4.53 (m, 1H), 4.49 (td, J = 4.0, 2.3 Hz, 1H), 4.10- 3.84 (m, 2H), 3.47-3.33 (m, 1H), 2.96-2.84 (m, 1H), 2.22-1.86 (m, 3H), 1.83-1.26 (m, 10H), 1.25- 1.14 (m, 9H), 1.00-0.91 (m, 6H). MS (ESI) m/z 424.2 [C₂₂H₃₇N₃O₅ + H]⁺ |
| 21 | 6 | 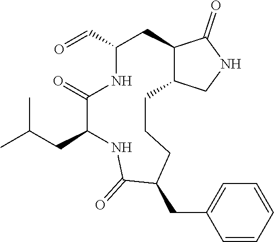 | ¹H NMR (400 MHz, MeOD) δ 7.30- 7.22 (m, 2H), 7.19 (dt, J = 9.8, 2.8 Hz, 3H), 4.61-4.37 (m, 1H), 4.15- 3.83 (m, 1H), 3.40-3.31 (m, 3H), 2.99-2.57 (m, 4H), 2.05 (dq, J = 51.0, 6.8 Hz, 1H), 1.85-1.24 (m, 6H), 0.99-0.68 (m, 6H). MS (ESI) m/z 422.1 [C₂₅H₃₅N₃O₄ + H]⁺ |
| 22 | 3 | 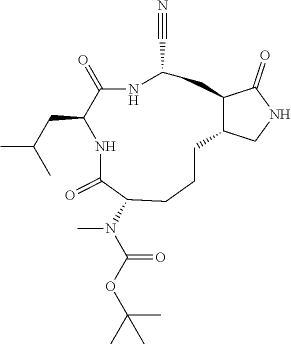 [SMILES: CC(C)C[C@@H]1NC(=O)[C@@H](N(C)C(=O)OC(C)(C)C)CCC[C@H]2CNC(=O)[C@@H]2C[C@@H](C#N)NC1=O] | 1H NMR (400 MHz, MeOD) δ 4.61 (d, J = 7.9 Hz, 1H), 4.34 (s, 1H), 3.47 (dd, J = 10.1, 7.4 Hz, 1H), 2.95 (s, 3H), 2.37-2.24 (m, 1H), 2.19 (ddd, J = 8.8, 6.2, 3.9 Hz, 1H), 2.09 (ddd, J = 14.3, 8.7, 3.2 Hz, 1H), 2.00 (q, J = 6.6 Hz, 1H), 1.85 (tt, J = 12.6, 6.3 Hz, 1H), 1.75-1.59 (m, 4H), 1.45 (s, 12H), 0.96 (dd, J = 15.8, 5.4 Hz, 7H). MS (ESI) m/z 478.2 [C₂₄H₃₉N₅O₅ + H]⁺ |
| 23 | 3 | 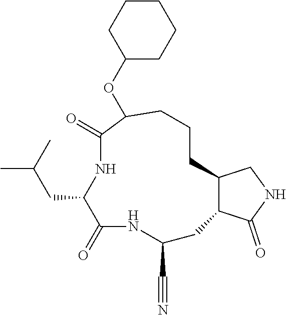 [SMILES: CC(C)C[C@@H]1NC(=O)C(OC2CCCCC2)CCC[C@H]2CNC(=O)[C@@H]2C[C@@H](C#N)NC1=O] | ¹H NMR (400 MHz, DMSO-d₆): δ (ppm) 4.84 (dd, J = 10.7, 3.0 Hz, 1H), 4.75- 4.53 (m, 1H), 4.03 (minor) (q, J = 4.6 Hz, 0.25H), 3.97 (major) (q, J = 4.8 Hz, 0.75H), 3.51-3.41 (m, 1H), 3.30- 3.21 (m, 1H), 3.00-2.91 (m, 1H), 2.36-2.25 (m, 1H), 2.25-2.09 (m, 2H), 2.08-1.50 (m, 12H), 1.49-1.19 (m, 8H), 1.05-0.96 (m, 6H). MS (ESI) m/z 447.2 [C₂₄H₃₈N₄O₄ + H]⁺ |
| 24 | 3 | 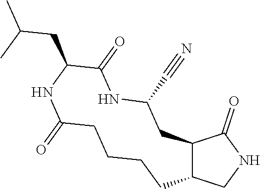 [SMILES: CC(C)C[C@@H]1NC(=O)CCCC[C@H]2CNC(=O)[C@@H]2C[C@@H](C#N)NC1=O] | ¹H NMR (400 MHz, MeOD) δ 4.86 (d, J = 3.1 Hz, 1H), 4.65-4.57 (m, 1H), 3.45 (dd, J = 10.0, 7.6 Hz, 1H), 2.95 (dd, J = 10.0, 6.2 Hz, 1H), 2.34 (ddd, J = 14.5, 9.6, 4.0 Hz, 1H), 2.29-2.15 (m, 3H), 2.13-1.96 (m, 2H), 1.77- 1.42 (m, 7H), 1.42-1.29 (m, 1H), 1.19-1.06 (m, 1H), 1.00 (d, J = 6.2 Hz, 3H), 0.97 (d, J = 6.1 Hz, 3H). MS (ESI) m/z 349.2 [C₁₈H₂₈N₄O₃ + H]⁺ |
| 25 | 3 | 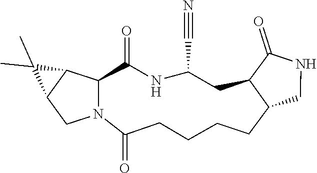 [SMILES: CC1(C)[C@@H]2[C@H]3C(=O)N[C@H](C#N)C[C@H]4C(=O)NC[C@@H]4CCCCC(=O)N3C[C@@H]21] | ¹H NMR (400 MHz, MeOD) δ 4.85- 4.71 (m, 1H), 4.34 (s, 1H), 3.75- 3.59 (m, 2H), 3.45-3.34 (m, 1H), 2.99-2.84 (m, 1H), 2.49 (ddd, J = 15.0, 5.2, 2.2 Hz, 1H), 2.43-2.28 (m, 1H), 2.28-2.04 (m, 3H), 2.01-1.87 (m, 1H), 1.70 (tdd, J = 11.7, 9.5, 6.0 Hz, 2H), 1.62-1.47 (m, 2H), 1.47- 1.24 (m, 4H), 1.10 (s, 3H), 0.98 (s, 3H). MS (ESI) m/z 373.1 [C₂₀H₂₈N₄O₃ + H]⁺ |
| 26 | 3 | 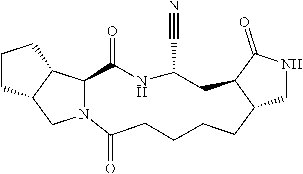 [SMILES: N#C[C@@H]1C[C@H]2C(=O)NC[C@@H]2CCCCC(=O)N2C[C@@H]3CCC[C@@H]3[C@H]2C(=O)N1] | ¹H NMR (400 MHz, MeOD) δ 4.71 (dd, J = 10.1, 2.3 Hz, 1H), 4.20 (d, J = 2.6 Hz, 1H), 3.67 (dd, J = 12.2, 8.6 Hz, 1H), 3.35 (dd, J = 12.2, 5.0 Hz, 1H), 3.32 (dd, J = 9.9, 7.1 Hz, 1H), 2.79 (dd, J = 9.9, 7.3 Hz, 1H), 2.77-2.69 (m, 1H), 2.56 (tdd, J = 8.2, 6.0, 2.6 Hz, 1H), 2.37 (ddd, J = 14.9, 5.4, 2.3 Hz, 1H), 2.27-2.08 (m, 3H), 2.06- 1.66 (m, 5H), 1.66-1.48 (m, 4H), 1.42 (ddt, J = 13.1, 6.7, 4.5 Hz, 2H), 1.37-1.19 (m, 3H). MS (ESI) m/z 373.1 [C₂₀H₂₈N₄O₃ + H]⁺ |
| 27 | 3 | 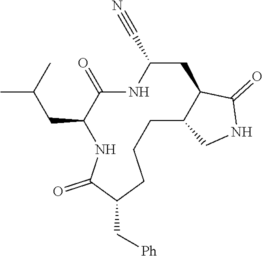 [SMILES: CC(C)C[C@@H]1NC(=O)[C@@H](Cc2ccccc2)CCC[C@H]2CNC(=O)[C@@H]2C[C@@H](C#N)NC1=O] | ¹H NMR (400 MHz, MeOD) δ 7.29-7.22 (m, 2H), 7.17 (ddt, J = 6.9, 2.8, 1.3 Hz, 3H), 4.83 (dd, J = 9.8, 3.1 Hz, 1H), 4.52 (dd, J = 9.5, 5.9 Hz, 1H), 3.46 (dd, J = 10.0, 7.4 Hz, 1H), 2.94 (dd, J = 10.1, 5.6 Hz, 1H), 2.83 (dd, J = 13.3, 10.4 Hz, 1H), 2.65 (dd, J = 13.3, 4.9 Hz, 1H), 2.61- 2.49 (m, 1H), 2.32 (ddd, J = 14.0, 9.8, 4.0 Hz, 1H), 2.20 (ddd, J = 8.1, 6.6, 4.0 Hz, 1H), 2.10 (ddd, J = 14.4, 8.1, 3.1 Hz, 1H), 1.95 (td, J = 7.3, 5.7 Hz, 1H), 1.73 (dtt, J = 12.8, 8.9, 3.9 Hz, 1H), 1.59-1.25 (m, 7H), 1.15-0.97 (m, 1H), 0.82 (d, J = 6.7 Hz, 3H), 0.79 (d, J = 6.5 Hz, 3H). MS (ESI) m/z 439.2 [C₂₅H₃₄N₄O₃ + H]⁺ |
| 28 | 3 | 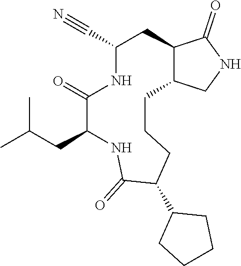 [SMILES: CC(C)C[C@@H]1NC(=O)[C@@H](C2CCCC2)CCC[C@H]2CNC(=O)[C@@H]2C[C@@H](C#N)NC1=O] | ¹H NMR (400 MHz, MeOD) δ 4.86- 4.75 (m, 1H), 4.67 (t, J = 7.3 Hz, 1H), 3.55-3.45 (m, 1H), 2.94 (dd, J = 10.1, 4.8 Hz, 1H), 2.32 (ddd, J = 14.1, 10.3, 3.8 Hz, 1H), 2.24-2.16 (m, 1H), 2.15-2.01 (m, 1H), 1.96 (m, 2H), 1.89-1.50 (m, 8H), 1.49-1.25 (m, 8H), 1.14 (s, 2H), 1.01 (d, J = 5.5 Hz, 3H), 0.97 (d, J = 5.6 Hz, 3H). MS (ESI) m/z 417.2 [C₂₃H₃₆N₄O₃ + H]⁺ |
| 29 | 3 | 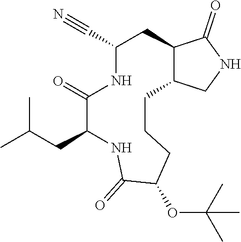 [SMILES: CC(C)C[C@@H]1NC(=O)[C@@H](OC(C)(C)C)CCC[C@H]2CNC(=O)[C@@H]2C[C@@H](C#N)NC1=O] | ¹H NMR (400 MHz, MeOD) δ 4.75- 4.51 (m, 1H), 4.01 (dd, J = 9.5, 5.0 Hz, 1H), 3.45 (td, J = 11.1, 8.0 Hz, 1H), 2.93 (dt, J = 10.0, 3.9 Hz, 1H), 2.29 (t, J = 12.4 Hz, 1H), 2.24-1.92 (m, 3H), 1.77-1.46 (m, 6H), 1.45-1.25 (m, 4H), 1.18 (s, 9H), 0.98 (d, J = 5.8 Hz, 3H), 0.95 (d, J = 5.8 Hz, 3H). MS (ESI) m/z 421.2 [C₂₂H₃₆N₄O₄ + H]⁺. |
| 30 | 6 | 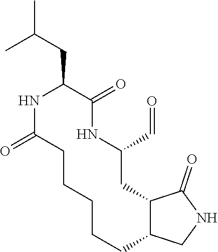 [SMILES: CC(C)C[C@@H]1NC(=O)CCCCC[C@H]2CNC(=O)[C@H]2C[C@@H](C=O)NC1=O] | ¹H NMR (400 MHz, MeOD) δ 4.65- 4.45 (m, 2H), 4.25-4.09 (m, 1H), 3.29-3.22 (m, 1H), 2.98 (ddd, J = 10.0, 5.8, 4.2 Hz, 1H), 2.47-2.32 (m, 2H), 2.32-2.14 (m, 2H), 1.96-1.79 (m, 2H), 1.73-1.48 (m, 4H), 1.48- 1.38 (m, 3H), 1.34 (ddd, J = 20.0, 9.5, 3.3 Hz, 2H), 1.16 (dd, J = 12.7, 6.2 Hz, 1H), 1.02-0.86 (m, 6H). MS (ESI) m/z 366.2 [C₁₉H₃₁N₃O₄ + H]⁺ |
| 31 | 6 | 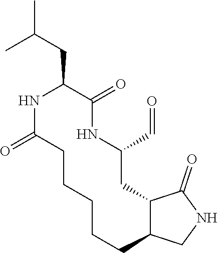 [SMILES: CC(C)C[C@@H]1NC(=O)CCCCC[C@@H]2CNC(=O)[C@H]2C[C@@H](C=O)NC1=O] | ¹H NMR (400 MHz, MeOD) δ 4.62- 4.45 (m, 1H), 4.45-4.29 (m, 1H), 3.47 (ddd, J = 35.0, 9.9, 7.9 Hz, 1H), 2.97-2.86 (m, 1H), 2.37-2.19 (m, 2H), 2.19-1.92 (m, 3H), 1.82 (dtt, J = 10.3, 7.0, 3.7 Hz, 1H), 1.72-1.21 (m, 11H), 1.02-0.89 (m, 6H). MS (ESI) m/z 366.1 [C₁₉H₃₁N₃O₄ + H]⁺ |
| 32 | 6 | 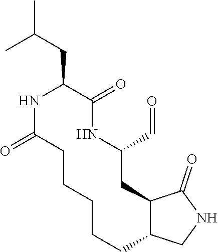 [SMILES: CC(C)C[C@@H]1NC(=O)CCCCC[C@H]2CNC(=O)[C@@H]2C[C@@H](C=O)NC1=O] | ¹H NMR (400 MHz, MeOD) δ 4.62- 4.40 (m, 2H), 3.90 (ddt, J = 21.1, 11.0, 3.8 Hz, 1H), 3.57-3.45 (m, 1H), 3.04-2.88 (m, 1H), 2.26 (ddt, J = 13.5, 7.0, 3.5 Hz, 2H), 2.18-2.04 (m, 2H), 1.97-1.72 (m, 3H), 1.71- 1.47 (m, 4H), 1.45-1.27 (m, 4H), 1.13 (s, 1H), 1.04-0.88 (m, 6H). MS (ESI) m/z 366.2 [C₁₉H₃₁N₃O₄ + H]⁺ |
| 33 | 6 | 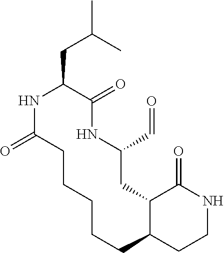 [SMILES: CC(C)C[C@@H]1NC(=O)CCCCC[C@@H]2CCNC(=O)[C@H]2C[C@@H](C=O)NC1=O] | ¹H NMR (400 MHz, MeOD) δ 8.11 (s, 1H), 4.64-4.53 (m, 1H), 4.43-4.34 (m, 1H), 3.30-3.15 (m, 2H), 2.37- 2.26 (m, 1H), 2.26-1.97 (m, 3H), 1.97-1.82 (m, 2H), 1.74-1.69 (m, 1H), 1.68-1.22 (m, 13H), 0.96 (d, J = 6.4 Hz, 3H), 0.93 (d, J = 6.3 Hz, 3H). MS (ESI) m/z 380.1 [C₂₀H₃₃N₃O₄ + H]⁺ |
| 34 | 6 | 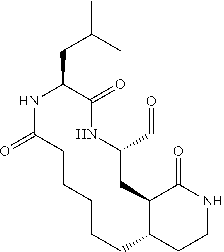 [SMILES: CC(C)C[C@@H]1NC(=O)CCCCC[C@H]2CCNC(=O)[C@@H]2C[C@@H](C=O)NC1=O] | ¹H NMR (400 MHz, MeOD) δ 8.09 (s, 1H), 4.66-4.52 (m, 1H), 4.41 (t, J = 4.2 Hz, 1H), 4.06-3.93 (m, 1H), 3.36- 3.27 (m, 1H), 3.25-3.15 (m, 1H), 2.36-2.29 (m, 1H), 2.28-2.09 (m, 2H), 2.02-1.76 (m, 4H), 1.74-1.46 (m, 6H), 1.33 (dtd, J = 19.1, 9.0, 4.0 Hz, 3H), 1.26-1.08 (m, 3H), 0.99 (d, J = 6.3 Hz, 3H), 0.94 (d, J = 6.3 Hz, 3H). MS (ESI) m/z 380.1 [C₂₀H₃₃N₃O₄ + H]⁺ |
| 35 | 6 | 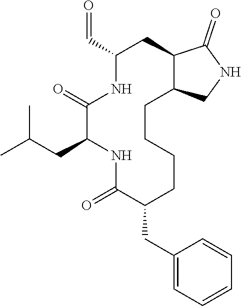 [SMILES: CC(C)C[C@@H]1NC(=O)[C@@H](Cc2ccccc2)CCCC[C@@H]2CNC(=O)[C@@H]2C[C@@H](C=O)NC1=O] | ¹H NMR (400 MHz, MeOD) δ 7.29- 7.13 (m, 5H), 4.61 (d, J = 6.0 Hz, 1H), 4.55 (dd, J = 9.3, 5.9 Hz, 1H), 4.07- 3.85 (m, 1H), 3.38 (d, J = 7.8 Hz, 2H), 2.98 (dd, J = 9.9, 5.3 Hz, 1H), 2.81 (dd, J = 13.2, 10.5 Hz, 1H), 2.70- 2.53 (m, 3H), 2.37 (s, 1H), 1.93 (s, 2H), 1.76 (s, 1H), 1.57 (s, 3H), 1.42- 1.12 (m, 7H), 0.83-0.72 (m, 6H). MS (ESI) m/z 456.2 [C₂₆H₃₇N₃O₄ + H]⁺ |
| 36 | 6 | 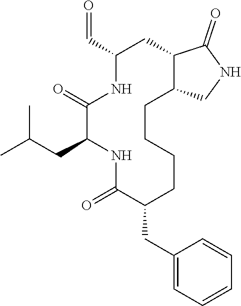 [SMILES: CC(C)C[C@@H]1NC(=O)[C@@H](Cc2ccccc2)CCCC[C@H]2CNC(=O)[C@H]2C[C@@H](C=O)NC1=O] | ¹H NMR (400 MHz, MeOD) δ 7.33- 7.22 (m, 2H), 7.19 (tq, J = 4.9, 1.7 Hz, 3H), 4.48 (dd, J = 3.4, 1.6 Hz, 1H), 4.37 (tdd, J = 8.6, 7.2, 4.1 Hz, 1H), 4.25-4.10 (m, 1H), 3.27 (td, J = 6.5, 3.1 Hz, 1H), 2.99 (ddd, J = 9.8, 5.9, 4.0 Hz, 1H), 2.85-2.68 (m, 2H), 2.66-2.56 (m, 1H), 2.34 (p, J = 7.5 Hz, 2H), 1.96-1.81 (m, 1H), 1.77 (qd, J = 9.4, 4.6 Hz, 1H), 1.67-1.53 (m, 2H), 1.53-1.30 (m, 6H), 1.21 (dtd, J = 16.7, 8.7, 3.7 Hz, 2H), 0.91 (ddt, J = 18.6, 12.0, 4.0 Hz, 1H), 0.84- 0.67 (m, 6H). MS (ESI) m/z 456.2 [C₂₆H₃₇N₃O₄ + H]⁺ |
| 37 | 6 | 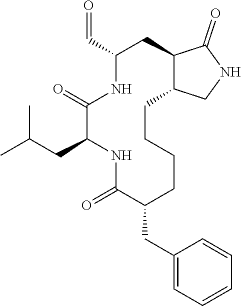 [SMILES: CC(C)C[C@@H]1NC(=O)[C@@H](Cc2ccccc2)CCCC[C@H]2CNC(=O)[C@@H]2C[C@@H](C=O)NC1=O] | ¹H NMR (400 MHz, MeOD) δ 7.96 (d, J = 8.9 Hz, 1H), 7.28-7.19 (m, 2H), 7.19-7.11 (m, 3H), 4.49-4.36 (m, 2H), 3.93-3.77 (m, 1H), 3.49-3.37 (m, 1H), 3.00-2.90 (m, 1H), 2.90- 2.59 (m, 2H), 2.57-2.44 (m, 1H), 2.34-2.23 (m, 1H), 2.14 (dp, J = 12.0, 6.5 Hz, 1H), 1.93 (ddt, J = 14.9, 11.2, 3.9 Hz, 1H), 1.86-1.55 (m, 3H), 1.52-0.85 (m, 9H), 0.83-0.64 (m, 6H). MS (ESI) m/z 456.2 [C₂₆H₃₇N₃O₄ + H]⁺. |
| 38 | 3 | 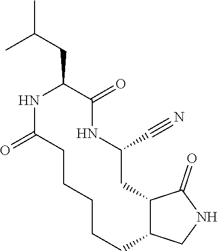 | ¹H NMR (400 MHz, MeOD) δ 5.29 (dd, J = 9.5, 2.8 Hz, 1H), 4.61-4.49 (m, 1H), 3.32-3.22 (m, 1H), 2.98 (dd, J = 9.8, 7.6 Hz, 1H), 2.44 (dtd, J = 11.8, 6.2, 3.3 Hz, 1H), 2.35-2.25 (m, 2H), 2.25-2.15 (m, 1H), 2.12-1.94 (m, 2H), 1.83 (dddd, J = 16.9, 14.4, 6.8, 4.7 Hz, 1H), 1.75-1.50 (m, 4H), 1.49-1.21 (m, 6H), 1.04-0.86 (m, 6H). MS (ESI) m/z 521.3 [C₁₉H₃₀N₄O₃ + H]⁺ |
| 39 | 3 | 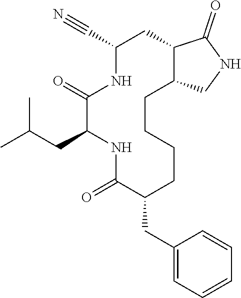 [SMILES: CC(C)C[C@@H]1NC(=O)[C@@H](Cc2ccccc2)CCCC[C@H]2CNC(=O)[C@H]2C[C@@H](C#N)NC1=O] | ¹H NMR (400 MHz, MeOD) δ 8.07 (d, J = 9.2 Hz, 1H), 7.19-7.10 (m, 2H), 7.07 (td, J = 6.3, 1.2 Hz, 3H), 5.17 (dd, J = 10.4, 1.8 Hz, 1H), 4.34 (td, J = 9.2, 6.0 Hz, 1H), 3.18 (dd, J = 9.8, 7.1 Hz, 1H), 2.86 (dd, J = 9.9, 8.0 Hz, 1H), 2.68 (dd, J = 13.2, 10.4 Hz, 1H), 2.60 (dd, J = 13.2, 5.1 Hz, 1H), 2.46 (tdd, J = 10.7, 5.1, 3.9 Hz, 1H), 2.35-2.23 (m, 1H), 2.15 (ddd, J = 9.4, 7.5, 4.2 Hz, 1H), 2.02 (ddd, J = 14.6, 10.4, 4.3 Hz, 1H), 1.84 (ddd, J = 14.6, 9.5, 1.9 Hz, 1H), 1.68-1.56 (m, 1H), 1.52-1.43 (m, 1H), 1.24 (qdd, J = 10.3, 7.8, 4.5 Hz, 7H), 1.08 (ddd, J = 14.0, 9.3, 5.2 Hz, 1H), 0.85-0.72 (m, 1H), 0.66 (d, J = 6.5 Hz, 3H), 0.63 (d, J = 6.4 Hz, 3H). MS (ESI) m/z 453.2 [M + H⁺; C₂₆H₃₆N₄O₃ + H]⁺ |
| 40 | 3 | 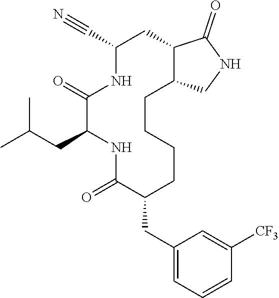 [SMILES: CC(C)C[C@@H]1NC(=O)[C@@H](Cc2cccc(C(F)(F)F)c2)CCCC[C@H]2CNC(=O)[C@H]2C[C@@H](C#N)NC1=O] | ¹H NMR (400 MHz, MeOD-d₄): δ (ppm) 7.45-7.31 (m, 4H), 5.16 (dd, J = 10.3, 1.6 Hz, 1H), 4.32 (dd, J = 9.0, 6.3 Hz, 1H), 2.87 (dd, J = 9.8, 8.1 Hz, 1H), 2.80-2.67 (m, 2H), 2.58-2.48 (m, 1H), 2.35-2.24 (m, 1H), 2.20-2.12 (m, 1H), 2.07-1.97 (m, 1H), 1.82 (qd, J = 12.9, 1.7 Hz, 1H), 1.68-1.56 (m, 1H), 1.56-1.46 (m, 1H), 1.39-1.17 (m, 8H), 1.06-0.97 (m, 1H), 0.82-0.66 (m, 1H), 0.61 (dd, J = 14.4, 6.1 Hz, 6H). ¹⁹F NMR (376 MHz, MeOD-d₄): δ (ppm) −63.8 (s) MS (ESI) m/z 521.3 [C₂₇H₃₅F₃N₄O₃ + H]⁺ |
| 41 | 3 | 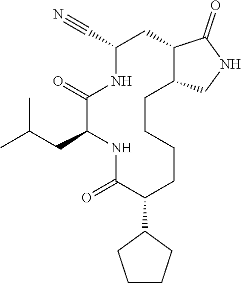 [SMILES: CC(C)C[C@@H]1NC(=O)[C@@H](C2CCCC2)CCCC[C@H]2CNC(=O)[C@H]2C[C@@H](C#N)NC1=O] | ¹H NMR (400 MHz, MeOD) δ 4.76 (dd, J = 11.4, 2.6 Hz, 1H), 4.48 (dd, J = 9.2, 5.6 Hz, 1H), 3.27 (dd, J = 9.9, 6.4 Hz, 1H), 3.03 (dd, J = 9.9, 6.0 Hz, 1H), 2.53-2.36 (m, 4H), 2.07-1.86 (m, 4H), 1.81-1.49 (m, 6H), 1.47- 1.26 (m, 4H), 1.24-1.06 (m, 2H), 0.99 (d, J = 6.1 Hz, 3H), 0.95 (d, J = 6.2 Hz, 3H). MS (ESI) m/z 432.2 [C₂₄H₃₈N₄O₃ + H]⁺ |
| 42 | 3 | 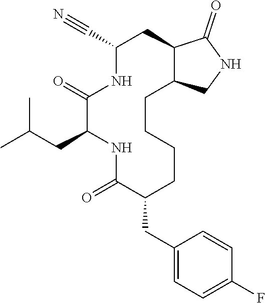 [SMILES: CC(C)C[C@@H]1NC(=O)[C@@H](Cc2ccc(F)cc2)CCCC[C@@H]2CNC(=O)[C@@H]2C[C@@H](C#N)NC1=O] | ¹H NMR (400 MHz, MeOD) δ 7.21- 7.13 (m, 2H), 7.02-6.92 (m, 2H), 5.31-5.22 (m, 1H), 4.42 (dd, J = 9.5, 5.9 Hz, 1H), 3.30-3.25 (m, 1H), 2.97 (dd, J = 9.9, 7.9 Hz, 1H), 2.78-2.66 (m, 2H), 2.60-2.50 (m, 1H), 2.40 (d, J = 9.4 Hz, 1H), 2.31-2.22 (m, 1H), 2.11 (ddd, J = 14.7, 10.5, 4.4 Hz, 1H), 2.00-1.91 (m, 1H), 1.70 (s, 2H), 1.59 (s, 2H), 1.53-1.30 (m, 6H), 1.21 (dt, J = 9.2, 4.7 Hz, 1H), 0.75 (dd, J = 13.0, 6.1 Hz, 6H). ¹⁹F NMR (376 MHz, MeOD) δ −119.20 (tt, J = 9.0, 5.5 Hz). MS (ESI) m/z 471.2 [C₂₆H₃₅N₄O₃F + H]⁺. |
| 43 | 3 | 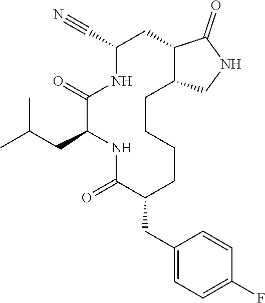 [SMILES: CC(C)C[C@@H]1NC(=O)[C@@H](Cc2ccc(F)cc2)CCCC[C@H]2CNC(=O)[C@H]2C[C@@H](C#N)NC1=O] | ¹H NMR (400 MHz, MeOD) δ 8.20 (d, J = 9.1 Hz, 1H), 7.22-7.14 (m, 2H), 7.03-6.95 (m, 2H), 5.29 (dd, J = 10.4, 1.9 Hz, 1H), 4.44 (ddd, J = 9.0, 5.9, 3.0 Hz, 1H), 3.29 (dd, J = 9.9, 7.1 Hz, 1H), 2.99 (dd, J = 9.9, 8.0 Hz, 1H), 2.82-2.74 (m, 1H), 2.71 (dd, J = 13.3, 5.2 Hz, 1H), 2.56 (tdd, J = 10.6, 5.3, 4.0 Hz, 1H), 2.47-2.35 (m, 1H), 2.28 (ddd, J = 9.4, 7.5, 4.3 Hz, 1H), 2.13 (ddd, J = 14.7, 10.4, 4.4 Hz, 1H), 1.96 (ddd, J = 14.6, 9.5, 1.9 Hz, 1H), 1.78-1.66 (m, 1H), 1.60 (tt, J = 8.2, 5.0 Hz, 1H), 1.52-1.29 (m, 7H), 1.22 (ddd, J = 14.1, 9.5, 4.7 Hz, 1H), 0.91-0.81 (m, 1H), 0.79 (d, J = 6.0 Hz, 3H), 0.76 (d, J = 6.0 Hz, 3H). ¹⁹F NMR (376 MHz, MeOD) δ −119.19 (tt, J = 14.5, 5.5 Hz). MS (ESI) m/z 471.2 [C₂₆H₃₅N₄O₃F + H]⁺. |
| 44 | 4 | 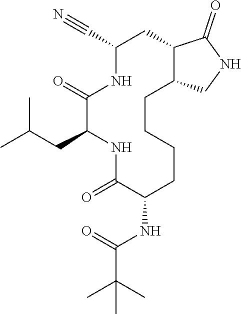 | ¹H NMR (400 MHz, MeOD) δ 5.25 (dd, J = 8.2, 4.1 Hz, 1H), 4.58 (t, J = 7.3 Hz, 1H), 4.28 (dd, J = 9.9, 4.4 Hz, 1H), 3.01 (dd, J = 9.8, 7.0 Hz, 1H), 2.51-2.35 (m, 2H), 2.19-2.00 (m, 2H), 1.94-1.80 (m, 1H), 1.76-1.52 (m, 5H), 1.50-1.25 (m, 6H), 1.19 (m, 9H), 0.99 (d, J = 6.1 Hz, 3H), 0.94 (d, J = 6.1 Hz, 3H). MS (ESI) m/z 471.2 [C₂₄H₃₉N₅O₄ + H]⁺. |
| 45 | 6 | 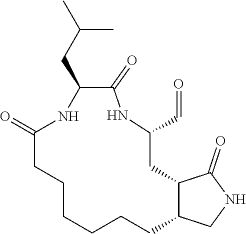 [SMILES: CC(C)C[C@@H]1NC(=O)CCCCCC[C@H]2CNC(=O)[C@H]2C[C@@H](C=O)NC1=O] | ¹H NMR (400 MHz, MeOD) δ 4.65- 4.38 (m, 2H), 3.96-3.83 (m, 1H), 3.46-3.30 (m, 1H), 3.14-3.02 (m, 1H), 2.73 (tdd, J = 11.2, 7.5, 3.8 Hz, 1H), 2.52-2.41 (m, 1H), 2.34-2.10 (m, 3H), 1.79-1.67 (m, 1H), 1.67- 1.50 (m, 4H), 1.50-1.34 (m, 4H), 1.34-1.17 (m, 4H), 1.14-1.04 (m, 1H), 1.02-0.89 (m, 6H). MS (ESI) m/z 380.2 [C₂₀H₃₃N₃O₄ + H]⁺ |
| 46 | 6 | 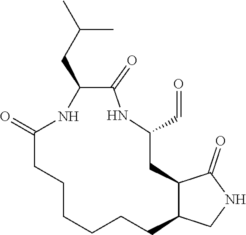 [SMILES: CC(C)C[C@@H]1NC(=O)CCCCCC[C@@H]2CNC(=O)[C@@H]2C[C@@H](C=O)NC1=O] | ¹H NMR (400 MHz, MeOD) δ 8.19- 8.08 (m, 1H), 4.73-4.52 (m, 1H), 4.03-3.87 (m, 1H), 3.44-3.30 (m, 2H), 3.11-2.97 (m, 1H), 2.75 (dp, J = 7.9, 4.2 Hz, 1H), 2.47 (s, 1H), 2.37- 2.14 (m, 2H), 2.10-1.91 (m, 2H), 1.71-1.48 (m, 4H), 1.45-1.24 (m, 7H), 1.10 (dh, J = 11.3, 3.5 Hz, 1H), 1.03-0.88 (m, 6H). MS (ESI) m/z 380.2 [C₂₀H₃₃N₃O₄ + H]⁺ |
| 47 | 6 | 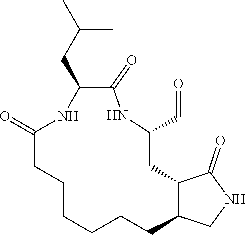 [SMILES: CC(C)C[C@@H]1NC(=O)CCCCCC[C@@H]2CNC(=O)[C@H]2C[C@@H](C=O)NC1=O] | ¹H NMR (400 MHz, MeOD) δ 4.84 (d, J = 3.8 Hz, 1H), 4.62-4.50 (m, 1H), 3.98 (dddd, J = 14.6, 8.5, 4.3, 2.2 Hz, 1H), 3.49-3.33 (m, 1H), 2.94 (ddd, J = 9.4, 6.7, 2.6 Hz, 1H), 2.34 (ddt, J = 12.6, 4.5, 2.3 Hz, 1H), 2.27-2.16 (m, 3H), 2.16-2.02 (m, 1H), 1.81 (tt, J = 14.2, 6.7 Hz, 1H), 1.74-1.48 (m, 5H), 1.43-1.19 (m, 8H), 1.03-0.90 (m, 6H). MS (ESI) m/z 380.2 [C₂₀H₃₃N₃O₄ + H]⁺ |
| 48 | 6 | 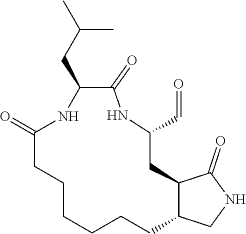 [SMILES: CC(C)C[C@@H]1NC(=O)CCCCCC[C@H]2CNC(=O)[C@@H]2C[C@@H](C=O)NC1=O] | ¹H NMR (400 MHz, MeOD) δ 4.55- 4.40 (m, 2H), 4.01 (ddd, J = 11.4, 4.3, 3.0 Hz, 1H), 3.49-3.38 (m, 1H), 3.05 (ddd, J = 10.1, 5.0, 2.0 Hz, 1H), 2.21 (dddt, J = 24.2, 16.9, 10.5, 3.2 Hz, 4H), 1.95 (ddt, J = 14.2, 11.5, 2.7 Hz, 1H), 1.77 (dddd, J = 16.7, 12.4, 6.2, 2.9 Hz, 2H), 1.72-1.63 (m, 1H), 1.63-1.39 (m, 6H), 1.31 (q, J = 6.2 Hz, 4H), 1.18 (d, J = 12.9 Hz, 1H), 1.06-0.92 (m, 6H). MS (ESI) m/z 380.2 [C₂₀H₃₃N₃O₄ + H]⁺ |
| 49 | 3 | 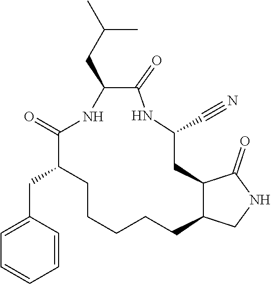 [SMILES: CC(C)C[C@@H]1NC(=O)[C@@H](Cc2ccccc2)CCCCC[C@@H]2CNC(=O)[C@@H]2C[C@@H](C#N)NC1=O] | ¹H NMR (400 MHz, MeOD) δ 7.30- 7.21 (m, 2H), 7.21-7.13 (m, 3H), 5.15 (dd, J = 5.6, 3.8 Hz, 1H), 4.51- 4.41 (m, 1H), 3.42 (dd, J = 10.2, 6.2 Hz, 1H), 3.17 (dd, J = 10.2, 2.6 Hz, 1H), 2.83-2.46 (m, 5H), 2.27-2.09 (m, 2H), 1.72-1.58 (m, 1H), 1.53 (dddd, J = 13.0, 9.5, 6.4, 2.9 Hz, 1H), 1.47-1.12 (m, 9H), 1.07-0.88 (m, 2H), 0.77 (dd, J = 7.8, 6.5 Hz, 6H). MS (ESI) m/z 467.2 [C₂₇H₃₈N₄O₃ + H]⁺ |
| 50 | 3 | 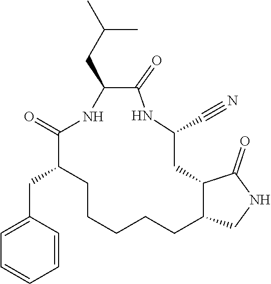 [SMILES: CC(C)C[C@@H]1NC(=O)[C@@H](Cc2ccccc2)CCCCC[C@H]2CNC(=O)[C@H]2C[C@@H](C#N)NC1=O] | ¹H NMR (400 MHz, MeOD) δ 7.29- 7.21 (m, 2H), 7.17 (ddt, J = 6.8, 2.7, 1.2 Hz, 3H), 4.82 (dd, J = 9.8, 3.1 Hz, 1H), 4.36 (dd, J = 9.4, 6.0 Hz, 1H), 3.43 (dd, J = 10.2, 6.3 Hz, 2H), 3.19- 3.10 (m, 1H), 2.81 (dd, J = 13.2, 10.3 Hz, 1H), 2.73-2.62 (m, 2H), 2.62-2.43 (m, 3H), 1.80 (ddd, J = 14.8, 11.9, 3.1 Hz, 1H), 1.67-1.10 (m, 12H), 0.81 (dd, J = 20.3, 6.5 Hz, 6H). MS (ESI) m/z 467.2 [C₂₇H₃₈N₄O₃ + H]⁺ |
| 51 | 6 | 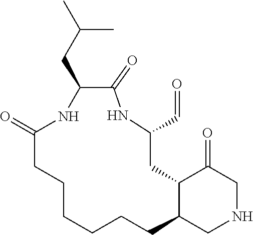 [SMILES: CC(C)C[C@@H]1NC(=O)CCCCCC[C@@H]2CNCC(=O)[C@H]2C[C@@H](C=O)NC1=O] | ¹H NMR (400 MHz, MeOD) δ 4.48- 4.31 (m, 2H), 4.07-3.93 (m, 1H), 3.19-3.04 (m, 2H), 2.26-1.96 (m, 4H), 1.87-1.74 (m, 1H), 1.74-1.61 (m, 1H), 1.61-1.02 (m, 15H), 0.86 (dd, J = 6.5, 1.2 Hz, 3H), 0.83 (dd, J = 6.4, 1.2 Hz, 3H). MS (ESI) m/z 394.1 [C₂₁H₃₅N₃O₄ + H]⁺ |
| 52 | 6 | 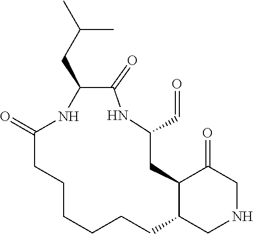 [SMILES: CC(C)C[C@@H]1NC(=O)CCCCCC[C@H]2CNCC(=O)[C@@H]2C[C@@H](C=O)NC1=O] | ¹H NMR (400 MHz, MeOD) δ 8.22- 8.06 (m, 1H), 7.89-7.82 (m, 1H), 4.60-4.37 (m, 1H), 4.32 (dd, J = 4.2, 1.5 Hz, 1H), 3.97-3.84 (m, 1H), 3.22- 3.07 (m, 1H), 2.26-2.16 (m, 1H), 2.13-1.72 (m, 6H), 1.71-1.37 (m, 7H), 1.36-1.08 (m, 8H), 0.92-0.82 (m, 6H). MS (ESI) m/z 394.2 [C₂₁H₃₅N₃O₄ + H]⁺ |
| 53 | 4 | 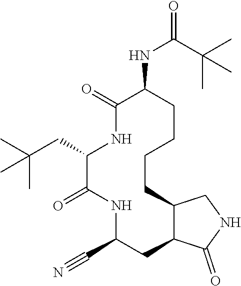 [SMILES: CC(C)(C)C[C@@H]1NC(=O)[C@@H](NC(=O)C(C)(C)C)CCCC[C@H]2CNC(=O)[C@H]2C[C@@H](C#N)NC1=O] | ¹H NMR (400 MHz, MeOD-d4) δ 7.19 (d, J = 7.59 Hz, 1H), 5.29 (dd, J = 9.49, 2.69, 1H), 4.64 (t, J = 6.57 Hz, 1H), 4.37 (ddd, J = 10.28, 7.60, 4.35 Hz, 1H), 3.35, (dd, J = 9.90, 6.94 Hz, 1H), 3.01 (dd, J = 9.83, 7.69 Hz, 1H), 2.44 (m, 1H), 2.37 (m, 1H), 2.15 (m, 1H), 2.02 (m, 1H), 2.00, (dd, J = 14.18, 6.93 Hz, 1H), 1.85 (m, 1H), 1.62 (m, 1H), 1.54 (dd, J = 14.18, 6.19 Hz, 1H), 1.25-1.49 (m, 6H), 1.19 (s, 9H), 0.97 (s, 9H). MS (ESI) m/z = 476.3 [C₂₅H₄₁N₅O₄ + H]⁺ |
| 54 | 4 | 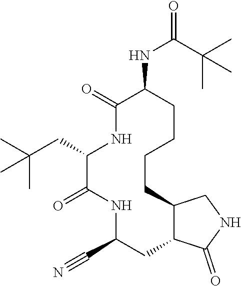 | ¹H NMR (400 MHz, MeOD-d4) δ 8.67 (d, J = 8.76 Hz, 1H), 7.16 (d, J = 6.83, 1H), 4.94 (dd, J = 12.82, 3.37 Hz, 1H), 4.62 (m, 1H), 4.37 (sext, J = 3.47 Hz, 1H), 3.56, (dd, J = 10.22, 7.70 Hz, 1H), 2.97 (dd, J = 10.28, 3.12 Hz, 1H), 2.34 (dt, J = 19.94, 2.92 Hz, 1H), 2.25 (dt, J = 12.21, 3.17 Hz, 1H), 2.15 (m, 1H), 2.00 (m, 1H), 1.94, (m, 1H), 1.60-1.78 (m, 4H), 1.25- 1.44 (m, 4H), 1.21 (s, 9H), 1.00-1.17 (m, 2H), 1.02 (d, J = 6.24 Hz, 3H), 0.97 (d, J = 6.20 Hz, 3H). MS (ESI) m/z = 462.2 [C₂₄H₃₉N₅O₄ + H]⁺ |
| 55 | 4 | 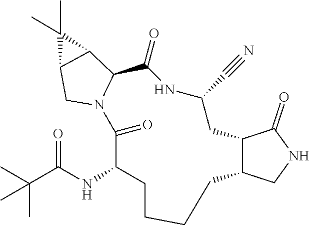 [SMILES: CC(C)(C)C(=O)N[C@H]1CCCC[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ 5.13 (dd, J = 7.5, 4.2 Hz, 1H), 4.53 (d, J = 7.5 Hz, 2H), 4.02 (d, J = 10.8 Hz, 1H), 3.83 (dd, J = 10.7, 5.1 Hz, 1H), 3.35 (dd, J = 9.9, 6.6 Hz, 1H), 2.99 (dd, J = 9.9, 6.1 Hz, 1H), 2.51-2.34 (m, 2H), 2.16 (ddd, J = 14.9, 7.4, 4.3 Hz, 1H), 2.08-1.92 (m, 2H), 1.68 (d, J = 7.7 Hz, 1H), 1.65-1.55 (m, 2H), 1.47-1.22 (m, 5H), 1.16 (s, 10H), 1.09 (s, 3H), 0.92 (s, 3H). ¹³C NMR (101 MHz, MeOD) δ 179.57, 179.12, 172.04, 170.75, 118.29, 60.03, 51.13, 48.23, 48.02, 47.81, 47.59, 47.38, 47.17, 46.96, 45.93, 42.60, 38.68, 38.61, 38.15, 30.77, 30.13, 28.38, 27.63, 27.13, 26.91, 26.26, 25.28, 23.95, 18.59, 11.79. MS (ESI) m/z 486.2 [C₂₆H₃₉N₅O₄ + H]⁺ |
| 56 | 3 | 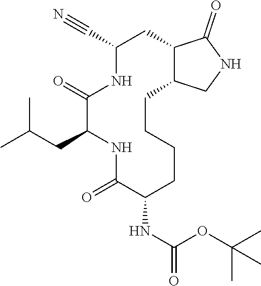 [SMILES: CC(C)C[C@@H]1NC(=O)[C@@H](NC(=O)OC(C)(C)C)CCCC[C@H]2CNC(=O)[C@H]2C[C@@H](C#N)NC1=O] | ¹H NMR (400 MHz, MeOD) δ 5.13 (dd, J = 7.5, 4.2 Hz, 1H), 4.53 (d, J = 7.5 Hz, 2H), 4.02 (d, J = 10.8 Hz, 1H), 3.83 (dd, J = 10.7, 5.1 Hz, 1H), 3.35 (dd, J = 9.9, 6.6 Hz, 1H), 2.99 (dd, J = 9.9, 6.1 Hz, 1H), 2.51-2.35 (m, 2H), 2.16 (ddd, J = 14.9, 7.4, 4.3 Hz, 1H), 2.08-1.92 (m, 2H), 1.68 (d, J = 7.7 Hz, 1H), 1.65-1.55 (m, 2H), 1.47-1.22 (m, 5H), 1.16 (s, 10H), 1.09 (s, 3H), 0.92 (s, 3H). ¹³C NMR (101 MHz, MeOD) δ 179.57, 179.12, 172.04, 170.75, 118.29, 60.03, 51.13, 48.23, 48.02, 47.81, 47.59, 47.38, 47.17, 46.96, 45.93, 42.60, 38.68, 38.61, 38.15, 30.77, 30.13, 28.38, 27.63, 27.13, 26.91, 26.26, 25.28, 23.95, 18.59, 11.79. MS (ESI) m/z 478.1 [C₂₄H₃₉N₅O₅ + H]⁺ |
| 57 | 4 | 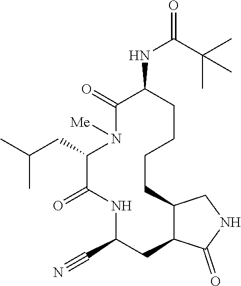 [SMILES: CC(C)C[C@H]1C(=O)N[C@H](C#N)C[C@@H]2C(=O)NC[C@@H]2CCCC[C@H](NC(=O)C(C)(C)C)C(=O)N1C] | ¹H NMR (400 MHz, MeOD-d₄): δ (ppm) 7.32 (d, J = 7.0 Hz, 1H), 5.37-5.28 (m, 2H), 4.86-4.81 (m, 1H), 3.14 (s, 3H), 2.99 (dd, J = 9.8, 8.6 Hz, 1H), 2.52-2.46 (m, 1H), 2.17-2.09 (m, 2H), 1.98-1.89 (m, 2H), 1.75-1.66 (m, 2H), 1.64-1.57 (m, 1H), 1.54-1.28 (m, 7H), 1.19 (s, 9H), 0.97 (d, J = 6.6 Hz, 3H), 0.92 (d, J = 6.5 Hz, 3H). MS (ESI) m/z 476.3 [C₂₅H₄₁N₅O₄ + H]⁺ |
| 58 | 3 | 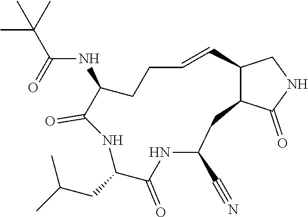 [SMILES: CC(C)C[C@@H]1NC(=O)[C@@H](NC(=O)C(C)(C)C)CC/C=C/[C@H]2CNC(=O)[C@H]2C[C@@H](C#N)NC1=O] | ¹H NMR (400 MHz, MeOD): δ (ppm) 8.59-8.41 (m, 1H), 7.19 (d, J = 7.0 Hz, 1H), 5.61 (ddd, J = 15.4, 7.8, 4.2 Hz, 1H), 5.47-5.33 (m, 1H), 4.78 (dd, J = 8.0, 4.8 Hz, 2H), 4.39-4.27 (m, 2H), 3.55 (dd, J = 10.0, 5.9 Hz, 1H), 3.20-3.05 (m, 1H), 3.03 (dd, J = 10.0, 1.5 Hz, 1H), 2.65 (ddd, J = 12.1, 7.4, 2.4 Hz, 1H), 2.49 (ddd, J = 14.6, 8.0, 2.4 Hz, 1H), 2.32-2.05 (m, 3H), 1.91-1.52 (m, 5H), 1.18 (s, 9H), 0.95 (dd, J = 20.8, 6.2 Hz, 6H). MS (ESI) m/z 460.2, [C₂₄H₃₇N₅O₄ + H]⁺ |
| 59 | 3 | 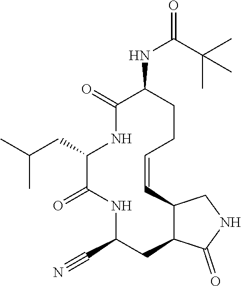 [SMILES: CC(C)C[C@@H]1NC(=O)[C@@H](NC(=O)C(C)(C)C)CC/C=C\[C@H]2CNC(=O)[C@H]2C[C@@H](C#N)NC1=O] | ¹H NMR (400 MHz, MeOD): δ (ppm) 7.23 (d, J = 7.2 Hz, 1H), 5.53-5.38 (m, 2H), 5.26 (dd, J = 11.1, 2.0 Hz, 1H), 4.59-4.49 (m, 1H), 4.21 (ddd, J = 10.9, 7.1, 4.6 Hz, 1H), 3.49 (dd, J = 9.6, 6.9 Hz, 1H), 3.28 (ddd, J = 8.0, 6.6, 4.6 Hz, 1H), 3.11 (dd, J = 9.6, 4.9 Hz, 1H), 2.61 (td, J = 7.8, 6.0 Hz, 1H), 2.31-2.15 (m, 2H), 2.15-2.00 (m, 2H), 2.00-1.77 (m, 2H), 1.71-1.51 (m, 3H), 1.20 (s, 9H), 0.96 (dd, J = 16.8, 6.1 Hz, 6H). MS (ESI) m/z 460.2, [C₂₄H₃₇N₅O₄ + H]⁺ |
| 60 | 3 | 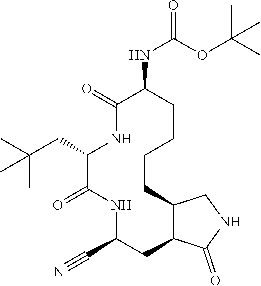 [SMILES: CC(C)(C)C[C@@H]1NC(=O)[C@@H](NC(=O)OC(C)(C)C)CCCC[C@H]2CNC(=O)[C@H]2C[C@@H](C#N)NC1=O] | ¹H NMR (400 MHz, MeOD): δ (ppm) 5.27-5.21 (m, 1H), 4.59 (dd, J = 7.2, 5.8 Hz, 1H), 3.99 (dd, J = 10.0, 4.5 Hz, 1H), 3.32 (under MeOD, 1H), 3.00 (dd, J = 9.9, 6.9 Hz, 1H), 2.49-2.34 (m, 1H), 2.13-1.91 (m, 3H), 1.83- 1.69 (m, 1H), 1.69-1.56 (m, 1H), 1.52 (dd, J = 14.2, 6.0 Hz, 1H), 1.44 (s, 9H), 1.42-1.21 (m, 6H), 0.97 (s, 9H). MS (ESI) m/z 492.2 [C₂₅H₄₁N₅O₅ + H]⁺ |
| 61 | 3 | 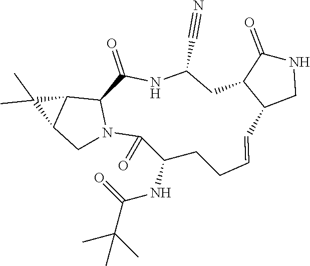 [SMILES: CC(C)(C)C(=O)N[C@H]1CC/C=C/[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ (ppm) 7.31 (d, J = 7.0 Hz, 1H), 5.49 (ddd, J = 15.3, 7.9, 4.1 Hz, 1H), 5.42-5.31 (m, 1H), 4.55 (ddd, J = 9.1, 7.0, 2.6 Hz, 1H), 4.35 (s, 1H), 4.05-3.92 (m, 2H), 3.58 (dd, J = 10.0, 5.6 Hz, 1H), 3.14 (ddd, J = 9.8, 7.1, 5.4 Hz, 1H), 3.00 (d, J = 10.1 Hz, 1H), 2.66 (ddd, J = 12.3, 7.1, 2.1 Hz, 1H), 2.55 (ddd, J = 14.5, 8.5, 2.2 Hz, 1H), 2.38-2.26 (m, 1H), 2.22 (ddd, J = 13.8, 8.7, 2.9 Hz, 1H), 2.15-2.05 (m, 1H), 1.90- 1.72 (m, 2H), 1.67 (dd, J = 7.7, 4.9 Hz, 1H), 1.60 (d, J = 7.7 Hz, 1H), 1.18 (s, 9H), 1.10 (s, 3H), 0.96 (s, 3H) MS (ESI) m/z = 484.2, [M + H⁺, C₂₆H₃₇N₅O₄ + H]⁺ |
| 62 | 4 | 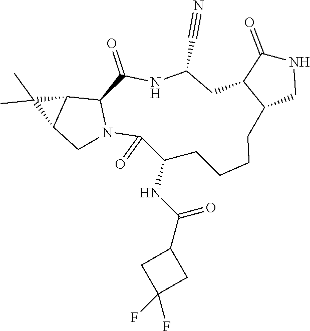 [SMILES: CC1(C)[C@@H]2[C@H]3C(=O)N[C@H](C#N)C[C@@H]4C(=O)NC[C@@H]4CCCC[C@H](NC(=O)C4CC(F)(F)C4)C(=O)N3C[C@@H]21] | ¹H NMR (400 MHz, MeOD) δ 5.06 (dd, J = 7.1 4.4 Hz, 1H), 4.48-4.40 (m, 2H), 3.96 (d, J = 10.6 Hz, 1H), 3.77 (dd, J = 10.6, 5.0 Hz, 1H), 3.32-3.25 (m, 1H), 2.96-2.80 (m, 2H), 2.77-2.52 (m, 4H), 2.46-2.28 (m, 2H), 2.10 (ddd, J = 14.8, 7.6, 4.5 Hz, 1H), 1.99-1.78 (m, 2H), 1.64-1.50 (m, 3H), 1.43-1.32 (m, 1H), 1.32-1.15 (m, 4H), 1.11 (dt, J = 9.8, 4.5 Hz, 1H), 1.02 (s, 3H), 0.88 (s, 3H). ¹³C NMR (101 MHz, MeOD) δ 179.55, 173.03 (t, J = 2.3 Hz), 171.81, 170.90, 118.90 (dd, J = 284.4, 268.6 Hz), 118.36, 60.09, 51.34 46.95, 46.01, 42.68, 38.75, 38.66, 37.89 (td, J = 24.2, 17.1 Hz), 30.92, 30.03, 28.48, 27.73, 27.38, 27.10, 26.53 (dd, J = 14.8, 4.3 Hz)., 25.31, 23.78, 18.64, 11.74. ¹⁹F NMR (376 MHz, MeOD) δ −83.48 (dtd, J = 192.8, 10.8, 5.3 Hz, 1F), −99.68 (ddt, J = 192.8, 31.7, 16.5 Hz, 1F). MS (ESI) m/z = 520.2 [C₂₆H₃₅F₂N₅O₄ + H]⁺ |
| 63 | 3 | 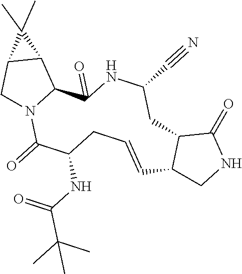 [SMILES: CC(C)(C)C(=O)N[C@H]1C/C=C/[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ (ppm) 7.58 (dd, J = 33.4, 7.6 Hz, 1H), 5.73 (ddt, J = 15.2, 9.5, 1.5 Hz, 1H), 5.60 (dddd, J = 15.1, 10.6, 3.4, 1.3 Hz, 1H), 4.78 (ddd, J = 11.7, 7.6, 5.1 Hz, 1H), 4.74-4.67 (m, 1H), 4.47 (s, 1H), 4.23 (d, J = 10.7 Hz, 1H), 3.91 (dd, J = 10.7, 5.4 Hz, 1H), 3.49 (dd, J = 9.9, 6.7 Hz, 1H), 3.19 (ddd, J = 9.4, 7.1, 4.3 Hz, 1H), 3.07 (dd, J = 9.9, 4.3 Hz, 1H), 2.68-2.54 (m, 2H), 2.52- 2.35 (m, 2H), 2.21-2.07 (m, 1H), 1.81-1.75 (m, 1H), 1.56 (dd, J = 7.6, 5.2 Hz, 1H), 1.17 (s, 9H), 1.10 (s, 3H), 0.92 (s, 3H). MS (ESI) m/z 470.2 [C₂₅H₃₅N₅O₄ + H]⁺ |
| 64 | 3 | 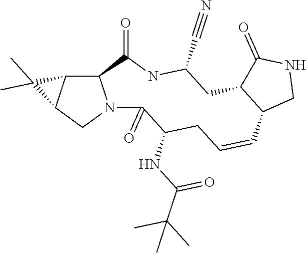 [SMILES: CC(C)(C)C(=O)N[C@H]1C/C=C\[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ (ppm) 7.57 (d, J = 9.2 Hz, 1H), 7.25 (d, J = 6.2 Hz, 2H), 5.76-5.60 (m, 4H), 5.03 (dtd, J = 11.9, 4.5, 2.6 Hz, 2H), 4.88- 4.82 (m, 11H), 4.65 (s, 2H), 3.95- 3.84 (m, 4H), 3.56 (dd, J = 9.7, 6.0 Hz, 2H), 3.19-3.09 (m, 2H), 2.95 (dd, J = 9.8, 1.4 Hz, 2H), 2.69 (ddd, J = 14.1, 11.2, 2.6 Hz, 2H), 2.62-2.51 (m, 4H), 2.47 (dt, J = 15.0, 2.4 Hz, 2H), 2.10-1.99 (m, 2H), 1.98 (d, J = 4.3 Hz, 2H), 1.59 (ddd, J = 7.8, 3.8, 1.9 Hz, 2H), 1.25 (s, 18H), 1.12 (s, 6H), 0.94 (s, 6H). MS (ESI) m/z 470.1 [C₂₅H₃₅N₅O₄ + H]⁺ |
| 65 | 4 | 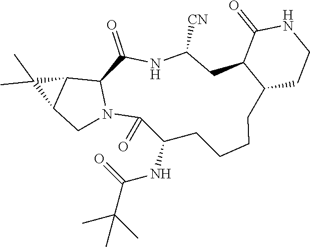 [SMILES: CC(C)(C)C(=O)N[C@H]1CCCC[C@H]2CCNC(=O)[C@@H]2C[C@@H](C#N)NC(=O)[C@@H]2[C@@H]3[C@H](CN2C1=O)C3(C)C] | ¹H NMR (400 MHz, MeOD) δ 7.31 (d, J = 7.4 Hz, 1H), 5.09 (dd, J = 12.5, 3.7 Hz, 1H), 4.63-4.55 (m, 1H), 4.50 (s, 1H), 4.01-3.91 (m, 2H), 3.39-3.36 (m, 1H), 3.28-3.18 (m, 1H), 2.41 (d, J = 1.4 Hz, 1H), 2.38-2.28 (m, 1H), 2.24-2.13 (m, 1H), 2.08-1.91 (m, 2H), 1.71-1.53 (m, 4H), 1.47 (d, J = 7.6 Hz, 1H), 1.40-1.22 (m, 4H), 1.17 (s, 9H), 1.15-1.12 (m, 1H), 1.10 (s, 3H), 1.08-1.00 (m, 1H), 0.94 (s, 3H). 13C NMR (101 MHz, MeOD) δ 178.87, 174.38, 171.29, 171.27, 118.50, 59.68, 50.92, 40.41, 38.22, 37.63, 36.61, 34.06, 31.65, 31.18, 30.02, 29.00, 27.89, 26.25, 26.00, 25.25, 23.37, 22.83, 18.83, 11.72. MS (ESI) m/z 500.2 [C₂₇H₄₁N₅O₄ + H]⁺ |
| 66 | 4 | 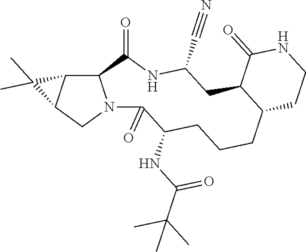 [SMILES: CC(C)(C)C(=O)N[C@H]1CCC[C@H]2CCNC(=O)[C@@H]2C[C@@H](C#N)NC(=O)[C@@H]2[C@@H]3[C@H](CN2C1=O)C3(C)C] | ¹H NMR (400 MHz, MeOD) δ 4.87- 4.82 (m, 1H), 4.58 (s, 1H), 4.50- 4.43 (m, 1H), 4.16 (d, J = 10.5 Hz, 1H), 3.83 (dd, J = 10.5, 5.0 Hz, 1H), 3.32-3.16 (m, 4H), 2.46-2.28 (m, 3H), 2.06-1.94 (m, 1H), 1.84-1.68 (m, 3H), 1.68-1.55 (m, 3H), 1.52- 1.19 (m, 3H), 1.17 (s, 9H), 1.10 (s, 3H), 0.94 (s, 3H), 0.88-0.71 (m, 1H). MS (ESI) m/z 486.1 [C₂₆H₃₉N₅O₄ + H]⁺ |
| 67 | 4 | 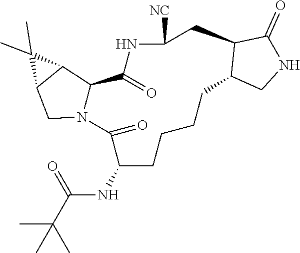 [SMILES: CC(C)(C)C(=O)N[C@H]1CCCC[C@H]2CNC(=O)[C@@H]2C[C@@H](C#N)NC(=O)[C@@H]2[C@@H]3[C@H](CN2C1=O)C3(C)C] | ¹H NMR (400 MHz, DMSO) δ 9.12 (d, J = 8.5 Hz, 1H), 7.57 (s, 1H), 7.35 (d, J = 7.2 Hz, 1H), 4.78 (ddd, J = 12.2, 8.4, 3.2 Hz, 1H), 4.40-4.34 (m, 2H), 3.96 (d, J = 10.4 Hz, 1H), 3.82 (dd, J = 10.3, 5.1 Hz, 1H), 3.38 (dd, J = 9.9, 7.4 Hz, 1H), 2.78-2.72 (m, 1H), 2.17- 2.04 (m, 2H), 2.00-1.80 (m, 3H), 1.55 (dd, J = 7.7, 5.0 Hz, 1H), 1.48- 1.40 (m, 1H), 1.36-1.26 (m, 2H), 1.24-1.13 (m, 3H), 1.06 (s, 9H), 1.02 (s, 3H), 0.97-0.86 (m, 2H), 0.83 (s, 3H). MS (ESI) m/z 486.3, [C₂₆H₃₉N₅O₄ + H]⁺ |
| 68 | 4 | 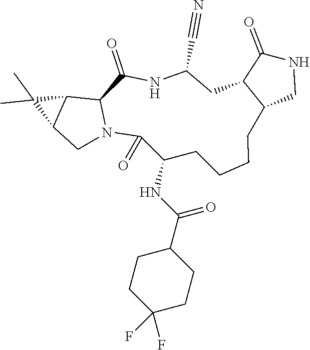 [SMILES: CC1(C)[C@@H]2[C@H]3C(=O)N[C@H](C#N)C[C@@H]4C(=O)NC[C@@H]4CCCC[C@H](NC(=O)C4CCC(F)(F)CC4)C(=O)N3C[C@@H]21] | ¹H NMR (400 MHz, MeOD): δ (ppm) 8.17 (d, J = 7.1 Hz, 1H), 5.14 (dd, J = 7.4, 4.3 Hz, 1H), 4.56- 4.47 (m, 2H), 4.05 (d, J = 10.7 Hz, 1H), 3.85 (dd, J = 10.6, 5.1 Hz, 1H), 3.39-3.35 (m, 1H), 3.01 (dd, J = 9.9, 6.1 Hz, 1H), 2.53-2.32 (m, 3H), 2.23-1.89 (m, 5H), 1.88-1.61 (m, 9H), 1.52-1.41 (m, 1H), 1.39- 1.17 (m, 5H), 1.10 (s, 3H), 0.95 (s, 3H). ¹³C NMR (101 MHz, MeOD) δ (ppm) 179.55, 175.42 (d, J = 2.2 Hz, 1C), 171.96, 170.85, 122.49 (dd, J = 241.3, 239.2 Hz), 118.35, 60.06, 50.96, 47.00, 45.96, 42.65, 41.63, 38.69, 38.66, 32.40 (dd, J = 25.1, 23.8 Hz, 2C), 30.78, 30.22, 28.44, 27.69, 27.25, 26.96, 25.58 (dd, J = 9.5, 2.9 Hz, 2C), 25.31, 23.92, 18.63, 11.80. ¹⁹F NMR (376 MHz, MeOD) δ −93.32 (d, J = 237.4 Hz), −103.00 (d, J = 237.5 Hz). MS (ESI) m/z 548.2 [C₂₈H₃₉F₂N₅O₄ + H]⁺ |
| 69 | 3 | 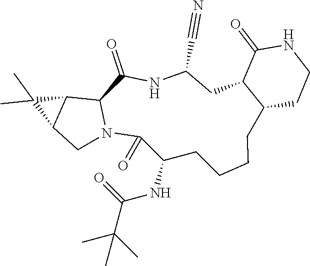 [SMILES: CC(C)(C)C(=O)N[C@H]1CCCC[C@H]2CCNC(=O)[C@H]2C[C@@H](C#N)NC(=O)[C@@H]2[C@@H]3[C@H](CN2C1=O)C3(C)C] | ¹H NMR (400 MHz, MeOD) δ 7.36 (d, J = 7.1 Hz, 1H), 5.41 (dd, J = 7.5, 3.6 Hz, 1H), 4.59 (td, J = 7.4, 3.1 Hz, 1H), 4.50 (s, 1H), 3.93 (d, J = 10.8 Hz, 1H), 3.85 (dd, J = 10.8, 5.0 Hz, 1H), 3.32-3.23 (m, 2H), 2.51-2.42 (m, 1H), 2.26-2.16 (m, 1H), 2.16-2.05 (m, 1H), 2.04- 1.88 (m, 2H), 1.71 (d, J = 7.6 Hz, 1H), 1.68- 1.57 (m, 4H), 1.50-1.24 (m, 5H), 1.20 (s, 9H), 1.17-1.12 (m, 2H), 1.10 (s, 3H), 0.93 (s, 3H). 13C NMR (101 MHz, MeOD) δ 178.99, 175.03, 171.69, 170.69, 118.64, 60.38, 51.09, 46.87, 40.37, 40.09, 38.47, 38.25, 34.86, 32.10, 29.85, 29.29, 28.44, 27.62, 26.28, 26.02, 25.27, 24.65, 23.96, 18.56, 11.75 MS (ESI) m/z 500.1 [C₂₇H₄₁N₅O₄ + H]⁺ |
| 70 | 3 | 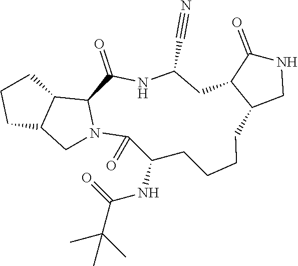 | ¹H NMR (400 MHz, MeOD-d₄): δ (ppm) 7.39 (d, J = 7.0 Hz, 1H), 5.21 (dd, J = 7.6, 4.2 Hz, 1H), 4.65-4.53 (m, 2H), 3.84-3.66 (m, 2H), 3.33 (under MeOD, 1H), 3.08-2.84 (m, 3H), 2.46-2.33 (m, 2H), 2.16 (ddd, J = 14.8, 7.2, 4.2 Hz, 1H), 2.06-1.86 (m, 4H), 1.79-1.20 (m, 11H), 1.18 (s, 9H). MS (ESI) m/z 486.2 [C₂₆H₃₉N₅O₄ + H]⁺ |
| 71 | 3 | 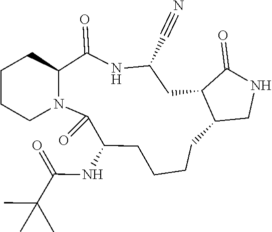 [SMILES: CC(C)(C)C(=O)N[C@H]1CCCC[C@H]2CNC(=O)[C@H]2C[C@@H](C#N)NC(=O)[C@@H]2CCCCN2C1=O] | ¹H NMR (400 MHz, MeOD) δ 7.44 (d, J = 7.1 Hz, 1H), 5.26 (d, J = 5.7 Hz, 1H), 5.22 (dd, J = 9.9, 2.9 Hz, 1H), 4.93 (td, J = 7.7, 4.5 Hz, 1H), 4.10 (d, J = 13.4 Hz, 1H), 3.37- 3.30 (m, 1H), 3.15 (td, J = 13.1, 2.9 Hz, 1H), 2.99 (dd, J = 9.9, 8.2 Hz, 1H), 2.53-2.39 (m, 1H), 2.23 (ddd, J = 9.3, 7.5, 4.0 Hz, 1H), 2.17-1.86 (m, 5H), 1.80-1.23 (m, 11H), 1.20 (s, 9H). ¹³C NMR (101 MHz, MeOD) δ 180.19, 178.98, 172.11, 171.39, 118.42, 51.77, 45.96, 43.76, 41.95, 39.17, 38.21, 38.18, 37.96, 31.51, 31.48, 29.83, 27.45, 26.31, 26.30, 26.17, 25.24, 24.45, 23.90, 19.54. MS (ESI) m/z 460.2 [C₂₄H₃₇N₅O₄ + H]⁺ |
| 72 | 3 | 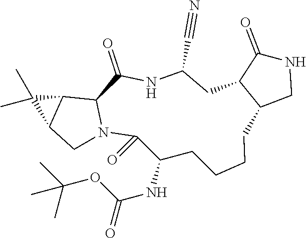 [SMILES: CC(C)(C)OC(=O)N[C@H]1CCCC[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ (ppm) 5.14 (dd, J = 7.5, 4.2 Hz, 1H), 4.54 (s, 1H), 4.28 (dd, J = 8.9, 3.6 Hz, 1H), 4.08 (d, J = 10.7 Hz, 1H), 3.84 (dd, J = 10.7, 5.1 Hz, 1H), 3.84 (dd, J = 10.7, 5.1 Hz, 1H), 3.38-3.34 (m, 1H), 3.00 (dd, J = 9.9, 6.1 Hz, 1H), 2.52-2.36 (m, 2H), 2.17 (ddd, J = 14.9, 7.4, 4.3 Hz, 1H), 2.10-1.98 (m, 1H), 1.95-1.81 (m, 1H), 1.72-1.57 (m 3H), 1.43 (s, 9H), 1.52-1.39 (m, 1H), 1.39-1.14 (m, 5H), 1.11 (s, 3H), 0.96 (s, 3H). ¹³C NMR (101 MHz, MeOD) δ (ppm) 179.59, 172.74, 170.87, 155.98, 118.35, 79.12, 60.00, 52.05, 46.97 (HSQC), 45.90, 42.63, 38.70, 38.62, 30.76, 30.64, 28.42, 27.70, 27.28, 27.18, 26.81, 25.36, 23.89, 18.62, 11.77. MS (ESI) m/z 502.2 [C₂₆H₃₉N₅O₅ + H]⁺ |
| 73 | 4 | 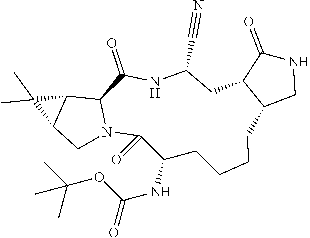 [SMILES: CC(C)(C)OC(=O)N[C@H]1CCCC[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ (ppm) 8.70 (d, J = 8.6 Hz, 0.3H), 8.09 (d, J = 7.2 Hz, 1H), 5.18- 5.08 (m, 1H), 4.59-4.47 (m, 2H), 4.09 (d, J = 10.7 Hz, 1H), 3.86 (dd, J = 10.6, 5.0 Hz, 1H), 3.42-3.35 (m, 1H), 3.01 (dd, J = 10.0, 6.0 Hz, 1H), 2.47 (dq, J = 23.4, 7.1 Hz, 2H), 2.19 (ddd, J = 14.8, 7.4, 4.4 Hz, 1H), 2.12-1.99 (m, 3H), 1.98-1.86 (m, 1H), 1.72-1.59 (m, 3H), 1.56-1.40 (m, 1H), 1.40-1.18 (m, 5H), 1.10 (s, 3H), 1.01 (s, 9H), 0.97 (s, 3H). ¹³C NMR (101 MHz, MeOD) δ (ppm) 179.52, 172.61, 172.04, 170.90, 118.34, 60.04, 51.03, 48.38, 47.06, 45.87, 42.75, 38.70, 38.66, 30.78, 30.56, 30.48, 28.82, 28.52, 27.76, 27.20, 26.81, 25.32, 23.92, 18.69, 11.91. MS (ESI) m/z 500.2 [C₂₇H₄₁N₅O₄ + H]⁺ |
| 74 | 3 | 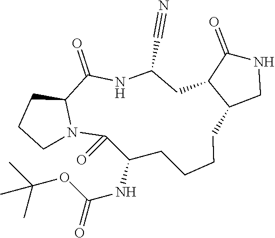 | ¹H NMR (400 MHz, MeOD-d4): δ (ppm) 8.55-7.40 (m, 1H), 5.21 (dd, J = 8.7, 3.5 Hz, 1H), 4.71 (dd, J = 8.1, 2.8 Hz, 1H), 4.54 (ddd, J = 8.0, 6.2, 3.5 Hz, 1H), 3.89-3.79 (m, 1H), 3.64 (ddd, J = 9.9, 7.6, 4.2 Hz, 1H), 3.33 (under MeOD, 1H), 2.99 (dd, J = 10.0, 5.8 Hz, 1H), 2.47-2.37 (m, 2H), 2.37- 2.29 (m, 1H), 2.18 (ddd, J = 15.0, 7.0, 3.5 Hz, 1H), 2.14-1.87 (m, 5H), 1.81-1.70 (m, 1H), 1.51-1.21 (m, 6H), 1.19 (s, 9H). MS (ESI) m/z 446.2 [C₂₃H₃₅N₅O₄ + H]⁺ |
| 75 | 4 | 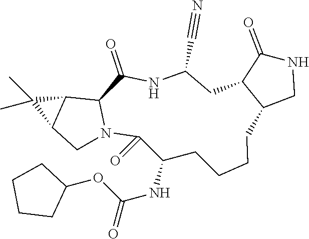 [SMILES: CC1(C)[C@@H]2[C@H]3C(=O)N[C@H](C#N)C[C@@H]4C(=O)NC[C@@H]4CCCC[C@H](NC(=O)OC4CCCC4)C(=O)N3C[C@@H]21] | ¹H NMR (400 MHz, MeOD): δ (ppm) 7.09 (d, J = 7.4 Hz, 1H), 5.12 (dd, J = 7.2, 4.4 Hz, 1H), 4.99 (tt, J = 5.7, 2.6 Hz, 1H), 4.51 (s, 1H), 4.34- 4.26 (m, 1H), 4.00 (d, J = 10.7 Hz, 1H), 3.82 (dd, J = 10.6, 5.0 Hz, 1H), 3.33 (under MeOD, 1H), 2.98 (dd, J = 9.9, 6.3 Hz, 1H), 2.49-2.35 (m, 2H), 2.20-2.08 (m, 1H), 2.07-1.95 (m, 1H), 1.93-1.76 (m, 3H), 1.76-1.53 (m, 9H), 1.47-1.12 (m, 6H), 1.08 (s, 3H), 0.95 (s, 3H). MS (ESI) m/z 514.2 [C₂₇H₃₉N₅O₅ + H]⁺ |
| 76 | 4 | 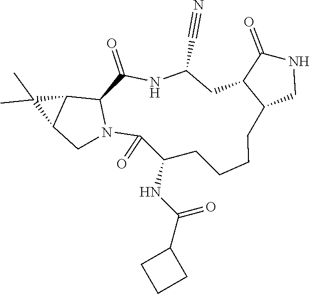 | ¹H NMR (400 MHz, MeOD) δ 7.96 (d, J = 7.2 Hz, 1H), 5.14 (dd, J = 7.4, 4.3 Hz, 1H), 4.56-4.47 (m, 2H), 4.06 (d, J = 10.7 Hz, 1H), 3.85 (dd, J = 10.7, 5.1 Hz, 1H), 3.39-3.34 (m, 1H), 3.19-3.09 (m, 1H), 3.00 (dd, J = 9.9, 6.1 Hz, 1H), 2.51-2.40 (m, 2H), 2.29-2.09 (m, 5H), 2.09-1.79 (m, 4H), 1.69 (d, J = 7.6 Hz, 1H), 1.66- 1.59 (m, 2H), 1.51-1.42 (m, 1H), 1.39-1.25 (m, 4H), 1.23-1.15 (m, 1H), 1.11 (s, 3H), 0.96 (s, 3H). ¹³C NMR (101 MHz, MeOD) δ 179.58, 175.62, 171.97, 170.89, 118.31, 60.03, 50.97, 48.31, 48.09, 45.94, 42.61, 38.85, 38.68, 38.62, 30.80, 30.19, 28.43, 27.68, 27.20, 26.94, 25.30, 24.74, 24.62, 23.94, 18.61, 17.66, 11.76. MS (ESI) m/z 500.1 [C₂₆H₃₇N₅O₄ + H]⁺ |
| 77 | 4 | 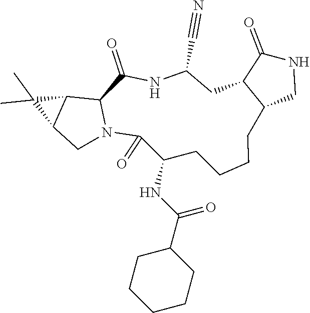 [SMILES: CC1(C)[C@@H]2[C@H]3C(=O)N[C@H](C#N)C[C@@H]4C(=O)NC[C@@H]4CCCC[C@H](NC(=O)C4CCCCC4)C(=O)N3C[C@@H]21] | ¹H NMR (400 MHz, MeOD) δ 5.12 (dd, J = 7.5, 4.2 Hz, 1H), 4.52-4.47 (m, 2H), 4.04 (d, J = 10.7 Hz, 1H), 3.82 (dd, J = 10.7, 5.1 Hz, 1H), 3.36-3.33 (m, 1H), 2.99 (dd, J = 9.9, 6.1 Hz, 1H), 2.49-2.36 (m, 2H), 2.25-2.10 (m, 2H), 2.07-1.86 (m, 2H), 1.82- 1.55 (m, 8H), 1.49-1.13 (m, 11H), 1.08 (s, 3H), 0.92 (s, 3H). MS (ESI) m/z 512.3, [M + H⁺, C₂₈H₄₁N₅O₄ + H]⁺ |
| 78 | 3 | 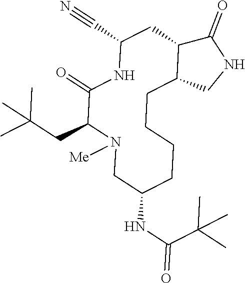 | ¹H NMR (400 MHz, DMSO) δ 8.48 (d, J = 9.1 Hz, 1H), 7.62 (s, 1H), 7.48 (d, J = 7.8 Hz, 1H), 5.18 (t, J = 6.5 Hz, 1H), 5.10 (t, J = 1.5 Hz, 1H), 4.76-4.68 (m, 1H), 3.17-3.08 (m, 1H), 2.99 (s, 3H), 2.81 (t, J = 9.0 Hz, 1H), 2.30- 2.21 (m, 1H), 2.08 (ddd, J = 14.1, 10.5, 3.4 Hz, 1H), 1.98-1.82 (m, 2H), 1.79-1.69 (m, 2H), 1.44-1.12 (m, 8H), 1.05 (s, 9H), 0.85 (s, 9H). MS (ESI) m/z 490.4 [C₂₆H₄₃N₅O₄ + H]⁺ |
| 79 | 3 | 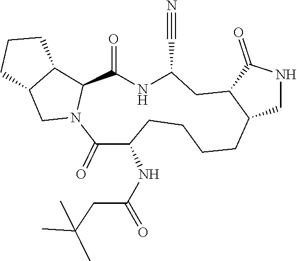 [SMILES: CC(C)(C)CC(=O)N[C@H]1CCCC[C@H]2CNC(=O)[C@H]2C[C@@H](C#N)NC(=O)[C@@H]2[C@H]3CCC[C@H]3CN2C1=O] | ¹H NMR (400 MHz, MeOD) δ 5.25 (dd, J = 7.9, 3.8 Hz, 1H), 4.60 (d, J = 2.3 Hz, 1H), 4.38 (dd, J = 8.5, 3.7 Hz, 1H), 3.88 (dd, J = 10.9, 3.8 Hz, 1H), 3.71 (dd, J = 10.9, 8.1 Hz, 1H), 3.11- 2.85 (m, 3H), 2.48-2.33 (m, 2H), 2.14 (ddd, J = 14.8, 7.5, 3.9 Hz, 1H), 2.05-1.83 (m, 4H), 1.80-1.09 (m, 21H). MS (ESI) m/z 502.2 [C₂₆H₃₉N₅O₅ + H]⁺ |
| 80 | 3 | 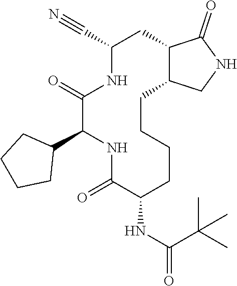 [SMILES: CC(C)(C)C(=O)N[C@H]1CCCC[C@H]2CNC(=O)[C@H]2C[C@@H](C#N)NC(=O)[C@H](C2CCCC2)NC1=O] | ¹H NMR (400 MHz, MeOD) δ 7.16 (d, J = 7.3 Hz, 1H), 5.25 (dd, J = 9.0, 3.1 Hz, 1H), 4.31 (dd, J = 10.0, 4.5 Hz, 1H), 4.25 (d, J = 10.8 Hz, 1H), 2.99 (dd, J = 9.8, 7.1 Hz, 1H), 2.49-2.32 (m, 3H), 2.05 (tddd, J = 14.5, 11.4, 8.6, 4.0 Hz, 2H), 1.88-1.73 (m, 3H), 1.63 (ddq, J = 27.8, 14.1, 6.4 Hz, 5H), 1.47-1.19 (m, 8H), (s, 9H). ¹³C NMR (101 MHz, MeOD) δ 180.19, 178.98, 172.11, 171.39, 118.42, 51.77, 45.96, 43.76, 41.95, 39.17, 38.21, 38.18, 37.96, 31.51, 31.48, 29.83, 27.45, 26.31, 26.30, 26.17, 25.24, 24.45, 23.90, 19.54. MS (ESI) m/z 474.2 [C₂₅H₃₉N₅O₄ + H]⁺ |
| 81 | 4 | 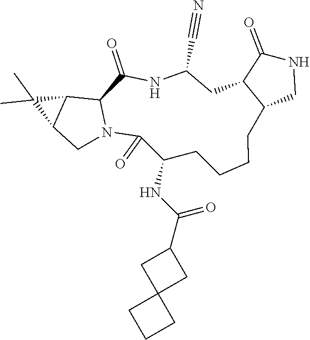 [SMILES: CC1(C)[C@@H]2[C@H]3C(=O)N[C@H](C#N)C[C@@H]4C(=O)NC[C@@H]4CCCC[C@H](NC(=O)C4CC5(CCC5)C4)C(=O)N3C[C@@H]21] | ¹H NMR (400 MHz, MeOD) δ 5.13 (dd, J = 7.4, 4.2 Hz, 1H), 4.50 (d, J = 9.0 Hz, 2H), 4.03 (d, J = 10.7 Hz, 1H), 3.83 (dd, J = 10.7, 5.1 Hz, 1H), 3.38- 3.32 (m, 1H), 3.04-2.88 (m, 2H), 2.51-2.35 (m, 2H), 2.23-2.10 (m, 5H), 2.10-1.97 (m, 3H), 1.97-1.87 (m, 3H), 1.87-1.75 (m, 2H), 1.70- 1.55 (m, 3H), 1.50-1.12 (m, 7H), 1.09 (s, 3H), 0.93 (s, 3H). ¹³C NMR (101 MHz, MeOD) δ 179.58, 175.56, 171.96, 170.89, 118.31, 60.02, 51.02, 45.93, 42.59, 39.72, 38.67, 38.62, 37.38, 37.21, 34.74, 33.82, 33.38, 30.79, 30.20, 28.42, 27.67, 27.19, 26.92, 25.30, 23.93, 18.61, 15.59, 11.79. MS (ESI) m/z 524.3 [C₂₉H₄₁N₅O₄ + H]⁺ |
| 82 | 4 | 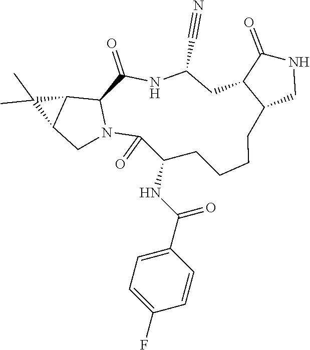 [SMILES: CC1(C)[C@@H]2[C@H]3C(=O)N[C@H](C#N)C[C@@H]4C(=O)NC[C@@H]4CCCC[C@H](NC(=O)c4ccc(F)cc4)C(=O)N3C[C@@H]21] | ¹H NMR (400 MHz, MeOD) δ 7.16 (d, J = 7.3 Hz, 1H), 5.25 (dd, J = 9.0, 3.1 Hz, 1H), 4.31 (dd, J = 10.0, 4.5 Hz, 1H), 4.25 (d, J = 10.8 Hz, 1H), 2.99 (dd, J = 9.8, 7.1 Hz, 1H), 2.49-2.32 (m, 3H), 2.05 (tddd, J = 14.5, 11.4, 8.6, 4.0 Hz, 2H), 1.88-1.73 (m, 3H), 1.63 (ddq, J = 27.8, 14.1, 6.4 Hz, 5H), 1.47-1.19 (m, 8H), 1.18 (s, 9H). ¹³C NMR (101 MHz, MeOD) δ 180.19, 178.98, 172.11, 171.39, 118.42, 51.77, 45.96, 43.76, 41.95, 39.17, 38.21, 38.18, 37.96, 31.51, 31.48, 29.83, 27.45, 26.31, 26.30, 26.17, 25.24, 24.45, 23.90, 19.54. MS (ESI) m/z 524.2 [C₂₈H₃₄FN₅O₄ + H]⁺ |
| 83 | 4 | 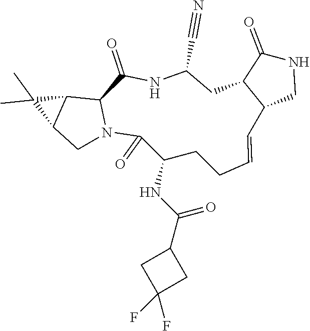 [SMILES: CC1(C)[C@@H]2[C@H]3C(=O)N[C@H](C#N)C[C@@H]4C(=O)NC[C@@H]4/C=C/CC[C@H](NC(=O)C4CC(F)(F)C4)C(=O)N3C[C@@H]21] | ¹H NMR (400 MHz, MeOD): δ (ppm) 8.24 (d, J = 6.5 Hz, 1H), 5.45 (ddd, J = 15.5, 8.0, 3.4 Hz, 1H), 5.32 (dd, J = 15.4, 9.8 Hz, 1H), 4.84-4.82 (m, 1H), 4.53-4.45 (m, 1H), 4.30 (s, 1H), 4.00-3.89 (m, 2H), 3.56 (dd, J = 10.0, 5.6 Hz, 1H), 3.16-3.07 (m, 1H), 3.00-2.89 (m, 2H), 2.78-2.59 (m, 5H), 2.52 (ddd, J = 14.5, 8.7, 2.1 Hz, 1H), 2.40-2.31 (m, 1H), 2.18-2.05 (m, 2H), 1.85-1.74 (m, 2H), 1.67-1.62 (m, 1H), 1.56 (dd, J = 7.7, 1.1 Hz, 1H), 1.08 (s, 3H), 0.97 (s, 3H). ¹⁹F NMR (376 MHz, MeOD): δ (ppm) −83.15 −83.95 (m, 1F), −99.79 (dtt, J = 193.0, 18.0, 14.4 Hz, 1F). MS (ESI) m/z 518.2 [C₂₆H₃₃N₅O₄F₂ + H]⁺ |
| 84 | 4 | 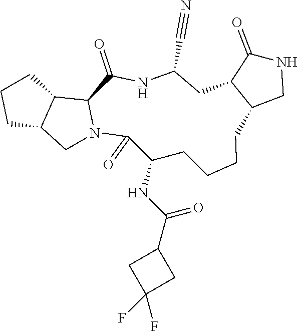 [SMILES: N#C[C@@H]1C[C@@H]2C(=O)NC[C@@H]2CCCC[C@H](NC(=O)C2CC(F)(F)C2)C(=O)N2C[C@@H]3CCC[C@@H]3[C@H]2C(=O)N1] | ¹H NMR (400 MHz, DMSO-d₆) δ 8.396 (d, J = 7.36 Hz, 1H), 8.3048 (d, J = 8.96 Hz, 1H), 7.627 (s, 1H), 5.0556 (sex, J = 4.13 Hz, 1H), 4.5023 (sex, J = 3.97 Hz, 1H), 4.4689 (d, J = 2.21 Hz, 1H), 3.7151-3.7508 (m, 1H), 3.57-3.6170 (m, 1H), 3.1570 (q, J = 5.40 Hz, 1H), 2.8838-2.9898 (m, 2H), 2.8097 (q, J = 5.40 Hz, 1H), 2.7208-2.7721 (m, 1H), 2.6190-2.7030 (m, 4H), 2.2586 (q, J = 6.58 Hz, 1H), 2.1350 (q, J = 7.06 Hz, 1H), 1.9541-2.0203 (m, 1H), 1.7406-1.8781 (m, 4H), 1.4473-1.5858 (m, 3H), 1.2957-1.4290 (m, 3H), 1.1843-1.2612 (m, 4H), 1.0623- 1.0767 (m, 1H). ¹⁹F NMR (376 MHz, DMSO-d₆) δ −80.0731 (d, J = 188.8 Hz, 1F), −95.6377 (d, J = 189.5 Hz, 1F). MS (ESI) m/z 520.2 [C₂₆H₃₅N₅O₄F₂ + H]⁺ |
| 85 | 4 | 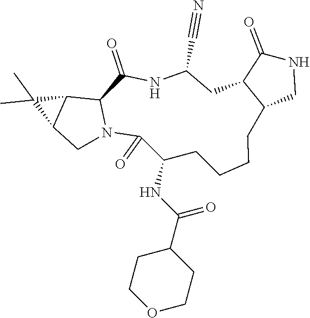 [SMILES: CC1(C)[C@@H]2[C@H]3C(=O)N[C@H](C#N)C[C@@H]4C(=O)NC[C@@H]4CCCC[C@H](NC(=O)C4CCOCC4)C(=O)N3C[C@@H]21] | ¹H NMR (400 MHz, MeOD): δ (ppm) 8.12 (d, J = 7.1 Hz, 1H), 5.11 (dd, J = 7.4, 4.3 Hz, 1H), 4.54-4.45 (m, 2H), 4.03 (d, J = 10.7 Hz, 1H), 3.98-3.89 (m, 2H), 3.83 (dd, J = 10.6, 5.1 Hz, 1H), 3.41 (tt, J = 11.7, 2.5 Hz, 2H), 3.36-3.32 (m, 1H), 2.98 (dd, J = 9.9, 6.1 Hz, 1H), 2.54-2.35 (m, 3H), 2.16 (ddd, J = 14.9, 7.5, 4.4 Hz, 1H), 2.05-1.86 (m, 2H), 1.79- 1.56 (m, 7H), 1.49-1.39 (m, 1H), 1.38-1.23 (m, 4H), 1.21-1.13 (m, 1H), 1.08 (s, 3H), 0.93 (s, 3H). MS (ESI) m/z 514.2 [C₂₇H₃₉N₅O₅ + H]⁺ |
| 86 | 3 | 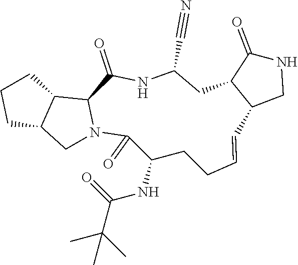 [SMILES: CC(C)(C)C(=O)N[C@H]1CC/C=C/[C@H]2CNC(=O)[C@H]2C[C@@H](C#N)NC(=O)[C@@H]2[C@H]3CCC[C@H]3CN2C1=O] | ¹H NMR (400 MHz, MeOD): δ (ppm) 5.49 (ddd, J = 15.1, 8.1, 3.6 Hz, 1H), 5.36 (dd, J = 15.3, 9.8 Hz, 1H), 4.87- 4.81 (m, 2H), 4.65-4.58 (m, 2H), 4.29 (d, J = 3.3 Hz, 1H), 3.90 (dd, J = 10.5, 7.3 Hz, 1H), 3.81 (dd, J = 10.6, 3.5 Hz, 1H), 3.57 (dd, J = 10.0, 5.6 Hz, 1H), 3.18-3.08 (m, 1H), 2.91 (m, 2H), 2.65 (ddd, J = 12.2, 7.2, 2.1 Hz, 1H), 2.54 (ddd, J = 14.5, 8.3, 2.1 Hz, 1H), 2.38-2.27 (m, 1H), 2.26-2.10 (m, 2H), 2.01-1.86 (m, 3H), 1.86- 1.74 (m, 3H), 1.74-1.50 (m, 3H), 1.21 (s, 10H). MS (ESI) m/z 485.3 [C₂₆H₃₇N₅O₄ + H]⁺ |
| 87 | 4 | 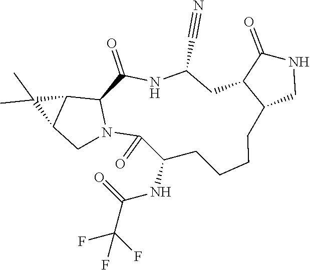 [SMILES: CC1(C)[C@@H]2[C@H]3C(=O)N[C@H](C#N)C[C@@H]4C(=O)NC[C@@H]4CCCC[C@H](NC(=O)C(F)(F)F)C(=O)N3C[C@@H]21] | ¹H NMR (400 MHz, MeOD) δ 5.15 (dd, J = 7.0, 4.4 Hz, 1H), 4.56 (dd, J = 8.7, 3.7 Hz, 1H), 4.54 (s, 1H), 4.03 (d, J = 10.7 Hz, 1H), 3.87 (dd, J = 10.6, 4.7 Hz, 1H), 3.40-3.34 (m, 1H), 3.01 (dd, J = 9.9, 6.1 Hz, 1H), 2.55-2.40 (m, 2H), 2.24-2.15 (m, 1H), 2.05-1.97 (m, 2H), 1.78-1.61 (m, 3H), 1.55-1.45 (m, 1H), 1.41-1.16 (m, 5H), 1.11 (s, 3H), 0.97 (s, 3H). 13C NMR (101 MHz, MeOD) δ 181.59, 172.83, 172.56, 159.14 (q, J = 38.38 Hz), 120.38, 118.06 (q, J = 286.84 Hz), 62.22, 53.87, 47.92, 44.84, 40.80, 40.68, 33.06, 31.60, 30.61, 29.80, 29.24, 28.90, 27.35, 25.76, 20.73, 13.65, 1.42. ¹⁹F NMR (376 MHz, MeOD) δ −76.83. MS (ESI) m/z 498.1 [C₂₃H₃₀N₅O₄F₃ + H]⁺ |
| 88 | 4 | 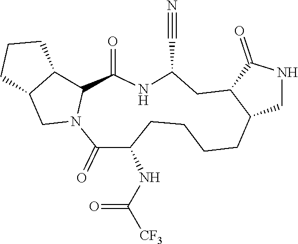 | ¹H NMR (400 MHz, MeOD) δ 5.23 (dd, J = 7.6, 4.1 Hz, 1H), 4.65 (dd, J = 8.7, 3.8 Hz, 1H), 4.60 (d, J = 2.3 Hz, 1H), 3.86 (dd, J = 10.9, 3.9 Hz, 1H), 3.76 (dd, J = 10.9, 8.0 Hz, 1H), 3.38-3.34 (m, 1H), 3.11-2.87 (m, 3H), 2.43 (h, J = 7.1 Hz, 2H), 2.19 (ddd, J = 14.9, 7.1, 4.1 Hz, 1H), 2.09-1.88 (m, 4H), 1.84-1.18 (m, 11H). MS (ESI) m/z 498.2 [C₂₃H₃₀F₃N₅O₄ + H]⁺ |
| 89 | 4 | 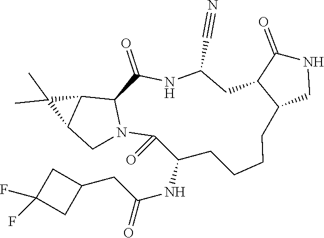 [SMILES: CC1(C)[C@@H]2[C@H]3C(=O)N[C@H](C#N)C[C@@H]4C(=O)NC[C@@H]4CCCC[C@H](NC(=O)CC4CC(F)(F)C4)C(=O)N3C[C@@H]21] | ¹H NMR (400 MHz, MeOD) δ (ppm) 5.13 (dd, J = 7.1, 4.4 Hz, 1H), 4.52 (s, 1H), 4.52-4.47 (m, 1H), 4.04 (d, J = 10.7 Hz, 1H), 3.85 (dd, J = 10.6, 4.9 Hz, 1H), 3.40-3.34 (m, 1H), 3.01 (dd, J = 10.0, 6.1 Hz, 1H), 2.73-2.58 (m, 2H), 2.54-2.39 (m, 5H), 2.35-2.15 (m, 3H), 2.03 (dt, J = 14.9, 6.8 Hz, 1H), 1.97-1.85 (m, 1H), 1.72-1.60 (m, 3H), 1.53- 1.40 (m, 1H), 1.40-1.16 (m, 5H), 1.11 (s, 3H), 0.96 (s, 3H). ¹³C NMR (101 MHz, MeOD) δ (ppm) 179.53, 172.09, 171.95, 170.90, 118.33, 60.05, 51.07, 46.99, 45.96, 42.74, 40.24, 39.76 (d, J = 3.6 Hz), 39.76 (dd, J = 44.8, 4.2 Hz), 38.73, 38.66, 30.88, 30.19, 28.50, 27.74, 27.32, 27.00, 25.30, 23.80, 20.24, (dd, J = 13.3, 6.3 Hz), 18.65, 11.76. |
| | | | ¹⁹F NMR (376 MHz, MeOD) δ (ppm) −83.97-−84.84 (m), |
| | | | −96.71-−97.62 (m). |
| | | | MS (APCI) m/z 534.3 [C₂₇H₃₇F₂N₅O₄ + H]⁺ |
| 90 | 4 | 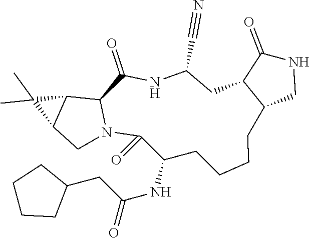 [SMILES: CC1(C)[C@@H]2[C@H]3C(=O)N[C@H](C#N)C[C@@H]4C(=O)NC[C@@H]4CCCC[C@H](NC(=O)CC4CCCC4)C(=O)N3C[C@@H]21] | ¹H NMR (400 MHz, MeOD) δ (ppm) 8.19 (d, J = 7.2 Hz, 1H), 5.13 (dd, J = 7.2, 4.4 Hz, 1H), 4.57-4.48 (m, 2H), 4.06 (d, J = 10.7 Hz, 1H), 3.85 (dd, J = 10.6, 5.0 Hz, 1H), 3.37 (d, J = 6.6 Hz, 1H), 3.01 (dd, J = 9.9, 6.0 Hz, 1H), 2.54-2.37 (m, 2H), 2.24-2.14 (m, 4H), 2.08- 2.00 (m, 1H), 1.93 (ddt, J = 13.1, 8.7, 6.3 Hz, 1H), 1.82- 1.72 (m, 2H), 1.70-1.42 (m, 8H), 1.39-1.13 (m, 7H), 1.10 (s, 3H), 0.96 (s, 3H). ¹³C NMR (101 MHz, MeOD) δ (ppm) 179.54, 173.88, 171.97, 170.91, 118.32, 60.03, 51.03, 47.09 (HSQC) 45.91, 42.71, 41.09, 38.70, 38.65, 37.22, 31.94, 31.86, 30.82, 30.35, 28.50, 27.73, 27.22, 26.90, 25.30, 24.44, 23.91, 18.65, 11.80. MS (APCI) m/z 512.3 [C₂₈H₄₁N₅O₄ + H]⁺ |
| 91 | 4 | 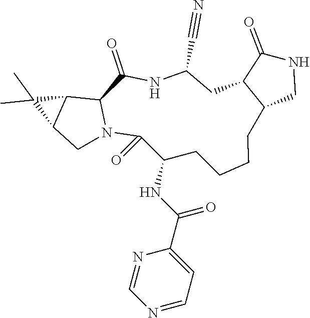 [SMILES: CC1(C)[C@@H]2[C@H]3C(=O)N[C@H](C#N)C[C@@H]4C(=O)NC[C@@H]4CCCC[C@H](NC(=O)c4ccncn4)C(=O)N3C[C@@H]21] | ¹H NMR (400 MHz, MeOD) δ 9.28 (d, J = 1.4 Hz, 1H), 9.02 (d, J = 5.1 Hz, 1H), 8.06 (dd, J = 5.1, 1.4 Hz, 1H), 5.15 (dt, J = 6.5, 4.8 Hz, 1H), 4.79 (dd, J = 7.4, 3.4 Hz, 1H), 4.55 (s, 1H), 4.02 (d, J = 10.7 Hz, 1H), 3.91 (dd, J = 10.9, 3.7 Hz, 1H), 3.38-3.34 (m, 1H), 2.99 (dd, J = 9.9, 6.4 Hz, 1H), 2.55-2.39 (m, 2H), 2.25-2.07 (m, 2H), 2.04-1.95 (m, 1H), 1.82-1.74 (m, 1H), 1.69-1.63 (m, 2H), 1.52-1.41 (m, 1H), 1.41-1.27 (m, 4H), 1.25-1.15 (m, 1H), 1.09 (s, 3H), 0.93 (s, 3H). LCMS m/z = 508.3, [M + H⁺, C₂₆H₃₃N₇O₄ + H]⁺ |
| 92 | 3 | 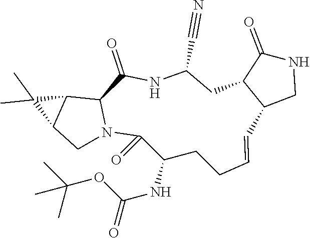 [SMILES: CC(C)(C)OC(=O)N[C@H]1CC/C=C/[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ 5.45 (ddd, J = 15.4, 7.9, 3.5 Hz, 1H), 5.33 (dd, J = 15.4, 9.7 Hz, 1H), 4.86-4.79 (m, 1H), 4.33 (s, 1H), 4.31-4.24 (m, 1H), 4.01 (d, J = 10.4 Hz, 1H), 3.91 (dd, J = 10.4, 5.2 Hz, 1H), 3.55 (dd, J = 10.0, 5.7 Hz, 1H), 3.14-3.06 (m, 1H), 2.97 (dd, J = 10.1, 0.8 Hz, 1H), 2.63 (ddd, J = 12.3, 7.2, 2.1 Hz, 1H), 2.51 (ddd, J = 14.5, 8.5, 2.2 Hz, 1H), 2.41-2.27 (m, 1H), 2.15-2.03 (m, 2H), 1.87-1.68 (m, 2H), 1.64 (dd, J = 7.7, 5.1 Hz, 1H), 1.58 (d, J = 7.7 Hz, 1H), 1.41 (s, 9H), 1.09 (s, 3H), 0.97 (s, 3H). MS (ESI) m/z 500.2 [C₂₆H₃₇N₅O₅ + H]⁺ |
| 93 | 4 | 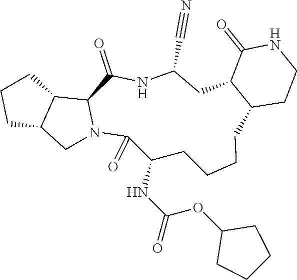 [SMILES: N#C[C@@H]1C[C@@H]2C(=O)NCC[C@@H]2CCCC[C@H](NC(=O)OC2CCCC2)C(=O)N2C[C@@H]3CCC[C@@H]3[C@H]2C(=O)N1] | ¹H NMR (400 MHz, MeOD): δ (ppm) 8.15 (s, 1H), 5.52- 5.44 (m, 1H), 5.07-5.02 (m, 1H), 4.55 (d, J = 2.3 Hz, 1H), 4.44 (dd, J = 7.1, 3.6 Hz, 1H), 3.79-3.68 (m, 2H), 3.28-3.25 (m, 1H), 3.06 (q, J = 7.6 Hz, 1H), 2.98-2.87 (m, 1H), 2.39 (dd, J = 9.4, 4.5 Hz, 1H), 2.14 (ddd, J = 13.0, 9.3, 3.2 Hz, 1H), 2.06-1.81 (m, 8H), 1.80-1.56 (m, 12H), 1.51 (dq, J = 19.7, 6.5 Hz, 3H), 1.39 (dd, J = 8.0, 4.1 Hz, 1H), 1.34-1.27 (m, 2H), 1.15-1.10 (m, 1H). MS (ESI) m/z 528.3 [C₂₈H₄₁N₅O₅ + H]⁺ |
| 94 | 4 | 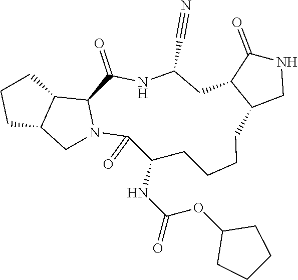 [SMILES: N#C[C@@H]1C[C@@H]2C(=O)NC[C@@H]2CCCC[C@H](NC(=O)OC2CCCC2)C(=O)N2C[C@@H]3CCC[C@@H]3[C@H]2C(=O)N1] | ¹H NMR (400 MHz, MeOD): δ (ppm) 5.23 (dd, J = 7.6, 4.1 Hz, 1H), 5.07-5.01 (m, 1H), 4.59 (d, J = 2.3 Hz, 1H), 4.41 (dd, J = 8.1, 3.8 Hz, 1H), 3.84 (dd, J = 10.9, 3.9 Hz, 1H), 3.73 (dd, J = 10.8, 8.0 Hz, 1H), 3.38- 3.34 (m, 1H), 3.09-2.96 (m, 2H), 2.96-2.87 (m, 1H), 2.41 (s, 2H), 2.16 (ddd, J = 14.8, 7.4, 4.1 Hz, 1H), 2.06-1.80 (m, 6H), 1.80-1.54 (m, 11H), 1.50 (dd, J = 12.9, 6.5 Hz, 1H), 1.38-1.29 (m, 3H), 1.29-1.13 (m, 2H). MS (ESI) m/z 514.2 [C₂₇H₃₉N₅O₅ + H]⁺ |
| 95 | 4 | 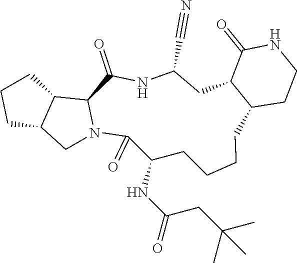 [SMILES: CC(C)(C)CC(=O)N[C@H]1CCCC[C@H]2CCNC(=O)[C@H]2C[C@@H](C#N)NC(=O)[C@@H]2[C@H]3CCC[C@H]3CN2C1=O] | ¹H NMR (400 MHz, DMSO-d₆) δ 8.3921 (d, J = 9.01 Hz, 1H), 8.0478 (d, J = 7.72 Hz, 1H), 7.3820 (s, 1H), 5.1517- 5.2014 (m, 1H), 4.4661-4.5075 (m, 1H), 4.4047 (d, J = 2.22 Hz, 1H), 3.7302-3.766 (m, 1H), 3.5356- 3.5825 (m, 1H), 3.0947 (t, J = 5.96 Hz, 2H), 2.8507-2.9117 (m, 1H), 2.7065-2.7861 (m, 1H), 2.0859- 2.1947 (m, 2H), 1.9898 (s, 2H), 1.7431-1.9230 (m, 5H), 1.4793- 1.6289 (m, 4H), 1.2945-1.4618 (m, 4H), 1.1037-1.2438 (m, 3H), 1.0043- 1.0799 (m, 2H), 0.9254 (s, 9H). MS (ESI) m/z 514.3 [C₂₈H₄₃N₅O₄ + H]⁺ |
| 96 | 3 | 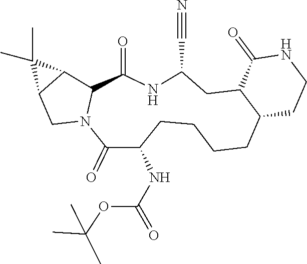 | ¹H NMR (400 MHz, MeOD) δ 6.80 (d, J = 7.5 Hz, 1H), 5.41 (dd, J = 7.5, 3.6 Hz, 1H), 4.49 (s, 1H), 4.32 (dq, J = 7.4, 3.2 Hz, 1H), 3.97 (d, J = 10.8 Hz, 1H), 3.83 (dd, J = 10.7, 5.2 Hz, 1H), 3.31-3.22 (m, 2H), 2.45 (dt, J = 9.1, 4.3 Hz, 1H), 2.19 (ddd, J = 14.8, 9.3, 3.7 Hz, 1H), 2.07-1.85 (m, 3H), 1.71 (d, J = 7.6 Hz, 1H), 1.64 (td, J = 8.0, 4.7 Hz, 4H), 1.53-1.21 (m, 13H), 1.19-1.06 (m, 5H), 0.96 (s, 3H). MS (ESI) m/z 516.4 [C₂₇H₄₁N₅O₅ + H]⁺ |
| 97 | 3 | 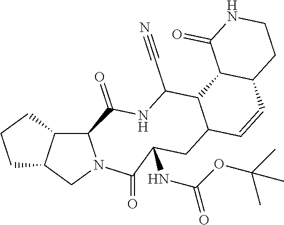 | ¹H NMR (400 MHz, MeOD) δ 5.45- 5.28 (m, 2H), 5.01 (dd, J = 7.2, 4.2 Hz, 1H), 4.39 (dd, J = 8.2, 2.4 Hz, 1H), 4.32 (d, J = 2.3 Hz, 1H), 3.82 (d, J = 4.4 Hz, 2H), 3.39-3.34 (m, 1H), 3.28 (ddt, J = 12.6, 6.7, 3.0 Hz, 1H), 3.05-2.69 (m, 4H), 2.47 (dt, J = 10.7, 4.0 Hz, 1H), 2.36 (dt, J = 15.8, 8.1 Hz, 1H), 2.27-1.51 (m, 12H), 1.45 (s, 9H). MS (ESI) m/z 514.3 [C₂₇H₃₉N₅O₅ + H]⁺ |
| 98 | 3 | 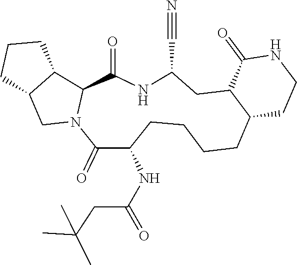 | ¹H NMR (400 MHz, MeOD) δ 6.86 (d, J = 7.4 Hz, 1H), 5.47 (dd, J = 8.1, 3.2 Hz, 1H), 4.56 (d, J = 2.2 Hz, 1H), 4.41 (tt, J = 7.4, 3.2 Hz, 1H), 3.74 (qd, J = 10.9, 5.9 Hz, 2H), 3.31-3.22 (m, 2H), 3.07 (tdd, J = 8.4, 6.2, 2.2 Hz, 1H), 2.92 (tq, J = 8.2, 4.3 Hz, 1H), 2.39 (dt, J = 8.8, 4.0 Hz, 1H), 2.14 (ddd, J = 14.8, 9.6, 3.3 Hz, 1H), 2.08- 1.84 (m, 5H), 1.82-1.24 (m, 19H), 1.13 (p, J = 7.8 Hz, 2H). MS (ESI) m/z 516.3 [C₂₇H₄₁N₅O₅ + H]⁺ |
| 99 | 3 | 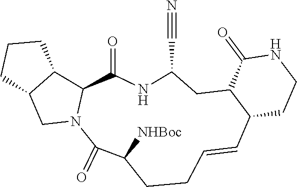 | ¹H NMR (400 MHz, MeOD) δ 5.43 (dd, J = 15.2, 8.5 Hz, 1H), 5.27 (ddd, J = 17.6, 10.4, 4.9 Hz, 2H), 4.61 (s, 1H), 4.43-4.35 (m, 1H), 4.33 (d, J = 3.1 Hz, 1H), 3.97 (dd, J = 10.5, 7.8 Hz, 1H), 3.69 (dd, J = 10.5, 3.3 Hz, 1H), 3.32-3.26 (m, 1H), 3.20 (ddd, J = 12.6, 5.9, 2.3 Hz, 1H), 2.97 (tq, J = 11.3, 4.3 Hz, 1H), 2.73 (tdd, J = 8.1, 5.0, 3.0 Hz, 1H), 2.51 (ddt, J = 22.9, 8.3, 3.9 Hz, 2H), 2.35-1.86 (m, 8H), 1.85-1.58 (m, 4H), 1.46 (s, 10H). MS (ESI) m/z 514.3 [C₂₇H₃₉N₅O₅ + H]⁺ |
| 100 | 3 | 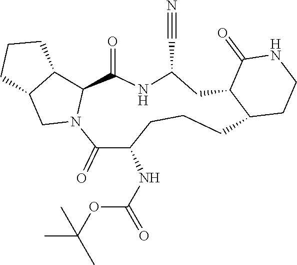 [SMILES: CC(C)(C)OC(=O)N[C@H]1CCC[C@H]2CCNC(=O)[C@H]2C[C@@H](C#N)NC(=O)[C@@H]2[C@H]3CCC[C@H]3CN2C1=O] | ¹H NMR (400 MHz, MeOD) δ 5.62 (dd, J = 9.3, 2.6 Hz, 1H), 4.85-4.81 (m, 1H), 4.33 (dd, J = 11.5, 4.2 Hz, 1H), 3.99 (dd, J = 10.9, 3.8 Hz, 1H), 3.61 (dd, J = 11.0, 8.5 Hz, 1H), 3.37 (m, 4H), 3.30-3.23 (m, 2H), 3.22-3.14 (m, 1H), 2.90 (dq, J = 8.3, 4.2 Hz, 1H), 2.17 (dt, J = 8.5, 3.9 Hz, 1H), 2.12-1.35 (m, 19H), 1.35-1.17 (m, 2H), 1.01 (q, J = 11.3 Hz, 1H). MS (ESI) m/z 502.3 [C₂₆H₃₉N₅O₅ + H]⁺ |
| 101 | 4 | 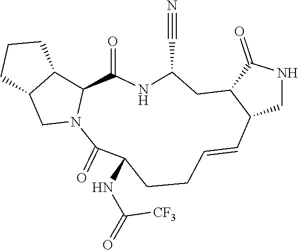 [SMILES: N#C[C@@H]1C[C@@H]2C(=O)NC[C@@H]2/C=C/CC[C@H](NC(=O)C(F)(F)F)C(=O)N2C[C@@H]3CCC[C@@H]3[C@H]2C(=O)N1] | ¹H NMR (400 MHz, MeOD) δ 5.51 (ddd, J = 15.3, 7.8, 4.2 Hz, 1H), 5.45-5.34 (m, 1H), 4.85 (dd, J = 8.1, 4.5 Hz, 1H), 4.64 (dd, J = 8.9, 2.7 Hz, 1H), 4.32 (d, J = 2.8 Hz, 1H), 4.00-3.82 (m, 2H), 3.58 (dd, J = 10.0, 5.7 Hz, 1H), 3.18-3.04 (m, 1H), 3.00 (dd, J = 10.1, 0.8 Hz, 1H), 2.92 (qd, J = 8.7, 4.4 Hz, 2H), 2.66 (ddd, J = 12.2, 7.2, 2.1 Hz, 1H), 2.53 (ddd, J = 14.6, 8.1, 2.2 Hz, 1H), 2.46-2.32 (m, 1H), 2.31- 2.08 (m, 2H), 2.04-1.52 (m, 8H). MS (ESI) m/z 496.2 [C₂₃H₂₈F₃N₅O₄ + H]⁺ |
| 102 | 3 | 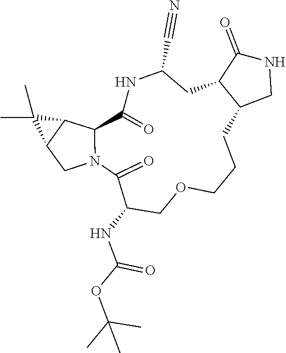 [SMILES: CC(C)(C)OC(=O)N[C@H]1COCCC[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ (ppm) 4.84 (dd, J = 9.6, 2.7 Hz, 1H), 4.47 (t, J = 5.7 Hz, 1H), 4.38 (s, 1H), 4.01- 3.83 (m, 2H), 3.62-3.49 (m, 3H), 3.49-3.34 (m, 2H), 3.13 (dd, J = 10.2, 2.9 Hz, 1H), 2.69-2.42 (m, 3H), 1.87 (ddd, J = 14.3, 11.0, 2.8 Hz, 1H), 1.64 (dd, J = 7.6, 5.0 Hz, 1H), 1.60-1.36 (m, 4H), 1.44 (s, 9H), 1.25 (td, J = 10.2, 5.9 Hz, 1H), 1.10 (s, 3H), 0.99 (s, 3H). ¹³C NMR (101 MHz, MeOD) δ (ppm) 178.43, 171.79, 170.00, 156.08, 118.40, 79.42, 70.87, 69.30, 60.45, 51.59, 46.83, 44.49, 42.58, 38.81, 36.44, 30.73, 29.79, 27.81, 27.22, 26.53, 25.13, 24.75, 18.86, 11.69. MS (ESI) m/z 518.2 [C₂₆H₃₉N₅O₆ + H]⁺ |
| 103 | 4 | 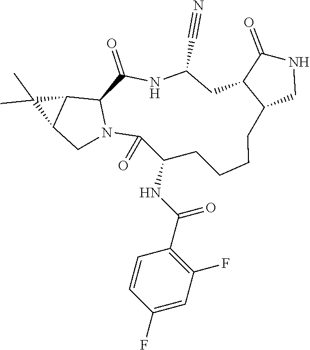 [SMILES: CC1(C)[C@@H]2[C@H]3C(=O)N[C@H](C#N)C[C@@H]4C(=O)NC[C@@H]4CCCC[C@H](NC(=O)c4ccc(F)cc4F)C(=O)N3C[C@@H]21] | ¹H NMR (400 MHz, MeOD) δ 7.85-7.74 (m, 1H), 7.15-7.04 (m, 2H), 5.16 (dd, J = 6.9, 4.6 Hz, 1H), 4.75 (dd, J = 8.1, 3.5 Hz, 1H), 4.57 (s, 1H), 4.09 (d, J = 10.7 Hz, 1H), 3.91 (dd, J = 10.7, 4.6 Hz, 1H), 3.40-3.34 (m, 1H), 3.01 (dd, J = 9.9, 6.3 Hz, 1H), 2.57-2.41 (m, 2H), 2.27-2.15 (m, 1H), 2.13-1.99 (m, 2H), 1.83- 1.72 (m, 1H), 1.72-1.64 (m, 2H), 1.58-1.45 (m, 1H), 1.42-1.28 (m, 4H), 1.28-1.17 (m, 1H), 1.11 (s, 3H), 0.99 (s, 3H). ¹⁹F NMR (376 MHz, MeOD) δ −106.86 (d, J = 10.3 Hz), −110.98 (d, J = 9.8 Hz) ¹³C NMR (101 MHz, MeOD) δ 179.58, 171.47, 170.88, 163.75, 162.6 (dq, J = 407, 12.2 Hz), 131.93 (dd, J = 10.4, 4.0 Hz), 118.85 (dd, J = 8.5, 4.0 Hz), 118.34, 111.53 (dd, J = 21.8, 31.6 Hz), 104.07 (t, J = 26.6 Hz), 60.14, 51.76, 48.31, 47.04, 46.04, 42.76, 38.84, 38.68, 31.08, 29.96, 28.53, 27.77, 27.46, 27.20, 25.30, 23.64, 18.66, 11.72. MS (ESI) m/z 542.1 [C₂₈H₃₃N₅O₄F₂ + H]⁺ |
| 104 | 4 | 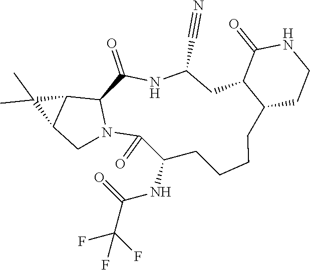 [SMILES: CC1(C)[C@@H]2[C@H]3C(=O)N[C@H](C#N)C[C@@H]4C(=O)NCC[C@@H]4CCCC[C@H](NC(=O)C(F)(F)F)C(=O)N3C[C@@H]21] | ¹H NMR (400 MHz, MeOD) δ 5.43 (dd, J = 7.0, 3.8 Hz, 1H), 4.60 (dd, J = 7.4, 3.3 Hz, 1H), 4.49 (s, 1H), 3.95 (d, J = 10.7 Hz, 1H), 3.86 (dd, J = 10.7, 4.9 Hz, 1H), 3.32-3.22 (m, 2H), 2.54-2.45 (m, 1H), 2.29-2.19 (m, 1H), 2.19-2.07 (m, 1H), 2.02-1.87 (m, 2H), 1.75-1.61 (m, 5H), 1.57- 1.47 (m, 1H), 1.46-1.35 (m, 1H), 1.35-1.24 (m, 2H), 1.21-1.11 (m, 2H), 1.11 (s, 3H), 0.97 (s, 3H). ¹⁹F NMR (376 MHz, MeOD) δ −76.82, −76.98. ¹³C NMR (101 MHz, MeOD) δ 175.04, 170.75, 170.16, 157.07 (q, J = 37.37 Hz), 118.67, 115.92 (q, J = 303 Hz), 60.46, 51.88, 40.61, 40.04, 38.46, 34.95, 32.46, 29.64, 28.72, 28.64, 27.75, 26.11, 25.28, 24.60, 23.78, 18.63, 16.07, 11.59. MS (ESI) m/z 512.2 [C₂₄H₃₂N₅O₄F₃ + H]⁺ |
| 105 | 3 | 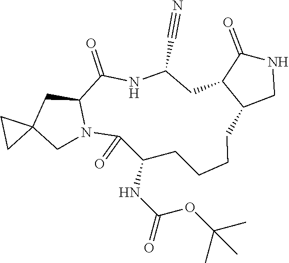 [SMILES: CC(C)(C)OC(=O)N[C@H]1CCCC[C@H]2CNC(=O)[C@H]2C[C@@H](C#N)NC(=O)[C@@H]2CC3(CC3)CN2C1=O] | ¹H NMR (400 MHz, MeOD): δ (ppm) 6.96 (d, J = 7.0 Hz, 1H), 5.22 (dd, J = 9.4, 3.2 Hz, 1H), 4.89 (under H₂O, 1H), 4.34-4.26 (m, 1H), 3.94 (d, J = 9.9 Hz, 1H), 3.41 (d, J = 9.9 Hz, 1H), 3.32 (under MeOD, 1H), 3.03 (dd, J = 10.0, 5.7 Hz, 1H), 2.47-2.28 (m, 3H), 2.20 (ddd, J = 15.0, 6.8, 3.2 Hz, 1H), 2.15-2.01 (m, 2H), 1.96- 1.85 (m, 1H), 1.79-1.67 (m, 1H), 1.46 (s, 10H), 1.43-1.20 (m, 5H), 0.79-0.59 (m, 4H). MS (ESI) m/z 488.2 [C₂₅H₃₇N₅O₅ + H]⁺ |
| 106 | 4 | 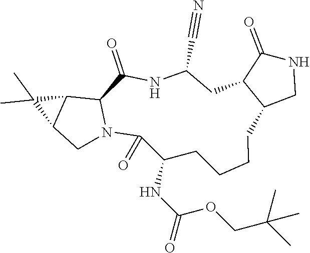 [SMILES: CC(C)(C)COC(=O)N[C@H]1CCCC[C@H]2CNC(=O)[C@H]2C[C@@H](C#N)NC(=O)[C@@H]2[C@@H]3[C@H](CN2C1=O)C3(C)C] | ¹H NMR (400 MHz, MeOD): δ (ppm) 5.13 (dd, J = 7.2, 4.3 Hz, 1H), 4.52 (s, 1H), 4.31 (dd, J = 8.6, 3.8 Hz, 1H), 4.00 (d, J = 10.7 Hz, 1H), 3.83 (dd, J = 10.6, 5.0 Hz, 1H), 3.77-3.65 (m, 2H), 3.35 (s, 6H), 2.98 (dd, J = 9.9, 6.2 Hz, 1H), 2.49-2.38 (m, 2H), 2.15 (m, 1H), 2.06-1.97 (m, 1H), 1.95- 1.84 (m, 1H), 1.70-1.59 (m, 3H), 1.44 (m, 1H), 1.38-1.12 (m, 6H), 1.09 (s, 3H), 0.94 (s, 3H), 0.92 (s, 9H). LCMS m/z = 516.2, [M + H⁺, C₂₇H₄₁N₅O₅ + H]⁺ |
| 107 | 4 | 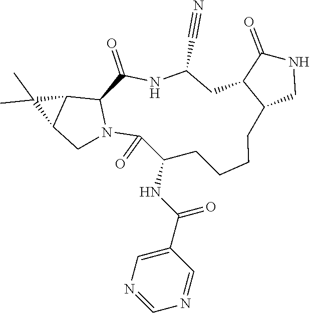 [SMILES: CC1(C)[C@@H]2[C@H]3C(=O)N[C@H](C#N)C[C@@H]4C(=O)NC[C@@H]4CCCC[C@H](NC(=O)c4cncnc4)C(=O)N3C[C@@H]21] | ¹H NMR (400 MHz, MeOD) δ 9.27 (s, 1H), 9.14 (s, 2H), 5.13 (dd, J = 6.7, 4.7 Hz, 1H), 4.68 (dd, J = 8.4, 3.7 Hz, 1H), 4.54 (s, 1H), 4.13 (d, J = 10.7 Hz, 1H), 3.90 (ddd, J = 10.6, 3.7, 1.4 Hz, 1H), 3.36 (dd, J = 10.0, 6.7 Hz, 1H), 3.00 (dd, J = 9.9, 6.0 Hz, 1H), 2.52 (q, J = 7.3 Hz, 1H), 2.44 (q, J = 6.7 Hz, 1H), 2.25-2.17 (m, 1H), 2.10- 1.97 (m, 2H), 1.85-1.74 (m, 1H), 1.69-1.64 (m, 2H), 1.60-1.47 (m, 1H), 1.42-1.19 (m, 5H), 1.10 (s, 3H), 1.01 (s, 3H). MS (ESI) m/z 506.0 [C₂₆H₃₃N₇O₄ + H]⁺ |
| 108 | 4 | 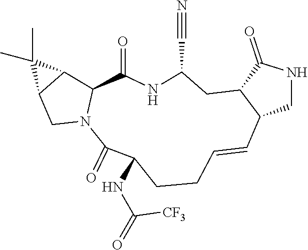 [SMILES: CC1(C)[C@@H]2[C@H]3C(=O)N[C@H](C#N)C[C@@H]4C(=O)NC[C@@H]4/C=C/CC[C@H](NC(=O)C(F)(F)F)C(=O)N3C[C@@H]21] | ¹H NMR (400 MHz, MeOD) δ 5.50 (ddd, J = 15.4, 7.3, 4.2 Hz, 1H), 5.37 (ddt, J = 15.5, 9.9, 1.6 Hz, 1H), 4.87 (d, J = 3.9 Hz, 1H), 4.54 (dd, J = 9.1, 3.0 Hz, 1H), 4.36 (s, 1H), 4.04- 3.91 (m, 2H), 3.59 (dd, J = 10.0, 5.7 Hz, 1H), 3.23-3.07 (m, 1H), 3.00 (dd, J = 10.1, 0.8 Hz, 1H), 2.67 (ddd, J = 12.5, 7.3, 2.2 Hz, 1H), 2.54 (ddd, J = 14.5, 8.8, 2.2 Hz, 1H), 2.47-2.33 (m, 1H), 2.25 (dtd, J = 14.0, 8.7, 3.1 Hz, 1H), 2.17-2.03 (m, 1H), 1.94-1.77 (m, 2H), 1.68 (ddd, J = 7.7, 5.2, 0.9 Hz, 1H), 1.59 (d, J = 7.7 Hz, 1H), 1.11 (s, 3H), 0.99 (s, 3H). MS (ESI) m/z 496.1 [C₂₃H₂₈F₃N₅O₄ + H]⁺ |
| 109 | 4 | 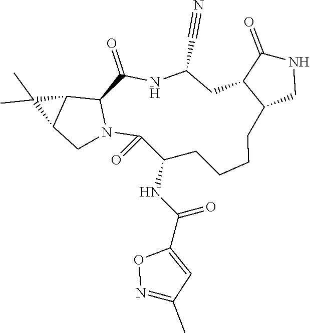 [SMILES: Cc1cc(C(=O)N[C@H]2CCCC[C@H]3CNC(=O)[C@H]3C[C@@H](C#N)NC(=O)[C@@H]3[C@@H]4[C@H](CN3C2=O)C4(C)C)on1] | ¹H NMR (400 MHz, MeOD) δ 6.87 (s, 1H), 5.16 (dd, J = 6.9, 4.5 Hz, 1H), 4.68 (dd, J = 8.5, 3.6 Hz, 1H), 4.55 (s, 1H), 4.09 (d, J = 10.7 Hz, 1H), 3.90 (dd, J = 10.6, 4.6 Hz, 1H), 3.39-3.35 (m, 1H), 3.02 (dd, J = 9.9, 6.1 Hz, 1H), 2.58-2.39 (m, 2H), 2.35 (s, 3H), 2.27-2.15 (m, 1H), 2.12-1.96 (m, 2H), 1.82- 1.71 (m, 1H), 1.71-1.61 (m, 2H), 1.59-1.45 (m, 1H), 1.43-1.19 (m, 5H), 1.11 (s, 3H), 0.98 (s, 3H). ¹³C NMR (101 MHz, MeOD) δ 179.54, 171.25, 170.86, 162.42, 160.83, 156.47, 118.34, 107.22, 60.16, 51.50, 45.92, 42.82, 38.77, 38.66, 31.05, 29.79, 28.56, 27.78, 27.30, 26.98, 25.30, 23.70, 18.68, 11.71, 9.78. MS (ESI) m/z 511.0 [C₂₆H₃₄N₆O₅ + H]⁺ |
| 110 | 4 | 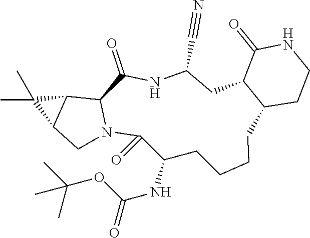 [SMILES: CC(C)(C)OC(=O)N[C@H]1CCCC[C@H]2CCNC(=O)[C@H]2C[C@@H](C#N)NC(=O)[C@@H]2[C@@H]3[C@H](CN2C1=O)C3(C)C] | ¹H NMR (400 MHz, DMSO-d₆) δ 8.7506 (d, J = 8.8 Hz, 1H), 7.9922 (d, J = 7.54 Hz, 1H), 7.3740 (s, 1H), 5.0784- 5.126 (m, 1H), 4.3585-4.3941 (m, 1H), 4.2958 (s, 1H), 3.8973 (d, J = 10.66 Hz, 1H), 3.7318-3.7684 (m, 1H), 3.0908-3.1208 (m, 2H), 2.2277- 2.2962 (m, 2H), 1.9306-2.0063 (m, 2H), 1.7878-1.8884 (m, 2H), 1.6872-1.7297 (m, 1H), 1.5058- 1.5851 (m, 4H), 1.3924-1.4148 (m, 2H), 1.1545-1.1939 (m, 1H), 1.0656- 1.082 (m, 4H), 1.0124 (s, 3H), 0.9034 (s, 9H), 0.8465 (s, 3H). MS (ESI) m/z 514.2 [C₂₈H₄₃N₅O₄ + H]⁺ |
| 111 | 3 | 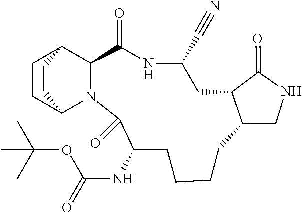 [SMILES: CC(C)(C)OC(=O)N[C@H]1CCCC[C@H]2CNC(=O)[C@H]2C[C@@H](C#N)NC(=O)[C@@H]2[C@H]3CC[C@H](CC3)N2C1=O] | ¹H NMR (400 MHz, MeOD): δ (ppm) 4.90 (dd, J = 9.4, 3.5 Hz, 1H), 4.42 (dd, J = 8.2, 3.6 Hz, 1H), 4.39 (d, J = 2.0 Hz, 1H), 4.02 (s, 1H), 2.92 (dd, J = 10.0, 5.3 Hz, 1H), 2.35-2.21 (m, 3H), 2.15-2.05 (m, 1H), 2.04-1.90 (m, 3H), 1.83-1.67 (m, 4H), 1.66- 1.37 (m, 6H), 1.34 (s, 11H), 1.28- 1.11 (m, 5H). MS (ESI) m/z 502.2 [C₂₆H₃₉N₅O₅ + H]⁺ |
| 112 | 3 | 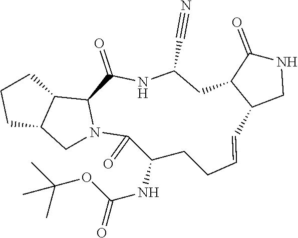 [SMILES: CC(C)(C)OC(=O)N[C@H]1CC/C=C/[C@H]2CNC(=O)[C@H]2C[C@@H](C#N)NC(=O)[C@@H]2[C@H]3CCC[C@H]3CN2C1=O] | ¹H NMR (400 MHz, MeOD): δ (ppm) 5.40-5.17 (m, 2H), 4.74-4.69 (m, 2H), 4.31-4.23 (m, 1H), 4.19 (d, J = 2.6 Hz, 1H), 3.75 (d, J = 4.6 Hz, 2H), 3.45 (dd, J = 10.0, 5.6 Hz, 1H), 3.02- 2.93 (m, 1H), 2.88 (d, J = 10.0 Hz, 1H), 2.84-2.74 (m, 2H), 2.52 (ddd, J = 12.1, 7.1, 2.1 Hz, 1H), 2.40 (ddd, J = 14.7, 8.0, 2.1 Hz, 1H), 2.30-2.16 (m, 1H), 2.09-1.93 (m, 2H), 1.91- 1.75 (m, 3H), 1.74-1.62 (m, 3H), 1.61-1.42 (m, 3H), 1.33 (s, 9H). MS (ESI) m/z 500.1 [C₂₆H₃₇N₅O₅ + H]⁺ |
| 113 | 4 | 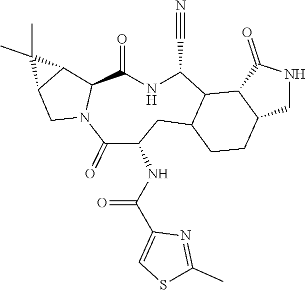 [SMILES: Cc1nc(C(=O)N[C@H]2CC3CC[C@H]4CNC(=O)[C@H]4C3[C@@H](C#N)NC(=O)[C@@H]3[C@@H]4[C@H](CN3C2=O)C4(C)C)cs1] | ¹H NMR (400 MHz, MeOD) δ 8.06 (s, 1H), 5.18 (dd, J = 6.6, 4.6 Hz, 1H), 4.79 (ddd, J = 7.6, 4.6, 3.3 Hz, 1H), 4.56 (s, 1H), 4.03 (d, J = 10.8 Hz, 1H), 3.97-3.86 (m, 1H), 3.40-3.34 (m, 1H), 3.01 (dd, J = 9.9, 6.5 Hz, 1H), 2.75 (s, 3H), 2.57-2.38 (m, 2H), 2.21 (ddd, J = 14.8, 7.9, 4.7 Hz, 1H), 2.16-1.97 (m, 2H), 1.77 (dtd, J = 14.0, 6.7, 3.3 Hz, 1H), 1.70-1.60 (m, 2H), 1.54-1.16 (m, 6H), 1.09 (s, 3H), 0.94 (s, 3H). MS (ESI) m/z 527.2 [C₂₆H₃₄N₆O₄S + H]⁺ |
| 114 | 3 | 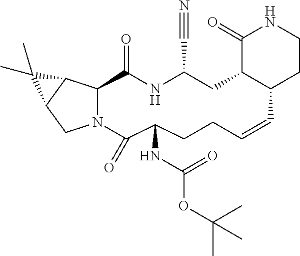 [SMILES: CC(C)(C)OC(=O)N[C@H]1CC/C=C\[C@H]2CCNC(=O)[C@H]2C[C@@H](C#N)NC(=O)[C@@H]2[C@@H]3[C@H](CN2C1=O)C3(C)C] | ¹H NMR (400 MHz, MeOD): δ (ppm) 5.43-5.35 (m, 1H), 5.32 (dd, J = 15.4, 8.3 Hz, 1H), 5.02 (dd, J = 7.5, 3.8 Hz, 1H), 4.36- 4.28 (m, 2H), 4.00-3.86 (m, 2H), 3.32 (under MeOD, 2H), 2.88-2.75 (m, 2H), 2.49 (dt, J = 11.1, 4.0 Hz, 1H), 2.35 (dt, J = 14.6, 8.2 Hz, 1H), 2.21-1.97 (m, 3H), 1.86-1.74 (m, 3H), 1.69-1.64 (m, 1H), 1.60 (d, J = 7.6 Hz, 1H), 1.43 (s, 9H), 1.11 (s, 3H), 0.97 (s, 3H). MS (ESI) m/z 514.2 [C₂₇H₃₉N₅O₅ + H]⁺ |
| 115 | 4 | 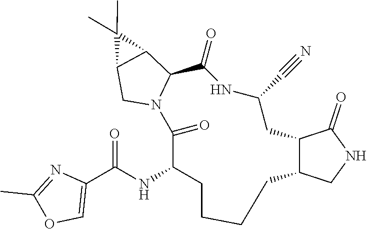 [SMILES: Cc1nc(C(=O)N[C@H]2CCCC[C@H]3CNC(=O)[C@H]3C[C@@H](C#N)NC(=O)[C@@H]3[C@@H]4[C@H](CN3C2=O)C4(C)C)co1] | ¹H NMR (400 MHz, MeOD): δ (ppm) 8.16 (s, 1H), 5.05 (dd, J = 6.7, 4.7 Hz, 1H), 4.62 (dd, J = 7.7, 3.5 Hz, 1H), 4.43 (s, 1H), 3.91 (d, J = 10.7 Hz, 1H), 3.78 (dd, J = 10.6, 4.6 Hz, 1H), 3.28-3.24 (m, 1H), 2.89 (dd, J = 9.9, 6.4 Hz, 1H), 2.44-2.39 (m, 1H), 2.38 (s, 3H), 2.36-2.30 (m, 1H), 2.09 (ddd, J = 14.9, 7.8, 4.7 Hz, 1H), 1.99- 1.85 (m, 2H), 1.68-1.59 (m, 1H), 1.59-1.51 (m, 2H), 1.42-1.30 (m, 1H), 1.30-1.17 (m, 5H), 1.16-1.02 (m, 2H), 0.99 (s, 3H), 0.83 (s, 3H). MS (ESI) m/z 511.2 [C₂₆H₃₄N₆O₅ + H]⁺ |
| 116 | 4 | 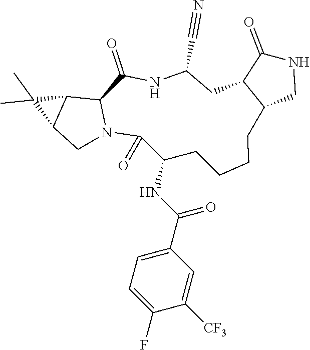 [SMILES: CC1(C)[C@@H]2[C@H]3C(=O)N[C@H](C#N)C[C@@H]4C(=O)NC[C@@H]4CCCC[C@H](NC(=O)c4ccc(F)c(C(F)(F)F)c4)C(=O)N3C[C@@H]21] | ¹H NMR (400 MHz, MeOD): δ (ppm) 8.21 (dd, J = 7.1, 2.3 Hz, 1H), 8.19-8.13 (m, 1H), 7.51-7.41 (m, 1H), 5.15 (dd, J = 6.9, 4.5 Hz, 1H), 4.69 (dd, J = 8.6, 3.7 Hz, 1H), 4.56 (s, 1H), 4.16 (d, J = 10.7 Hz, 1H), 3.91 (dd, J = 10.6, 4.4 Hz, 1H), 3.02 (dd, J = 9.9, 6.0 Hz, 1H), 2.50 (dq, J = 26.9, 7.1 Hz, 2H), 2.22 (ddd, J = 14.9, 7.4, 4.6 Hz, 1H), 2.14-1.98 (m, 2H), 1.86-1.75 (m, 1H), 1.73-1.64 (m, 2H), 1.62-1.50 (m, 1H), 1.45- 1.20 (m, 6H), 1.12 (s, 3H), 1.01 (s, 3H). ¹⁹F NMR (376 MHz, MeOD): δ (ppm) −63.03 (d, J = 12.7 Hz, 3F), −112.41 (ddd, J = 17.3, 11.6, 4.8 Hz, 1F). MS (ESI) m/z 593.2 [C₂₉H₃₃N₅O₄F₄ + H]⁺ |
| 117 | 4 | 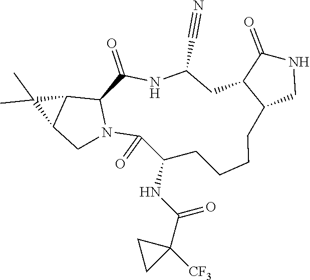 [SMILES: CC1(C)[C@@H]2[C@H]3C(=O)N[C@H](C#N)C[C@@H]4C(=O)NC[C@@H]4CCCC[C@H](NC(=O)C4(C(F)(F)F)CC4)C(=O)N3C[C@@H]21] | ¹H NMR (400 MHz, MeOD) δ 5.16 (dd, J = 7.0, 4.5 Hz, 1H), 4.60 (dd, J = 8.0, 3.4 Hz, 1H), 4.54 (s, 1H), 3.97 (d, J = 10.7 Hz, 1H), 3.85 (dd, J = 10.7, 5.0 Hz, 1H), 3.41-3.34 (m, 1H), 3.01 (dd, J = 9.9, 6.3 Hz, 1H), 2.55-2.37 (m, 2H), 2.24-2.11 (m, 1H), 2.09-1.93 (m, 2H), 1.74-1.61 (m, 3H), 1.48-1.24 (m, 9H), 1.24-1.14 (m, 1H), 1.10 (s, 3H), 0.94 (s, 3H). ¹⁹F NMR (376 MHz, MeOD) δ −67.98. ¹³C NMR (101 MHz, MeOD) δ 179.61, 171.26, 170.72, 166.16, 126.72, 124.01, 118.33, 60.09, 51.64, 46.06, 42.69, 38.87, 38.64, 31.07, 29.76, 28.44, 27.68, 27.43, 27.21, 25.29, 23.55, 18.59, 11.65, 10.05, 9.86. MS (ESI) m/z 538.1 [C₂₆H₃₄N₅O₄F₃ + H]⁺ |
| 118 | 4 | 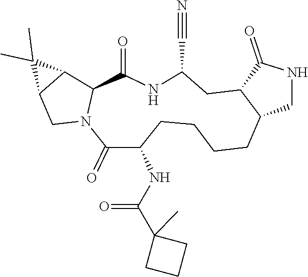 [SMILES: CC1(C(=O)N[C@H]2CCCC[C@H]3CNC(=O)[C@H]3C[C@@H](C#N)NC(=O)[C@@H]3[C@@H]4[C@H](CN3C2=O)C4(C)C)CCC1] | ¹H NMR (400 MHz, MeOD) δ 5.03 (dd, J = 7.6, 4.1 Hz, 1H), 4.48-4.38 (m, 2H), 3.96 (d, J = 10.8 Hz, 1H), 3.74 (dd, J = 10.7, 5.1 Hz, 1H), 3.27-3.22 (m, 1H), 2.89 (dd, J = 9.9, 6.0 Hz, 1H), 2.40-2.19 (m, 4H), 2.05 (ddd, J = 14.9, 7.3, 4.1 Hz, 1H), 1.99-1.79 (m, 3H), 1.79-1.68 (m, 2H), 1.68- 1.46 (m, 4H), 1.41-1.03 (m, 9H), 0.99 (s, 3H), 0.84 (s, 3H). MS (ESI) m/z 498.2 [C₂₇H₃₉N₅O₄ + H]⁺ |
| 119 | 4 | 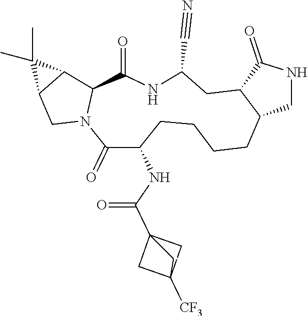 [SMILES: CC1(C)[C@@H]2[C@H]3C(=O)N[C@H](C#N)C[C@@H]4C(=O)NC[C@@H]4CCCC[C@H](NC(=O)C45CC(C(F)(F)F)(C4)C5)C(=O)N3C[C@@H]21] | ¹H NMR (400 MHz, MeOD) δ 5.03 (dd, J = 7.3, 4.3 Hz, 1H), 4.40 (d, J = 9.8 Hz, 2H), 3.92 (d, J = 10.7 Hz, 1H), 3.73 (dd, J = 10.7, 5.1 Hz, 1H), 3.27- 3.23 (m, 1H), 2.89 (dd, J = 9.9, 6.0 Hz, 1H), 2.41-2.25 (m, 2H), 2.11 (s, 7H), 1.96-1.79 (m, 2H), 1.61-1.48 (m, 3H), 1.41-1.04 (m, 6H), 0.99 (s, 3H), 0.83 (s, 3H). MS (ESI) m/z 564.2 [C₂₈H₃₆F₃N₅O₄ + H]⁺ |
| 120 | 4 | 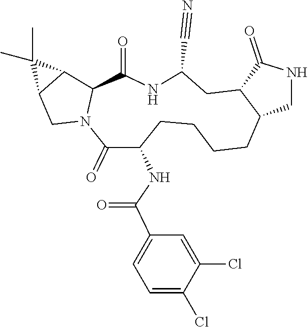 [SMILES: CC1(C)[C@@H]2[C@H]3C(=O)N[C@H](C#N)C[C@@H]4C(=O)NC[C@@H]4CCCC[C@H](NC(=O)c4ccc(Cl)c(Cl)c4)C(=O)N3C[C@@H]21] | ¹H NMR (400 MHz, MeOD) δ 8.00 (d, J = 2.0 Hz, 1H), 7.75 (dd, J = 8.4, 2.1 Hz, 1H), 7.64 (d, J = 8.4 Hz, 1H), 5.15 (dd, J = 7.0, 4.5 Hz, 1H), 4.67 (dd, J = 8.6, 3.7 Hz, 1H), 4.55 (s, 1H), 4.14 (d, J = 10.7 Hz, 1H), 3.90 (dt, J = 10.7, 2.4 Hz, 1H), 3.41-3.34 (m, 1H), 3.02 (dd, J = 9.9, 6.1 Hz, 1H), 2.58- 2.39 (m, 2H), 2.22 (ddd, J = 14.8, 7.5, 4.5 Hz, 1H), 2.13-1.98 (m, 2H), 1.78 (dtd, J = 13.8, 6.8, 3.5 Hz, 1H), 1.66 (d, J = 2.3 Hz, 2H), 1.61-1.48 (m, 1H), 1.44-1.19 (m, 5H), 1.10 (s, 3H), 1.00 (s, 3H). MS (ESI) m/z 574.2 [C₂₈H₃₃Cl₂N₅O₄ + H]⁺ |
| 121 | 4 | 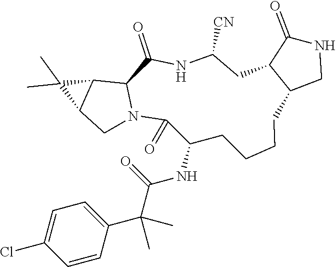 [SMILES: CC(C)(C(=O)N[C@H]1CCCC[C@H]2CNC(=O)[C@H]2C[C@@H](C#N)NC(=O)[C@@H]2[C@@H]3[C@H](CN2C1=O)C3(C)C)c1ccc(Cl)cc1] | ¹H NMR (400 MHz, MeOD) δ 7.32 (s, 4H), 5.10 (dd, J = 7.1, 4.4 Hz, 1H), 4.53 (dd, J = 8.6, 3.5 Hz, 1H), 4.49 (s, 1H), 3.97 (d, J = 10.7 Hz, 1H), 3.82 (dd, J = 10.7, 4.9 Hz, 1H), 2.97 (dd, J = 9.9, 6.2 Hz, 1H), 2.50-2.33 (m, 2H), 2.14 (ddd, J = 14.9, 7.5, 4.4 Hz, 1H), 1.92 (dddd, J = 37.7, 14.9, 7.9, 6.3 Hz, 2H), 1.71-1.52 (m, 4H), 1.51 (s, 6H), 1.39-1.21 (m, 5H), 1.21-1.10 (m, 2H), 1.08 (s, 3H), 0.88 (s, 3H). MS (ESI) m/z 582.2 [C₃₁H₄₀N₅O₄Cl + H]⁺ |
| 122 | 4 | 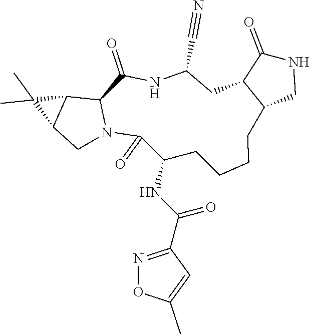 [SMILES: Cc1cc(C(=O)N[C@H]2CCCC[C@H]3CNC(=O)[C@H]3C[C@@H](C#N)NC(=O)[C@@H]3[C@@H]4[C@H](CN3C2=O)C4(C)C)no1] | ¹H NMR (400 MHz, MeOD) δ 6.45 (t, J = 0.9 Hz, 1H), 5.14 (dd, J = 6.8, 4.5 Hz, 1H), 4.70 (dd, J = 8.1, 3.6 Hz, 1H), 4.54 (s, 1H), 4.04 (d, J = 10.7 Hz, 1H), 3.88 (dd, J = 10.7, 4.7 Hz, 1H), 3.39-3.32 (m, 7H), 2.99 (dd, J = 9.9, 6.2 Hz, 1H), 2.57-2.45 (m, 4H), 2.45-2.36 (m, 1H), 2.18 (ddd, J = 14.9, 7.7, 4.6 Hz, 1H), 2.03 (ddt, J = 21.5, 14.9, 6.6 Hz, 2H), 1.80- 1.59 (m, 3H), 1.52-1.43 (m, 1H), 1.33 (ddd, J = 14.2, 11.6, 4.9 Hz, 5H), 1.28-1.15 (m, 2H), 1.09 (s, 3H), 0.95 (s, 3H). MS (ESI) m/z 511.1 [C₂₆H₃₄N₆O₅ + H]⁺ |
| 123 | 4 | 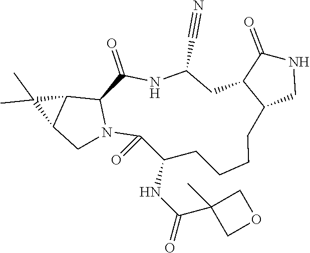 [SMILES: CC1(C(=O)N[C@H]2CCCC[C@H]3CNC(=O)[C@H]3C[C@@H](C#N)NC(=O)[C@@H]3[C@@H]4[C@H](CN3C2=O)C4(C)C)COC1] | ¹H NMR (400 MHz, MeOD) δ 5.12 (dd, J = 7.4, 4.3 Hz, 1H), 4.83 (t, 2H), 4.53 (d, J = 4.3 Hz, 2H), 4.37 (dd, J = 6.1, 2.1 Hz, 2H), 4.07 (d, J = 10.7 Hz, 1H), 3.85 (dd, J = 10.7, 5.0 Hz, 1H), 3.37- 3.33 (m, 1H), 2.99 (dd, J = 9.9, 6.0 Hz, 1H), 2.50-2.38 (m, 2H), 2.21- 2.12 (m, 1H), 2.07-1.89 (m, 2H), 1.70-1.60 (m, 3H), 1.55 (s, 3H), 1.51-1.40 (m, 1H), 1.39-1.16 (m, 5H), 1.09 (s, 3H), 0.96 (s, 3H). LCMS m/z = 500.3, [M + H⁺, C₂₆H₃₇N₅O₅ + H]⁺ |
| 124 | 4 | 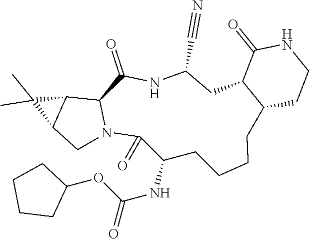 [SMILES: CC1(C)[C@@H]2[C@H]3C(=O)N[C@H](C#N)C[C@@H]4C(=O)NCC[C@@H]4CCCC[C@H](NC(=O)OC4CCCC4)C(=O)N3C[C@@H]21] | ¹H NMR (400 MHz, MeOD): δ (ppm) 6.97 (d, J = 7.1 Hz, 1H), 5.33 (dd, J = 7.5, 3.5 Hz, 1H), 4.90 (tt, J = 5.7, 2.5 Hz, 1H), 4.37 (s, 1H), 4.25 (tt, J = 7.1, 3.3 Hz, 1H), 3.81 (d, J = 10.8 Hz, 1H), 3.71 (dd, J = 10.7, 5.1 Hz, 1H), 3.19- 3.10 (m, 2H), 2.33 (dt, J = 9.0, 4.2 Hz, 1H), 2.05 (ddd, J = 14.8, 9.5, 3.6 Hz, 1H), 1.95-1.68 (m, 5H), 1.65- 1.54 (m, 5H), 1.54-1.46 (m, 5H), 1.38-1.28 (m, 1H), 1.28-1.12 (m, 4H), 1.04-0.94 (m, 5H), 0.85 (s, 3H). MS (ESI) m/z 528.2 [C₂₈H₄₁N₅O₅ + H⁺] |
| 125 | 3 | 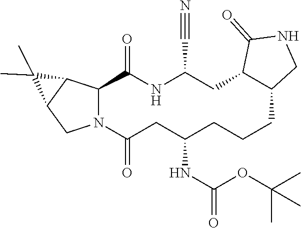 [SMILES: CC(C)(C)OC(=O)N[C@H]1CCC[C@H]2CNC(=O)[C@H]2C[C@@H](C#N)NC(=O)[C@@H]2[C@@H]3[C@H](CN2C(=O)C1)C3(C)C] | ¹H NMR (400 MHz, DMSO, major isomer) δ (ppm) 8.80 (d, J = 6.9 Hz, 1H), 7.68 (s, 1H), 6.82 (d, J = 7.8 Hz, 1H), 4.77-4.67 (m, 1H), 4.09 (s, 1H), 3.83 (s, 1H), 3.63 (dd, J = 12.2, 5.2 Hz, 1H), 3.52 (s, 1H), 3.19-3.13 (m, 1H), 2.85 (t, J = 9.0 Hz, 1H), 2.45 (dd, J = 15.8, 8.2 Hz, 1H), 2.37-2.27 (m, 1H), 2.24-2.02 (m, 3H), 1.78 (t, J = 12.4 Hz, 1H), 1.58-1.49 (m, 1H), 1.36 (s, 12H), 1.13 (s, 3H), 1.03 (s, 3H), 0.83 (s, 4H). ¹³C NMR (101 MHz, MeOD, major isomer) δ (ppm) 180.59, 172.14, 170.34, 156.13, 117.35, 78.57, 62.36, 59.46, 46.16, 459.93, 39.65, 39.03, 38.23, 33.49, 31.20, 27.38, 26.74, 26.28, 25.06, 22.83, 19.38, 18.34, 11.45, 11.03. MS (ESI) m/z 502.2 [C₂₆H₃₉N₅O₅ + H]⁺ |
| 126 | 3 | 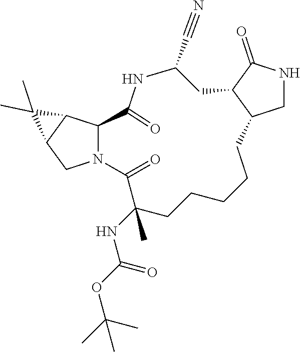 [SMILES: CC(C)(C)OC(=O)N[C@@]1(C)CCCCC[C@H]2CNC(=O)[C@H]2C[C@@H](C#N)NC(=O)[C@@H]2[C@@H]3[C@H](CN2C1=O)C3(C)C] | ¹H NMR (400 MHz, DMSO, 140 °C) δ (ppm) 8.74 (d, J = 8.5 Hz, 1H), 7.12 (s, 1H), 6.20 (s, 1H), 4.88 (ddd, J = 10.1, 8.5, 2.8 Hz, 1H), 4.59 (s, 1H), 3.81 (s, 2H), 3.33 (dd, J = 9.8, 6.2 Hz, 1H), 2.95 (ddd, J = 9.8, 2.5, 1.5 Hz, 2H), 2.48-2.28 (m, 3H), 1.80-1.67 (m, 1H), 1.59-1.48 (m, 4H), 1.48- 1.35 (m, 10H), 1.35-1.09 (m, 9H), 1.05 (s, 3H), 0.98 (s, 3H). MS (ESI) m/z 530.2 [C₂₈H₄₃N₅O₅ + H]⁺ |
| 127 | 3 | 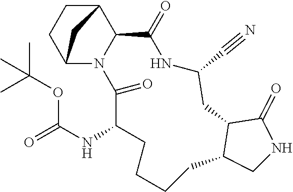 [SMILES: CC(C)(C)OC(=O)N[C@H]1CCCC[C@H]2CNC(=O)[C@H]2C[C@@H](C#N)NC(=O)[C@@H]2[C@H]3CC[C@H](C3)N2C1=O] | ¹H NMR (400 MHz, MeOD-d₄): δ 5.13 (d, 1H), 4.36 (s, 1H), 4.29 (dd, J = 7.5. 4.0 Hz, 1H), 4.17 (s, 1H), 2.97- 2.88 (m, 2H), 2.24-2.11 (m, 2H), 2.07-1.98 (m, 1H), 1.95-1.61 (m, 8H), 1.55-1.48 (m, 1H), 1.35 (s, 14H), 1.27-1.07 (m, 4H). MS (ESI) m/z 488.2, [M + H⁺, C₂₅H₃₇N₅O₅ + H]⁺ |
| 128 | 3 | 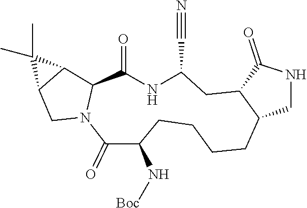 [SMILES: CC(C)(C)OC(=O)N[C@@H]1CCCC[C@H]2CNC(=O)[C@H]2C[C@@H](C#N)NC(=O)[C@@H]2[C@@H]3[C@H](CN2C1=O)C3(C)C] | ¹H NMR (400 MHz, MeOD) δ 6.81 (d, J = 8.5 Hz, 1H), 4.96 (dd, J = 7.6, 5.4 Hz, 1H), 4.90 (s, 1H), 4.03 (td, J = 8.9, 4.7 Hz, 1H), 3.66 (d, J = 3.1 Hz, 2H), 3.44 (dd, J = 10.0, 6.4 Hz, 1H), 3.03 (dd, J = 10.1, 3.6 Hz, 1H), 2.56 (hd, J = 7.6, 4.8 Hz, 2H), 2.43-2.32 (m, 1H), 2.10 (ddd, J = 14.7, 8.8, 5.4 Hz, 1H), 1.73-1.14 (m, 18H), 1.10 (s, 3H), 0.95 (s, 3H). MS (ESI): m/z = 502.2 [C₂₆H₃₉N₅O₅ + H]⁺ |
| 129 | 4 | 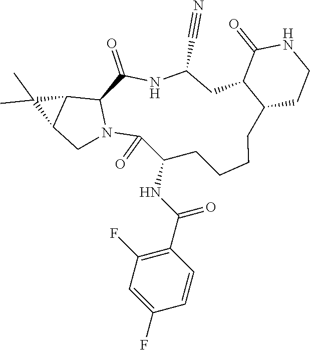 [SMILES: CC1(C)[C@@H]2[C@H]3C(=O)N[C@H](C#N)C[C@@H]4C(=O)NCC[C@@H]4CCCC[C@H](NC(=O)c4ccc(F)cc4F)C(=O)N3C[C@@H]21] | ¹H NMR (400 MHz, MeOD): δ (ppm) 7.83 (td, J = 8.8, 6.5 Hz, 1H), 7.16-7.07 (m, 2H), 5.47 (dd, J = 7.3, 3.6 Hz, 1H), 4.80 (dd, J = 6.9, 3.2 Hz, 1H), 4.53 (s, 1H), 4.00 (d, J = 10.8 Hz, 1H), 3.90 (dd, J = 10.8, 5.0 Hz, 1H), 3.31-3.25 (m, 2H), 2.49 (dt, J = 9.0, 4.3 Hz, 1H), 2.26-2.15 (m, 2H), 2.04-1.88 (m, 2H), 1.81-1.70 (m, 2H), 1.65 (ddd, J = 15.1, 7.4, 5.2 Hz, 3H), 1.56-1.38 (m, 2H), 1.36-1.27 (m, 2H), 1.22-1.13 (m, 2H), 1.11 (s, 3H), 0.99 (s, 3H). ¹⁹F NMR (376 MHz, MeOD): δ −106.74 (dt, J = 14.9, 8.8 Hz), −110.98 (q, J = 10.0 Hz). MS (ESI) m/z 556.2 [C₂₉H₃₅N₅O₄F₂ + H]⁺ |
| 130 | 3 | 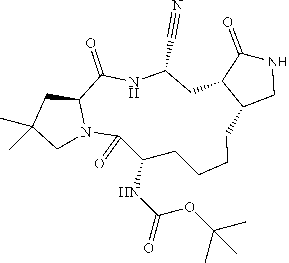 [SMILES: CC1(C)C[C@H]2C(=O)N[C@H](C#N)C[C@@H]3C(=O)NC[C@@H]3CCCC[C@H](NC(=O)OC(C)(C)C)C(=O)N2C1] | ¹H NMR (400 MHz, MeOD) δ (ppm) 5.18 (dd, J = 8.4, 3.7 Hz, 1H), 4.72 (dd, 8.5, 7.0 Hz, 1H), 4.37 (dd, J = 8.4, 3.3 Hz, 1H), 3.79 (d, J = 10.1 Hz, 1H), 3.36-3.27 (m, 2H), 3.02 (dd, J = 9.9, 5.6 Hz, 1H), 2.47 (q, J = 7.0 Hz, 1H), 2.33 (ddd, J = 16.2, 12.9, 6.7 Hz, 2H), 2.22 (ddd, J = 14.9, 6.8, 3.7 Hz, 1H), 2.03 (ddd, J = 15.1, 8.4, 6.7 Hz, 1H), 1.91 (tdd, J = 12.8, 8.1, 1.6 Hz, 2H), 1.75-1.63 (m, 1H), 1.54- 1.41 (m, 1H), 1.45 (s, 9H), 1.40-1.19 (m, 5H), 1.16 (s, 3H), 1.08 (s, 3H). ¹³C NMR (101 MHz, MeOD) δ (ppm) 179.32, 174.27, 171.96, 156.01, 118.46, 79.18, 59.93, 59.89, 51.86, 45.55, 43.33, 39.24, 39.20, 38.76, 38.16, 30.70, 30.40, 27.30, 26.70, 26.23, 24.88, 24.47, 23.61. MS (ESI) m/z 490.2 [C₂₅H₃₉N₅O₅ + H]⁺ |
| 131 | 3 | 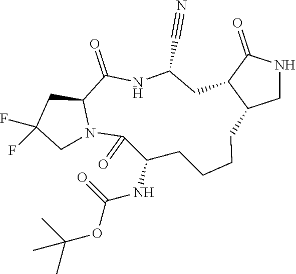 [SMILES: CC(C)(C)OC(=O)N[C@H]1CCCC[C@H]2CNC(=O)[C@H]2C[C@@H](C#N)NC(=O)[C@@H]2CC(F)(F)CN2C1=O] | ¹H NMR (400 MHz, MeOD-d₄): δ (ppm) 5.14 (dd, J = 8.4, 3.7 Hz, 1H), 4.89 (dd, J = 9.4, 4.9 Hz, 1H), 4.34 (q, J = 8.8 Hz, 1H), 4.18 (dd, J = 8.3, 3.5 Hz, 1H), 3.94-3.77 (m, 1H), 2.99-2.82 (m, 2H), 2.58-2.41 (m, 1H), 2.36- 2.19 (m, 2H), 2.11-2.01 (m, 1H), 1.96-1.86 (m, 1H), 1.85-1.73 (m, 1H), 1.63-1.52 (m, 1H), 1.34 (s, 10H), 1.29-1.04 (m, 5H). MS (ESI) m/z 498.2 [C₂₃H₃₃N₅O₅F₂ + H]⁺ |
| 132 | 3 | 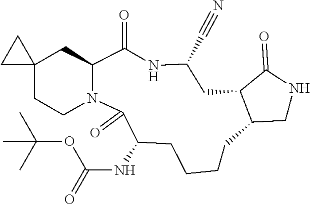 [SMILES: CC(C)(C)OC(=O)N[C@H]1CCCC[C@H]2CNC(=O)[C@H]2C[C@@H](C#N)NC(=O)[C@@H]2CC3(CCN2C1=O)CC3] | ¹H NMR (400 MHz, MeOD) δ 5.27 (d, J = 6.3 Hz, 1H), 5.17-5.07 (m, 1H), 4.70 (s, 1H), 4.14 (d, J = 13.4 Hz, 1H), 3.34 (d, J = 3.2 Hz, 1H), 2.99 (t, J = 9.1 Hz, 1H), 2.53-2.40 (m, 1H), 2.28 (s, 1H), 2.16-1.80 (m, 5H), 1.77-1.63 (m, 2H), 1.51-1.20 (m, 16H), 1.10 (d, J = 13.5 Hz, 1H), 0.79 (s, 1H), 0.48-0.28 (m, 3H). MS (ESI) m/z 502.3 [C₂₆H₃₉N₅O₅ + H]⁺ |
| 133 | 4 | 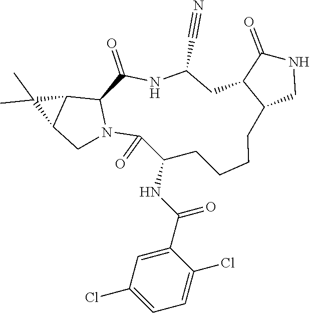 [SMILES: CC1(C)[C@@H]2[C@H]3C(=O)N[C@H](C#N)C[C@@H]4C(=O)NC[C@@H]4CCCC[C@H](NC(=O)c4cc(Cl)ccc4Cl)C(=O)N3C[C@@H]21] | ¹H NMR (400 MHz, MeOD-d₄): δ (ppm), 7.50-7.43 (m, 3H), 5.15 (dd, J = 7.0, 4.5 Hz, 1H), 4.70 (dd, J = 8.6, 4.2 Hz, 1H), 4.57 (s, 1H), 4.15 (d, J = 10.7 Hz, 1H), 3.92 (dd, J = 10.6, 4.5 Hz, 1H), 3.39-3.35 (m, 1H), 3.01 (dd, J = 9.9, 6.1 Hz, 1H), 2.48 (dq, J = 26.4, 7.1 Hz, 2H), 2.20 (ddd, J = 14.9, 7.5, 4.5 Hz, 1H), 2.11-1.97 (m, 2H), 1.77 (dtd, J = 13.7, 6.7, 3.7 Hz, 1H), 1.72-1.65 (m, 2H), 1.52 (dt, J = 14.2, 7.2 Hz, 1H), 1.44-1.19 (m, 5H), 1.12 (s, 3H), 1.03 (s, 3H). MS (ESI) m/z 574.1 [C₂₈H₃₃N₅O₄Cl₂ + H]⁺ |
| 134 | 4 | 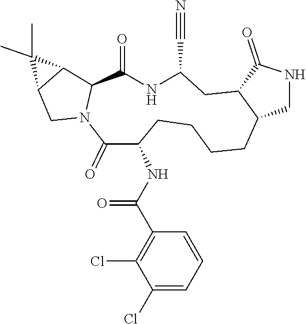 [SMILES: CC1(C)[C@@H]2[C@H]3C(=O)N[C@H](C#N)C[C@@H]4C(=O)NC[C@@H]4CCCC[C@H](NC(=O)c4cccc(Cl)c4Cl)C(=O)N3C[C@@H]21] | ¹H NMR (400 MHz, MeOD) δ 7.63 (dd, J = 6.8, 2.8 Hz, 1H), 7.41-7.35 (m, 2H), 5.15 (dd, J = 7.1, 4.4 Hz, 1H), 4.71 (dd, J = 8.8, 3.6 Hz, 1H), 4.58 (s, 1H), 4.16 (d, J = 10.7 Hz, 1H), 3.93 (dd, J = 10.6, 4.6 Hz, 1H), 3.39- 3.35 (m, 1H), 3.01 (dd, J = 10.0, 6.1 Hz, 1H), 2.48 (m, 2H), 2.21-2.15 (m, 1H), 2.11-1.96 (m, 2H), 1.83-1.74 (m, 1H), 1.73-1.67 (m, 2H), 1.52 (dd, J = 7.3, 5.4 Hz, 1H), 1.41-1.20 (m, 5H), 1.12 (s, 3H), 1.03 (s, 3H). MS (ESI) m/z 574.1 [C₂₈H₃₃Cl₂N₅O₄ + H]⁺ |
| 135 | 4 | 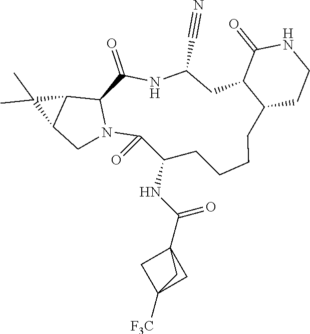 [SMILES: CC1(C)[C@@H]2[C@H]3C(=O)N[C@H](C#N)C[C@@H]4C(=O)NCC[C@@H]4CCCC[C@H](NC(=O)C45CC(C(F)(F)F)(C4)C5)C(=O)N3C[C@@H]21] | ¹H NMR (400 MHz, MeOD) δ 5.40 (dd, J = 7.2, 3.9 Hz, 1H), 4.54 (dd, J = 7.5, 3.4 Hz, 1H), 4.47 (s, 1H), 3.94 (d, J = 10.8 Hz, 1H), 3.84 (dd, J = 10.7, 5.1 Hz, 1H), 3.31-3.23 (m, 2H), 2.48 (dt, J = 9.1, 4.5 Hz, 1H), 2.29-2.18 (m, 7H), 2.12-2.02 (m, 1H), 2.01-1.87 (m, 2H), 1.72-1.59 (m, 5H), 1.54- 1.44 (m, 1H), 1.44-1.35 (m, 1H), 1.34-1.25 (m, 2H), 1.14 (dd, J = 8.8, 6.4 Hz, 2H), 1.10 (s, 3H), 0.96 (s, 3H). MS (ESI) m/z 578.3, [C₂₉H₃₈N₅O₅F₃ + H]⁺ |
| 136 | 4 | 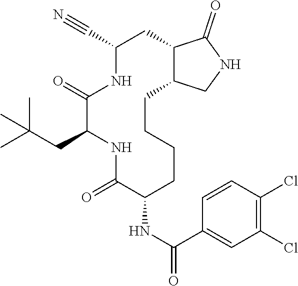 [SMILES: CC(C)(C)C[C@@H]1NC(=O)[C@@H](NC(=O)c2ccc(Cl)c(Cl)c2)CCCC[C@H]2CNC(=O)[C@H]2C[C@@H](C#N)NC1=O] | ¹H NMR (400 MHz, MeOD) δ (ppm) 8.01 (d, J = 2.1 Hz, 1H), 7.76 (dd, J = 8.4, 2.1 Hz, 1H), 7.64 (d, J = 8.4 Hz, 1H), 5.27 (dd, J = 8.9, 3.2 Hz, 1H), 4.64 (dd, J = 7.5, 5.5 Hz, 1H), 4.47 (dd, J = 10.4, 4.6 Hz, 1H), 3.39-3.35 (m, 1H), 3.02 (dd, J = 9.9, 6.9 Hz, 1H), 2.51-2.37 (m, 2H), 2.18-2.01 (m, 3H), 2.00-1.92 (m, 1H), 1.81- 1.72 (m, 1H), 1.53 (dd, J = 14.1, 5.5 Hz, 1H), 1.49-1.29 (m, 6H), 0.97 (s, 9H). ¹³C NMR (101 MHz, MeOD) δ (ppm) 179.96, 171.68, 171.65, 165.87, 135.39, 134.12, 132.23, 130.37, 129.36, 126.92, 118.13, 54.06, 50.44, 45.86, 43.84, 42.20, 38.59, 38.07, 31.29, 30.39, 29.52, 28.57, 27.11, 26.93, 24.94. MS (ESI) m/z 564.2 [C₂₇H₃₅Cl₂N₅O₄ + H]⁺ |
| 137 | 3 | 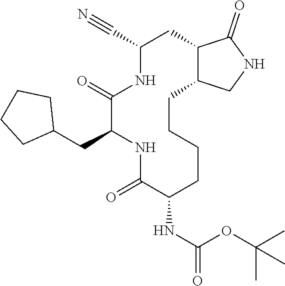 [SMILES: CC(C)(C)OC(=O)N[C@H]1CCCC[C@H]2CNC(=O)[C@H]2C[C@@H](C#N)NC(=O)[C@H](CC2CCCC2)NC1=O] | ¹H NMR (400 MHz, MeOD): δ (ppm) δ 5.27 (dd, J = 9.3, 3.0 Hz, 1H), 4.51 (t, J = 7.4 Hz, 1H), 3.97 (dd, J = 10.2, 4.6 Hz, 1H), 3.00 (dd, J = 9.9, 7.2 Hz, 1H), 2.50-2.32 (m, 2H), 2.12-2.05 (m, 1H), 1.94-1.75 (m, 6H), 1.69- 1.50 (m, 6H), 1.44 (s, 10H), 1.41- 1.27 (m, 8H). MS (ESI) m/z 504.3 [C₂₆H₄₁N₅O₅ + H]⁺ |
| 138 | 3 | 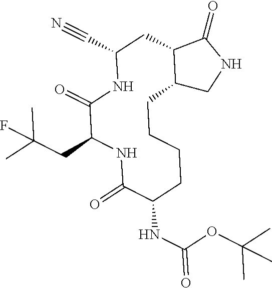 [SMILES: CC(C)(F)C[C@@H]1NC(=O)[C@@H](NC(=O)OC(C)(C)C)CCCC[C@H]2CNC(=O)[C@H]2C[C@@H](C#N)NC1=O] | ¹H NMR (400 MHz, DMSO-d₆) δ 8.3551 (d, J = 8.27 Hz, 1H), 8.2884 (d, J = 8.81 Hz, 1H), 7.3341 (s, 1H), 6.2791 (bs, 1H), 5.0585-5.1097 (m, 1H), 4.5031-4.557 (m, 1H), 3.9174- 3.9711 (m, 1H), 2.8221-2.8645 (m, 1H), 2.2879-2.4012 (m, 2H), 2.203- 2.2422 (m, 1H), 2.014-2.0635 (m, 1H), 1.9903-2.0635 (m, 1H), 1.7825- 1.8976 (m, 2H), 1.381 (s, 9H), 1.2109-1.3645 (m, 14H). ¹⁹F NMR (376 MHz, DMSO-d₆) δ −132.46 (bs, 1F). MS (ESI) m/z 496.2 [C₂₄H₃₈N₅O₅F + H]⁺ |
| 139 | 4 | 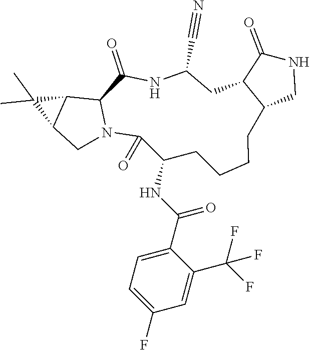 [SMILES: CC1(C)[C@@H]2[C@H]3C(=O)N[C@H](C#N)C[C@@H]4C(=O)NC[C@@H]4CCCC[C@H](NC(=O)c4ccc(F)cc4C(F)(F)F)C(=O)N3C[C@@H]21] | ¹H NMR (400 MHz, MeOD) δ 7.60- 7.50 (m, 2H), 7.45 (td, J = 8.3, 2.6 Hz, 1H), 5.13 (dd, J = 7.1, 4.4 Hz, 1H), 4.68 (dd, J = 8.6, 3.7 Hz, 1H), 4.55 (s, 1H), 4.11 (d, J = 10.7 Hz, 1H), 3.90 (dd, J = 10.6, 4.7 Hz, 1H), 3.37-3.34 (m, 1H), 2.99 (dd, J = 9.9, 6.0 Hz, 1H), 2.52-2.39 (m, 2H), 2.22- 2.13 (m, 1H), 2.07-1.93 (m, 2H), 1.79-1.63 (m, 3H), 1.55-1.46 (m, 1H), 1.40-1.16 (m, 5H), 1.10 (s, 3H), 1.00 (s, 3H). MS (ESI) m/z 592.3 [C₂₉H₃₃N₅O₄F₄ + H]⁺ |
| 140 | 3 | 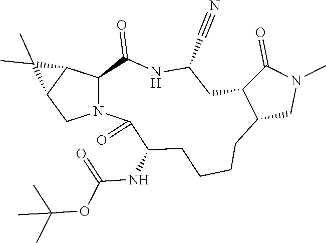 | ¹H NMR (400 MHz, MeOD) δ 5.12 (dd, J = 7.3, 4.3 Hz, 1H), 4.52 (s, 1H), 4.25 (dd, J = 8.9, 3.6 Hz, 1H), 4.06 (d, J = 10.7 Hz, 1H), 3.81 (dd, J = 10.6, 5.0 Hz, 1H), 3.38 (dd, J = 9.9, 7.0 Hz, 1H), 3.05 (dd, J = 9.9, 6.4 Hz, 1H), 2.82 (s, 3H), 2.51 (q, J = 7.3 Hz, 1H), 2.34 (h, J = 7.0 Hz, 1H), 2.17-2.08 (m, 1H), 2.04-1.95 (m, 1H), 1.90- 1.78 (m, 1H), 1.69-1.55 (m, 3H), 1.48-1.11 (m, 15H), 1.09 (s, 3H), 0.95 (s, 3H). MS (ESI) m/z 516.3 [C₂₇H₄₁N₅O₅ + H]⁺ |
| 141 | 4 | 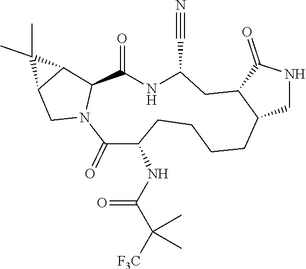 [SMILES: CC1(C)[C@@H]2[C@H]3C(=O)N[C@H](C#N)C[C@@H]4C(=O)NC[C@@H]4CCCC[C@H](NC(=O)C(C)(C)C(F)(F)F)C(=O)N3C[C@@H]21] | ¹H NMR (400 MHz, MeOD) δ 5.15 (dd, J = 7.3, 4.3 Hz, 1H), 4.59 (dq, J = 6.2, 3.6 Hz, 1H), 4.54 (s, 1H), 4.02 (d, J = 10.7 Hz, 1H), 3.86 (dd, J = 10.7, 5.0 Hz, 1H), 3.36 (dd, J = 9.3, 6.0 Hz, 1H), 3.01 (dd, J = 9.9, 6.1 Hz, 1H), 2.54-2.37 (m, 2H), 2.19 (ddd, J = 14.9, 7.5, 4.4 Hz, 1H), 2.09-1.96 (m, 2H), 1.72-1.58 (m, 3H), 1.50-1.16 (m, 12H), 1.11 (s, 3H), 0.93 (s, 3H). MS (ESI): m/z 540.2 [C₂₆H₃₆F₃N₅O₄ + H]⁺ |
| 142 | 4 | 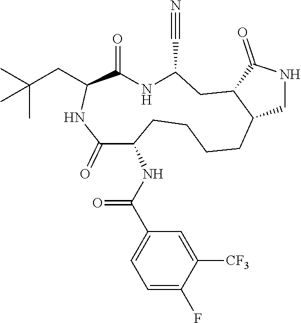 [SMILES: CC(C)(C)C[C@@H]1NC(=O)[C@@H](NC(=O)c2ccc(F)c(C(F)(F)F)c2)CCCC[C@H]2CNC(=O)[C@H]2C[C@@H](C#N)NC1=O] | ¹H NMR (400 MHz, MeOD) δ 8.25- 8.20 (m, 1H), 8.16 (ddd, J = 8.2, 4.7, 2.4 Hz, 1H), 7.46 (dd, J = 10.2, 8.8 Hz, 1H), 5.26 (dd, J = 8.7, 3.4 Hz, 1H), 4.64 (dd, J = 7.5, 5.5 Hz, 1H), 4.47 (dd, J = 10.4, 4.6 Hz, 1H), 3.39- 3.35 (m, 1H), 3.02 (dd, J = 9.9, 6.8 Hz, 1H), 2.51-2.38 (m, 2H), 2.19- 1.92 (m, 4H), 1.78 (dd, J = 13.6, 5.3 Hz, 1H), 1.57-1.25 (m, 7H), 0.97 (s, 9H). MS (ESI): m/z 582.1 [C₂₈H₃₅F₄N₅O₄ + H⁺] |
| 143 | 3 | 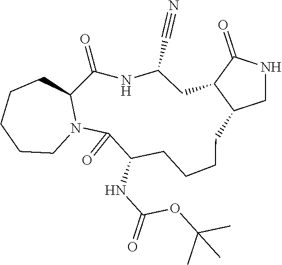 [SMILES: CC(C)(C)OC(=O)N[C@H]1CCCC[C@H]2CNC(=O)[C@H]2C[C@@H](C#N)NC(=O)[C@@H]2CCCCCN2C1=O] | ¹H NMR (400 MHz, MeOD) δ (ppm) 5.24 (dd, J = 7.5, 4.0 Hz, 1H), 4.95- 4.91 (m, 1H), 4.65-4.58 (m, 1H), 3.84 (d, J = 15.7 Hz, 1H), 3.43-3.36 (m, 1H), 2.98 (dd, J = 9.9, 7.0 Hz, 1H), 2.49-2.32 (m, 2H), 2.16-1.82 (m, 9H), 1.75-1.57 (m, 2H), 1.46 (s, 9H), 1.41-1.25 (m, 7H), 1.21-1.12 (m, 1H). ¹³C NMR (101 MHz, MeOD) δ (ppm) 179.83, 173.55, 171.80, 155.80, 118.50, 79.20, 56.95, 49.99, 46.24, 43.63, 42.36, 38.87, 37.99, 32.09, 31.13, 29.94, 28.71, 27.92, 27.32, 27.22, 24.37, 23.49. MS (ESI) m/z 490.2 [C₂₅H₃₉N₅O₅ + H]⁺ |
| 144 | 3 | 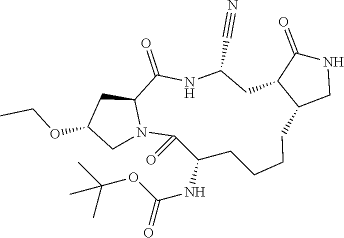 [SMILES: CCO[C@@H]1C[C@H]2C(=O)N[C@H](C#N)C[C@@H]3C(=O)NC[C@@H]3CCCC[C@H](NC(=O)OC(C)(C)C)C(=O)N2C1] | ¹H NMR (400 MHz, MeOD) δ (ppm) 5.23 (dd, J = 7.5, 4.1 Hz, 1H), 4.78 (dd, J = 8.3, 6.0 Hz, 1H), 4.39 (dd, J = 8.2, 3.4 Hz, 1H), 4.31- 4.23 (m, 1H), 3.99 (dd, J = 11.1, 3.2 Hz, 1H), 3.71-3.46 (m, 3H), 3.37-3.34 (m, 1H), 3.00 (dd, J = 9.9, 6.2 Hz, 1H), 2.57 (dt, J = 13.4, 5.7 Hz, 1H), 2.41 (ddq, J = 27.3, 13.7, 7.1 Hz, 2H), 2.19 (ddd, J = 15.0, 7.4, 4.1 Hz, 1H), 2.14-1.97 (m, 2H), 1.97-1.87 (m, 1H), 1.74-1.62 (m, 1H), 1.46 (s, 9H), 1.50-1.40 (m, 1H), 1.32 (tt, J = 14.4, 6.5 Hz, 4H), 1.19 (t, J = 7.0 Hz, 4H). ¹³C NMR (101 MHz, MeOD) δ (ppm) 179.59, 173.43, 171.31, 155.94, 118.46, 79.21, 77.24, 64.24, 58.42, 51.77, 51.72, 45.81, 42.88, 38.87, 38.75, 32.33, 31.01, 30.74, 27.31, 27.12, 26.73, 23.65, 14.28. MS (ESI) m/z 506.3 [C₂₅H₃₉N₅O₆ + H]⁺ |
| 145 | 3 | 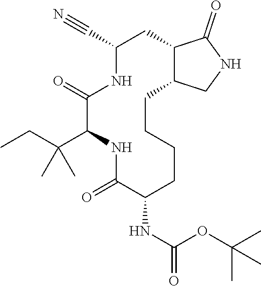 [SMILES: CCC(C)(C)[C@@H]1NC(=O)[C@@H](NC(=O)OC(C)(C)C)CCCC[C@H]2CNC(=O)[C@H]2C[C@@H](C#N)NC1=O] | ¹H NMR (400 MHz, MeOD) δ (ppm) 8.34 (d, J = 9.8 Hz, 1H), 6.58 (d, J = 7.4 Hz, 1H), 5.34 (dd, J = 8.9, 2.4 Hz, 1H), 4.38 (d, J = 9.8 Hz, 1H), 4.10 (ddd, J = 9.5, 7.6, 3.9 Hz, 1H), 2.99 (dd, J = 9.8, 6.2 Hz, 1H), 2.53-2.39 (m, 2H), 2.15-2.04 (m, 1H), 1.97 (ddd, J = 14.8, 8.8, 5.6 Hz, 1H), 1.76-1.63 (m, 2H), 1.44 (s, 9H), 1.47-1.22 (m, 8H), 1.04 (s, 3H), 1.02 (s, 3H), 0.91 (t, J = 7.4 Hz, 3H). ¹³C NMR (101 MHz, MeOD) δ (ppm) 179.89, 173.48, 170.36, 155.92, 118.45, 78.97, 59.46, 54.28, 45.58, 42.72, 38.05, 37.64, 37.14, 31.49, 31.43, 31.36, 27.36, 27.36, 27.28, 26.73, 24.92, 21.96, 21.38, 7.00. MS (ESI) m/z 492.2 [C₂₅H₄₁N₅O₅ + H]⁺ |
| 146 | 3 | 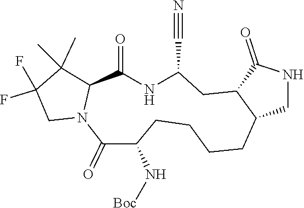 [SMILES: CC(C)(C)OC(=O)N[C@H]1CCCC[C@H]2CNC(=O)[C@H]2C[C@@H](C#N)NC(=O)[C@H]2N(CC(F)(F)C2(C)C)C1=O] | ¹H NMR (400 MHz, DMSO-d₆) δ 8.46 (s, 1H), 7.19 (s, 1H), 6.41 (s, 1H), 4.78 (q, J = 6.1 Hz, 1H), 4.47 (s, 1H), 4.30 (d, J = 13.7 Hz, 1H), 4.20-4.02 (m, 2H), 3.23 (dd, J = 9.6, 6.4 Hz, 1H), 2.87 (ddd, J = 9.8, 4.2, 1.3 Hz, 1H), 2.46 (d, J = 8.2 Hz, 1H), 2.40-2.32 (m, 1H), 2.28 (dt, J = 15.0, 5.4 Hz, 1H), 1.80 (dt, J = 14.5, 8.2 Hz, 2H), 1.40 (m, 11H), 1.33-1.08 (m, 11H). MS (ESI) m/z 526.2 [C₂₅H₃₇F₂N₅O₅ + H⁺] |
| 147 | 3 | 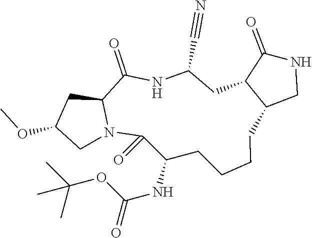 [SMILES: CO[C@@H]1C[C@H]2C(=O)N[C@H](C#N)C[C@@H]3C(=O)NC[C@@H]3CCCC[C@H](NC(=O)OC(C)(C)C)C(=O)N2C1] | ¹H NMR (400 MHz, MeOD) δ (ppm) 5.14 (dd, J = 7.4, 4.1 Hz, 1H), 4.68 (dd, J = 8.3, 6.2 Hz, 1H), 4.32 (dd, J = 8.0, 3.3 Hz, 1H), 4.12-4.03 (m, 1H), 3.94 (dd, J = 11.3, 2.6 Hz, 1H), 3.56 (dd, J = 11.3, 4.6 Hz, 1H), 3.28 (s, 3H), 3.27-3.25 (m, 1H), 2.91 (dd, J = 9.9, 6.2 Hz, 1H), 2.48 (dt, J = 13.4, 5.8 Hz, 1H), 2.42-2.23 (m, 2H), 2.15-1.77 (m, 4H), 1.65- 1.50 (m, 1H), 1.37 (s, 9H), 1.34-1.14 (m, 5H), 1.15-1.05 (m, 1H). ¹³C NMR (101 MHz, MeOD) δ (ppm) 179.61, 173.42, 171.26, 155.93, 118.47, 79.23, 78.96, 58.38, 55.63, 51.74, 51.40, 45.84, 42.88, 38.91, 38.76, 31.93, 31.03, 30.73, 27.30, 27.18, 26.78, 23.66. MS (ESI) m/z 492.2 [M + H⁺; C₂₄H₃₇N₅O₆ + H]⁺ |
| 148 | 4 | 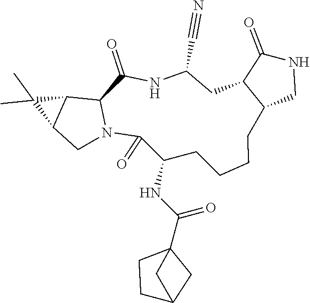 | ¹H NMR (400 MHz, MeOD) δ 7.62 (d, J = 7.3 Hz, 1H), 5.15 (dd, J = 7.5, 4.2 Hz, 1H), 4.62-4.55 (m, 1H), 4.54 (s, 1H), 4.07 (d, J = 10.7 Hz, 1H), 3.85 (dd, J = 10.7, 5.1 Hz, 1H), 3.40-3.34 (m, 1H), 3.01 (dd, J = 9.9, 5.9 Hz, 1H), 2.51-2.38 (m, 3H), 2.23-2.13 (m, 1H), 2.09-1.95 (m, 2H), 1.86-1.73 (m, 6H), 1.70 (d, J = 7.6 Hz, 1H), 1.67-1.58 (m, 2H), 1.52- 1.16 (m, 8H), 1.10 (s, 3H), 0.95 (s, 3H). ¹³C NMR (101 MHz, MeOD) δ 179.55, 174.88, 171.86, 170.79, 118.30, 60.05, 53.24, 50.85, 45.89, 42.56, 41.02, 40.93, 38.62, 38.59, 35.73, 30.74, 30.11, 29.29, 28.39, 27.63, 27.23, 27.04, 26.84, 25.30, 24.02, 18.60, 11.81. MS (ESI) m/z 501.2, [C₂₈H₃₉N₅O₄ + H]⁺ |
| 149 | 4 | 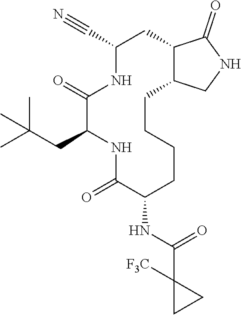 [SMILES: CC(C)(C)C[C@@H]1NC(=O)[C@@H](NC(=O)C2(C(F)(F)F)CC2)CCCC[C@H]2CNC(=O)[C@H]2C[C@@H](C#N)NC1=O] | ¹H NMR (400 MHz, MeOD): δ (ppm) 7.60 (d, J = 7.1 Hz, 1H), 5.21 (dd, J = 7.6, 4.5 Hz, 1H), 4.60 (under H₂O, 1H), 4.35 (dq, J = 7.0, 4.1 Hz, 1H), 3.32 (under MeOD, 1H), 3.01 (dd, J = 9.9, 6.4 Hz, 1H), 2.51-2.38 (m, 2H), 2.11-2.06 (m 2H), 2.05-1.96 (m, 1H), 1.93-1.82 (m, 1H), 1.71-1.61 (m, 1H), 1.49 (dd, J = 14.2, 5.9 Hz, 1H), 1.44-1.23 (m, 10H), 0.96 (s, 9H). ¹⁹F NMR (376 MHz, MeOD-d₄): δ (ppm) −67.87. MS (ESI) m/z 528.2 [C₂₅H₃₆N₅O₄F₃ + H]⁺ |
| 150 | 4 | 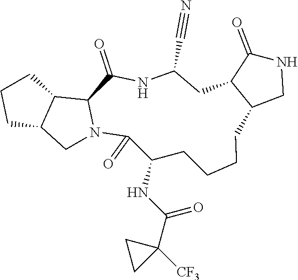 [SMILES: N#C[C@@H]1C[C@@H]2C(=O)NC[C@@H]2CCCC[C@H](NC(=O)C2(C(F)(F)F)CC2)C(=O)N2C[C@@H]3CCC[C@@H]3[C@H]2C(=O)N1] | ¹H NMR (400 MHz, DMSO-d₆) δ 8.3651 (d, J = 8.86 Hz, 1H), 7.8211 (d, J = 6.55 Hz, 1H), 7.6309 (s, 1H), 5.0121-5.0632 (m, 1H), 4.5098-4.5542 (m, 1H), 4.4807 (d, J = 2.08 Hz, 1H), 3.6513-3.6875 (m, 1H), 3.5636-3.6106 (m, 1H), 3.1384-3.1784 (m, 1H), 2.8709-2.9316 (m, 1H), 2.7447-2.8305 (m, 2H), 2.2541 (q, J = 6.66 Hz, 1H), 2.1725 (q, J = 7.01 Hz, 1H), 1.9839-2.0502 (m, 1H), 1.7404-1.8953 (m, 4H), 1.332-1.6194 (m, 5H), 1.1603-1.3039 (m, 9H), 1.0067-1.0475 (m, 1H). ¹⁹F NMR (376 MHz, DMSO-d₆) δ −65.35 (s, 3F). MS (ESI) m/z 538.1 [C₂₆H₃₄N₅O₄F₃ + H]⁺ |
| 151 | 4 | 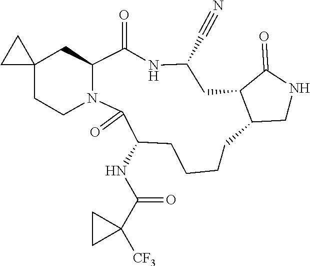 [SMILES: N#C[C@@H]1C[C@@H]2C(=O)NC[C@@H]2CCCC[C@H](NC(=O)C2(C(F)(F)F)CC2)C(=O)N2CCC3(CC3)C[C@H]2C(=O)N1] | ¹H NMR (400 MHz, MeOD): δ (ppm) δ 5.29 (d, J = 6.2 Hz, 1H), 5.13 (t, J = 6.2 Hz, 1H), 5.02 (dd, J = 7.4, 4.3 Hz, 1H), 4.09 (d, J = 13.4 Hz, 1H), 3.47- 3.37 (m, 1H), 3.38-3.34 (m, 1H), 3.01 (dd, J = 9.9, 7.7 Hz, 1H), 2.54- 2.42 (m, 1H), 2.37 (q, J = 6.7 Hz, 1H), 2.09 (t, J = 6.6 Hz, 2H), 2.04-1.90 (m, 3H), 1.80-1.63 (m, 2H), 1.53- 1.23 (m, 11H), 1.11 (d, J = 13.4 Hz, 1H), 0.85-0.75 (m, 1H), 0.52-0.42 (m, 1H), 0.40-0.29 (m, 2H). MS (ESI) m/z 538.3, [C₂₆H₃₄N₅O₄F₃ + H]⁺ |
| 152 | 4 | 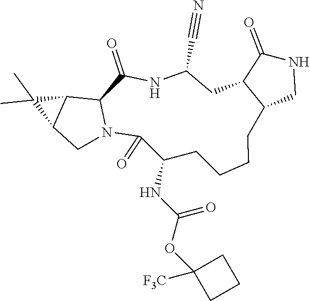 [SMILES: CC1(C)[C@@H]2[C@H]3C(=O)N[C@H](C#N)C[C@@H]4C(=O)NC[C@@H]4CCCC[C@H](NC(=O)OC4(C(F)(F)F)CCC4)C(=O)N3C[C@@H]21] | ¹H NMR (400 MHz, MeOD) δ 5.12 (dd, J = 7.2, 4.4 Hz, 1H), 4.53 (s, 1H), 4.28 (dd, J = 8.8, 3.7 Hz, 1H), 4.03 (d, J = 10.7 Hz, 1H), 3.83 (dd, J = 10.6, 5.0 Hz, 1H), 3.34 (d, J = 6.7 Hz, 1H), 2.99 (dd, J = 9.9, 6.1 Hz, 1H), 2.79-2.64 (m, 2H), 2.56-2.36 (m, 4H), 2.21- 2.10 (m, 1H), 2.06-1.78 (m, 4H), 1.70-1.57 (m, 3H), 1.51-1.38 (m, 1H), 1.37-1.14 (m, 5H), 1.09 (s, 3H), 0.95 (s, 3H). MS (APCI) m/z 568.2 [C₂₇H₃₆N₅O₅F₃ + H]⁺ |
| 153 | 4 | 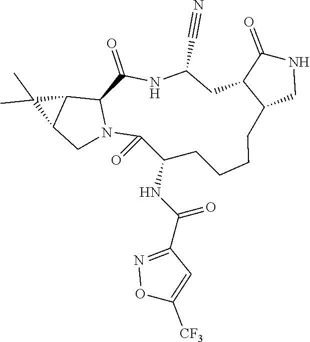 [SMILES: CC1(C)[C@@H]2[C@H]3C(=O)N[C@H](C#N)C[C@@H]4C(=O)NC[C@@H]4CCCC[C@H](NC(=O)c4cc(C(F)(F)F)on4)C(=O)N3C[C@@H]21] | ¹H NMR (400 MHz, MeOD) δ 7.41 (q, J = 1.0 Hz, 1H), 5.14 (dd, J = 6.8, 4.6 Hz, 1H), 4.71 (dd, J = 8.2, 3.6 Hz, 1H), 4.54 (s, 1H), 4.06 (d, J = 10.7 Hz, 1H), 3.89 (dd, J = 10.6, 4.3 Hz, 1H), 3.39-3.33 (m, 1H), 3.00 (dd, J = 10.0, 6.2 Hz, 1H), 2.57-2.37 (m, 2H), 2.19 (ddd, J = 14.9, 7.6, 4.7 Hz, 1H), 2.04 (ddt, J = 30.6, 14.9, 6.4 Hz, 2H), 1.77 (dtd, J = 13.9, 6.9, 3.6 Hz, 1H), 1.66 (d, J = 4.6 Hz, 2H), 1.48 (dt, J = 14.9, 7.6 Hz, 1H), 1.43-1.23 (m, 5H), 1.09 (s, 3H), 0.97 (s, 3H). ¹⁹F NMR (376 MHz, MeOD) δ −65.76. MS (ESI) m/z 565.2 [C₂₆H₃₁F₃N₆O₅ + H]⁺ |
| 154 | 5 | 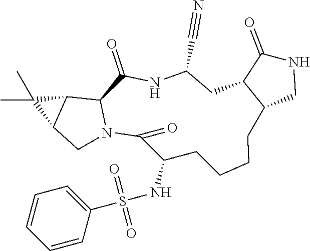 [SMILES: CC1(C)[C@@H]2[C@H]3C(=O)N[C@H](C#N)C[C@@H]4C(=O)NC[C@@H]4CCCC[C@H](NS(=O)(=O)c4ccccc4)C(=O)N3C[C@@H]21] | ¹H NMR (400 MHz, MeOD) δ (ppm) 7.87 (dd, J = 7.2, 1.7 Hz, 2H), 7.67- 7.59 (m, 1H), 7.59-7.50 (m, 2H), 5.12 (dd, J = 6.5, 4.9 Hz, 1H), 4.39 (s, 1H), 4.04 (dd, J = 7.5, 3.2 Hz, 1H), 3.76 (dd, J = 10.6, 4.8 Hz, 1H), 3.68 (d, J = 10.6 Hz, 1H), 3.37-3.34 (m, 1H), 2.97 (dd, J = 9.9, 6.2 Hz, 1H), 2.41 (dp, J = 27.2, 7.0 Hz, 2H), 2.26-2.11 (m, 1H), 1.96 (dt, J = 14.9, 6.4 Hz, 1H), 1.85 (dq, J = 13.4, 6.6 Hz, 1H), 1.66-1.54 (m, 2H), 1.54-1.39 (m, 1H), 1.39- 1.14 (m, 5H), 1.12-0.96 (m, 4H), 0.70 (s, 3H). ¹³C NMR (101 MHz, MeOD) δ (ppm) 179.51, 170.73, 170.47, 140.93, 132.44, 128.93, 126.62, 118.36, 60.03, 54.02, 48.38 (HSQC), 46.73, 46.28, 42.63, 38.93, 38.57, 31.38, 31.10, 28.35, 27.64, 27.57, 25.17, 23.32, 18.42, 11.86. MS (ESI) m/z 542.1 [C₂₇H₃₅N₅O₅S + H]⁺ |
| 155 | 5 | 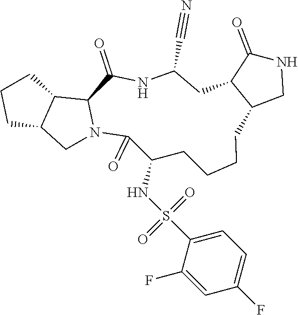 [SMILES: N#C[C@@H]1C[C@@H]2C(=O)NC[C@@H]2CCCC[C@H](NS(=O)(=O)c2ccc(F)cc2F)C(=O)N2C[C@@H]3CCC[C@@H]3[C@H]2C(=O)N1] | ¹H NMR (400 MHz, MeOD): δ (ppm) 7.94 (m, 1H), 7.27-7.09 (m, 2H), 5.19-5.13 (m, 1H), 4.35 (d, J = 2.2 Hz, 1H), 4.28 (dd, J = 7.1, 3.7 Hz, 1H), 3.71-3.61 (m, 1H), 3.50 (dd, J = 10.9, 3.7 Hz, 1H), 3.36 (m, 1H), 2.96 (m, 2H), 2.88 (m, 1H), 2.47- 2.33 (m, 2H), 2.17 (m, 1H), 2.01- 1.82 (m, 4H), 1.71-1.54 (m, 3H), 1.42-1.19 (m, 7H), 1.16-1.03 (m, 1H). MS (ESI) m/z 578.3 [C₂₇H₃₃N₅O₅F₂S + H]⁺ |
| 156 | 5 | 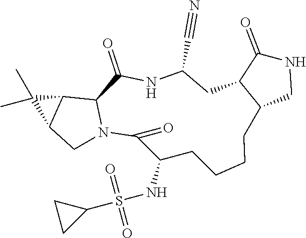 [SMILES: CC1(C)[C@@H]2[C@H]3C(=O)N[C@H](C#N)C[C@@H]4C(=O)NC[C@@H]4CCCC[C@H](NS(=O)(=O)C4CC4)C(=O)N3C[C@@H]21] | ¹H NMR (400 MHz, MeOD) δ 5.15 (dd, J = 6.9, 4.6 Hz, 1H), 4.54 (s, 1H), 4.21 (dd, J = 8.0, 3.5 Hz, 1H), 3.93 (d, J = 10.7 Hz, 1H), 3.83 (dd, J = 10.6, 5.0 Hz, 1H), 3.37 (d, J = 6.7 Hz, 1H), 3.00 (dd, J = 9.9, 6.2 Hz, 1H), 2.56-2.39 (m, 3H), 2.23-2.15 (m, 1H), 2.05- 1.94 (m, 2H), 1.85-1.76 (m, 1H), 1.69 (d, J = 7.7 Hz, 1H), 1.66-1.62 (m, 1H), 1.45 (t, J = 10.4 Hz, 1H), 1.38-1.24 (m, 5H), 1.13 (s, 3H), 1.09-1.00 (m, 3H), 0.98 (s, 3H), 0.97-0.85 (m, 1H). MS (ESI) m/z 506.0 [C₂₄H₃₅N₅O₅S + H]⁺ |
| 157 | 5 | 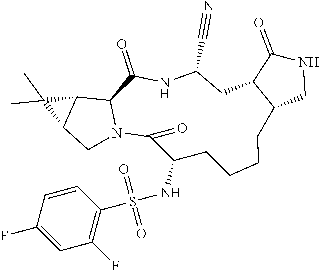 [SMILES: CC1(C)[C@@H]2[C@H]3C(=O)N[C@H](C#N)C[C@@H]4C(=O)NC[C@@H]4CCCC[C@H](NS(=O)(=O)c4ccc(F)cc4F)C(=O)N3C[C@@H]21] | ¹H NMR (400 MHz, DMSO-d₆) δ 8.7047 (d, J = 8.53 Hz, 1H), 8.3192 (d, J = 8.35 Hz, 1H), 7.8109-7.8698 (m, 1H), 7.633 (s, 1H), 7.4754-7.5306 (m, 1H), 7.2282-7.2762 (m, 1H), 4.8643-4.9137 (m, 1H), 4.2544 (s, 1H), 3.9986-4.0305 (m, 1H), 3.7093-3.7468 (m, 1H), 3.5561 (d, J = 10.64 Hz, 1H), 3.1737- 3.1962 (m, 1H), 2.7842-2.818 (m, 1H), 2.2776- 2.2929 (m, 2H), 2.0955-2.1312 (m, 1H), 1.6697- 1.754 (m, 2H), 1.3952-1.533 (m, 4H), 1.2688- 1.3179 (m, 1H), 1.054-1.1499 (m, 4H), 0.9939 (s, 3H), 0.6494 (s, 3H). ¹⁹F NMR (376 MHz, DMSO-d₆) δ −102.2958 (d, J = 12.25 Hz, 1F), −104.3033 (d, J = 12.23 Hz, 1F). MS (ESI) m/z 578.1, [C₂₇H₃₃N₅O₅F₂S + H]⁺ |
| 158 | 5 | 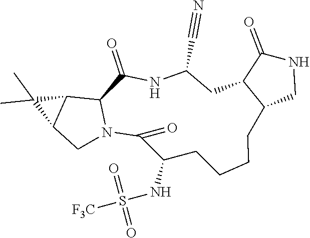 [SMILES: CC1(C)[C@@H]2[C@H]3C(=O)N[C@H](C#N)C[C@@H]4C(=O)NC[C@@H]4CCCC[C@H](NS(=O)(=O)C(F)(F)F)C(=O)N3C[C@@H]21] | ¹H NMR (400 MHz, MeOD): δ (ppm) 5.15 (t, J = 5.7 Hz, 1H), 4.54 (s, 1H), 4.33 (dd, J = 7.4, 3.6 Hz, 1H), 3.88-3.77 (m, 2H), 3.40-3.36 (m, 1H), 3.00 (dd, J = 9.9, 6.2 Hz, 1H), 2.51 (q, J = 7.3 Hz, 1H), 2.47-2.39 (m, 1H), 2.21 (ddd, J = 14.9, 7.7, 5.1 Hz, 1H), 2.05-1.91 (m, 2H), 1.84- 1.73 (m, 1H), 1.71-1.62 (m, 2H), 1.52-1.41 (m, 1H), 1.38-1.24 (m, 5H), 1.11 (s, 3H), 0.96 (s, 3H). ¹⁹F NMR (376 MHz, MeOD): δ −79.65. MS (ESI) m/z 534.1 [C₂₂H₃₀N₅O₅F₃S + H⁺] |
| 159 | 5 | 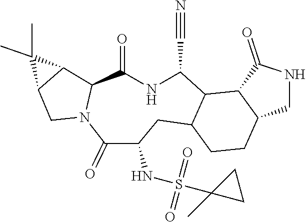 [SMILES: CC1(C)[C@@H]2[C@H]3C(=O)N[C@H](C#N)C4C(CC[C@H]5CNC(=O)[C@@H]45)C[C@H](NS(=O)(=O)C4(C)CC4)C(=O)N3C[C@@H]21] | ¹H NMR (400 MHz, MeOD) δ 5.15 (dd, J = 6.6, 4.8 Hz, 1H), 4.55 (s, 1H), 4.21- 4.13 (m, 1H), 3.92-3.79 (m, 2H), 3.37 (dd, J = 10.0, 6.6 Hz, 1H), 3.00 (dd, J = 9.9, 6.0 Hz, 1H), 2.46 (dq, J = 24.5, 7.0 Hz, 2H), 2.21 (dddd, J = 14.5, 7.1, 4.9, 1.7 Hz, 1H), 2.00 (ddt, J = 13.8, 10.4, 6.9 Hz, 2H), 1.85- 1.60 (m, 3H), 1.53-1.23 (m, 10H), 1.11 (s, 4H), 0.99 (s, 3H), 0.88-0.74 (m, 2H). MS (ESI) m/z 520.2 [C₂₅H₃₇N₅O₅S + H⁺] |
| 160 | 5 | 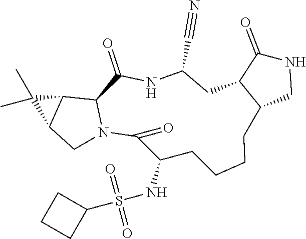 | ¹H NMR (400 MHz, MeOD) δ 5.14 (dd, J = 6.5, 4.9 Hz, 1H), 4.54 (s, 1H), 4.17 (dd, J = 7.9, 3.6 Hz, 1H), 3.97-3.88 (m, 1H), 3.88-3.72 (m, 2H), 3.40- 3.34 (m, 1H), 3.00 (dd, J = 10.0, 6.1 Hz, 1H), 2.55-2.35 (m, 4H), 2.35- 2.16 (m, 3H), 2.07-1.87 (m, 4H), 1.74-1.62 (m, 3H), 1.49-1.38 (m, 1H), 1.36-1.21 (m, 5H), 1.20-1.13 (m, 1H), 1.11 (s, 3H), 1.00 (s, 3H). MS (ESI) m/z 538.1, [C₂₅H₃₇N₅O₅S + H]⁺ |
| 161 | 5 | 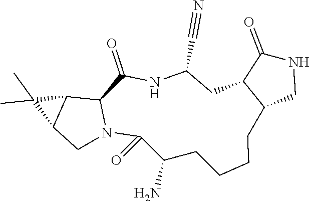 [SMILES: CC1(C)[C@@H]2[C@H]3C(=O)N[C@H](C#N)C[C@@H]4C(=O)NC[C@@H]4CCCC[C@H](N)C(=O)N3C[C@@H]21] | ¹H NMR (400 MHz, MeOD) δ 5.21 (dd, J = 6.5, 4.9 Hz, 1H), 4.57 (s, 1H), 3.96 (t, J = 4.4 Hz, 1H), 3.83 (dd, J = 10.7, 5.1 Hz, 1H), 3.75 (d, J = 10.7 Hz, 1H), 3.42-3.35 (m, 1H), 2.99 (dd, J = 9.9, 6.8 Hz, 1H), 2.57-2.33 (m, 2H), 2.25- 2.11 (m, 1H), 2.00-1.91 (m, 1H), 1.91-1.76 (m, 2H), 1.72 (d, J = 7.6 Hz, 1H), 1.65 (dd, J = 7.7, 4.9 Hz, 1H), 1.41-1.28 (m, 5H), 1.19- 1.13 (m, 1H), 1.12 (s, 3H), 0.98 (s, 3H). MS (ESI) m/z 402.1, [C₂₁H₃₁N₅O₃ + H]⁺ |
| 162 | 6 | 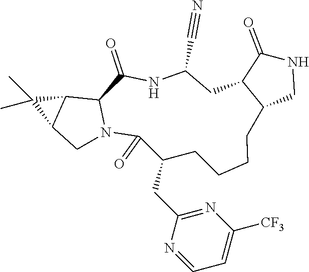 [SMILES: CC1(C)[C@@H]2[C@H]3C(=O)N[C@H](C#N)C[C@@H]4C(=O)NC[C@@H]4CCCC[C@H](Cc4nccc(C(F)(F)F)n4)C(=O)N3C[C@@H]21] | ¹H NMR (400 MHz, MeOD) δ 8.67- 8.31 (m, 1H), 6.96 (d, J = 4.9 Hz, 1H), 5.27-5.05 (m, 1H), 4.83-4.59 (m, 1H), 4.52 (s, 1H), 4.15 (d, J = 10.6 Hz, 1H), 3.92 (dt, J = 10.6, 2.6 Hz, 1H), 3.42-3.35 (m, 1H), 3.01 (dd, J = 9.9, 6.3 Hz, 1H), 2.63-2.40 (m, 2H), 2.31-2.12 (m, 1H), 2.12-1.94 (m, 2H), 1.87-1.72 (m, 1H), 1.67 (s, 2H), 1.60-1.45 (m, 1H), 1.45-1.19 (m, 6H), 1.15-1.01 (m, 4H), 0.97- 0.75 (m, 2H). MS (ESI) m/z 548.1, [C₂₆H₃₂N₇O₃F₃ + H]⁺ |
| 163 | 6 | 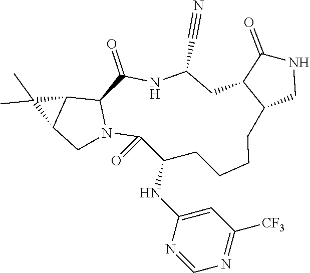 [SMILES: CC1(C)[C@@H]2[C@H]3C(=O)N[C@H](C#N)C[C@@H]4C(=O)NC[C@@H]4CCCC[C@H](Nc4cc(C(F)(F)F)ncn4)C(=O)N3C[C@@H]21] | 1H NMR (400 MHz, MeOD) δ 8.45 (s, 1H), 6.96 (s, 1H), 5.12 (t, J = 5.5 Hz, 1H), 4.71 (dd, J = 7.3, 3.7 Hz, 1H), 4.50 (s, 1H), 4.17 (d, J = 10.6 Hz, 1H), 3.93 (dd, J = 10.5, 5.1 Hz, 1H), 3.41-3.36 (m, 1H), 3.02 (dd, J = 9.9, 6.0 Hz, 1H), 2.59 (q, J = 7.3 Hz, 1H), 2.54-2.40 (m, 1H), 2.33- 2.21 (m, 1H), 2.11-1.90 (m, 2H), 1.87-1.75 (m, 1H), 1.75-1.64 (m, 2H), 1.64-1.52 (m, 1H), 1.42-1.19 (m, 5H), 1.13 (s, 3H), 1.07 (s, 3H). MS (ESI) m/z 548.2, [C₂₆H₃₂N₇O₃F₃ + H]⁺ |
| 164 | 6 | 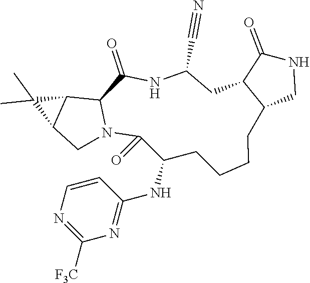 [SMILES: CC1(C)[C@@H]2[C@H]3C(=O)N[C@H](C#N)C[C@@H]4C(=O)NC[C@@H]4CCCC[C@H](Nc4ccnc(C(F)(F)F)n4)C(=O)N3C[C@@H]21] | ¹H NMR (400 MHz, MeOD): δ (ppm) 8.16 (d, J = 6.1 Hz, 1H), 6.75 (d, J = 6.1 Hz, 1H), 5.16 (dd, J = 6.7, 4.7 Hz, 1H), 4.53 (s, 1H), 4.89 (under H₂O, 1H), 4.23 (d, J = 10.7 Hz, 1H), 3.94 (ddd, J = 10.6, 3.4, 1.6 Hz, 1H), 3.41-3.35 (m, 1H), 3.01 (dd, J = 9.9, 6.3 Hz, 1H), 2.58-2.41 (m, 2H), 2.25-2.15 (m, 1H), 2.02 (dt, J = 14.8, 6.7 Hz, 2H), 1.85-1.74 (m, 1H), 1.70-1.64 (m, 2H), 1.58-1.47 (m, 1H), 1.45-1.29 (m, 4H), 1.29-1.18 (m, 1H), 1.09 (s, 3H), 0.86 (s, 3H). ¹⁹F NMR (376 MHz, MeOD): δ (ppm) −72.45. MS (ESI) m/z 548.2 [C₂₆H₃₂F₃N/₇O₃ + H]⁺ |
| 165 | 6 | 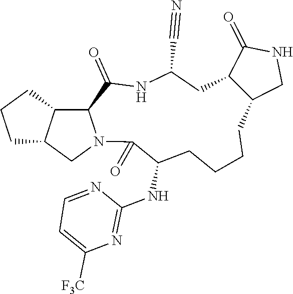 [SMILES: N#C[C@@H]1C[C@@H]2C(=O)NC[C@@H]2CCCC[C@H](Nc2nccc(C(F)(F)F)n2)C(=O)N2C[C@@H]3CCC[C@@H]3[C@H]2C(=O)N1] | ¹H NMR (400 MHz, MeOD) δ (ppm) 8.47 (s, 1H), 6.88 (d, J = 4.9 Hz, 1H), 5.16-5.08 (m, 1H), 4.68 (s, 1H), 4.47 (s, 1H), 3.75 (s, 1H), 3.30-3.25 (m, 1H), 3.00-2.82 (m, 3H), 2.45- 2.29 (m, 2H), 2.18-2.07 (m, 1H), 2.03-1.81 (m, 4H), 1.81-1.62 (m, 2H), 1.60-1.07 (m, 10H). MS (ESI) m/z 548.2 [C₂₆H₃₂F₃N₇O₃ + H]⁺ |
| 166 | 3 | 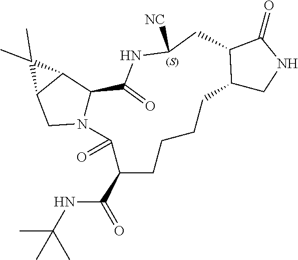 | ¹H NMR (400 MHz, MeOD) δ 4.73 (dd, J = 10.3, 3.8 Hz, 1H), 4.35 (s, 1H), 3.79-3.67 (m, 2H), 3.50-3.44 (m, 1H), 3.36-3.33 (m, 1H), 3.00 (t, J = 9.0 Hz, 1H), 2.59-2.47 (m, 2H), 2.21- 2.08 (m, 2H), 1.92-1.82 (m, 1H), 1.68-1.59 (m, 2H), 1.54 (d, J = 7.4 Hz, 1H), 1.52-1.34 (m, 3H), 1.33- 1.23 (m, 11H), 1.12-1.06 (m, 4H), 0.90 (s, 3H). MS (ESI) m/z 486.1 [C₂₆H₃₉N₅O₄ + H]⁺ |
| 167 | 3 | 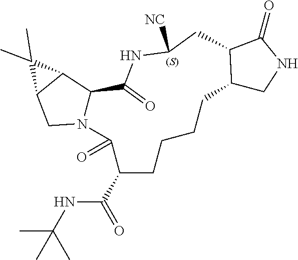 | ¹H NMR (400 MHz, MeOD) δ 7.39 (s, 1H), 5.15 (t, J = 5.8 Hz, 1H), 4.52 (s, 1H), 3.85 (dd, J = 10.6, 5.3 Hz, 1H), 3.65 (d, J = 10.6 Hz, 1H), 3.23 (dd, J = 10.6, 2.4 Hz, 1H), 3.03-2.97 (m, 1H), 2.41-2.31 (m, 2H), 2.28-2.17 (m, 1H), 2.11-2.06 (m, 2H), 1.73- 1.66 (m, 2H), 1.62-1.49 (m, 2H), 1.46-1.33 (m, 3H), 1.30 (s, 9H), 1.22-1.05 (m, 5H), 0.90 (s, 3H). MS (ESI) m/z 486.2 [C₂₆H₃₉N₅O₄ + H]⁺ |
| 168 | 3 | 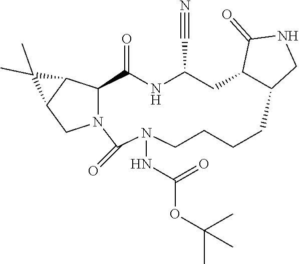 [SMILES: CC(C)(C)OC(=O)NN1CCCC[C@H]2CNC(=O)[C@H]2C[C@@H](C#N)NC(=O)[C@@H]2[C@@H]3[C@H](CN2C1=O)C3(C)C] | ¹H NMR (400 MHz, DMSO-d₆) δ 9.1045 (bs, 1H), 8.6427-8.689 (m, 1H), 7.681 (bs, 1H), 4.6067 (bs, 1H), 4.2923 (s, 1H), 3.7606- 3.7998 (m, 1H), 3.4321-3.497 (m, 1H), 3.0411 (d, J = 10.76 Hz, 1H), 2.8418 (d, J = 9.60 Hz, 1H), 2.7005 (bs, 1H), 2.356-2.3962 (m, 2H), 2.253-2.311 (m, 1H), 1.9244-1.9833 (m, 1H), 1.3678 (s, 9H), 1.147-1.3322 (m, 7H), 1.0074 (s, 3H), 0.9356 (s, 3H), 0.87-0.911 (m, 2H). MS (ESI) m/z 503.3, [C₂₅H₃₈N₆O₅ + H]⁺ |

### Biochemical Data

### Protein Expression and Purification

The full-length gene encoding SARS-CoV-2 3CLpro from strain BetaCoV/Wuhan/WIV04/2019 (Accession NC_045512) was synthesized and cloned into NdeI and XhoI site of pET29a(+) vector by Genscript (Piscataway, NJ, USA), as described previously in Huang Y et. al; Acta Pharmacol Sin. 2020September; 41(9):1141-1149, the reference of which is incorporated herein. Similarly, the full-length gene encoding a Human Coronavirus 229E (HCoV-229E, Accession X69721.1) was cloned in pET29a(+) with a C-terminus His tag. The codon optimized plasmid for E. coli expression was transformed into competent BL21(DE3) cells. A single colony was picked to inoculate 10 mL of Terrific Broth (TB) supplemented with 50 mg/L Kanamycin and grown at 37° C. with shaking at 200 rpm. The 10-mL inoculum was added to 1 L of TB with 50 mg/L Kanamycin and grown to an optical density at 600 nm of 2.5. The culture was induced using 0.5 mM Isopropyl p-D-1-thiogalactopyranoside (IPTG) and grown at 37° C. for an additional 2 hrs. The cell pellet was resuspended in lysis buffer (20 mM Tris, pH 7.5, 100 mM NaCl, 2 mM dithiothreitol (DTT) and 10 μg/mL DNase I), and lysed by sonication (25% amplitude, 2 seconds on/2 seconds off). Cell debris were removed by centrifugation at 39,191×g for 30 min at 4° C. The supernatant was loaded onto equilibrated HisTrap HP column (Cytiva). The column was washed with lysis buffer containing 5 mM imidazole, followed by another wash with 30 mM imidazole. Protein was eluted using buffer with 300 mM imidazole and further purified by gel filtration chromatography on HiLoad 16/600Superdex 200 prep grade column (Cytiva) with 20 mM Tris, pH 7.5, 100 mM NaCl, 2 mM DTT. Fractions from resulted peak were pooled and concentrated using centrifugal filter unit of 10,000-molecular-weight-cutoff. The purity and molecular weight of the protein was confirmed with SDS-PAGE and Mass Spectrometry (MS). A predominant peak at 34863 Da was observed by MS which matched with the calculated molecular weight of SARS-CoV-2 C-His-3CLpro without the N-terminal Methionine residue.

A similar expression and purification condition were followed for HCoV-229E except that the cells were transformed in Rosetta (DE3), cultured in Luria Broth and after induction the cells were grown overnight at 18° C. The molecular weight of 34120 Da was observed using MS and matched with the calculated mass without the N-terminal Methionine residue.

Recombinant MERS-CoV 3CLpro (accession K9N638) expressed in E. coli was purchased from R&D Systems (Cat No. E-719). The purity and molecular size of commercial MERS-CoV 3CL was confirmed in our lab by SDS-PAGE. Further validation by MS showed a predominant peak at 33361 Da that matched with theoretical molecular weight of 33360.3 Da.

### Biochemical Assay Method for SARS-CoV-2 3CL, HCoV-229E 3CL and MERS 3CL Proteases

A highly sensitive FRET based protease assay was developed to identify inhibitors of 3CL proteases. The substrate, Peptide 1 (Dabcyl)KTSAVLQSGFRKM(Glu)(EDANS) (2) was synthesized by Genscript. The test compounds were 3-fold serially diluted in 100% DMSO to 15 concentrations, starting at 3.33 mM. 1.5 μl of the serially diluted compounds were transferred to a black 384 well assay plate (Cat. 781900, Greiner). 23.5 μl of 2.13× concentration of SARS-CoV-2 Chis-3CLpro, H229E-CoV Chis-3CLpro or MERS-CoV 3CLpro prepared in assay buffer was added to the compounds and incubated for 30 mins at 25° C. 25 μl of 2× concentration of Peptide 1 substrate was added to the assay plate. The SARS-CoV-2 Chis-3CLpro assay plate was incubated at 37° C., the H229E-CoV Chis-3CLpro assay plate and MERS-CoV 3CLpro assay plate were incubated at 25° C. for 1.5 hrs. The final assay contained 12.5 nM of SARS-CoV-2 Chis-3CLpro or 6 nM H229E-CoV Chis-3CLpro or 75 nM MERS-CoV 3CLpro with 6 μM peptide 1 substrate and 3% DMSO in assay buffer containing 50 mM HEPES at pH 7.5, 100 mM NaCl, and 0.01% Triton X-100 and 1 mM DTT.

The FRET signal was measured using an excitation wavelength of 340 nm (UV(TRF) 340/60 nm, Barcode 101), emission wavelength of 490 nm (DSPPsion 486/10 filter, Barcode 220) and Lance/DELFIA D400 single mirror (Barcode 412) on Envision plate reader (2104 EnVision Multilabel Plate Readers, Perkin Elmer). The dose-dependent inhibition curves were fitted with a variable slope using GraphPad Prism software (GraphPad, USA) to determine a compound's IC50.
+: >20 μM
++: 1-20 μM
+++: 100 nM-1 μM
++++: <100 nM

| Cpd No. | SARS CoV-2 IC₅₀ | 229E IC₅₀ | MERS IC₅₀ |
|---|---|---|---|
| 1 | ++ | ++ | + |
| 2 | ++ | ++ | + |
| 3 | ++ | +++ | + |
| 4 | ++++ | ++++ | +++ |
| 5 | ++ | ++ | |
| 6 | + | ++ | |
| 7 | + | ++ | + |
| 8 | +++ | +++ | ++ |
| 9 | +++ | +++ | ++ |
| 10 | +++ | ++ | ++ |
| 11 | ++ | +++ | ++ |
| 12 | +++ | ++++ | ++ |
| 13 | ++ | +++ | ++ |
| 14 | +++ | +++ | ++ |
| 15 | +++ | +++ | +++ |
| 16 | +++ | ++ | ++ |
| 17 | +++ | +++ | +++ |
| 18 | +++ | +++ | +++ |
| 19 | ++++ | ++++ | +++ |
| 20 | ++ | ++ | + |
| 21 | ++++ | ++++ | +++ |
| 22 | + | ++ | + |
| 23 | + | + | + |
| 24 | ++ | +++ | ++ |
| 25 | ++ | +++ | ++ |
| 26 | + | ++ | + |
| 27 | ++ | ++ | ++ |
| 28 | ++ | ++ | ++ |
| 29 | + | + | + |
| 30 | +++ | ++++ | ++ |
| 31 | + | ++ | + |
| 32 | +++ | ++++ | +++ |
| 33 | + | ++ | + |
| 34 | +++ | +++ | ++ |
| 35 | ++ | ++ | |
| 36 | ++++ | ++++ | +++ |
| 37 | ++++ | ++++ | +++ |
| 38 | ++ | +++ | + |
| 39 | +++ | +++ | ++ |
| 40 | ++ | ++ | ++ |
| 41 | + | + | |
| 42 | ++ | +++ | ++ |
| 43 | ++ | +++ | ++ |
| 44 | ++++ | ++++ | +++ |
| 45 | +++ | ++++ | ++ |
| 46 | ++ | +++ | ++ |
| 47 | ++ | +++ | + |
| 48 | +++ | ++++ | +++ |
| 49 | + | + | + |
| 50 | ++ | ++ | ++ |
| 51 | ++ | ++ | + |
| 52 | +++ | +++ | ++ |
| 53 | ++++ | ++++ | +++ |
| 54 | +++ | ++ | +++ |
| 55 | ++++ | ++++ | +++ |
| 56 | +++ | ++++ | +++ |
| 57 | +++ | +++ | ++ |
| 58 | ++++ | ++++ | +++ |
| 59 | +++ | +++ | ++ |
| 60 | ++++ | ++++ | +++ |
| 61 | ++++ | ++++ | ++++ |
| 62 | ++++ | ++++ | ++++ |
| 63 | ++++ | ++++ | +++ |
| 64 | + | ++ | + |
| 65 | ++++ | ++ | +++ |
| 66 | ++++ | +++ | +++ |
| 67 | +++ | +++ | +++ |
| 68 | ++++ | ++++ | ++++ |
| 69 | ++++ | ++++ | +++ |
| 70 | ++++ | ++++ | +++ |
| 71 | ++ | ++ | ++ |
| 72 | ++++ | ++++ | ++++ |
| 73 | ++++ | ++++ | ++++ |
| 74 | ++ | ++ | ++ |
| 75 | ++++ | ++++ | ++++ |
| 76 | ++++ | ++++ | +++ |
| 77 | ++++ | ++++ | ++++ |
| 78 | ++++ | +++ | ++ |
| 79 | ++++ | ++++ | +++ |
| 80 | +++ | +++ | +++ |
| 81 | ++++ | ++++ | +++ |
| 82 | ++++ | ++++ | ++++ |
| 83 | ++++ | ++++ | ++++ |
| 84 | ++++ | ++++ | +++ |
| 85 | ++++ | ++++ | ++++ |
| 86 | ++++ | ++++ | +++ |
| 87 | ++++ | ++++ | ++++ |
| 88 | ++++ | ++++ | +++ |
| 89 | ++++ | ++++ | +++ |
| 90 | ++++ | ++++ | +++ |
| 91 | ++++ | ++++ | ++++ |
| 92 | ++++ | ++++ | ++++ |
| 93 | ++++ | ++++ | +++ |
| 94 | ++++ | ++++ | ++++ |
| 95 | ++++ | ++++ | +++ |
| 96 | ++++ | ++++ | ++++ |
| 97 | ++++ | ++++ | +++ |
| 98 | +++ | ++++ | +++ |
| 99 | +++ | ++ | ++ |
| 100 | ++ | +++ | ++ |
| 101 | ++++ | ++++ | +++ |
| 102 | ++++ | ++++ | +++ |
| 103 | ++++ | ++++ | ++++ |
| 104 | ++++ | ++++ | ++++ |
| 105 | +++ | ++++ | +++ |
| 106 | ++++ | ++++ | +++ |
| 107 | ++++ | ++++ | ++++ |
| 108 | ++++ | ++++ | ++++ |
| 109 | ++++ | ++++ | ++++ |
| 110 | ++++ | ++++ | +++ |
| 111 | +++ | +++ | +++ |
| 112 | ++++ | ++++ | +++ |
| 113 | +++ | +++ | ++++ |
| 114 | ++++ | ++++ | +++ |
| 115 | ++++ | ++++ | ++++ |
| 116 | ++++ | ++++ | ++++ |
| 117 | ++++ | ++++ | ++++ |
| 118 | ++++ | ++++ | +++ |
| 119 | ++++ | ++++ | ++++ |
| 120 | ++++ | ++++ | ++++ |
| 121 | ++++ | ++++ | +++ |
| 122 | ++++ | ++++ | ++++ |
| 123 | ++++ | ++++ | +++ |
| 124 | ++++ | ++++ | ++++ |
| 125 | ++ | +++ | + |
| 126 | + | + | + |
| 127 | ++ | ++ | ++ |
| 128 | + | + | + |
| 129 | ++++ | ++++ | ++++ |
| 130 | +++ | ++++ | +++ |
| 131 | +++ | ++ | +++ |
| 132 | ++++ | ++++ | +++ |
| 133 | ++++ | ++++ | ++++ |
| 134 | ++++ | ++++ | ++++ |
| 135 | +++ | +++ | ++++ |
| 136 | ++++ | ++++ | ++++ |
| 137 | ++++ | ++++ | ++ |
| 138 | +++ | +++ | +++ |
| 139 | ++++ | ++++ | ++++ |
| 140 | +++ | ++ | +++ |
| 141 | ++++ | ++++ | ++++ |
| 142 | +++ | +++ | +++ |
| 143 | +++ | +++ | +++ |
| 144 | ++++ | +++ | ++ |
| 145 | ++ | ++ | ++ |
| 146 | +++ | +++ | +++ |
| 147 | ++++ | +++ | +++ |
| 148 | ++++ | ++++ | +++ |
| 149 | ++++ | ++++ | +++ |
| 150 | ++++ | ++++ | ++++ |
| 151 | ++++ | ++++ | +++ |
| 152 | ++++ | ++++ | ++++ |
| 153 | ++++ | ++++ | +++ |
| 154 | ++++ | ++++ | ++++ |
| 155 | ++++ | ++++ | +++ |
| 156 | ++++ | ++++ | ++++ |
| 157 | ++++ | ++++ | ++++ |
| 158 | ++++ | ++++ | ++++ |
| 159 | ++++ | ++++ | ++++ |
| 160 | ++++ | ++++ | ++++ |
| 161 | +++ | ++++ | ++ |
| 162 | ++++ | ++++ | +++ |
| 163 | ++++ | ++++ | +++ |
| 164 | ++++ | ++++ | +++ |
| 165 | ++++ | +++ | +++ |
| 166 | ++ | ++ | + |
| 167 | +++ | +++ | ++ |
| 168 | ++ | ++ | ++ |

### Cellular Assays

### Drug Titration in BSL2 Human Coronaviruses (229E and OC43)

MRC-5 cells (ATCC®CCL-1711™) were seeded at a density of 10,000 cells per well into black, clear flat-bottom 96-well plates (Greiner) overnight. The next day, culture medium was removed from each well and cells were infected with 229E (ATCC® VR-740™) or OC43 (ATCC® VR-1558^{T1}) virus at multiplicity of infection (MOI) of 0.01 using 50 μl of virus inoculum per well. After 1 hour of virus infection in EMEM containing 2% FBS, virus inoculum was removed and 50 μl of diluted test compounds in EMEM+2% FBS+0.5% DMSO, were added to each well. Compounds were tested using 50 μM starting concentration, 8-point, 5-fold serial dilution. The cells were then incubated for 4 days (229E) or 5 days (OC43) before cell viability was measured with CellTiter Glo (Promega), according to manufacturer's protocol, using Tecan infinite M200 pro plate reader. Cytotoxicity of compounds was assessed in parallel on uninfected cells plated alongside the cells for virus infection. CCs values were determined by applying nonlinear fit of luminescence readouts from uninfected, compound-treated wells against compound concentration. Likewise, EC50 values were determined by nonlinear fit of luminescence readouts from virus-infected, compound-treated wells. Data analysis was performed using GraphPad Prism 8, and SI calculated as the ratio of CC₅₀ to EC₅₀.

### Drug Titration in BSL3 Human Coronaviruses (SARS-CoV-2 and MERS)

Vero E6 cells were seeded a 96-well plates at 20,000 cells/well in MEM+10% FBS overnight. The next day, culture medium was removed from each well before addition of virus with compounds. SARS-CoV-2 (Wuhan strain) or MERS viruses at 100TCID50 concentration were mixed with test compounds immediately before addition to the cells in MEM+2% FBS+0.5% DMSO. Compounds were tested using 50 μM starting concentration, 8-point, 5-fold serial dilution in presence of 2 μM efflux inhibitor, CP100356. Viral ToxGlo assay was performed after 96 hours (SARS-CoV-2) or 120 hours (MERS) incubation time, according to manufacturer's protocol. Assay readout was performed using luminescence measurement on a Tecan Spark plate reader.

CC₅₀ values were determined by applying nonlinear fit of luminescence readouts from uninfected, compound-treated wells against compound concentration. Likewise, EC₅₀ values were determined by nonlinear fit of luminescence readouts from virus-infected, compound-treated wells. Data analysis was performed using GraphPad Prism 8, and SI calculated as the ratio of CC₅₀ to EC₅₀.
+: >20 μM
++: 1-20 μM
+++: 100 nM-1 μM

| Cpd No. | 229E EC₅₀ | OC43 EC₅₀ |
|---|---|---|
| 4 | ++ | + |
| 7 | + | + |
| 8 | ++ | + |
| 9 | ++ | + |
| 10 | ++ | + |
| 11 | ++ | ++ |
| 12 | ++ | + |
| 13 | ++ | + |
| 14 | ++ | + |
| 17 | ++ | ++ |
| 18 | +++ | ++ |
| 19 | + | + |
| 21 | +++ | + |
| 22 | + | + |
| 23 | + | + |
| 24 | ++ | + |
| 26 | ++ | + |
| 27 | ++ | + |
| 28 | + | + |
| 30 | ++ | + |
| 32 | ++ | + |
| 33 | + | + |
| 34 | ++ | + |
| 35 | + | + |
| 36 | ++ | ++ |
| 38 | ++ | + |
| 39 | ++ | ++ |
| 40 | ++ | ++ |
| 44 | +++ | +++ |
| 45 | ++ | + |
| 47 | ++ | + |
| 48 | ++ | ++ |
| 49 | + | + |
| 50 | ++ | ++ |
| 51 | + | + |
| 52 | ++ | + |
| 53 | ++++ | +++ |
| 54 | + | ++ |
| 55 | ++++ | ++++ |
| 56 | +++ | +++ |
| 57 | ++ | ++ |
| 58 | ++++ | +++ |
| 59 | ++ | ++ |
| 60 | ++++ | +++ |
| 61 | ++++ | ++++ |
| 62 | ++++ | +++ |
| 63 | +++ | ++ |
| 65 | ++ | ++ |
| 66 | ++ | ++ |
| 67 | ++ | ++ |
| 68 | +++ | +++ |
| 69 | ++++ | +++ |
| 70 | ++++ | ++++ |
| 71 | + | + |
| 72 | ++++ | ++++ |
| 73 | ++++ | ++++ |
| 74 | + | + |
| 75 | ++++ | ++++ |
| 76 | +++ | ++++ |
| 77 | +++ | +++ |
| 78 | ++ | ++ |
| 79 | ++++ | ++++ |
| 80 | ++ | ++ |
| 81 | +++ | ++++ |
| 82 | ++++ | ++++ |
| 83 | +++ | +++ |
| 84 | +++ | +++ |
| 85 | ++ | ++ |
| 86 | ++++ | ++++ |
| 87 | ++++ | ++++ |
| 88 | ++++ | ++++ |
| 89 | +++ | +++ |
| 90 | +++ | ++++ |
| 91 | ++++ | ++++ |
| 92 | ++++ | ++++ |
| 93 | +++ | +++ |
| 94 | ++++ | ++++ |
| 95 | ++++ | +++ |
| 96 | ++++ | +++ |
| 97 | ++++ | +++ |
| 98 | +++ | ++ |
| 99 | ++ | ++ |
| 100 | ++ | ++ |
| 101 | ++++ | ++++ |
| 102 | +++ | +++ |
| 103 | ++++ | ++++ |
| 104 | ++++ | ++++ |
| 105 | ++ | ++ |
| 106 | ++++ | ++++ |
| 107 | ++ | ++ |
| 108 | ++++ | ++++ |
| 109 | ++++ | +++ |
| 110 | ++++ | +++ |
| 111 | +++ | +++ |
| 112 | ++++ | ++++ |
| 113 | ++ | +++ |
| 114 | ++++ | ++++ |
| 115 | +++ | +++ |
| 116 | ++++ | ++++ |
| 117 | ++++ | ++++ |
| 118 | ++++ | ++++ |
| 119 | +++ | +++ |
| 120 | ++++ | ++++ |
| 121 | ++++ | ++++ |
| 122 | +++ | +++ |
| 123 | ++ | ++ |
| 124 | ++++ | ++++ |
| 125 | ++ | + |
| 127 | ++ | ++ |
| 129 | ++++ | ++++ |
| 130 | ++++ | +++ |
| 131 | + | + |
| 132 | +++ | +++ |
| 133 | ++++ | ++++ |
| 134 | +++ | +++ |
| 135 | ++ | +++ |
| 136 | +++ | +++ |
| 137 | +++ | ++ |
| 138 | ++ | ++ |
| 139 | ++++ | ++++ |
| 141 | ++++ | ++++ |
| 142 | +++ | +++ |
| 143 | +++ | +++ |
| 144 | ++ | ++ |
| 145 | + | ++ |
| 146 | +++ | +++ |
| 147 | ++ | ++ |
| 148 | ++++ | +++ |
| 149 | ++++ | +++ |
| 150 | ++++ | ++++ |
| 151 | +++ | +++ |
| 152 | ++++ | ++++ |
| 153 | ++++ | ++++ |
| 154 | ++++ | ++++ |
| 155 | ++++ | ++++ |
| 156 | ++++ | ++++ |
| 157 | ++++ | ++++ |
| 158 | +++ | ++ |
| 159 | ++++ | ++++ |
| 160 | ++++ | ++++ |
| 161 | +++ | ++ |
| 162 | ++++ | ++++ |
| 163 | +++ | +++ |
| 164 | +++ | ++ |
| 165 | ++++ | +++ |
| 166 | + | ++ |
| 167 | ++ | ++ |

| Cpd No. | SARS CoV-2 EC₅₀ |
|---|---|
| 44 | +++ |
| 55 | ++++ |
| 61 | ++++ |
| 69 | ++++ |
| 70 | ++++ |
| 72 | ++++ |
| 75 | ++++ |
| 79 | ++++ |
| 82 | +++ |
| 87 | ++++ |
| 88 | ++++ |
| 92 | ++++ |
| 96 | +++ |
| 101 | ++++ |
| 102 | +++ |
| 154 | ++++ |

| Cpd No. | MERS EC₅₀ |
|---|---|
| 55 | ++++ |
| 69 | ++++ |
| 70 | ++++ |
| 72 | ++++ |
| 75 | ++++ |
| 77 | ++++ |
| 79 | ++++ |
| 80 | +++ |
| 81 | ++++ |
| 82 | ++++ |
| 83 | ++++ |
| 86 | ++++ |
| 101 | ++++ |
| 102 | ++++ |
| 103 | ++++ |
| 104 | ++++ |
| 105 | ++ |
| 107 | +++ |
| 108 | ++++ |
| 154 | ++++ |
| 156 | ++++ |

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A compound of Formula (I) or a salt, solvate, stereoisomer or prodrug thereof: 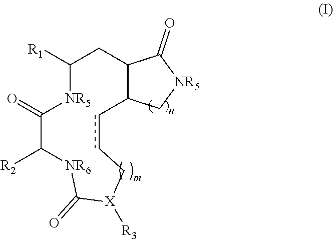
wherein R₁ is selected from optionally substituted acyl and cyano;
R₂ is selected from optionally substituted alkyl, and optionally substituted alkenyl;
X is selected from N or C—R₄;
R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino;
R₄ is selected from H, and optionally substituted alkyl;
R₅ is independently selected from H, and optionally substituted alkyl;
R₆ is selected from H and optionally substituted alkyl;
or R₂ and R₆ are linked to form an optionally substituted heterocyclyl;
m is an integer selected from 1 to 4; and
n is an integer selected from 1 to 4.

2. The compound according to claim 1, wherein R₁ is selected from HC(O)— and cyano.

3. The compound according to claim 1, wherein R₂ is C₁-C₅ alkyl.

4. The compound according to claim 1, wherein R₂ is selected from butyl, iso-butyl, pentyl, iso-pentyl, neo-pentyl, halopentyl, haloiso-pentyl, haloneo-pentyl, cyclopentylmethyl, and cyclopentyl.

5. The compound according to claim 1, wherein R₃ is selected from H, optionally substituted phenylmethyl, optionally substituted halophenylmethyl, optionally substituted trihalomethylphenylmethyl, optionally substituted cyclopentyl, optionally substituted cyclopentylmethyl, optionally substituted tert-butyloxy, optionally substituted cyclohexyloxy, optionally substituted tetrahydropyranylacylamino, optionally substituted tert-butylacylamino, optionally substituted trihalotert-butylacylamino, optionally substituted trihalomethylacylamino, optionally substituted neo-pentylacylamino, optionally substituted dihalocyclobutylmethylacylamino, optionally substituted cyclopentylmethylacylamino, optionally substituted pyrimidinylacylamino, optionally substituted phenylacylamino, optionally substituted halophenylacylamino, optionally substituted dihalophenylacylamino, optionally substituted isoxazolylacylamino, optionally substituted methylisoxazolylacylamino, optionally substituted trihalomethylisoxazolylacylamino, optionally substituted thiazolylacylamino, optionally substituted methylthiazolylacylamino, optionally substituted oxazolylacylamino, optionally substituted methyloxazolylacylamino, optionally substituted phenylacylamino, optionally substituted halophenylacylamino, optionally substituted dihalophenylacylamino, optionally substituted trihalomethylphenylacylamino, optionally substituted halotrihalomethylphenylacylamino, optionally substituted halophenylpropylacylamino, optionally substituted cyclopropylacylamino, optionally substituted trihalomethylcyclopropylacylamino, optionally substituted cyclobutylacylamino, optionally substituted methylcyclobutylacylamino, optionally substituted dihalocyclobutylacylamino, optionally substituted bicyclo[1.1.1]pentylacylamino, optionally substituted trihalomethylbicyclo[1.1.1]pentylacylamino, optionally substituted oxetanylacylamino, optionally substituted methyloxetanylacylamino, optionally substituted bicycl[2.1.1]hexylacylamino, optionally substituted cyclohexylacylamino, optionally substituted dihalocyclohexylacylamino, optionally substituted cyclohexylacylamino, optionally substituted spiro[3.3]heptylacylamino, optionally substituted tert-butyloxyacylamino (or Boc-NH or Boc-N(methyl)), optionally substituted cyclopentyloxyacyamino, optionally substituted neo-pentyloxyacylamino, optionally substituted cyclobutyloxyacylamino, optionally substituted trihalomethylcyclobutyloxyacylamino, optionally substituted phenylsulfonylamino, optionally substituted dihalophenylsulfonylamino, optionally substituted cyclopropylsulfonylamino, optionally substituted methylsulfonylamino, optionally substituted trihalomethylsulfonylamino, optionally substituted cyclobutylsulfonylamino, optionally substituted amino, optionally substituted pyrimidinylamino, optionally substituted trihalomethylpyrimidinylamino, and optionally substituted tert-butylaminoacyl.

6. The compound according to claim 1, wherein R₄ and R₅ are independently H or methyl.

7. (canceled)

8. The compound according to claim 1, wherein the compound is a compound of Formula (Ia): 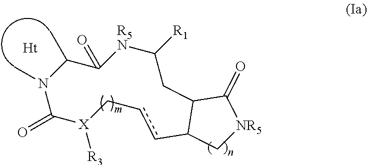
wherein R₁ is selected from optionally substituted acyl and cyano;
Ht is an optionally substituted heterocyclyl;
X is selected from N or C—R₄;
R₃ is selected from H, optionally substituted alkyl, optionally substituted cycloalkyl, optionally substituted amino, optionally substituted aryl, optionally substituted alkyoxy, optionally substituted cycloalkyoxy, optionally substituted oxyacylamino, optionally substituted aminoacyloxy, optionally substituted acylamino, optionally substituted aminoacyl, and optionally substituted sulfonylamino;
R₄ is selected from H, and optionally substituted alkyl;
R₅ is independently selected from H, and optionally substituted alkyl;
m is an integer selected from 1 to 4; and
n is an integer selected from 1 to 4.

9. The compound according to claim 1, wherein when R₂ and R₆ are linked to form an optionally substituted heterocyclyl or is Ht, the heterocyclyl is an optionally substituted 5-7 membered heterocyclyl.

10. The compound according to claim 1, wherein the optional substituent on the heterocyclyl is selected from halo, methyl, methoxy, ethoxy, cyclopropyl, fused dimethylcyclopropyl, fused cyclopentyl, bridged methylene, and bridged ethylene.

11. The compound according to claim 1, wherein m is an integer selected from 1 to 3 and/or wherein n is an integer selected from 1 to 2.

12. (canceled)

13. The compound according to claim 1, wherein the compound of Formula (I) or a salt, solvate, stereoisomer or prodrug thereof is represented by Formula (I^{iix}b):

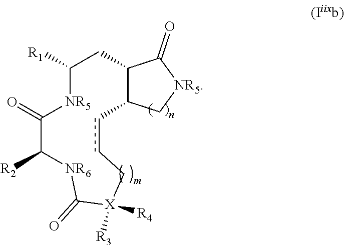

14. The compound according to claim 1, wherein the compound of Formula (Ia) or a salt, solvate, stereoisomer or prodrug thereof is represented by Formula (Ia^{iix}b):

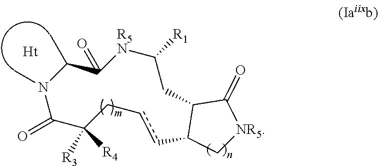

15. The compound according to claim 1 selected from one of the following:

| Cpd No. | Structure | Cpd No. | Structure |
|---|---|---|---|
| 1 | 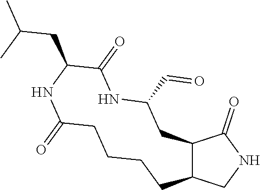 [SMILES: CC(C)C[C@@H]1NC(=O)CCCC[C@@H]2CNC(=O)[C@@H]2C[C@@H](C=O)NC1=O] | 85 | 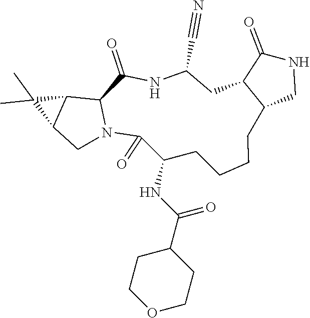 [SMILES: CC1(C)[C@@H]2[C@H]3C(=O)N[C@H](C#N)C[C@@H]4C(=O)NC[C@@H]4CCCC[C@H](NC(=O)C4CCOCC4)C(=O)N3C[C@@H]21] |
| 2 | 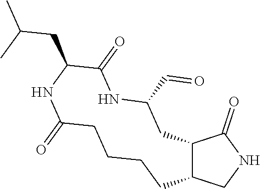 [SMILES: CC(C)C[C@@H]1NC(=O)CCCC[C@H]2CNC(=O)[C@H]2C[C@@H](C=O)NC1=O] | 86 | 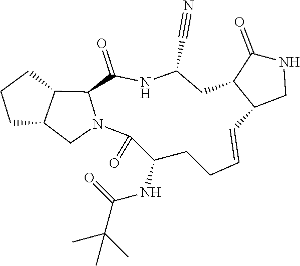 [SMILES: CC(C)(C)C(=O)N[C@H]1CC/C=C/[C@H]2CNC(=O)[C@H]2C[C@@H](C#N)NC(=O)[C@@H]2[C@H]3CCC[C@H]3CN2C1=O] |
| 3 | 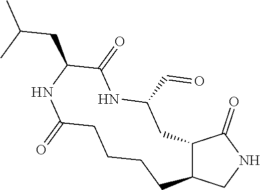 [SMILES: CC(C)C[C@@H]1NC(=O)CCCC[C@@H]2CNC(=O)[C@H]2C[C@@H](C=O)NC1=O] | 87 | 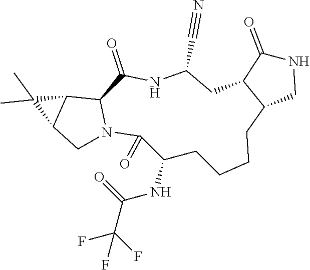 [SMILES: CC1(C)[C@@H]2[C@H]3C(=O)N[C@H](C#N)C[C@@H]4C(=O)NC[C@@H]4CCCC[C@H](NC(=O)C(F)(F)F)C(=O)N3C[C@@H]21] |
| 4 | 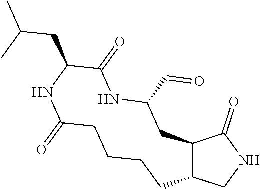 [SMILES: CC(C)C[C@@H]1NC(=O)CCCC[C@H]2CNC(=O)[C@@H]2C[C@@H](C=O)NC1=O] | 88 | 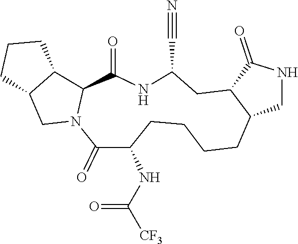 [SMILES: N#C[C@@H]1C[C@@H]2C(=O)NC[C@@H]2CCCC[C@H](NC(=O)C(F)(F)F)C(=O)N2C[C@@H]3CCC[C@@H]3[C@H]2C(=O)N1] |
| 5 | 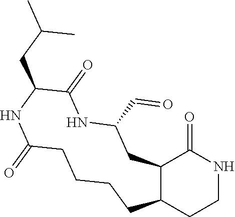 [SMILES: CC(C)C[C@@H]1NC(=O)CCCC[C@@H]2CCNC(=O)[C@@H]2C[C@@H](C=O)NC1=O] | 89 | 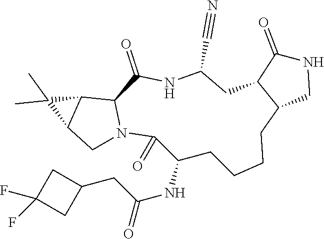 [SMILES: CC1(C)[C@@H]2[C@H]3C(=O)N[C@H](C#N)C[C@@H]4C(=O)NC[C@@H]4CCCC[C@H](NC(=O)CC4CC(F)(F)C4)C(=O)N3C[C@@H]21] |
| 6 | 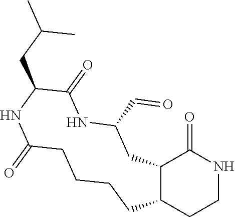 [SMILES: CC(C)C[C@@H]1NC(=O)CCCC[C@H]2CCNC(=O)[C@H]2C[C@@H](C=O)NC1=O] | 90 | 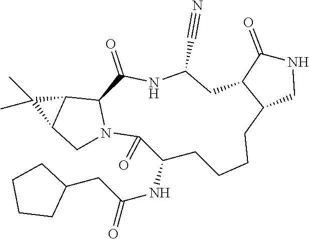 [SMILES: CC1(C)[C@@H]2[C@H]3C(=O)N[C@H](C#N)C[C@@H]4C(=O)NC[C@@H]4CCCC[C@H](NC(=O)CC4CCCC4)C(=O)N3C[C@@H]21] |
| 7 | 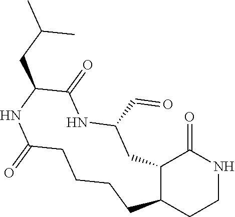 [SMILES: CC(C)C[C@@H]1NC(=O)CCCC[C@@H]2CCNC(=O)[C@H]2C[C@@H](C=O)NC1=O] | 91 | 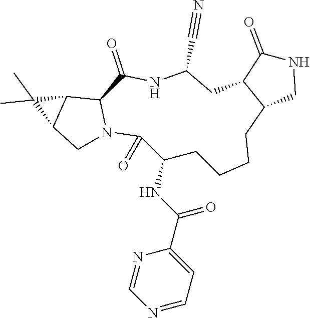 [SMILES: CC1(C)[C@@H]2[C@H]3C(=O)N[C@H](C#N)C[C@@H]4C(=O)NC[C@@H]4CCCC[C@H](NC(=O)c4ccncn4)C(=O)N3C[C@@H]21] |
| 8 | 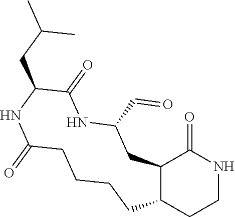 [SMILES: CC(C)C[C@@H]1NC(=O)CCCC[C@H]2CCNC(=O)[C@@H]2C[C@@H](C=O)NC1=O] | 92 | 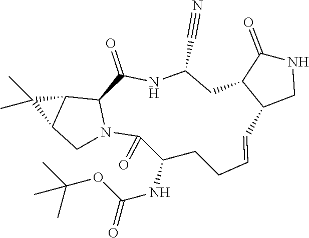 [SMILES: CC(C)(C)OC(=O)N[C@H]1CC/C=C/[C@H]2CNC(=O)[C@H]2C[C@@H](C#N)NC(=O)[C@@H]2[C@@H]3[C@H](CN2C1=O)C3(C)C] |
| 9 | 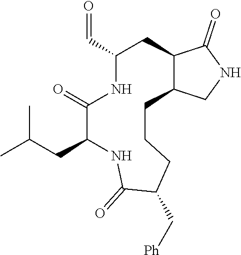 [SMILES: CC(C)C[C@@H]1NC(=O)[C@@H](Cc2ccccc2)CCC[C@@H]2CNC(=O)[C@@H]2C[C@@H](C=O)NC1=O] | 93 | 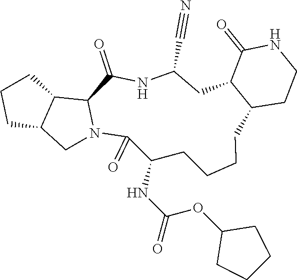 [SMILES: N#C[C@@H]1C[C@@H]2C(=O)NCC[C@@H]2CCCC[C@H](NC(=O)OC2CCCC2)C(=O)N2C[C@@H]3CCC[C@@H]3[C@H]2C(=O)N1] |
| 10 | 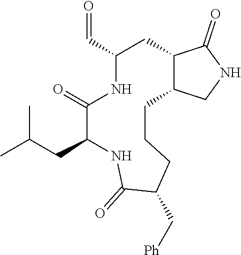 [SMILES: CC(C)C[C@@H]1NC(=O)[C@@H](Cc2ccccc2)CCC[C@H]2CNC(=O)[C@H]2C[C@@H](C=O)NC1=O] | 94 | 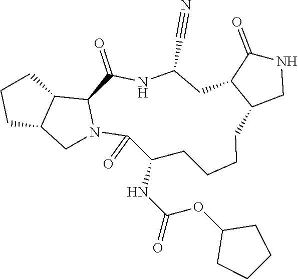 [SMILES: N#C[C@@H]1C[C@@H]2C(=O)NC[C@@H]2CCCC[C@H](NC(=O)OC2CCCC2)C(=O)N2C[C@@H]3CCC[C@@H]3[C@H]2C(=O)N1] |
| 11 | 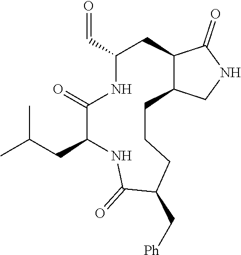 [SMILES: CC(C)C[C@@H]1NC(=O)[C@H](Cc2ccccc2)CCC[C@@H]2CNC(=O)[C@@H]2C[C@@H](C=O)NC1=O] | 95 | 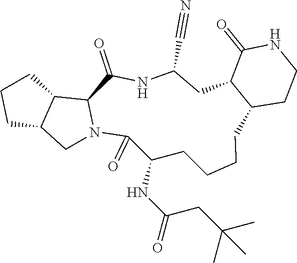 [SMILES: CC(C)(C)CC(=O)N[C@H]1CCCC[C@H]2CCNC(=O)[C@H]2C[C@@H](C#N)NC(=O)[C@@H]2[C@H]3CCC[C@H]3CN2C1=O] |
| 12 | 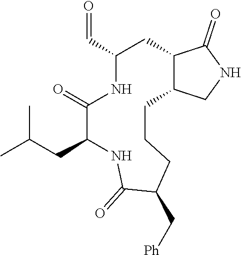 [SMILES: CC(C)C[C@@H]1NC(=O)[C@H](Cc2ccccc2)CCC[C@H]2CNC(=O)[C@H]2C[C@@H](C=O)NC1=O] | 96 | 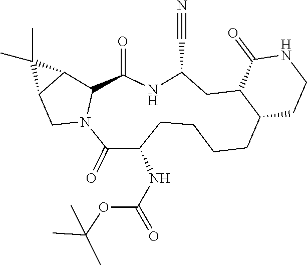 |
| 13 | 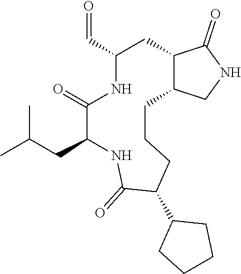 [SMILES: CC(C)C[C@@H]1NC(=O)[C@@H](C2CCCC2)CCC[C@H]2CNC(=O)[C@H]2C[C@@H](C=O)NC1=O] | 97 | 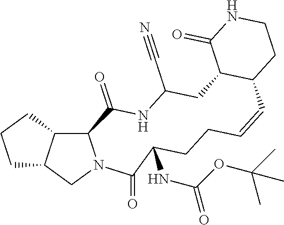 [SMILES: CC(C)(C)OC(=O)N[C@H]1CC/C=C\[C@H]2CCNC(=O)[C@H]2CC(C#N)NC(=O)[C@@H]2[C@H]3CCC[C@H]3CN2C1=O] |
| 14 | 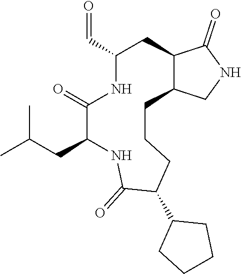 [SMILES: CC(C)C[C@@H]1NC(=O)[C@@H](C2CCCC2)CCC[C@@H]2CNC(=O)[C@@H]2C[C@@H](C=O)NC1=O] | 98 | 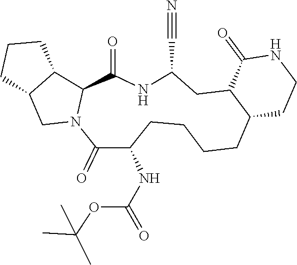 |
| 15 | 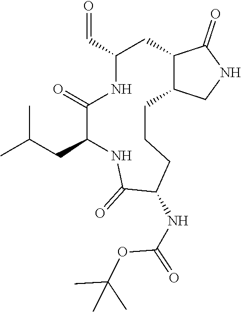 [SMILES: CC(C)C[C@@H]1NC(=O)[C@@H](NC(=O)OC(C)(C)C)CCC[C@H]2CNC(=O)[C@H]2C[C@@H](C=O)NC1=O] | 99 | 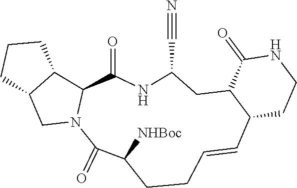 |
| 16 | 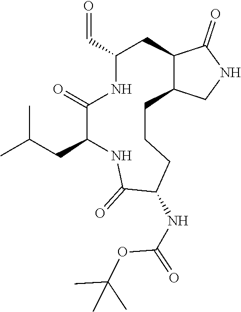 [SMILES: CC(C)C[C@@H]1NC(=O)[C@@H](NC(=O)OC(C)(C)C)CCC[C@@H]2CNC(=O)[C@@H]2C[C@@H](C=O)NC1=O] | 100 | 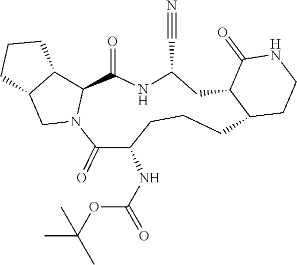 [SMILES: CC(C)(C)OC(=O)N[C@H]1CCC[C@H]2CCNC(=O)[C@H]2C[C@@H](C#N)NC(=O)[C@@H]2[C@H]3CCC[C@H]3CN2C1=O] |
| 17 | 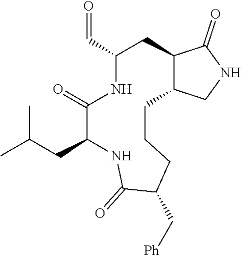 [SMILES: CC(C)C[C@@H]1NC(=O)[C@@H](Cc2ccccc2)CCC[C@H]2CNC(=O)[C@@H]2C[C@@H](C=O)NC1=O] | 101 | 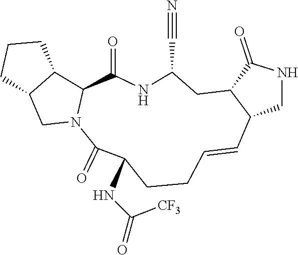 [SMILES: N#C[C@@H]1C[C@@H]2C(=O)NC[C@@H]2/C=C/CC[C@H](NC(=O)C(F)(F)F)C(=O)N2C[C@@H]3CCC[C@@H]3[C@H]2C(=O)N1] |
| 18 | 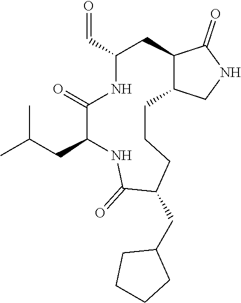 [SMILES: CC(C)C[C@@H]1NC(=O)[C@@H](CC2CCCC2)CCC[C@H]2CNC(=O)[C@@H]2C[C@@H](C=O)NC1=O] | | |
| 19 | 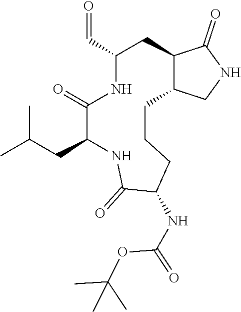 [SMILES: CC(C)C[C@@H]1NC(=O)[C@@H](NC(=O)OC(C)(C)C)CCC[C@H]2CNC(=O)[C@@H]2C[C@@H](C=O)NC1=O] | 103 | 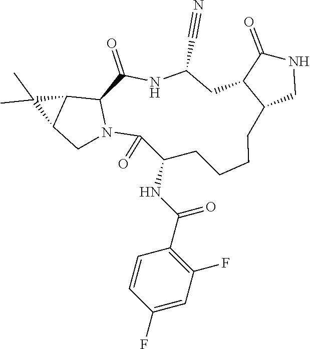 [SMILES: CC1(C)[C@@H]2[C@H]3C(=O)N[C@H](C#N)C[C@@H]4C(=O)NC[C@@H]4CCCC[C@H](NC(=O)c4ccc(F)cc4F)C(=O)N3C[C@@H]21] |
| 20 | 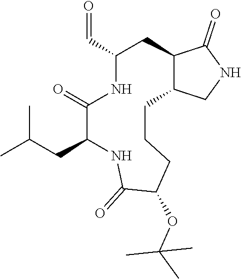 [SMILES: CC(C)C[C@@H]1NC(=O)[C@@H](OC(C)(C)C)CCC[C@H]2CNC(=O)[C@@H]2C[C@@H](C=O)NC1=O] | 104 | 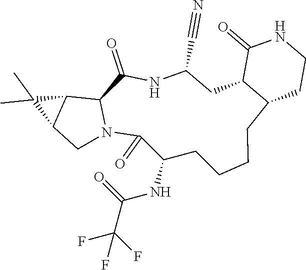 [SMILES: CC1(C)[C@@H]2[C@H]3C(=O)N[C@H](C#N)C[C@@H]4C(=O)NCC[C@@H]4CCCC[C@H](NC(=O)C(F)(F)F)C(=O)N3C[C@@H]21] |
| 21 | 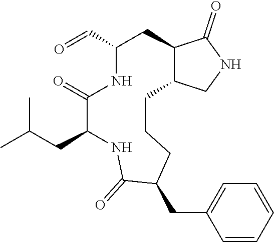 [SMILES: CC(C)C[C@@H]1NC(=O)[C@H](Cc2ccccc2)CCC[C@H]2CNC(=O)[C@@H]2C[C@@H](C=O)NC1=O] | 105 | 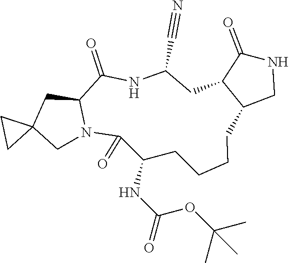 [SMILES: CC(C)(C)OC(=O)N[C@H]1CCCC[C@H]2CNC(=O)[C@H]2C[C@@H](C#N)NC(=O)[C@@H]2CC3(CC3)CN2C1=O] |
| 22 | 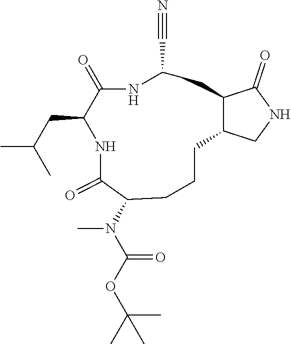 [SMILES: CC(C)C[C@@H]1NC(=O)[C@@H](N(C)C(=O)OC(C)(C)C)CCC[C@H]2CNC(=O)[C@@H]2C[C@@H](C#N)NC1=O] | 106 | 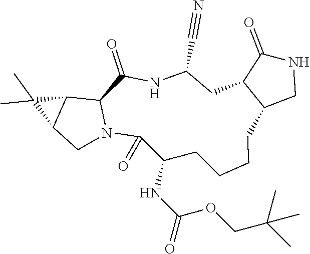 [SMILES: CC(C)(C)COC(=O)N[C@H]1CCCC[C@H]2CNC(=O)[C@H]2C[C@@H](C#N)NC(=O)[C@@H]2[C@@H]3[C@H](CN2C1=O)C3(C)C] |
| 23 | 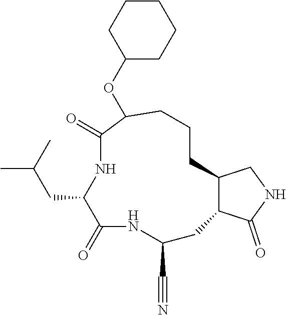 [SMILES: CC(C)C[C@@H]1NC(=O)C(OC2CCCCC2)CCC[C@H]2CNC(=O)[C@@H]2C[C@@H](C#N)NC1=O] | 107 | 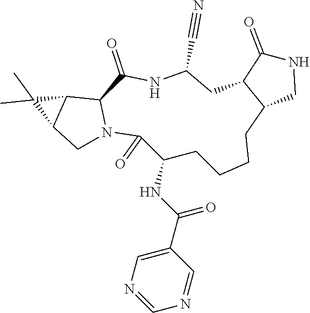 [SMILES: CC1(C)[C@@H]2[C@H]3C(=O)N[C@H](C#N)C[C@@H]4C(=O)NC[C@@H]4CCCC[C@H](NC(=O)c4cncnc4)C(=O)N3C[C@@H]21] |
| 24 | 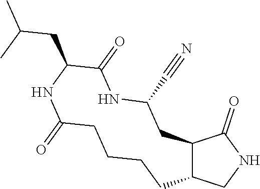 [SMILES: CC(C)C[C@@H]1NC(=O)CCCC[C@H]2CNC(=O)[C@@H]2C[C@@H](C#N)NC1=O] | 108 | 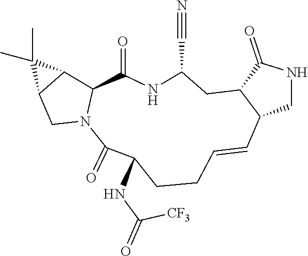 [SMILES: CC1(C)[C@@H]2[C@H]3C(=O)N[C@H](C#N)C[C@@H]4C(=O)NC[C@@H]4/C=C/CC[C@H](NC(=O)C(F)(F)F)C(=O)N3C[C@@H]21] |
| 25 | 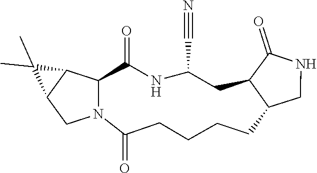 [SMILES: CC1(C)[C@@H]2[C@H]3C(=O)N[C@H](C#N)C[C@H]4C(=O)NC[C@@H]4CCCCC(=O)N3C[C@@H]21] | 109 | 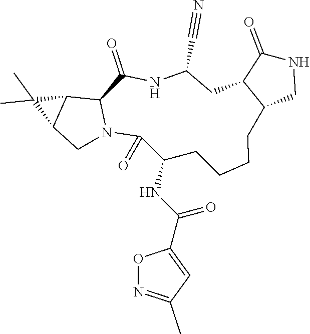 [SMILES: Cc1cc(C(=O)N[C@H]2CCCC[C@H]3CNC(=O)[C@H]3C[C@@H](C#N)NC(=O)[C@@H]3[C@@H]4[C@H](CN3C2=O)C4(C)C)on1] |
| 26 | 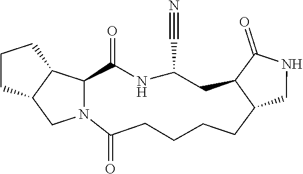 [SMILES: N#C[C@@H]1C[C@H]2C(=O)NC[C@@H]2CCCCC(=O)N2C[C@@H]3CCC[C@@H]3[C@H]2C(=O)N1] | 110 | 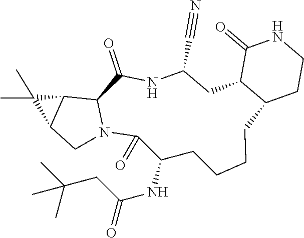 [SMILES: CC(C)(C)CC(=O)N[C@H]1CCCC[C@H]2CCNC(=O)[C@H]2C[C@@H](C#N)NC(=O)[C@@H]2[C@@H]3[C@H](CN2C1=O)C3(C)C] |
| 27 | 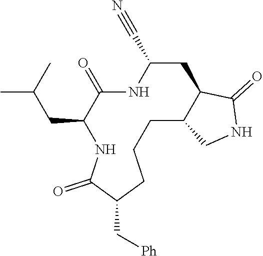 [SMILES: CC(C)C[C@@H]1NC(=O)[C@@H](Cc2ccccc2)CCC[C@H]2CNC(=O)[C@@H]2C[C@@H](C#N)NC1=O] | 111 | 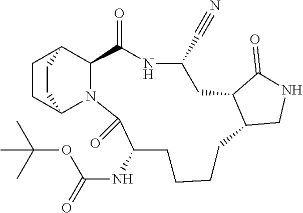 [SMILES: CC(C)(C)OC(=O)N[C@H]1CCCC[C@H]2CNC(=O)[C@H]2C[C@@H](C#N)NC(=O)[C@@H]2[C@H]3CC[C@H](CC3)N2C1=O] |
| 28 | 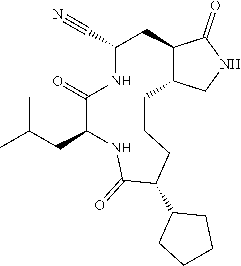 [SMILES: CC(C)C[C@@H]1NC(=O)[C@@H](C2CCCC2)CCC[C@H]2CNC(=O)[C@@H]2C[C@@H](C#N)NC1=O] | 112 | 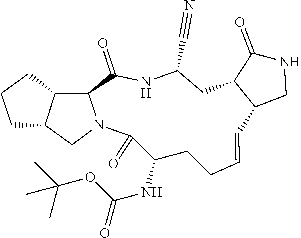 [SMILES: CC(C)(C)OC(=O)N[C@H]1CC/C=C/[C@H]2CNC(=O)[C@H]2C[C@@H](C#N)NC(=O)[C@@H]2[C@H]3CCC[C@H]3CN2C1=O] |
| 29 | 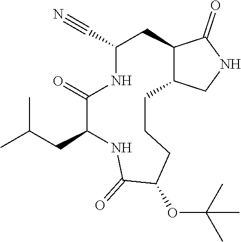 [SMILES: CC(C)C[C@@H]1NC(=O)[C@@H](OC(C)(C)C)CCC[C@H]2CNC(=O)[C@@H]2C[C@@H](C#N)NC1=O] | 113 | 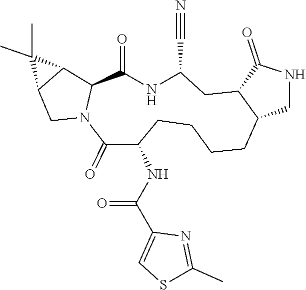 [SMILES: Cc1nc(C(=O)N[C@H]2CCCC[C@H]3CNC(=O)[C@H]3C[C@@H](C#N)NC(=O)[C@@H]3[C@@H]4[C@H](CN3C2=O)C4(C)C)cs1] |
| 30 | 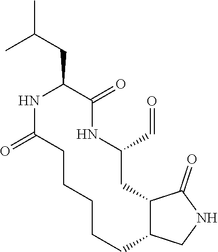 [SMILES: CC(C)C[C@@H]1NC(=O)CCCCC[C@H]2CNC(=O)[C@H]2C[C@@H](C=O)NC1=O] | 114 | 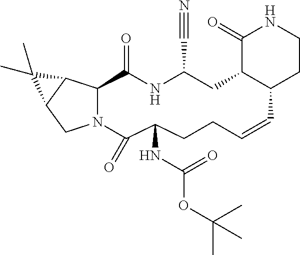 [SMILES: CC(C)(C)OC(=O)N[C@H]1CC/C=C\[C@H]2CCNC(=O)[C@H]2C[C@@H](C#N)NC(=O)[C@@H]2[C@@H]3[C@H](CN2C1=O)C3(C)C] |
| 31 | 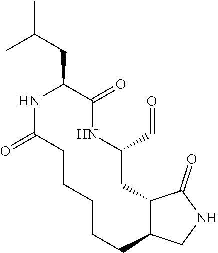 [SMILES: CC(C)C[C@@H]1NC(=O)CCCCC[C@@H]2CNC(=O)[C@H]2C[C@@H](C=O)NC1=O] | 115 | 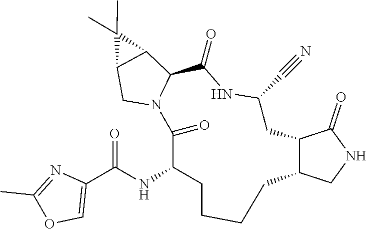 [SMILES: Cc1nc(C(=O)N[C@H]2CCCC[C@H]3CNC(=O)[C@H]3C[C@@H](C#N)NC(=O)[C@@H]3[C@@H]4[C@H](CN3C2=O)C4(C)C)co1] |
| 32 | 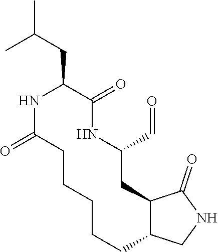 [SMILES: CC(C)C[C@@H]1NC(=O)CCCCC[C@H]2CNC(=O)[C@@H]2C[C@@H](C=O)NC1=O] | 116 | 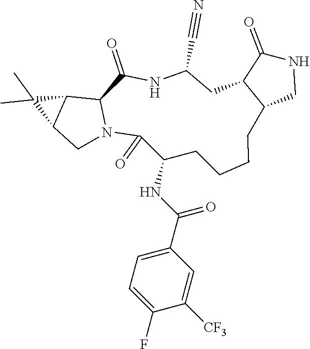 [SMILES: CC1(C)[C@@H]2[C@H]3C(=O)N[C@H](C#N)C[C@@H]4C(=O)NC[C@@H]4CCCC[C@H](NC(=O)c4ccc(F)c(C(F)(F)F)c4)C(=O)N3C[C@@H]21] |
| 33 | 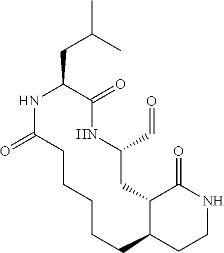 [SMILES: CC(C)C[C@@H]1NC(=O)CCCCC[C@@H]2CCNC(=O)[C@H]2C[C@@H](C=O)NC1=O] | 117 | 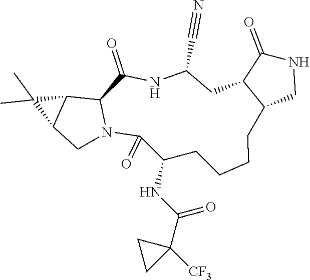 [SMILES: CC1(C)[C@@H]2[C@H]3C(=O)N[C@H](C#N)C[C@@H]4C(=O)NC[C@@H]4CCCC[C@H](NC(=O)C4(C(F)(F)F)CC4)C(=O)N3C[C@@H]21] |
| 34 | 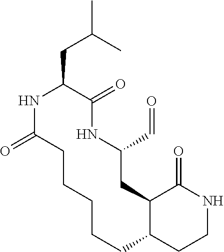 [SMILES: CC(C)C[C@@H]1NC(=O)CCCCC[C@H]2CCNC(=O)[C@@H]2C[C@@H](C=O)NC1=O] | 118 | 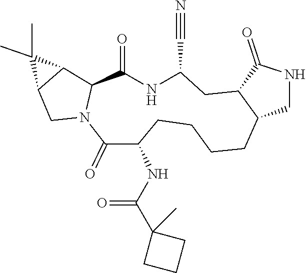 [SMILES: CC1(C(=O)N[C@H]2CCCC[C@H]3CNC(=O)[C@H]3C[C@@H](C#N)NC(=O)[C@@H]3[C@@H]4[C@H](CN3C2=O)C4(C)C)CCC1] |
| 35 | 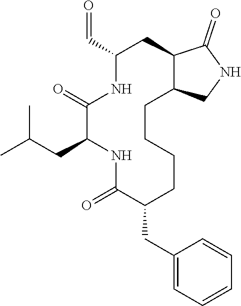 [SMILES: CC(C)C[C@@H]1NC(=O)[C@@H](Cc2ccccc2)CCCC[C@@H]2CNC(=O)[C@@H]2C[C@@H](C=O)NC1=O] | 119 | 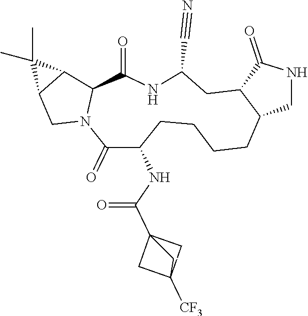 [SMILES: CC1(C)[C@@H]2[C@H]3C(=O)N[C@H](C#N)C[C@@H]4C(=O)NC[C@@H]4CCCC[C@H](NC(=O)C45CC(C(F)(F)F)(C4)C5)C(=O)N3C[C@@H]21] |
| 36 | 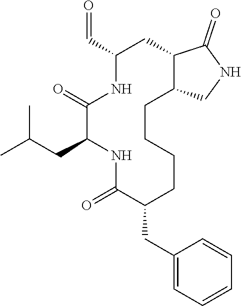 [SMILES: CC(C)C[C@@H]1NC(=O)[C@@H](Cc2ccccc2)CCCC[C@H]2CNC(=O)[C@H]2C[C@@H](C=O)NC1=O] | 120 | 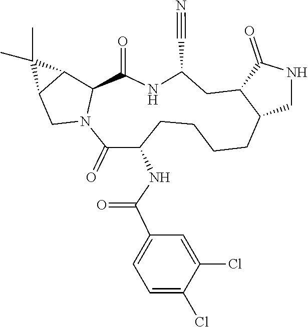 [SMILES: CC1(C)[C@@H]2[C@H]3C(=O)N[C@H](C#N)C[C@@H]4C(=O)NC[C@@H]4CCCC[C@H](NC(=O)c4ccc(Cl)c(Cl)c4)C(=O)N3C[C@@H]21] |
| 37 | 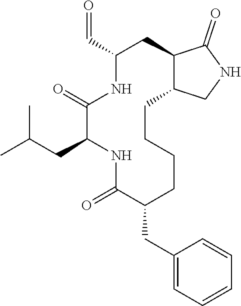 [SMILES: CC(C)C[C@@H]1NC(=O)[C@@H](Cc2ccccc2)CCCC[C@H]2CNC(=O)[C@@H]2C[C@@H](C=O)NC1=O] | 121 | 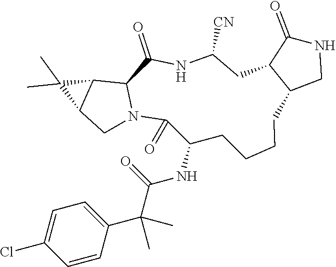 [SMILES: CC(C)(C(=O)N[C@H]1CCCC[C@H]2CNC(=O)[C@H]2C[C@@H](C#N)NC(=O)[C@@H]2[C@@H]3[C@H](CN2C1=O)C3(C)C)c1ccc(Cl)cc1] |
| 38 | 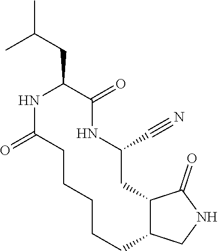 [SMILES: CC(C)C[C@@H]1NC(=O)CCCCC[C@H]2CNC(=O)[C@H]2C[C@@H](C#N)NC1=O] | 122 | 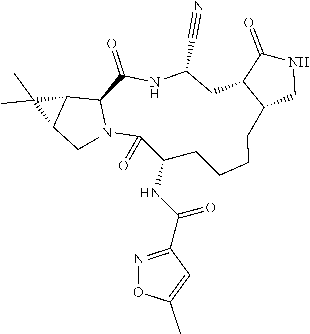 [SMILES: Cc1cc(C(=O)N[C@H]2CCCC[C@H]3CNC(=O)[C@H]3C[C@@H](C#N)NC(=O)[C@@H]3[C@@H]4[C@H](CN3C2=O)C4(C)C)no1] |
| 39 | 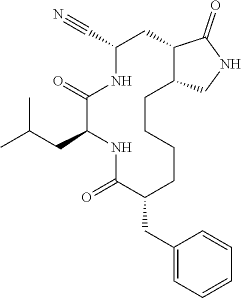 [SMILES: CC(C)C[C@@H]1NC(=O)[C@@H](Cc2ccccc2)CCCC[C@H]2CNC(=O)[C@H]2C[C@@H](C#N)NC1=O] | 123 | 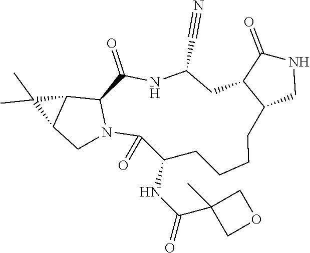 [SMILES: CC1(C(=O)N[C@H]2CCCC[C@H]3CNC(=O)[C@H]3C[C@@H](C#N)NC(=O)[C@@H]3[C@@H]4[C@H](CN3C2=O)C4(C)C)COC1] |
| 40 | 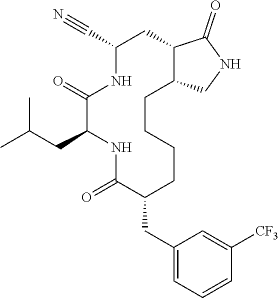 [SMILES: CC(C)C[C@@H]1NC(=O)[C@@H](Cc2cccc(C(F)(F)F)c2)CCCC[C@H]2CNC(=O)[C@H]2C[C@@H](C#N)NC1=O] | 124 | 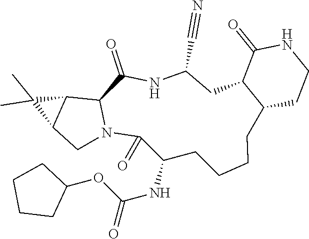 [SMILES: CC1(C)[C@@H]2[C@H]3C(=O)N[C@H](C#N)C[C@@H]4C(=O)NCC[C@@H]4CCCC[C@H](NC(=O)OC4CCCC4)C(=O)N3C[C@@H]21] |
| 41 | 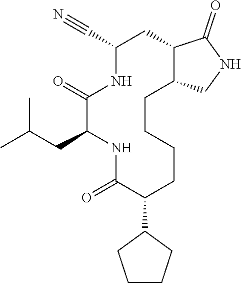 [SMILES: CC(C)C[C@@H]1NC(=O)[C@@H](C2CCCC2)CCCC[C@H]2CNC(=O)[C@H]2C[C@@H](C#N)NC1=O] | 125 | 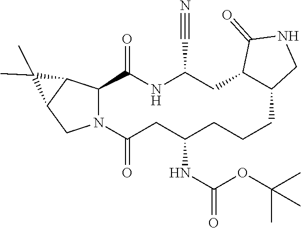 [SMILES: CC(C)(C)OC(=O)N[C@H]1CCC[C@H]2CNC(=O)[C@H]2C[C@@H](C#N)NC(=O)[C@@H]2[C@@H]3[C@H](CN2C(=O)C1)C3(C)C] |
| 42 | 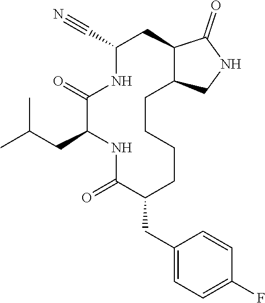 [SMILES: CC(C)C[C@@H]1NC(=O)[C@@H](Cc2ccc(F)cc2)CCCC[C@@H]2CNC(=O)[C@@H]2C[C@@H](C#N)NC1=O] | 126 | 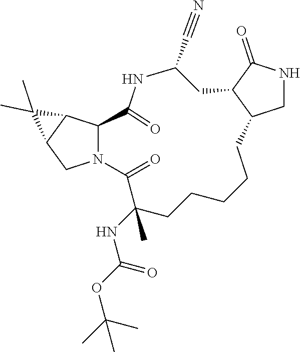 [SMILES: CC(C)(C)OC(=O)N[C@@]1(C)CCCCC[C@H]2CNC(=O)[C@H]2C[C@@H](C#N)NC(=O)[C@@H]2[C@@H]3[C@H](CN2C1=O)C3(C)C] |
| 43 | 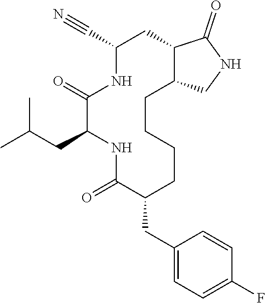 [SMILES: CC(C)C[C@@H]1NC(=O)[C@@H](Cc2ccc(F)cc2)CCCC[C@H]2CNC(=O)[C@H]2C[C@@H](C#N)NC1=O] | 127 | 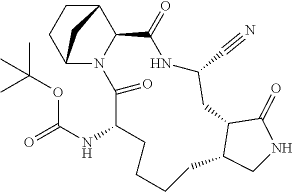 [SMILES: CC(C)(C)OC(=O)N[C@H]1CCCC[C@H]2CNC(=O)[C@H]2C[C@@H](C#N)NC(=O)[C@@H]2[C@H]3CC[C@H](C3)N2C1=O] |
| 44 | 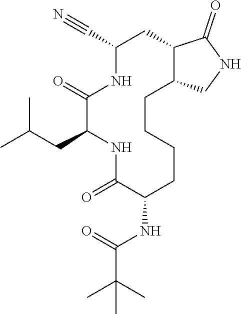 [SMILES: CC(C)C[C@@H]1NC(=O)[C@@H](NC(=O)C(C)(C)C)CCCC[C@H]2CNC(=O)[C@H]2C[C@@H](C#N)NC1=O] | 128 | 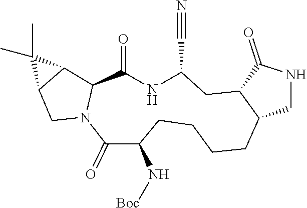 [SMILES: CC(C)(C)OC(=O)N[C@@H]1CCCC[C@H]2CNC(=O)[C@H]2C[C@@H](C#N)NC(=O)[C@@H]2[C@@H]3[C@H](CN2C1=O)C3(C)C] |
| 45 | 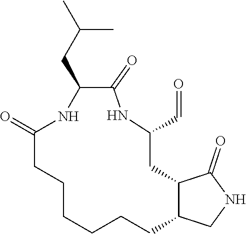 [SMILES: CC(C)C[C@@H]1NC(=O)CCCCCC[C@H]2CNC(=O)[C@H]2C[C@@H](C=O)NC1=O] | 129 | 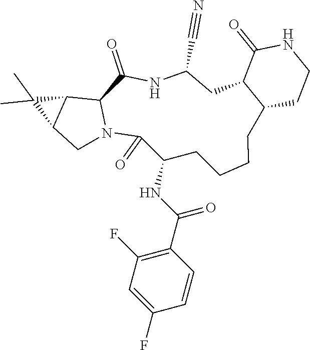 [SMILES: CC1(C)[C@@H]2[C@H]3C(=O)N[C@H](C#N)C[C@@H]4C(=O)NCC[C@@H]4CCCC[C@H](NC(=O)c4ccc(F)cc4F)C(=O)N3C[C@@H]21] |
| 46 | 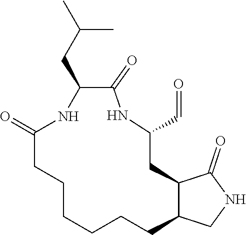 [SMILES: CC(C)C[C@@H]1NC(=O)CCCCCC[C@@H]2CNC(=O)[C@@H]2C[C@@H](C=O)NC1=O] | 130 | 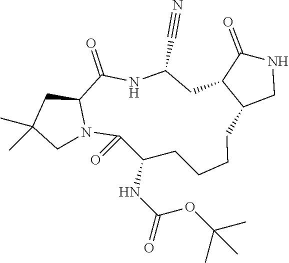 [SMILES: CC1(C)C[C@H]2C(=O)N[C@H](C#N)C[C@@H]3C(=O)NC[C@@H]3CCCC[C@H](NC(=O)OC(C)(C)C)C(=O)N2C1] |
| 47 | 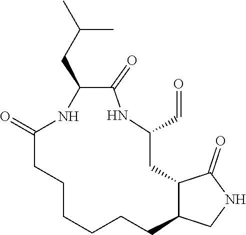 [SMILES: CC(C)C[C@@H]1NC(=O)CCCCCC[C@@H]2CNC(=O)[C@H]2C[C@@H](C=O)NC1=O] | 131 | 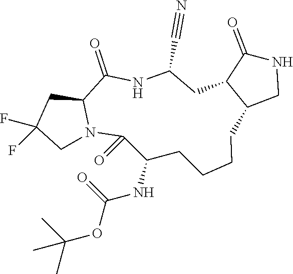 [SMILES: CC(C)(C)OC(=O)N[C@H]1CCCC[C@H]2CNC(=O)[C@H]2C[C@@H](C#N)NC(=O)[C@@H]2CC(F)(F)CN2C1=O] |
| 48 | 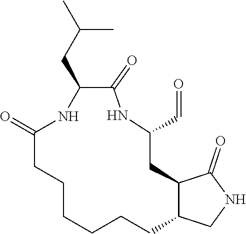 [SMILES: CC(C)C[C@@H]1NC(=O)CCCCCC[C@H]2CNC(=O)[C@@H]2C[C@@H](C=O)NC1=O] | 132 | 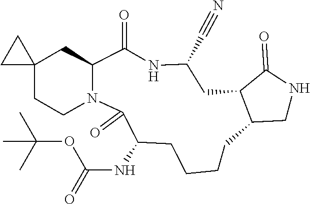 [SMILES: CC(C)(C)OC(=O)N[C@H]1CCCC[C@H]2CNC(=O)[C@H]2C[C@@H](C#N)NC(=O)[C@@H]2CC3(CCN2C1=O)CC3] |
| 49 | 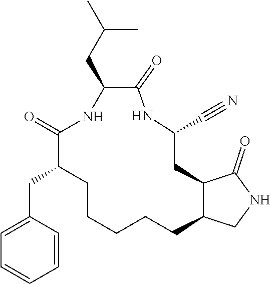 [SMILES: CC(C)C[C@@H]1NC(=O)[C@@H](Cc2ccccc2)CCCCC[C@@H]2CNC(=O)[C@@H]2C[C@@H](C#N)NC1=O] | 133 | 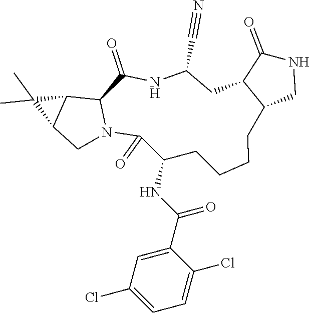 [SMILES: CC1(C)[C@@H]2[C@H]3C(=O)N[C@H](C#N)C[C@@H]4C(=O)NC[C@@H]4CCCC[C@H](NC(=O)c4cc(Cl)ccc4Cl)C(=O)N3C[C@@H]21] |
| 50 | 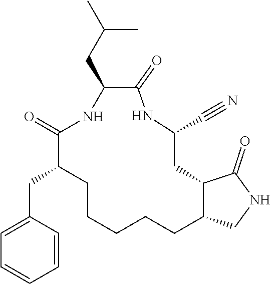 [SMILES: CC(C)C[C@@H]1NC(=O)[C@@H](Cc2ccccc2)CCCCC[C@H]2CNC(=O)[C@H]2C[C@@H](C#N)NC1=O] | 134 | 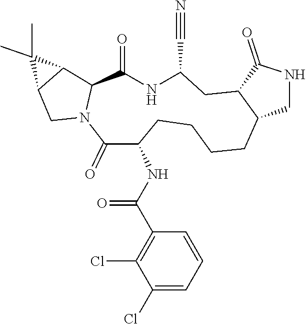 [SMILES: CC1(C)[C@@H]2[C@H]3C(=O)N[C@H](C#N)C[C@@H]4C(=O)NC[C@@H]4CCCC[C@H](NC(=O)c4cccc(Cl)c4Cl)C(=O)N3C[C@@H]21] |
| 51 | 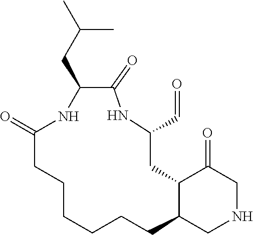 [SMILES: CC(C)C[C@@H]1NC(=O)CCCCCC[C@@H]2CNCC(=O)[C@H]2C[C@@H](C=O)NC1=O] | 135 | 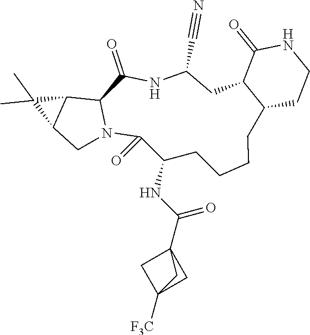 [SMILES: CC1(C)[C@@H]2[C@H]3C(=O)N[C@H](C#N)C[C@@H]4C(=O)NCC[C@@H]4CCCC[C@H](NC(=O)C45CC(C(F)(F)F)(C4)C5)C(=O)N3C[C@@H]21] |
| 52 | 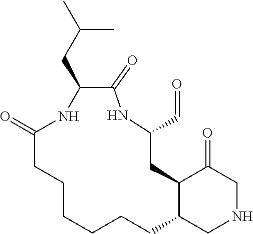 [SMILES: CC(C)C[C@@H]1NC(=O)CCCCCC[C@H]2CNCC(=O)[C@@H]2C[C@@H](C=O)NC1=O] | 136 | 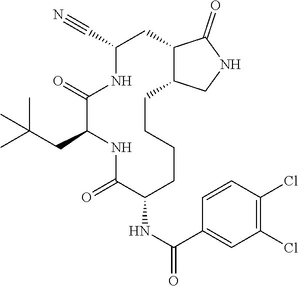 [SMILES: CC(C)(C)C[C@@H]1NC(=O)[C@@H](NC(=O)c2ccc(Cl)c(Cl)c2)CCCC[C@H]2CNC(=O)[C@H]2C[C@@H](C#N)NC1=O] |
| 53 | 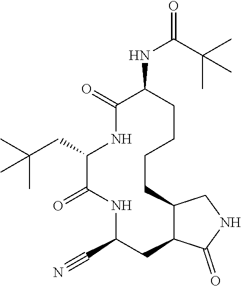 [SMILES: CC(C)(C)C[C@@H]1NC(=O)[C@@H](NC(=O)C(C)(C)C)CCCC[C@H]2CNC(=O)[C@H]2C[C@@H](C#N)NC1=O] | 137 | 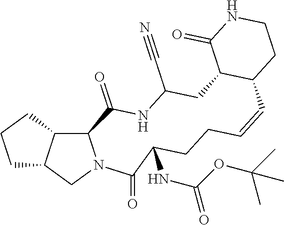 [SMILES: CC(C)(C)OC(=O)N[C@H]1CC/C=C\[C@H]2CCNC(=O)[C@H]2CC(C#N)NC(=O)[C@@H]2[C@H]3CCC[C@H]3CN2C1=O] |
| 54 | 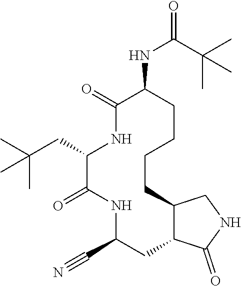 [SMILES: CC(C)(C)C[C@@H]1NC(=O)[C@@H](NC(=O)C(C)(C)C)CCCC[C@H]2CNC(=O)[C@@H]2C[C@@H](C#N)NC1=O] | 138 | 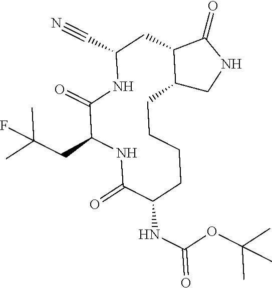 [SMILES: CC(C)(F)C[C@@H]1NC(=O)[C@@H](NC(=O)OC(C)(C)C)CCCC[C@H]2CNC(=O)[C@H]2C[C@@H](C#N)NC1=O] |
| 55 | 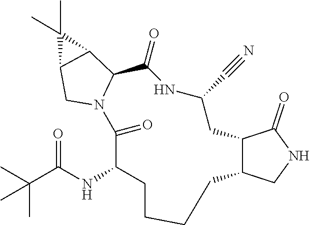 [SMILES: CC(C)(C)C(=O)N[C@H]1CCCC[C@H]2CNC(=O)[C@H]2C[C@@H](C#N)NC(=O)[C@@H]2[C@@H]3[C@H](CN2C1=O)C3(C)C] | 139 | 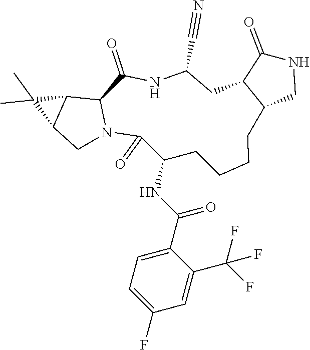 [SMILES: CC1(C)[C@@H]2[C@H]3C(=O)N[C@H](C#N)C[C@@H]4C(=O)NC[C@@H]4CCCC[C@H](NC(=O)c4ccc(F)cc4C(F)(F)F)C(=O)N3C[C@@H]21] |
| 56 | 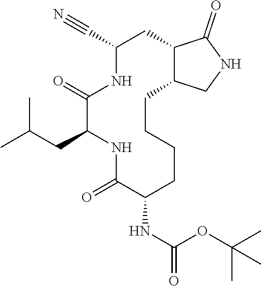 [SMILES: CC(C)C[C@@H]1NC(=O)[C@@H](NC(=O)OC(C)(C)C)CCCC[C@H]2CNC(=O)[C@H]2C[C@@H](C#N)NC1=O] | 140 | 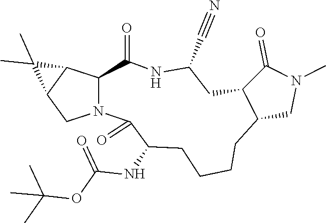 |
| 57 | 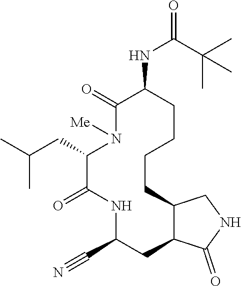 [SMILES: CC(C)C[C@H]1C(=O)N[C@H](C#N)C[C@@H]2C(=O)NC[C@@H]2CCCC[C@H](NC(=O)C(C)(C)C)C(=O)N1C] | 141 | 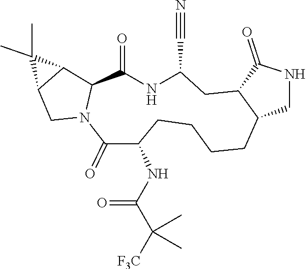 [SMILES: CC1(C)[C@@H]2[C@H]3C(=O)N[C@H](C#N)C[C@@H]4C(=O)NC[C@@H]4CCCC[C@H](NC(=O)C(C)(C)C(F)(F)F)C(=O)N3C[C@@H]21] |
| 58 | 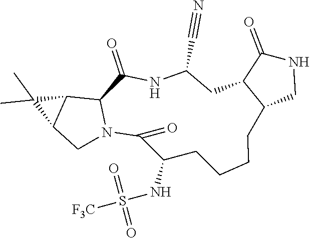 [SMILES: CC1(C)[C@@H]2[C@H]3C(=O)N[C@H](C#N)C[C@@H]4C(=O)NC[C@@H]4CCCC[C@H](NS(=O)(=O)C(F)(F)F)C(=O)N3C[C@@H]21] | 142 | 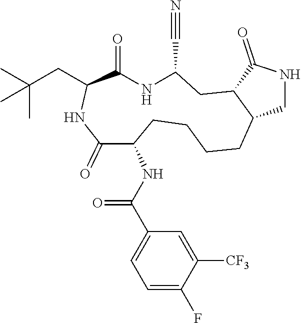 [SMILES: CC(C)(C)C[C@@H]1NC(=O)[C@@H](NC(=O)c2ccc(F)c(C(F)(F)F)c2)CCCC[C@H]2CNC(=O)[C@H]2C[C@@H](C#N)NC1=O] |
| 59 | 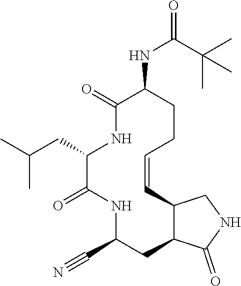 [SMILES: CC(C)C[C@@H]1NC(=O)[C@@H](NC(=O)C(C)(C)C)CC/C=C\[C@H]2CNC(=O)[C@H]2C[C@@H](C#N)NC1=O] | 143 | 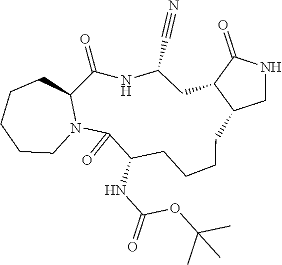 [SMILES: CC(C)(C)OC(=O)N[C@H]1CCCC[C@H]2CNC(=O)[C@H]2C[C@@H](C#N)NC(=O)[C@@H]2CCCCCN2C1=O] |
| 60 | 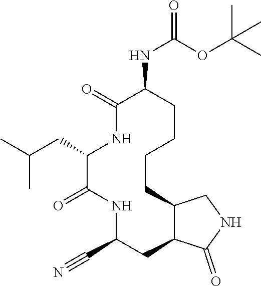 [SMILES: CC(C)C[C@@H]1NC(=O)[C@@H](NC(=O)OC(C)(C)C)CCCC[C@H]2CNC(=O)[C@H]2C[C@@H](C#N)NC1=O] | 144 | 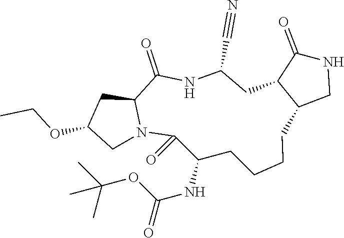 [SMILES: CCO[C@@H]1C[C@H]2C(=O)N[C@H](C#N)C[C@@H]3C(=O)NC[C@@H]3CCCC[C@H](NC(=O)OC(C)(C)C)C(=O)N2C1] |
| 61 | 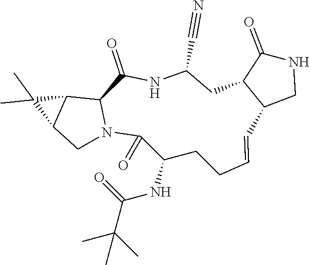 [SMILES: CC(C)(C)C(=O)N[C@H]1CC/C=C/[C@H]2CNC(=O)[C@H]2C[C@@H](C#N)NC(=O)[C@@H]2[C@@H]3[C@H](CN2C1=O)C3(C)C] | 145 | 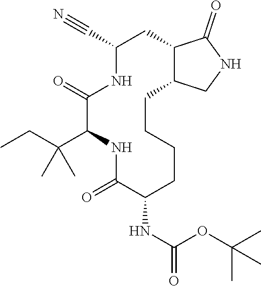 [SMILES: CCC(C)(C)[C@@H]1NC(=O)[C@@H](NC(=O)OC(C)(C)C)CCCC[C@H]2CNC(=O)[C@H]2C[C@@H](C#N)NC1=O] |
| 62 | 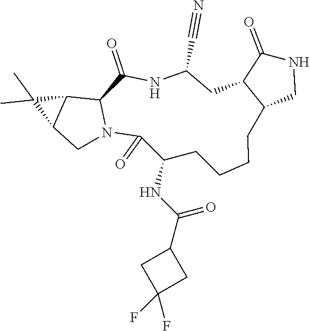 [SMILES: CC1(C)[C@@H]2[C@H]3C(=O)N[C@H](C#N)C[C@@H]4C(=O)NC[C@@H]4CCCC[C@H](NC(=O)C4CC(F)(F)C4)C(=O)N3C[C@@H]21] | 146 | 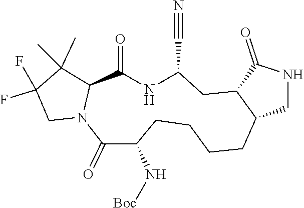 [SMILES: CC(C)(C)OC(=O)N[C@H]1CCCC[C@H]2CNC(=O)[C@H]2C[C@@H](C#N)NC(=O)[C@H]2N(CC(F)(F)C2(C)C)C1=O] |
| 63 | 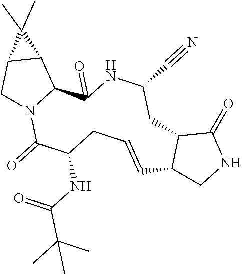 [SMILES: CC(C)(C)C(=O)N[C@H]1C/C=C/[C@H]2CNC(=O)[C@H]2C[C@@H](C#N)NC(=O)[C@@H]2[C@@H]3[C@H](CN2C1=O)C3(C)C] | 147 | 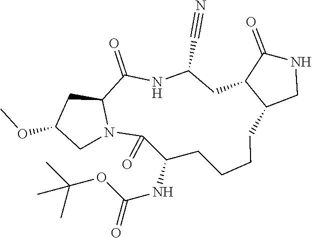 [SMILES: CO[C@@H]1C[C@H]2C(=O)N[C@H](C#N)C[C@@H]3C(=O)NC[C@@H]3CCCC[C@H](NC(=O)OC(C)(C)C)C(=O)N2C1] |
| 64 | 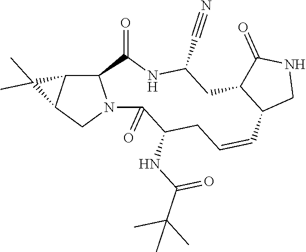 [SMILES: CC(C)(C)C(=O)N[C@H]1C/C=C\[C@H]2CNC(=O)[C@H]2C[C@@H](C#N)NC(=O)[C@@H]2[C@@H]3[C@H](CN2C1=O)C3(C)C] | 148 | 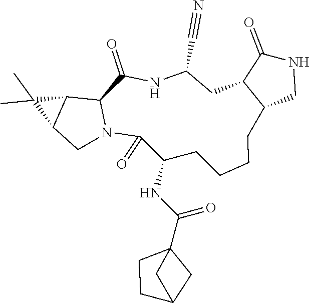 [SMILES: CC1(C)[C@@H]2[C@H]3C(=O)N[C@H](C#N)C[C@@H]4C(=O)NC[C@@H]4CCCC[C@H](NC(=O)C45CCC(C4)C5)C(=O)N3C[C@@H]21] |
| 65 | 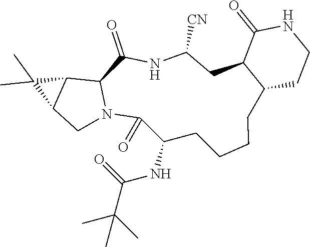 [SMILES: CC(C)(C)C(=O)N[C@H]1CCCC[C@H]2CCNC(=O)[C@@H]2C[C@@H](C#N)NC(=O)[C@@H]2[C@@H]3[C@H](CN2C1=O)C3(C)C] | 149 | 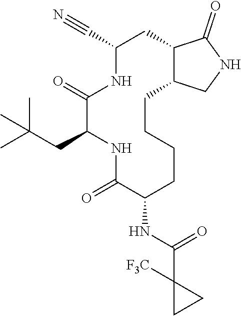 [SMILES: CC(C)(C)C[C@@H]1NC(=O)[C@@H](NC(=O)C2(C(F)(F)F)CC2)CCCC[C@H]2CNC(=O)[C@H]2C[C@@H](C#N)NC1=O] |
| 66 | 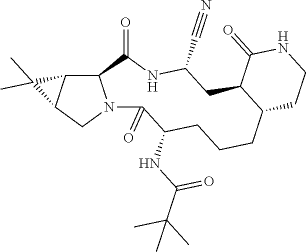 [SMILES: CC(C)(C)C(=O)N[C@H]1CCC[C@H]2CCNC(=O)[C@@H]2C[C@@H](C#N)NC(=O)[C@@H]2[C@@H]3[C@H](CN2C1=O)C3(C)C] | 150 | 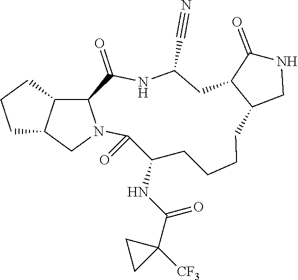 [SMILES: N#C[C@@H]1C[C@@H]2C(=O)NC[C@@H]2CCCC[C@H](NC(=O)C2(C(F)(F)F)CC2)C(=O)N2C[C@@H]3CCC[C@@H]3[C@H]2C(=O)N1] |
| 67 | 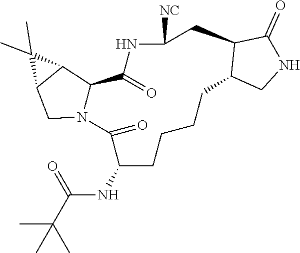 | 151 | 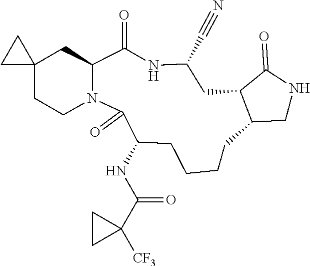 [SMILES: N#C[C@@H]1C[C@@H]2C(=O)NC[C@@H]2CCCC[C@H](NC(=O)C2(C(F)(F)F)CC2)C(=O)N2CCC3(CC3)C[C@H]2C(=O)N1] |
| 68 | 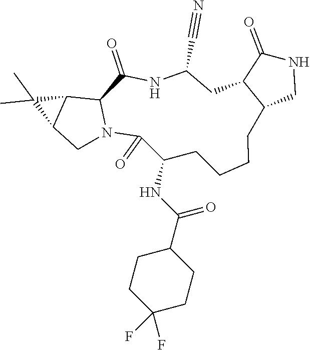 [SMILES: CC1(C)[C@@H]2[C@H]3C(=O)N[C@H](C#N)C[C@@H]4C(=O)NC[C@@H]4CCCC[C@H](NC(=O)C4CCC(F)(F)CC4)C(=O)N3C[C@@H]21] | 152 | 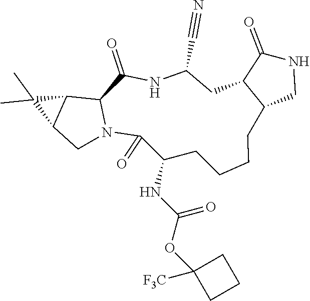 [SMILES: CC1(C)[C@@H]2[C@H]3C(=O)N[C@H](C#N)C[C@@H]4C(=O)NC[C@@H]4CCCC[C@H](NC(=O)OC4(C(F)(F)F)CCC4)C(=O)N3C[C@@H]21] |
| 69 | 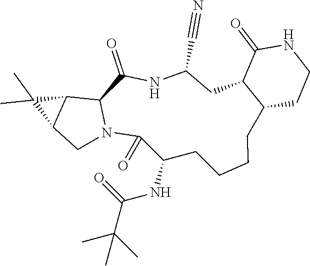 [SMILES: CC(C)(C)C(=O)N[C@H]1CCCC[C@H]2CCNC(=O)[C@H]2C[C@@H](C#N)NC(=O)[C@@H]2[C@@H]3[C@H](CN2C1=O)C3(C)C] | 153 | 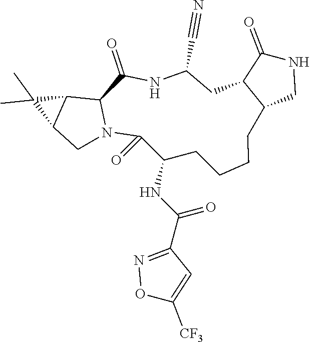 [SMILES: CC1(C)[C@@H]2[C@H]3C(=O)N[C@H](C#N)C[C@@H]4C(=O)NC[C@@H]4CCCC[C@H](NC(=O)c4cc(C(F)(F)F)on4)C(=O)N3C[C@@H]21] |
| 70 | 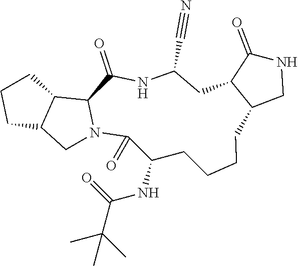 | 154 | 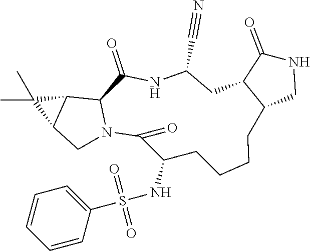 [SMILES: CC1(C)[C@@H]2[C@H]3C(=O)N[C@H](C#N)C[C@@H]4C(=O)NC[C@@H]4CCCC[C@H](NS(=O)(=O)c4ccccc4)C(=O)N3C[C@@H]21] |
| 71 | 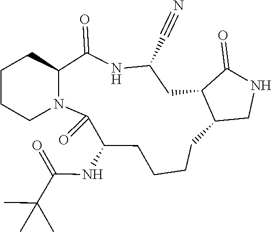 [SMILES: CC(C)(C)C(=O)N[C@H]1CCCC[C@H]2CNC(=O)[C@H]2C[C@@H](C#N)NC(=O)[C@@H]2CCCCN2C1=O] | 155 | 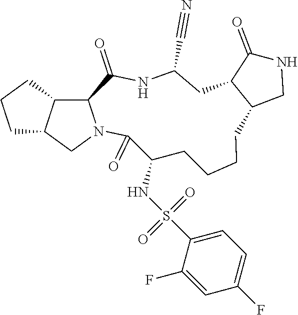 [SMILES: N#C[C@@H]1C[C@@H]2C(=O)NC[C@@H]2CCCC[C@H](NS(=O)(=O)c2ccc(F)cc2F)C(=O)N2C[C@@H]3CCC[C@@H]3[C@H]2C(=O)N1] |
| 72 | 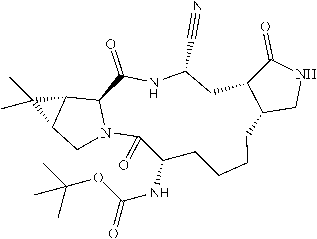 [SMILES: CC(C)(C)OC(=O)N[C@H]1CCCC[C@H]2CNC(=O)[C@H]2C[C@@H](C#N)NC(=O)[C@@H]2[C@@H]3[C@H](CN2C1=O)C3(C)C] | 156 | 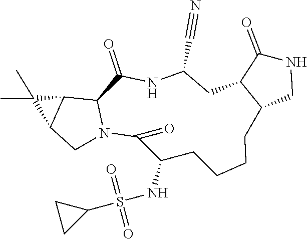 [SMILES: CC1(C)[C@@H]2[C@H]3C(=O)N[C@H](C#N)C[C@@H]4C(=O)NC[C@@H]4CCCC[C@H](NS(=O)(=O)C4CC4)C(=O)N3C[C@@H]21] |
| 73 | 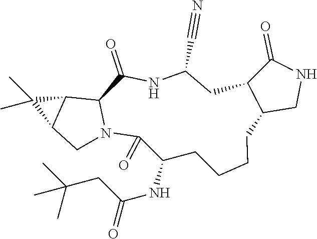 [SMILES: CC(C)(C)CC(=O)N[C@H]1CCCC[C@H]2CNC(=O)[C@H]2C[C@@H](C#N)NC(=O)[C@@H]2[C@@H]3[C@H](CN2C1=O)C3(C)C] | 157 | 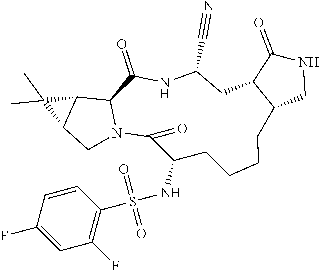 [SMILES: CC1(C)[C@@H]2[C@H]3C(=O)N[C@H](C#N)C[C@@H]4C(=O)NC[C@@H]4CCCC[C@H](NS(=O)(=O)c4ccc(F)cc4F)C(=O)N3C[C@@H]21] |
| 74 | 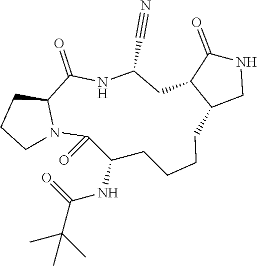 [SMILES: CC(C)(C)C(=O)N[C@H]1CCCC[C@H]2CNC(=O)[C@H]2C[C@@H](C#N)NC(=O)[C@@H]2CCCN2C1=O] | 158 | 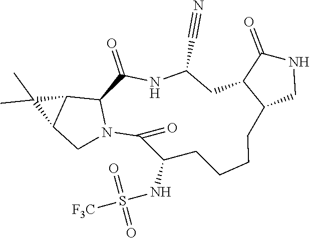 [SMILES: CC1(C)[C@@H]2[C@H]3C(=O)N[C@H](C#N)C[C@@H]4C(=O)NC[C@@H]4CCCC[C@H](NS(=O)(=O)C(F)(F)F)C(=O)N3C[C@@H]21] |
| 75 | 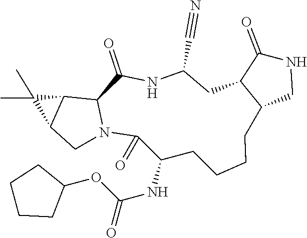 [SMILES: CC1(C)[C@@H]2[C@H]3C(=O)N[C@H](C#N)C[C@@H]4C(=O)NC[C@@H]4CCCC[C@H](NC(=O)OC4CCCC4)C(=O)N3C[C@@H]21] | 159 | 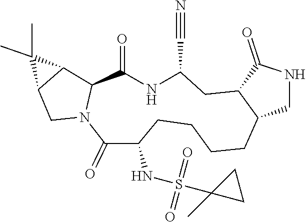 [SMILES: CC1(C)[C@@H]2[C@H]3C(=O)N[C@H](C#N)C[C@@H]4C(=O)NC[C@@H]4CCCC[C@H](NS(=O)(=O)C4(C)CC4)C(=O)N3C[C@@H]21] |
| 76 | 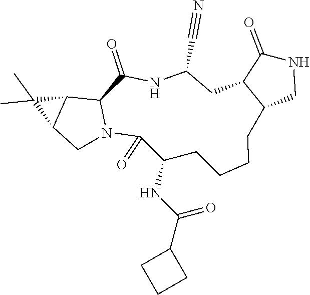 [SMILES: CC1(C)[C@@H]2[C@H]3C(=O)N[C@H](C#N)C[C@@H]4C(=O)NC[C@@H]4CCCC[C@H](NC(=O)C4CCC4)C(=O)N3C[C@@H]21] | 160 | 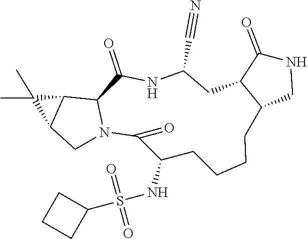 [SMILES: CC1(C)[C@@H]2[C@H]3C(=O)N[C@H](C#N)C[C@@H]4C(=O)NC[C@@H]4CCCC[C@H](NS(=O)(=O)C4CCC4)C(=O)N3C[C@@H]21] |
| 77 | 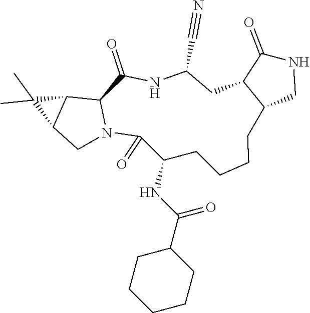 [SMILES: CC1(C)[C@@H]2[C@H]3C(=O)N[C@H](C#N)C[C@@H]4C(=O)NC[C@@H]4CCCC[C@H](NC(=O)C4CCCCC4)C(=O)N3C[C@@H]21] | 161 | 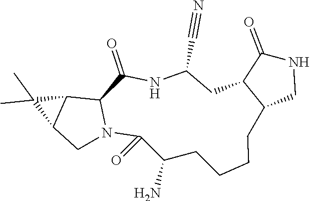 [SMILES: CC1(C)[C@@H]2[C@H]3C(=O)N[C@H](C#N)C[C@@H]4C(=O)NC[C@@H]4CCCC[C@H](N)C(=O)N3C[C@@H]21] |
| 78 | 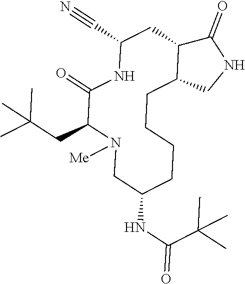 [SMILES: CN1C[C@@H](NC(=O)C(C)(C)C)CCCC[C@H]2CNC(=O)[C@H]2C[C@@H](C#N)NC(=O)[C@@H]1CC(C)(C)C] | 162 | 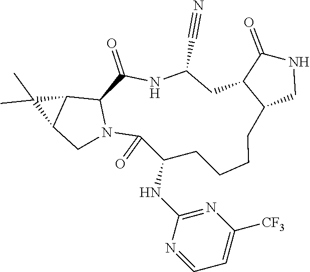 [SMILES: CC1(C)[C@@H]2[C@H]3C(=O)N[C@H](C#N)C[C@@H]4C(=O)NC[C@@H]4CCCC[C@H](Nc4nccc(C(F)(F)F)n4)C(=O)N3C[C@@H]21] |
| 79 | 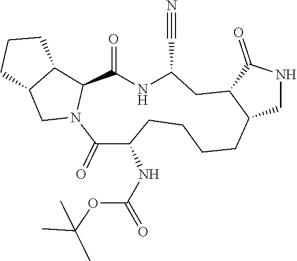 [SMILES: CC(C)(C)OC(=O)N[C@H]1CCCC[C@H]2CNC(=O)[C@H]2C[C@@H](C#N)NC(=O)[C@@H]2[C@H]3CCC[C@H]3CN2C1=O] | 163 | 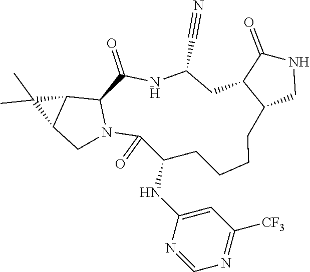 [SMILES: CC1(C)[C@@H]2[C@H]3C(=O)N[C@H](C#N)C[C@@H]4C(=O)NC[C@@H]4CCCC[C@H](Nc4cc(C(F)(F)F)ncn4)C(=O)N3C[C@@H]21] |
| 80 | 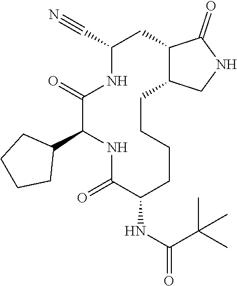 [SMILES: CC(C)(C)C(=O)N[C@H]1CCCC[C@H]2CNC(=O)[C@H]2C[C@@H](C#N)NC(=O)[C@H](C2CCCC2)NC1=O] | 164 | 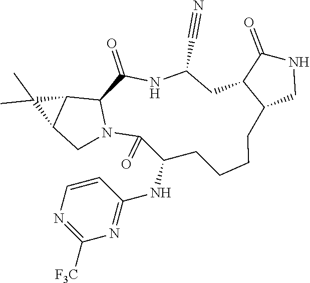 [SMILES: CC1(C)[C@@H]2[C@H]3C(=O)N[C@H](C#N)C[C@@H]4C(=O)NC[C@@H]4CCCC[C@H](Nc4ccnc(C(F)(F)F)n4)C(=O)N3C[C@@H]21] |
| 81 | 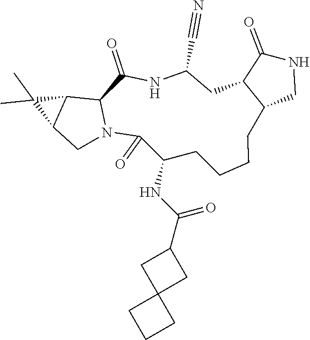 [SMILES: CC1(C)[C@@H]2[C@H]3C(=O)N[C@H](C#N)C[C@@H]4C(=O)NC[C@@H]4CCCC[C@H](NC(=O)C4CC5(CCC5)C4)C(=O)N3C[C@@H]21] | 165 | 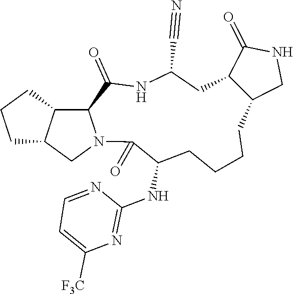 [SMILES: N#C[C@@H]1C[C@@H]2C(=O)NC[C@@H]2CCCC[C@H](Nc2nccc(C(F)(F)F)n2)C(=O)N2C[C@@H]3CCC[C@@H]3[C@H]2C(=O)N1] |
| 82 | 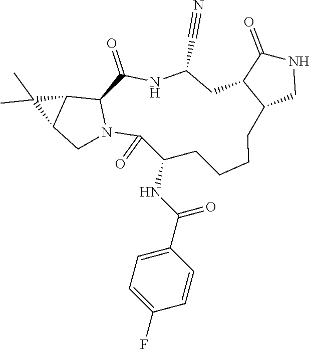 [SMILES: CC1(C)[C@@H]2[C@H]3C(=O)N[C@H](C#N)C[C@@H]4C(=O)NC[C@@H]4CCCC[C@H](NC(=O)c4ccc(F)cc4)C(=O)N3C[C@@H]21] | 166 | 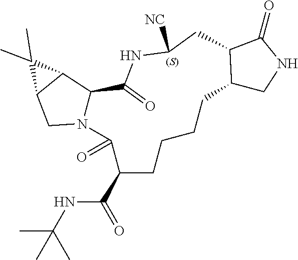 |
| 83 | 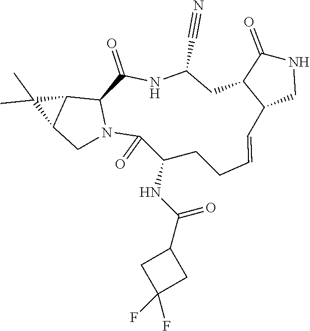 [SMILES: CC1(C)[C@@H]2[C@H]3C(=O)N[C@H](C#N)C[C@@H]4C(=O)NC[C@@H]4/C=C/CC[C@H](NC(=O)C4CC(F)(F)C4)C(=O)N3C[C@@H]21] | 167 | 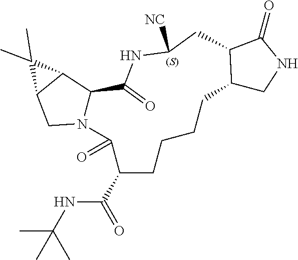 |
| 84 | 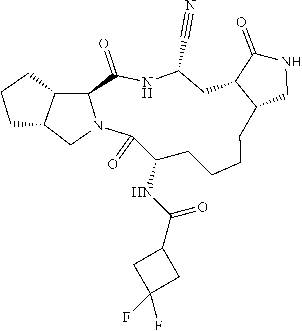 [SMILES: N#C[C@@H]1C[C@@H]2C(=O)NC[C@@H]2CCCC[C@H](NC(=O)C2CC(F)(F)C2)C(=O)N2C[C@@H]3CCC[C@@H]3[C@H]2C(=O)N1] | 168 | 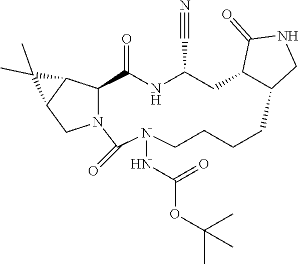 [SMILES: CC(C)(C)OC(=O)NN1CCCC[C@H]2CNC(=O)[C@H]2C[C@@H](C#N)NC(=O)[C@@H]2[C@@H]3[C@H](CN2C1=O)C3(C)C] |

16. A pharmaceutical composition comprising an effective amount of a compound of Formula (I) according to claim 1 or a pharmaceutically acceptable salt, solvate, stereoisomer or prodrug thereof, and optionally in combination with a pharmaceutically acceptable carrier, excipient or diluent.

17. A method of treating a coronavirus related disease or condition in a patient in need thereof, the method comprising administering to the patient a therapeutically effective amount of a compound of formula (I) according to claim 1 or a pharmaceutically acceptable salt, solvate, stereoisomer or prodrug thereof.

18-20. (canceled)

21. The method according to claim 17, wherein the coronavirus related disease or condition is caused by a virus selected from SARS-CoV-1, SARS-CoV-2, MERS-CoV, CoV-229E, and HCoV-OC43.

22. The method according to claim 17, wherein the compound of Formula (I) is characterised by a protease inhibition assay IC₅₀ value of less than 20 μM.

23. The method according to claim 17, wherein the compound of Formula (I) is characterised by a viral cytopathic effect assay EC₅₀ value of less than 20 μM.

24. A method of fabricating a compound of Formula (I) according to claim 1, comprising:
i) reacting a compound of Formula (II) with a compound of Formula (III) under dehydrating conditions in order to form an amide bond: 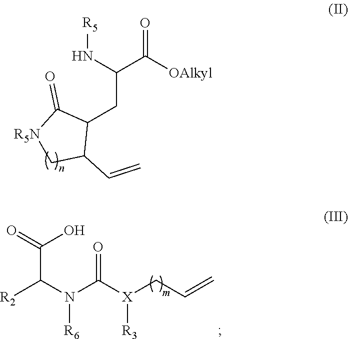
ii) performing a ring closing olefin metathesis reaction in order to form a compound of Formula (IV): 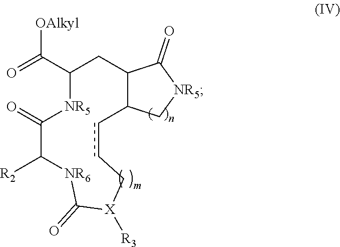
and
iii) converting the alkyloxyacyl moiety in Formula (IV) into a R₁ moiety in order to form a compound of Formula (I).

25. The method according to claim 24, wherein step ii) further comprises performing a hydrogenation reaction after the olefin metathesis reaction.
